(19) United States
(12) Patent Application Publication
(10) Pub. No.: US 2024/0024478 A1
(43) Pub. Date: Jan. 25, 2024

(54) Compositions and Methods for Reducing HLA-A in a Cell

(71) Applicant: Intellia Therapeutics, Inc., Cambridge, MA (US)

(72) Inventors: Surbhi Goel, Winchester, MA (US); Yong Zhang, Bedford, MA (US); Reynald Michael Lescarbeau, Medford, MA (US); Bradley Andrew Murray, Boston, MA (US); Srijani Sridhar, Cambridge, MA (US)

(73) Assignee: Intellia Therapeutics, Inc., Cambridge, MA (US)

(21) Appl. No.: 18/339,665

(22) Filed: Jun. 22, 2023

Related U.S. Application Data
(63) Continuation of application No. PCT/US2021/064930, filed on Dec. 22, 2021.
(60) Provisional application No. 63/288,492, filed on Dec. 10, 2021.
(60) Provisional application No. 63/254,970, filed on Oct. 12, 2021.
(60) Provisional application No. 63/250,996, filed on Sep. 30, 2021.
(60) Provisional application No. 63/130,095, filed on Dec. 23, 2020.

(51) Int. Cl. A61K 39/00; C12N 9/22; C12N 5/0783; C07K 14/74

(52) U.S. Cl. CPC A61K 39/4632; C12N 9/22; C12N 5/0636; C07K 14/70539; C12N 2510/00; C12N 2310/20; A61K 2239/15

(57) ABSTRACT

Compositions and methods for reducing HLA-A protein expression in a cell comprising genetically modifying HLA-A for use e.g., in adoptive cell transfer therapies.

This application is a Continuation of International Application No. PCT/US2021/064930, filed Dec. 22, 2021, which claims the benefit under 35 U.S.C. 119(e) of U.S. Provisional Application No. 63/130,095, filed Dec. 23, 2020, U.S. Provisional Application No. 63/250,996, filed Sep. 30, 2021, U.S. Provisional Application No. 63/254,970, filed Oct. 12, 2021, and U.S. Provisional Application No. 63/288,492, filed Dec. 10, 2021; each of which disclosures is herein incorporated by reference in its entirety.

This application is filed with a Sequence Listing in electronic format. The Sequence Listing is provided as a file entitled “01155-0036-00US.xml” created on Aug. 29, 2023, which is 995,679 bytes in size. The information in the electronic format of the sequence listing is incorporated herein by reference in its entirety.

### I. INTRODUCTION AND SUMMARY

The ability to downregulate MHC class I is critical for many in vivo and ex vivo utilities, e.g., when using allogeneic cells (originating from a donor) for transplantation and/or e.g., for creating a cell population in vitro that does not activate T cells. In particular, the transfer of allogeneic cells into a subject is of great interest to the field of cell therapy. The use of allogeneic cells has been limited due to the problem of rejection by the recipient subject's immune cells, which recognize the transplanted cells as foreign and mount an attack. To avoid the problem of immune rejection, cell-based therapies have focused on autologous approaches that use a subject's own cells as the cell source for therapy, an approach that is time-consuming and costly.

Typically, immune rejection of allogeneic cells results from a mismatching of major histocompatibility complex (MHC) molecules between the donor and recipient. Within the human population, MHC molecules exist in various forms, including e.g., numerous genetic variants of any given MHC gene, i.e., alleles, encoding different forms of MHC protein. The primary classes of MHC molecules are referred to as MHC class I and MHC class II. MHC class I molecules (e.g., HLA-A, HLA-B, and HLA-C in humans) are expressed on all nucleated cells and present antigens to activate cytotoxic T cells (CD8+ T cells or CTLs). MHC class II molecules (e.g., HLA-DP, HLA-DQ, and HLA-DR in humans) are expressed on only certain cell types (e.g., B cells, dendritic cells, and macrophages) and present antigens to activate helper T cells (CD4+ T cells or Th cells), which in turn provide signals to B cells to produce antibodies.

Slight differences, e.g., mismatches in MHC alleles between individuals can cause the T cells in a recipient to become activated. During T cell development, an individual's T cell repertoire is tolerized to one's own MHC molecules, but T cells that recognize another individual's MHC molecules may persist in circulation and are referred to as alloreactive T cells. Alloreactive T cells can become activated e.g., by the presence of another individual's cells expressing MHC molecules in the body, causing e.g., graft versus host disease and transplant rejection.

While fully matching HLA types between donor and recipient is theoretically possible as a means of reducing transplant rejection, such an approach is logistically and practically challenging given the diversity of HLA alleles across the population to fully match e.g., 10 out 10 alleles (i.e., 2 alleles for each of HLA-A, HLA-B, HLA-C, HLA-DRB1, and HLA-DQB1).

Methods and compositions for reducing the susceptibility of an allogeneic cell to rejection are of interest, including e.g., reducing the cell's expression of MHC protein to avoid recipient T cell responses. In practice, the ability to genetically modify an allogeneic cell for transplantation into a subject has been hampered by the requirement for multiple gene edits to reduce all MHC protein expression, while at the same time, avoiding other harmful recipient immune responses. For example, while strategies to deplete MHC class I protein may reduce activation of CTLs, cells that lack MHC class I on their surface are susceptible to lysis by natural killer (NK) cells of the immune system because NK cell activation is regulated by MHC class I-specific inhibitory receptors. Therefore, safely reducing or eliminating expression of MHC class I has proven challenging.

Gene editing strategies to deplete MHC class II molecules have also proven difficult particularly in certain cell types for reasons including low editing efficiencies and low cell survival rates, preventing practical application as a cell therapy.

Thus, there exists a need for improved methods and compositions for modifying allogeneic cells to overcome the problem of recipient immune rejection and the technical difficulties associated with the multiple genetic modifications required to produce a safer cell for transplant.

The present disclosure provides engineered human cells with reduced or eliminated surface expression of HLA-A relative to an unmodified cell, wherein the cell is homozygous for HLA-B and homozygous for HLA-C. The engineered human cells disclosed herein therefore provide a “partial matching” approach to the problem of allogeneic cell transfer and MHC class I compatibility. The use of cells that are homozygous for HLA-B and HLA-C, in addition to reducing or eliminating expression of HLA-A in the cells, limits the number of donors that are necessary to provide a therapy that covers a majority of recipients in population because the disclosed partial matching approach requires only one matching HLA-B allele (as opposed to two) and only one HLA-C allele (as opposed to two). Surprisingly, the engineered human cells that have reduced or eliminated surface expression of HLA-A relative to an unmodified cell, disclosed herein, demonstrate persistence and are protective against NK-mediated rejection, especially as compared to engineered cells with reduced or eliminated B2M expression. The disclosure provides methods and compositions for generating such engineered human cells with reduced or eliminated surface expression of HLA-A relative to an unmodified cell, wherein the cell is homozygous for HLA-B and homozygous for HLA-C. In some embodiments, the disclosure provides engineered human cells, and methods and compositions for generating engineered human cells, wherein the cell further has reduced expression of MHC class II protein on the surface of the cell, e.g., wherein the cell has a genetic modification in the CIITA gene. In some embodiments, the disclosure provides for further engineering of the cell, including to reduce or eliminate the expression of endogenous T cell receptor proteins (e.g., TRAC, TRBC), and to introduce an exogenous nucleic acid, e.g., encoding a polypeptide expressed on the cell surface or a polypeptide that is secreted by the cell. Thus, the disclosure thus provides a flexible platform for genetically engineering human cells for a variety of desired adoptive cell therapy purposes.

Provided herein is an engineered human cell, which has reduced or eliminated surface expression of HLA-A relative to an unmodified cell, comprising a genetic modification in the HLA-A gene, wherein the cell is homozygous for HLA-B and homozygous for HLA-C. Also provided is an engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942854-chr6:29942913 and chr6:29943518-chr6:29943619, wherein the cell is homozygous for HLA-B and homozygous for HLA-C.

Provided herein is an engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Provided herein is an engineered human cell, which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Provided herein is a method of making an engineered human cell, which has reduced or eliminated surface expression of HLA-A protein relative to an unmodified cell, wherein the cell is homozygous for HLA-B and homozygous for HLA-C, comprising contacting a cell with composition comprising: (a) an HLA-A guide RNA comprising (i) a guide sequence selected from SEQ ID NOs: 1-211; or (ii) at least 17, 18, 19, or 20 contiguous nucleotides of a sequence selected from SEQ ID NOs: 1-211; or (iii) a guide sequence at least 95%, 90%, or 85% identical to a sequence selected from SEQ ID NOs: 1-211; or (iv) a guide sequence that binds a target site comprising a genomic region listed in Tables 2-5; or (v) a guide sequence that is complementary to at least 17, 18, 19, or 20 contiguous nucleotides of a genomic region listed in Tables 1-2 and 5, or a guide sequence that is complementary to at least 17, 18, 19, 21, 22, 23, or 24 contiguous nucleotides of a genomic region listed in Table 4; or (vi) a guide sequence that is at least 95%, 90%, or 85% identical to a sequence selected from (v); and optionally (b) an RNA-guided DNA binding agent or a nucleic acid encoding an RNA-guided DNA binding agent.

Provided herein is a method of reducing surface expression of HLA-A protein in a human cell relative to an unmodified cell, comprising contacting a cell with composition comprising: (a) an HLA-A guide RNA comprising (i) a guide sequence selected from SEQ ID NOs: 1-211; or (ii) at least 17, 18, 19, or 20 contiguous nucleotides of a sequence selected from SEQ ID NOs: 1-211; or (iii) a guide sequence at least 95%, 90%, or 85% identical to a sequence selected from SEQ ID NOs: 1-211; or (iv) a guide sequence that binds a target site comprising a genomic region listed in Tables 2-5; or (v) a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or (vi) a guide sequence that is at least 95%, 90%, or 85% identical to a sequence selected from (v); and optionally (b) an RNA-guided DNA binding agent or a nucleic acid encoding an RNA-guided DNA binding agent.

Provided herein is a method of administering an engineered cell to a recipient subject in need thereof, the method comprising: (a) determining the HLA-B and HLA-C alleles of the recipient subject; (b) selecting an engineered cell or cell population of any one of the preceding embodiments, or engineered cell or cell population produced by the method of any one of the preceding embodiments, wherein the engineered cell comprises at least one of the same HLA-B or HLA-C alleles as the recipient subject; (c) administering the selected engineered cell to the recipient subject.

Further embodiments are provided throughout and described in the claims and Figures.

### II. BRIEF DESCRIPTION OF THE DRAWINGS

FIGS. 1A and 1B show the percentage of activated T cells negative for HLA-A2 by flow cytometry. FIG. 1A shows data for guides (G018997, G018998, G018999, G019000, G019008, G013006). FIG. 1B shows data for guides (G018091, G018933, G018935, G018954, G018995, G018996).

FIG. 2 shows resistance to NK-cell mediated killing of HLA-A knockout (HLA-B/C match) T cells versus B2M knockout T cells, optionally including an exogenous HLA-E construct, as percent T cell lysis. HLA-A knockout, HLA-A, CIITA double knockout, B2M knockout, B2M+HLA-E, and wild type cells are compared.

FIGS. 3A-F show results for sequential editing in CD8+ T cells. FIG. 3A shows the percentage of HLA-A positive cells. FIG. 3B shows the percentage of MHC class II positive cells. FIG. 3C shows the percentage of WT1 TCR positive CD3+, Vb8+ cells. FIG. 3D shows the percentage cells displaying mis-paired TCRs. FIG. 3E shows the percentage of CD3+, vb8− cells displaying only endogenous TCRs. FIG. 3F shows the percentage of CD3+, Vb8+, positive for the WT1 TCR and negative for HLA-A and MHC class II.

FIGS. 4A-F show results for sequential editing in CD4+ T cells. FIG. 4A shows the percentage of HLA-A positive cells. FIG. 4B shows the percentage of MHC class II positive cells. FIG. 4C shows the percentage of WT1 TCR positive CD3+, Vb8+ cells. FIG. 4D shows the percentage of cells displaying mis-paired TCRs. FIG. 4E shows the percentage of CD3+, vb8− cells displaying only endogenous TCRs. FIG. 4F shows the percentage of CD3+, Vb8+, positive for the WT1 TCR and negative for HLA-A and MHC class II.

FIGS. 5A-D show the percent indels following sequential editing of T cells for CIITA (FIG. 5A), HLA-A (FIG. 5B), TRBC1 (FIG. 5C), and TRBC2 (FIG. 5D) in T cells.

FIGS. 6A-B show luciferase expression from B2M, CIITA, HLA-A, or double (HLA-A, CIITA) knockout human T cells administered to mice inoculated with human natural killer cells. FIG. 6A shows radiance (photons/s/cm2/sr) from luciferase expressing T cells present at the various time points after injection. FIG. 6B shows radiance (photons/s/cm2/sr) from luciferase expressing T cells present in the various mice groups on Day 27.

FIGS. 7A-B show luciferase expression from B2M and AlloWT1 knockout human T cells administered to mice inoculated with human natural killer cells. FIG. 7A shows total flux (p/s) from luciferase expressing T cells present at the various time points after injection. FIG. 7B shows total flux (p/s) from luciferase expressing T cells present in the various mice groups after 31 days.

FIGS. 8A-B show the percent normalized proliferation of host CD4 (FIG. 8A) or host CD8 (FIG. 8B) T cells triggered by HLA class I+HLA class II double knockout or HLA-A and HLA class II double knockout engineered autologous or allogeneic T cells.

FIGS. 9A-F shows a panel of percent CD8+ (FIG. 9A), endogenous TCR+ (FIG. 9B), WT1 TCR+ (FIG. 9C), HLA-A2 knockout (FIG. 9D), HLA-DRDPDQ knockout (FIG. 9E), and % Allo WT1 (FIG. 9F).

FIG. 10 shows total flux (p/s) from luciferase expressing T cells present at the various time points after injection out to 18 days.

FIGS. 11A-11B respectively show release of IFN-γ and IL-2 in supernatants from a killing assay containing a co-culture of engineered T cells from the Allo-WT1, Auto-WT1, TCR KO, and Wildtype (WT) groups with target tumor cells.

FIGS. 12A-12B show CIITA, HLA-A, TRAC, and TRBC editing and WT1 TCR insertion rates in CD8+ T cells in three conditions. The percentage of cells expressing relevant cell surface proteins following sequential T cell engineering are shown in FIG. 12A for CD8+ T cells. The percent of T cells with all intended edits (insertion of the WT1-TCR, combined with knockout of HLA-A and CIITA) is shown in FIG. 12B.

FIG. 13 shows the percent lysis of T cells targeted by NK cells at different effector:target (E:T) ratios treated with sgRNA and base editor and UGI mRNAs.

FIG. 14 shows the mean percentage of CD8+ T cells that are negative for HLA-A surface receptors following treatment with sgRNAs in the 100-mer or 91-mer formats targeting HLA-A.

FIGS. 15A-15C respectively show HLA-A gene editing correlation to protein knockout in Donors A-C.

### III. DETAILED DESCRIPTION

The present disclosure provides engineered human cells, as well as methods and compositions for genetically modifying a human cell to make engineered human cells that are useful, for example, for adoptive cell transfer (ACT) therapies. The disclosure provides engineered human cells with reduced or eliminated surface expression of HLA-A relative to an unmodified cell, wherein the cell is homozygous for HLA-B and homozygous for HLA-C. Thus, the engineered human cells disclosed herein provide a “partial matching” solution to hurdles associated with allogeneic cell transfer.

In some embodiments, the disclosure provides engineered human cells with reduced or eliminated surface expression of HLA-A as a result of a genetic modification in the HLA-A gene, wherein the cell is homozygous for HLA-B and homozygous for HLA-C. In some embodiments, the disclosure provides compositions and methods for reducing or eliminating expression of HLA-A protein relative to an unmodified cell and compositions and methods to reduce the cell's susceptibility to immune rejection. In some embodiments, the engineered human cells with reduced or eliminated surface expression of HLA-A relative to an unmodified cell are not susceptible to lysis by NK cells, a problem observed with other approaches that reduce or eliminate MHC class I protein expression. In some embodiments, the methods and compositions comprise reducing or eliminating surface expression of HLA-A protein by genetically modifying HLA-A with a gene editing system, and inserting an exogenous nucleic acid encoding a targeting receptor, or other polypeptide (expressed on the cell surface or secreted) into the cell by genetic modification. The engineered cell compositions produced by the methods disclosed herein have desirable properties, including e.g., reduced expression of HLA-A, reduced immunogenicity in vitro and in vivo, increased survival, and increased genetic compatibility with greater subjects for transplant.

The term “about” or “approximately” means an acceptable error for a particular value as determined by one of ordinary skill in the art, which depends in part on how the value is measured or determined, or a degree of variation that does not substantially affect the properties of the described subject matter, or within the tolerances accepted in the art, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### A. Definitions

Unless stated otherwise, the following terms and phrases as used herein are intended to have the following meanings:

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, CBBA, CABA, and so forth. The skilled artisan will understand that typically there is no limit on the number of items or terms in any combination, unless otherwise apparent from the context.

As used herein, the term “kit” refers to a packaged set of related components, such as one or more polynucleotides or compositions and one or more related materials such as delivery devices (e.g., syringes), solvents, solutions, buffers, instructions, or desiccants.

An “allogeneic” cell, as used herein, refers to a cell originating from a donor subject of the same species as a recipient subject, wherein the donor subject and recipient subject have genetic dissimilarity, e.g., genes at one or more loci that are not identical. Thus, e.g., a cell is allogeneic with respect to the subject to be administered the cell. As used herein, a cell that is removed or isolated from a donor, that will not be re-introduced into the original donor, is considered an allogeneic cell.

An “autologous” cell, as used herein, refers to a cell derived from the same subject to whom the material will later be re-introduced. Thus, e.g., a cell is considered autologous if it is removed from a subject and it will then be re-introduced into the same subject.

“β2M” or “B2M,” as used herein, refers to nucleic acid sequence or protein sequence of “β-2 microglobulin”; the human gene has accession number NC_000015 (range 44711492..44718877), reference GRCh38.p13. The B2M protein is associated with MHC class I molecules as a heterodimer on the surface of nucleated cells and is required for MHC class I protein expression.

“CIITA” or “CIITA” or “C2TA,” as used herein, refers to the nucleic acid sequence or protein sequence of “class II major histocompatibility complex transactivator;” the human gene has accession number NC_000016.10 (range 10866208..10941562), reference GRCh38.p13. The CIITA protein in the nucleus acts as a positive regulator of MHC class II gene transcription and is required for MHC class II protein expression.

As used herein, “MHC” or “MHC molecule(s)” or “MHC protein” or “MHC complex(es),” refers to a major histocompatibility complex molecule (or plural), and includes e.g., MHC class I and MHC class II molecules. In humans, MHC molecules are referred to as “human leukocyte antigen” complexes or “HLA molecules” or “HLA protein.” The use of terms “MHC” and “HLA” are not meant to be limiting; as used herein, the term “MHC” may be used to refer to human MHC molecules, i.e., HLA molecules. Therefore, the terms “MHC” and “HLA” are used interchangeably herein.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain;” the human gene has accession number NC_000006.12 (29942532..29945870). The HLA-A gene is known to have thousands of different genotypic versions of the HLA-A gene across the population (and an individual may receive two different alleles of the HLA-A gene). A public database for HLA-A alleles, including sequence information, may be accessed at IPD-IMGT/HLA: www.ebi.ac.uk/ipd/imgt/hla/. All alleles of HLA-A are encompassed by the terms “HLA-A” and “HLA-A gene.”

“HLA-B” as used herein in the context of nucleic acids refers to the gene encoding the heavy chain of the HLA-B protein molecule. The HLA-B is also referred to as “HLA class I histocompatibility, B alpha chain;” the human gene has accession number NC_000006.12 (31353875..31357179).

“HLA-C” as used herein in the context of nucleic acids refers to the gene encoding the heavy chain of the HLA-C protein molecule. The HLA-C is also referred to as “HLA class I histocompatibility, C alpha chain;” the human gene has accession number NC_000006.12 (31268749..31272092).

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

As used herein, the term “homozygous” refers to having two identical alleles of a particular gene.

As used herein, an HLA “allele” can refer to a named HLA-A, HLA-B, or HLA-C gene wherein the first four digits (or the first two sets of digits separated by a colon, e.g., HLA-A*02:101:01:02N where the first two sets of digits are bolded and in italics) of the name following “HLA-A”, HLA-B″, or “HLA-C” are specified. As known in the art, the first four digits (or first two sets of digits separated by a colon) specify the protein of the allele. For example, HLA-A*02:01 and HLA-A*01:02 are distinct HLA-A alleles. Further genotypes of each allele exist, such as, e.g., HLA-A*02:01:02:01. Further genotypes of a given allele are considered to be identical alleles, e.g., HLA-A*02:01:02:01 and HLA-A*02:01 are identical alleles. Thus, HLA alleles are homozygous when the alleles are identical (i.e., when the alleles have the same first four digits or same first two sets of digits separated by a colon).

“Matching” or “matched” refers to shared alleles between the donor and the recipient, e.g., identical alleles.

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to, for example, the guide that directs an RNA-guided DNA binding agent to a target DNA and can be a single guide RNA, or the combination of a crRNA and a trRNA (also known as tracrRNA). Exemplary gRNAs include Class II Cas nuclease guide RNAs, in modified or unmodified forms. The crRNA and trRNA may be associated as a single RNA molecule (single guide RNA, sgRNA) or in two separate RNA strands (dual guide RNA, dgRNA). “Guide RNA” or “gRNA” refers to each type. The trRNA may be a naturally occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9 (SpCas9)) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, also called “Cas protein” as used herein, encompasses Cas cleavases, Cas nickases, and dCas DNA binding agents. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a UGI. In some embodiments, the editor lacks a UGI.

As used herein, a “cytidine deaminase” means a polypeptide or complex of polypeptides that is capable of cytidine deaminase activity, that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941) and is included herein as SEQ ID NO: 40. In some embodiments, the APOBEC3 protein is a human APOBEC3 protein and/or a wild-type protein. Variants include proteins having a sequence that differs from wild-type APOBEC3 protein by one or several mutations (i.e. substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to a APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of the DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables 51 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902).

As used herein, the term “uracil glycosylase inhibitor” or “UGI” refers to a protein that is capable of inhibiting a uracil-DNA glycosylase (UDG) base-excision repair enzyme.

As used herein, “open reading frame” or “ORF” of a gene refers to a sequence consisting of a series of codons that specify the amino acid sequence of the protein that the gene codes for. The ORF begins with a start codon (e.g., ATG in DNA or AUG in RNA) and ends with a stop codon, e.g., TAA, TAG or TGA in DNA or UAA, UAG, or UGA in RNA.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

“mRNA” is used herein to refer to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

As used herein, “indels” refer to insertion/deletion mutations consisting of a number of nucleotides that are either inserted or deleted, e.g. at the site of double-stranded breaks (DSBs), in a target nucleic acid.

As used herein, “reduced or eliminated” expression of a protein on a cell refers to a partial or complete loss of expression of the protein relative to an unmodified cell. In some embodiments, the surface expression of a protein on a cell is measured by flow cytometry and has “reduced or eliminated” surface expression relative to an unmodified cell as evidenced by a reduction in fluorescence signal upon staining with the same antibody against the protein. A cell that has “reduced or eliminated” surface expression of a protein by flow cytometry relative to an unmodified cell may be referred to as “negative” for expression of that protein as evidenced by a fluorescence signal similar to a cell stained with an isotype control antibody. The “reduction or elimination” of protein expression can be measured by other known techniques in the field with appropriate controls known to those skilled in the art.

As used herein, “knockdown” refers to a decrease in expression of a particular gene product (e.g., protein, mRNA, or both), e.g., as compared to expression of an unedited target sequence. Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include analyzing mRNA isolated from a sample of interest. In some embodiments, “knockdown” may refer to some loss of expression of a particular gene product, for example a decrease in the amount of mRNA transcribed or a decrease in the amount of protein expressed by a cell or population of cells (including in vivo populations such as those found in tissues).

As used herein, “knockout” refers to a loss of expression from a particular gene or of a particular protein in a cell. Knockout can result in a decrease in expression below the level of detection of the assay. Knockout can be measured either by detecting total cellular amount of a protein in a cell, a tissue or a population of cells.

As used herein, a “target sequence” or “genomic target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing one or more symptoms of the disease, including recurrence of the symptom.

Reference will now be made in detail to certain embodiments of the invention, examples of which are illustrated in the accompanying drawings. While the invention is described in conjunction with the illustrated embodiments, it will be understood that they are not intended to limit the invention to those embodiments. On the contrary, the invention is intended to cover all alternatives, modifications, and equivalents, which may be included within the invention as defined by the appended claims and included embodiments.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense, i.e., equivalent to “and/or,” unless the context clearly indicates otherwise.

The section headings used herein are for organizational purposes only and are not to be construed as limiting the desired subject matter in any way. In the event that any material incorporated by reference contradicts any term defined in this specification or any other express content of this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### B. Genetically Modified Cells

### 1. Engineered Human Cell Compositions

The present disclosure provides engineered human cell compositions which have reduced or eliminated surface expression of HLA-A relative to an unmodified cell, comprising a genetic modification in the HLA-A gene, wherein the cell is homozygous for HLA-B and homozygous for HLA-C. In some embodiments, the engineered human cell is an allogeneic cell. In some embodiments, the engineered human cell with reduced HLA-A expression is useful for adoptive cell transfer therapies. In some embodiments, the engineered human cell comprises additional genetic modifications in the genome of the cell (e.g., reducing or elimination of MHC class II proteins, and/or reducing or eliminating endogenous T cell receptor (TCR) proteins, and/or introduction of an exogenous nucleic acid for expression) to yield a cell that is desirable for allogeneic transplant purposes.

In some embodiments, the engineered human cell is an allogeneic cell therapy. In some embodiments, the engineered human cell is transferred to a recipient that has the same HLA-B allele as the engineered human cell. In some embodiments, the engineered human cell is transferred to a recipient that has the same HLA-C allele as the engineered human cell. In some embodiments, the engineered human cell is transferred to a recipient that has the same HLA-B and HLA-C alleles as the engineered human cell. Thus, the engineered human cells disclosed herein provide a partial HLA match to a recipient, thereby reducing the risk of an adverse immune response.

In some embodiments, an engineered human cell is provided which has reduced or eliminated surface expression of HLA-A relative to an unmodified cell, comprising a genetic modification in the HLA-A gene, wherein the cell is homozygous for HLA-B and homozygous for HLA-C.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942854-chr6:29942913 and chr6:29943518-chr6:29943619; wherein the cell is homozygous for HLA-B and homozygous for HLA-C.

In some embodiments, for each given range of genomic coordinates, a range may encompass+/−10 nucleotides on either end of the specified coordinates. For example, if chr6:29942854-chr6:29942913 is given, in some embodiments the genomic target sequence or genetic modification may fall within chr6:29942844-chr6:29942923. In some embodiments, for each given range of genomic coordinates, the range may encompass+/−5 nucleotides on either end of the range.

In some embodiments, a given range of genomic coordinates may comprise a target sequence on both strands of the DNA (i.e., the plus (+) strand and the minus (−) strand).

Genetic modifications in the HLA-A gene are described further herein. In some embodiments, a genetic modification in the HLA-a gene comprises any one or more of an insertion, deletion, substitution, or deamination of at least one nucleotide in a target sequence.

The engineered human cells described herein may comprise a genetic modification in any HLA-A allele of the HLA-A gene. The HLA gene is located in chromosome 6 in a genomic region referred to as the HLA superlocus; hundreds of HLA-A alleles have been reported in the art (see e.g., Shiina et al., Nature 54:15-39 (2009). Sequences for HLA-A alleles are available in the art (see e.g., IPD-IMGT/HLA database for retrieving sequences of specific HLA-A alleles https://www.ebi.ac.uk/ip d/imgt/hla/allele.html).

In some embodiments, the cell has reduced or eliminated expression of at least one HLA-A allele selected from: HLA-A1, HLA-A2, HLA-A3, HLA-A11, and HLA-A24. In some embodiments, the cell has reduced or eliminated expression of HLA-A1. In some embodiments, the cell has reduced or eliminated expression of HLA-A2. In some embodiments, the cell has reduced or eliminated expression of HLA-A3. In some embodiments, the cell has reduced or eliminated expression of HLA-A11. In some embodiments, the cell has reduced or eliminated expression of HLA-A24.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942864 to chr6:29942903.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942864-29942884; chr6:29942868-29942888; chr6:29942876-29942896; chr6:29942877-29942897; and chr6:29942883-29942903.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528-29943548; chr6:29943529-29943549; chr6:29943530-29943550; chr6:29943537-29943557; chr6:29943549-29943569; and chr6:29943589-29943609.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942876-29942897.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528-chr629943550.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942864-29942884, chr6:29942868-29942888, chr6:29942876-29942896, chr6:29942877-29942897.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528-29943548, chr6:29943529-29943549, chr6:29943530-29943550.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942864-29942884.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942868-29942888.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942876-29942896.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942877-29942897.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29942883-29942903.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943126-29943146.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528-29943548.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943529-29943549.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943530-29943550.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943537-29943557.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943549-29943569.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943589-29943609.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29944026-29944046.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942854-chr6:29942913 and chr6:29943518-chr6:29943619. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, wherein the genetic modification comprises at least 2, at least 3, at least 4, at least 5, at least 6, at least 7, at least 8, at least 9, or at least 10 contiguous nucleotides within the genomic coordinates. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, wherein the genetic modification comprises at least 5 contiguous nucleotides within the genomic coordinates. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, wherein the genetic modification comprises at least 6, 7, 8, 9, or contiguous nucleotides within the genomic coordinates. In some embodiments, the genetic modification comprises at least 6 contiguous nucleotides within the genomic coordinates. In some embodiments, the genetic modification comprises at least 7 contiguous nucleotides within the genomic coordinates. In some embodiments, the genetic modification comprises at least 8 contiguous nucleotides within the genomic coordinates. In some embodiments, the genetic modification comprises at least 9 contiguous nucleotides within the genomic coordinates. In some embodiments, the genetic modification comprises at least 10 contiguous nucleotides within the genomic coordinates. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, wherein the genetic modification comprises at least one C to T substitution or at least one A to G substitution within the genomic coordinates. In some embodiments, the cell is homozygous for HLA-B. In some embodiments, the cell is homozygous for HLA-C. In some embodiments, the cell is homozygous for HLA-B and homozygous for HLA-C.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870. In some embodiments, the HLA-A genomic target sequence comprises at least 10 contiguous nucleotides within the genomic coordinates. In some embodiments, the HLA-A genomic target sequence comprises at least contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046. In some embodiments, the HLA-A genomic target sequence comprises at least 10 contiguous nucleotides within the genomic coordinates. In some embodiments, the HLA-A genomic target sequence comprises at least contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943528-29943548; chr6:29943529-29943549; chr6:29943530-29943550; chr6:29943537-29943557; chr6:29943549-29943569; and chr6:29943589-29943609. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; and chr6:29942883-29942903. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29943528-29943548; chr6:29943529-29943549; chr6:29943530-29943550; chr6:29943537-29943557; chr6:29943549-29943569; and chr6:29943589-29943609. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942864-29942884, chr6:29942868-29942888, chr6:29942876-29942896, chr6:29942877-29942897. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29943528-29943548, chr6:29943529-29943549, chr6:29943530-29943550. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29945290-29945310, chr6:29945296-29945316, chr6:29945297-29945317, and chr6:29945300-29945320. Due to allelic polymorphism, in some embodiments, the target sequences may comprise 1, 2, or 3 mismatches from the genomic sequence of hg38. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29890117-29890137, chr6:29927058-29927078, chr6:29934330-29934350, chr6:29942541-29942561, chr6:29942542-29942562, chr6:29942543-29942563, chr6:29942543-29942563, chr6:29942550-29942570, chr6:29942864-29942884, chr6:29942868-29942888, chr6:29942876-29942896, chr6:29942876-29942896, chr6:29942877-29942897, chr6:29942883-29942903, chr6:29943062-29943082, chr6:29943063-29943083, chr6:29943092-29943112, chr6:29943115-29943135, chr6:29943118-29943138, chr6:29943119-29943139, chr6:29943120-29943140, chr6:29943126-29943146, chr6:29943128-29943148, chr6:29943129-29943149, chr6:29943134-29943154, chr6:29943134-29943154, chr6:29943135-29943155, chr6:29943136-29943156, chr6:29943140-29943160, chr6:29943142-29943162, chr6:29943143-29943163, chr6:29943188-29943208, chr6:29943528-29943548, chr6:29943529-29943549, chr6:29943530-29943550, chr6:29943536-29943556, chr6:29943537-29943557, chr6:29943538-29943558, chr6:29943549-29943569, chr6:29943556-29943576, chr6:29943589-29943609, chr6:29943590-29943610, chr6:29943590-29943610, chr6:29943599-29943619, chr6:29943600-29943620, chr6:29943601-29943621, chr6:29943602-29943622, chr6:29943603-29943623, chr6:29943774-29943794, chr6:29943779-29943799, chr6:29943780-29943800, chr6:29943822-29943842, chr6:29943824-29943844, chr6:29943857-29943877, chr6:29943858-29943878, chr6:29943859-29943879, chr6:29943860-29943880, chr6:29944026-29944046, chr6:29944077-29944097, chr6:29944078-29944098, chr6:29944458-29944478, chr6:29944478-29944498, chr6:29944597-29944617, chr6:29944772-29944792, chr6:29944907-29944927, chr6:29945104-29945124, chr6:29945118-29945138, chr6:29945176-29945196, chr6:29945180-29945200, chr6:29944642-29944662, chr6:29944782-29944802, chr6:29945024-29945044, chr6:29945105-29945125, chr6:29945119-29945139, chr6:29945177-29945197, chr6:29945187-29945207, chr6:29944643-29944663, chr6:29944850-29944870, chr6:29945097-29945117, chr6:29945116-29945136, chr6:29945124-29945144, chr6:29945177-29945197, chr6:29945188-29945208, chr6:29945228-29945248, chr6:29945230-29945250, chr6:29945231-29945251, chr6:29945232-29945252, chr6:29945308-29945328, chr6:29945361-29945381, chr6:29945362-29945382, and chr6:31382543-31382563. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as an S. pyogenes Cas9 or a base editor that comprises an S. pyogenes Cas9 nickase.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942815-29942835, chr6:29942816-29942836, chr6:29942817-29942837, chr6:29942817-29942837, chr6:29942828-29942848, chr6:29942837-29942857, chr6:29942885-29942905, chr6:29942895-29942915, chr6:29942896-29942916, chr6:29942898-29942918, chr6:29942899-29942919, chr6:29942900-29942920, chr6:29942904-29942924, chr6:29942905-29942925, chr6:29942912-29942932, chr6:29942913-29942933, chr6:29943490-29943510, chr6:29943497-29943517, chr6:29943498-29943518, chr6:29943502-29943522, chr6:29943502-29943522, chr6:29943511-29943531, chr6:29943520-29943540, chr6:29943521-29943541, chr6:29943566-29943586, chr6:29943569-29943589, chr6:29943569-29943589, chr6:29943570-29943590, chr6:29943573-29943593, chr6:29943578-29943598, chr6:29943585-29943605, chr6:29943589-29943609, chr6:29943568-29943588, and chr6:29942815-29942835. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as an S. pyogenes Cas9.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942884-29942904, chr6:29943519-29943539, chr6:29942863-29942883. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as an S. aureus Cas9.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29943517-29943537, and chr6:29943523-29943543. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as a CasX.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942845-29942869, chr6:29942852-29942876, chr6:29942865-29942889, chr6:29942891-29942915, chr6:29942895-29942919, chr6:29942903-29942927, chr6:29942904-29942928, chr6:29943525-29943549, chr6:29943535-29943559, chr6:29943539-29943563, chr6:29943547-29943571, chr6:29943548-29943572, chr6:29943555-29943579, chr6:29943557-29943581, chr6:29943558-29943582, chr6:29943518-29943542, chr6:29943538-29943562, chr6:29943547-29943571, chr6:29943556-29943580, chr6:29943559-29943583, chr6:29943563-29943587, chr6:29943564-29943588, chr6:29943565-29943589, chr6:29943568-29943592, chr6:29943571-29943595, chr6:29943572-29943596, chr6:29943595-29943619, chr6:29943596-29943620, and chr6:29943600-29943624. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as an Nme2 Cas9.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942885-29942905, chr6:29942895-29942915, chr6:29942896-29942916, chr6:29942898-29942918, chr6:29942899-29942919, chr6:29942900-29942920, chr6:29942904-29942924, chr6:29943511-29943531, chr6:29943520-29943540, chr6:29943521-29943541, chr6:29943529-29943549, chr6:29943566-29943586, chr6:29943568-29943588, chr6:29943569-29943589, chr6:29943569-29943589, chr6:29943570-29943590, chr6:29943573-29943593, chr6:29943578-29943598, chr6:29943585-29943605, and chr6:29943589-29943609. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates. In some embodiments, the gene editing system comprises an RNA-guided DNA binding agent, such as a base editor comprising a deaminase and an S. pyogenes Cas9 nickase.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942469-29942489, chr6:29943058-29943078, chr6:29943063-29943083, chr6:29943080-29943100, chr6:29943187-29943207, chr6:29943192-29943212, chr6:29943197-29943217, chr6:29943812-29943832, chr6:29944349-29944369, chr6:29944996-29945016, chr6:29945018-29945038, chr6:29945341-29945361, and chr6:29945526-29945546. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chosen from: chr6:29942854 to chr6:29942913 and chr6:29943518 to chr6:29943619. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates: chr6:29942876-29942897. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates: chr6:29943528-chr629943550. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29942864-29942884. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29942868-29942888. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29942876-29942896. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29942877-29942897. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29942883-29942903. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943126-29943146. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943528-29943548. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943529-29943549. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943530-29943550. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943537-29943557. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943549-29943569. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29943589-29943609. In some embodiments, an engineered human cell is provided wherein the HLA-A expression is reduced or eliminated by a gene editing system that binds to an HLA-A genomic target sequence comprising at least 5 contiguous nucleotides within the genomic coordinates chr6:29944026-29944046. In some embodiments, the HLA-A genomic target sequence comprises at least 10 contiguous nucleotides within the genomic coordinates. In some embodiments, the HLA-A genomic target sequence comprises at least 15 contiguous nucleotides within the genomic coordinates.

In some embodiments, the HLA-A genomic target sequence comprises at least 17, 19, 18, or 20 contiguous nucleotides within the genomic coordinates.

In some embodiments, the gene editing system comprises a transcription activator-like effector nuclease (TALEN). In some embodiments, the gene editing system comprises a zinc finger nuclease. In some embodiments, the gene editing system comprises a CRISPR/Cas system, such as a class 2 system. In some embodiments, the gene editing system comprises an RNA-guided DNA-binding agent or a nucleic acid encoding an RNA-guided DNA binding agent.

Exemplary RNA-guided DNA binding agents are shown in Table 1A below.

TABLE 1A
Exemplary RNA-guided DNA binding agents.

| RNA-guided DNA binding agent | PAM | Guide Length |
|---|---|---|
| Cas9 nuclease from S. pyogenes | NGG | 20 bp |
| Cas9 nuclease from Neisseria | NNNNG[A/C]TT | 20 bp |
| meningitidis | | |
| Cas9 nuclease from Streptococcus | NNAGAAW | 20 bp |
| thermophilus | | |
| Cas9 | NNG(A/G)(A/G)T | 20 bp |
| nuclease is from Staphylococcus | | |
| aureus | | |
| Cpf1 nuclease | TTTN | 23 bp |
| from Francisella novicida | | |
| Cpf1 nuclease | TTTV | 23 bp |
| from Acidaminococcus sp. | | |
| Cpf1 nuclease | TTTV | 23 bp |
| from Lachnospiraceae bacterium | | |
| C-to-T base editor* | NGG | 20 bp |
| A-to-G base editor* | NGG | 20 bp |
| Cas12a | same as Cpf1 | |
| CasX | TTCN | 20 bp |
| NME2 | NNNNCC | 24 bp |

*Exemplary base editor based on deaminase-SpyCas9 nickase. As is apparent, the base editor specificity, including PAM, will vary with its nickase.

In some embodiments, the RNA-guided DNA-binding agent or nucleic acid encoding the RNA-guided DNA binding agent comprises a Cas9 protein. In some embodiments, the RNA-guided DNA binding agent is selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, Mad7 nuclease, ARCUS nucleases, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX.

In some embodiments, the RNA-guided DNA-binding agent or nucleic acid encoding the RNA-guided DNA binding agent is S. pyogenes Cas9. In some embodiments, the RNA-guided DNA-binding agent or nucleic acid encoding the RNA-guided DNA binding agent is N. meningitidis Cas9, e.g. Nme2Cas9. In some embodiments, the RNA-guided DNA-binding agent or nucleic acid encoding the RNA-guided DNA binding agent is S. thermophilus Cas9. In some embodiments, the RNA-guided DNA-binding agent or nucleic acid encoding the RNA-guided DNA binding agent is S. aureus Cas9. In some embodiments, the RNA-guided DNA-binding agent or nucleic acid encoding the RNA-guided DNA binding agent is Cpf1 from F. novicida. In some embodiments, the RNA-guided DNA-binding agent or nucleic acid encoding the RNA-guided DNA binding agent is Cpf1 from Acidaminococcus sp. In some embodiments, the RNA-guided DNA-binding agent or nucleic acid encoding the RNA-guided DNA binding agent is Cpf1 from Lachnospiraceae bacterium ND2006. In some embodiments, the RNA-guided DNA-binding agent or nucleic acid encoding the RNA-guided DNA binding agent is a C to T base editor. In some embodiments, the RNA-guided DNA-binding agent or nucleic acid encoding the RNA-guided DNA binding agent is a A to G base editor. In some embodiments, the base editor comprises a deaminase and an RNA-guided nickase. In some embodiments, the RNA-guided DNA-binding agent or nucleic acid encoding the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase. In some embodiments, the RNA-guided DNA-binding agent or nucleic acid encoding the RNA-guided DNA binding agent is Cas12a. In some embodiments, the RNA-guided DNA-binding agent or nucleic acid encoding the RNA-guided DNA binding agent is CasX.

In any of the above embodiments, the gene editing system comprises an RNA-guided DNA binding agent, or a nucleic acid encoding an RNA-guided DNA binding agent. In some embodiments, the RNA-guided DNA binding agent comprises a Cas9. In some embodiments, the RNA-guided DNA binding agent is an S. pyogenes Cas9. In some embodiments, the RNA-guided DNA binding agent is a base editor. In some embodiments the base editor comprises a C to T deaminase and an RNA-guided nickase such as an S. pyogenes Cas9 nickase. In some embodiments the base editor comprises a A to G deaminase and an RNA-guided nickase such as an S. pyogenes Cas9 nickase.

In some embodiments, when the engineered cell is homozygous for HLA-B, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, when the engineered cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the engineered cell is homozygous for HLA-B and homozygous for HLA-C. In some embodiments, the HLA-B and HLA-C alleles of the engineered human cell are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the HLA-B and HLA-C alleles are HLA-B*07:02 and HLA-C*07:02. In some embodiments, the HLA-B and HLA-C alleles are HLA-B*08:01 and HLA-C*07:01. In some embodiments, the HLA-B and HLA-C alleles are HLA-B*44:02 and HLA-C*05:01. In some embodiments, the HLA-B and HLA-C alleles are HLA-B*35:01 and HLA-C*04:01. In some embodiments, the HLA-B and HLA-C alleles are HLA-B*40:01 and HLA-C*03:04. In some embodiments, the HLA-B and HLA-C alleles are HLA-B*57:01 and HLA-C*06:02. In some embodiments, the HLA-B and HLA-C alleles are HLA-B*14:02 and HLA-C*08:02. In some embodiments, the HLA-B and HLA-C alleles are HLA-B*15:01 and HLA-C*03:03. In some embodiments, the HLA-B and HLA-C alleles are HLA-B*13:02 and HLA-C*06:02. In some embodiments, the HLA-B and HLA-C alleles are HLA-B*44:03 and HLA-C*16:01. In some embodiments, the HLA-B and HLA-C alleles are HLA-B*38:01 and HLA-C*12:03. In some embodiments, the HLA-B and HLA-C alleles are HLA-B*18:01 and HLA-C*07:01. In some embodiments, the HLA-B and HLA-C alleles are HLA-B*44:03 and HLA-C*04:01. In some embodiments, the HLA-B and HLA-C alleles are HLA-B*51:01 and HLA-C*15:02. In some embodiments, the HLA-B and HLA-C alleles are HLA-B*49:01 and HLA-C*07:01. In some embodiments, the HLA-B and HLA-C alleles are HLA-B*15:01 and HLA-C*03:04. In some embodiments, the HLA-B and HLA-C alleles are HLA-B*18:01 and HLA-C*12:03. In some embodiments, the HLA-B and HLA-C alleles are HLA-B*27:05 and HLA-C*02:02. In some embodiments, the HLA-B and HLA-C alleles are HLA-B*35:03 and HLA-C*04:01. In some embodiments, the HLA-B and HLA-C alleles are HLA-B*18:01 and HLA-C*05:01. In some embodiments, the HLA-B and HLA-C alleles are HLA-B*52:01 and HLA-C*12:02. In some embodiments, the HLA-B and HLA-C alleles are HLA-B*51:01 and HLA-C*14:02. In some embodiments, the HLA-B and HLA-C alleles are HLA-B*37:01 and HLA-C*06:02. In some embodiments, the HLA-B and HLA-C alleles are HLA-B*53:01 and HLA-C*04:01. In some embodiments, the HLA-B and HLA-C alleles are HLA-B*55:01 and HLA-C*03:03. In some embodiments, the HLA-B and HLA-C alleles are HLA-B*44:02 and HLA-C*07:04. In some embodiments, the HLA-B and HLA-C alleles are HLA-B*44:03 and HLA-C*07:01. In some embodiments, the HLA-B and HLA-C alleles are HLA-B*35:02 and HLA-C*04:01. In some embodiments, the HLA-B and HLA-C alleles are HLA-B*15:01 and HLA-C*04:01. In some embodiments, the HLA-B and HLA-C alleles are and HLA-B*40:02 and HLA-C*02:02.

The HLA-B and HLA-C allele combinations disclosed herein cumulatively cover about 88% of the population. The cumulative frequency of HLA-B and HLA-C allele pairs is shown in Table 1B below.

TABLE 1B
Cumulative Frequency of HLA-A and
HLA-C Alleles in the Population.

| Cumulative Frequency | Alleles |
|---|---|
| 0.194 | HLA-B*07:02 and HLA-C*07:02 |
| 0.33 | HLA-B*08:01 and HLA-C*07:01 |
| 0.413 | HLA-B*44:02 and HLA-C*05:01 |
| 0.483 | HLA-B*35:01 and HLA-C*04:01 |
| 0.534 | HLA-B*40:01 and HLA-C*03:04 |
| 0.594 | HLA-B*57:01 and HLA-C*06:02 |
| 0.62 | HLA-B*14:02 and HLA-C*08:02 |
| 0.648 | HLA-B*15:01 and HLA-C*03:03 |
| 0.671 | HLA-B*13:02 and HLA-C*06:02 |
| 0.696 | HLA-B*44:03 and HLA-C*16:01 |
| 0.717 | HLA-B*38:01 and HLA-C*12:03 |
| 0.734 | HLA-B*18:01 and HLA-C*07:01 |
| 0.751 | HLA-B*44:03 and HLA-C*04:01 |
| 0.766 | HLA-B*51:01 and HLA-C*15:02 |
| 0.776 | HLA-B*49:01 and HLA-C*07:01 |
| 0.787 | HLA-B*15:01 and HLA-C*03:04 |
| 0.798 | HLA-B*18:01 and HLA-C*12:03 |
| 0.809 | HLA-B*27:05 and HLA-C*02:02 |
| 0.815 | HLA-B*35:03 and HLA-C*04:01 |
| 0.827 | HLA-B*18:01 and HLA-C*05:01 |
| 0.838 | HLA-B*52:01 and HLA-C*12:02 |
| 0.845 | HLA-B*51:01 and HLA-C*14:02 |
| 0.856 | HLA-B*37:01 and HLA-C*06:02 |
| 0.865 | HLA-B*53:01 and HLA-C*04:01 |
| 0.872 | HLA-B*55:01 and HLA-C*03:03 |
| 0.876 | HLA-B*44:02 and HLA-C*07:04 |
| 0.881 | HLA-B*44:03 and HLA-C*07:01 |
| 0.884 | HLA-B*35:02 and HLA-C*04:01 |
| 0.888 | HLA-B*15:01 and HLA-C*04:01 |

In some embodiments, an engineered human cell which has reduced or eliminated surface expression of HLA-A relative to an unmodified cell is provided, that is homozygous for HLA-B and homozygous for HLA-C, further has reduced or eliminated surface expression of MHC class II protein. In some embodiments, the engineered human cell has a genetic modification in a gene that reduces or eliminates surface expression of MHC class II. In some embodiments, the engineered human cell has a genetic modification in the CIITA gene. In some embodiments, the engineered human cell has a genetic modification in the HLA-DR gene. In some embodiments, the engineered human cell has a genetic modification in the HLA-DQ gene. In some embodiments, the engineered human cell has a genetic modification in the HLA-DP gene. In some embodiments, the engineered human cell has a genetic modification in the RFX gene. In some embodiments, the engineered human cell has a genetic modification in the CREB gene. In some embodiments, the engineered human cell has a genetic modification in the Nuclear Factor (NF)-gamma gene.

In some embodiments, an engineered human cell which has reduced or eliminated surface expression of HLA-A relative to an unmodified cell is provided, that is homozygous for HLA-B and homozygous for HLA-C, further has reduced or eliminated surface expression of TRAC protein. In some embodiments, an engineered human cell which has reduced or eliminated surface expression of HLA-A relative to an unmodified cell is provided, that is homozygous for HLA-B and homozygous for HLA-C, further has reduced or eliminated surface expression of TRBC protein.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609, and wherein the engineered cell further comprises a genetic modification in a gene that reduces or eliminates the surface expression of MHC class II. In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609, and wherein the engineered cell further comprises a genetic modification in the CIITA gene.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609, and wherein the engineered cell further comprises a genetic modification in the TRAC gene. In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609, and wherein the engineered cell further comprises a genetic modification in the TRBC gene.

In some embodiments, an engineered human cell is provided which has reduced or eliminated surface expression of HLA-A relative to an unmodified cell, comprising a genetic modification in the HLA-A gene, wherein the genetic modification comprises at least one nucleotide within the genomic coordinates chr6:29943528 to chr6:29943609, and wherein the engineered cell further comprises an exogenous nucleic acid. In some embodiments, the engineered cell comprises an exogenous nucleic acid encoding a targeting receptor that is expressed on the surface of the engineered cell. In some embodiments, the targeting receptor is a CAR or a universal CAR. In some embodiments, the targeting receptor is a TCR. In some embodiments, the targeting receptor is a WT1 TCR. In some embodiments, the targeting receptor is a ligand for the receptor. In some embodiments, the targeting receptor is a hybrid CAR/TCR. In some embodiments, the targeting receptor comprises an antigen recognition domain (e.g., a cancer antigen recognition domain) and a subunit of a TCR). In some embodiments, the targeting receptor is a cytokine receptor. In some embodiments, the targeting receptor is a chemokine receptor. In some embodiments, the targeting receptor is a B cell receptor (BCR). In some embodiments, the engineered cell further comprises an exogenous nucleic acid encoding a polypeptide that is secreted by the engineered cell (i.e., a soluble polypeptide). In some embodiments, the exogenous nucleic acid encodes a therapeutic polypeptide. In some embodiments, the secreted polypeptide is an antibody. In some embodiments, the secreted polypeptide is an enzyme. In some embodiments, the exogenous nucleic acid encodes an antibody encodes a cytokine. In some embodiments, the exogenous nucleic acid encodes a chemokine. In some embodiments, the exogenous nucleic acid encodes a fusion protein.

The engineered human cell may be any of the exemplary cell types disclosed herein. Further, because MHC class I molecules are expressed on all nucleated cells, the engineered human cell may be any nucleated cell. In some embodiments, the engineered cell is an immune cell. In some embodiments, the engineered cell is a stem cell such as a hematopoetic stem cell (HSC). In some embodiments, the engineered cell is an induced pluripotent stem cell (iPSC). In some embodiments, the engineered cell is a mesenchymal stem cell (MSC). In some embodiments, the engineered cell is a neural stem cell (NSC). In some embodiments, the engineered cell is a limbal stem cell (LSC). In some embodiments, the engineered cell is a progenitor cell, e.g. an endothelial progenitor cell or a neural progenitor cell. In some embodiments, the engineered cell is a tissue-specific primary cell. In some embodiments, the engineered cell is a chosen from: chondrocyte, myocyte, and keratinocyte. In some embodiments, the engineered cell is a monocyte, macrophage, mast cell, dendritic cell, or granulocyte. In some embodiments, the engineered cell is monocyte. In some embodiments, the engineered cell is a macrophage. In some embodiments, the engineered cell is a mast cell. In some embodiments, the engineered cell is a dendritic cell. In some embodiments, the engineered cell is a granulocyte. In some embodiments, the engineered cell is a lymphocyte. In some embodiments, the engineered cell is a T cell. In some embodiments, the engineered cell is a CD4+ T cell. In some embodiments, the engineered cell is a CD8+ T cell. In some embodiments, the engineered cell is a memory T cell. In some embodiments, the engineered cell is a B cell. In some embodiments, the engineered cell is a plasma B cell. In some embodiments, the engineered cell is a memory B cell. In some embodiments, the engineered cell is a macrophage.

In some embodiments, the disclosure provides a pharmaceutical composition comprising any one of the engineered human cells disclosed herein. In some embodiments, the pharmaceutical composition comprises a population of any one of the engineered cells disclosed herein. In some embodiments, the pharmaceutical composition comprises a population of engineered cells that is at least 65% HLA-A negative as measured by flow cytometry. In some embodiments, the pharmaceutical composition comprises a population of engineered cells that is at least 70% HLA-A negative as measured by flow cytometry. In some embodiments, the pharmaceutical composition comprises a population of engineered cells that is at least 80% HLA-A negative as measured by flow cytometry. In some embodiments, the pharmaceutical composition comprises a population of engineered cells that is at least 90% HLA-A negative as measured by flow cytometry. In some embodiments, the pharmaceutical composition comprises a population of engineered cells that is at least 91% negative as measured by flow cytometry. In some embodiments, the pharmaceutical composition comprises a population of engineered cells that is at least 92% HLA-A negative as measured by flow cytometry. In some embodiments, the pharmaceutical composition comprises a population of engineered cells that is at least 93% HLA-A negative as measured by flow cytometry. In some embodiments, the pharmaceutical composition comprises a population of engineered cells that is at least 94% HLA-A negative as measured by flow cytometry.

In some embodiments, the pharmaceutical composition comprises a population of engineered cells that is at least 95% endogenous TCR protein negative as measured by flow cytometry. In some embodiments, the pharmaceutical composition comprises a population of engineered cells that is at least 97% endogenous TCR protein negative as measured by flow cytometry. In some embodiments, the pharmaceutical composition comprises a population of engineered cells that is at least 98% endogenous TCR protein negative as measured by flow cytometry. In some embodiments, the pharmaceutical composition comprises a population of engineered cells that is at least 99% endogenous TCR protein negative as measured by flow cytometry. In some embodiments, the pharmaceutical composition comprises a population of engineered cells that is at least 99.5% endogenous TCR protein negative as measured by flow cytometry.

In some embodiments, methods are provided for administering the engineered human cells or pharmaceutical compositions disclosed herein to a subject in need thereof. In some embodiments, methods are provided for administering the engineered human cells or pharmaceutical compositions disclosed herein to a subject as an ACT therapy. In some embodiments, methods are provided for administering the engineered human cells or pharmaceutical compositions disclosed herein to a subject as a treatment for cancer. In some embodiments, methods are provided for administering the engineered human cells or pharmaceutical compositions disclosed herein to a subject as a treatment for an autoimmune disease. In some embodiments, methods are provided for administering the engineered human cells or pharmaceutical compositions disclosed herein to a subject as a treatment for an infectious disease.

### C. Methods and Compositions for Reducing or Eliminating Surface Expression of HLA-A

The present disclosure provides methods and compositions for reducing or eliminating surface expression of HLA-A protein relative to an unmodified cell by genetically modifying the HLA-A gene. The resultant genetically modified cell may also be referred to herein as an engineered cell. In some embodiments, an already-genetically modified (or engineered) cell may be the starting cell for further genetic modification using the methods or compositions provided herein. In some embodiments, the cell is an allogeneic cell. In some embodiments, a cell with reduced HLA-A expression is useful for adoptive cell transfer therapies. In some embodiments, editing of the HLA-A gene is combined with additional genetic modifications to yield a cell that is desirable for allogeneic transplant purposes.

In some embodiments, the methods comprise reducing surface expression of HLA-A protein in a human cell relative to an unmodified cell, comprising contacting a cell with composition comprising a) an HLA-A guide RNA comprising: i. a guide sequence selected from SEQ ID NOs: 1-211; or ii. at least 17, 18, 19, or 20 contiguous nucleotides of a sequence selected from SEQ ID NOs: 1-211; or iii. a guide sequence at least 95%, 90%, or 85% identical to a sequence selected from SEQ ID NOs: 1-211; or iv. a guide sequence that binds a target site comprising a genomic region listed in Tables 2-5; or v. a guide sequence that is complementary to at least 17, 18, 19, or 20 contiguous nucleotides of a genomic region listed in Tables 1-2 and 5, or a guide sequence that is complementary to at least 17, 18, 19, 21, 22, 23, or 24 contiguous nucleotides of a genomic region listed in Table 4; or vi. a guide sequence that is at least 95%, 90%, or 85% identical to a sequence selected from (v); and optionally b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent comprises a Cas9 protein. In some embodiments, the RNA-guided DNA binding agent is selected from one of: S. pyogenes Cas9, Neisseria meningitidis Cas9, e.g. an Nme2Cas9, S. thermophilus C as 9, S. aureus C as 9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments the RNA-guided DNA binding agent is N. meningitidis Cas9, e.g., Nme2Cas9. In some embodiments the RNA-guided DNA binding agent is S. thermophilus Cas9. In some embodiments the RNA-guided DNA binding agent is S. aureus Cas9. In some embodiments the RNA-guided DNA binding agent is Cpf1 from F. novicida. In some embodiments the RNA-guided DNA binding agent is Cpf1 from Acidaminococcus sp. In some embodiments the RNA-guided DNA binding agent is Cpf1 from Lachnospiraceae bacterium ND2006. In some embodiments the RNA-guided DNA binding agent is a C to T base editor. In some embodiments the RNA-guided DNA binding agent is a A to G base editor. In some embodiments, the base editor comprises a deaminase and an RNA-guided nickase. In some embodiments the RNA-guided DNA binding agent comprises a APOBEC3A deaminase (A3A) and an RNA-guided nickase. In some embodiments, the RNA-guided nickase is a SpyCas9 nickase. In some embodiments, the RNA-guided nickase comprises an NmeCas9 nickase. In some embodiments the RNA-guided DNA binding agent is Cas12a. In some embodiments the RNA-guided DNA binding agent is CasX. In some embodiments, the expression of HLA-A protein on the surface of the cell (i.e., engineered cell) is thereby reduced.

In some embodiments, the methods comprise making an engineered human cell, which has reduced or eliminated surface expression of HLA-A protein relative to an unmodified cell, wherein the cell is homozygous for HLA-B and homozygous for HLA-C, comprising contacting a cell with composition comprising a) an HLA-A guide RNA comprising: i. a guide sequence selected from SEQ ID NOs: 1-211; or ii. at least 17, 18, 19, or 20 contiguous nucleotides of a sequence selected from SEQ ID NOs: 1-211; or iii. a guide sequence at least 95%, 90%, or 85% identical to a sequence selected from SEQ ID NOs: 1-211; or iv. a guide sequence that binds a target site comprising a genomic region listed in Tables 2-5; or v. a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or vi. a guide sequence that is at least 95%, 90%, or 85% identical to a sequence selected from (v); and optionally b) an RNA-guided DNA binding agent or a nucleic acid encoding an RNA-guided DNA binding agent. In some embodiments, the methods further comprise contacting the cell with an RNA-guided DNA binding agent or a nucleic acid encoding an RNA-guided DNA binding agent. In some embodiments, the RNA-guided DNA binding agent is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase. In some embodiments, the expression of HLA-A protein on the surface of the cell (i.e., engineered cell) is thereby reduced.

In some embodiments, the methods of reducing or eliminating expression HLA-A protein on the surface of a cell comprise contacting a cell with any one or more of the HLA-A guide RNAs disclosed herein. In some embodiments, the CIITA guide RNA comprises a guide sequence selected from SEQ ID NO: 1-211.

In some embodiments, compositions are provided comprising a) an HLA-A guide RNA comprising: i. a guide sequence selected from SEQ ID NOs: 1-211; or ii. at least 17, 18, 19, or 20 contiguous nucleotides of a sequence selected from SEQ ID NOs: 1-211; or iii. a guide sequence at least 95%, 90%, or 85% identical to a sequence selected from SEQ ID NOs: 1-211; or iv. a guide sequence that binds a target site comprising a genomic region listed in Tables 2-5; or v. a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or vi. a guide sequence that is at least 95%, 90%, or 85% identical to a sequence selected from (v); and optionally b) an RNA-guided DNA binding agent or a nucleic acid encoding an RNA-guided DNA binding agent. In some embodiments, the composition further comprises an RNA-guided DNA binding agent or a nucleic acid encoding an RNA-guided DNA binding agent. In some embodiments, the composition comprises an RNA-guided DNA binding agent that is Cas9. In some embodiments, the RNA-guided DNA binding agent is S. pyogenes Cas9. In some embodiments, the CIITA guide RNA is a S. pyogenes Cas9 guide RNA. In some embodiments, the RNA-guided DNA binding agent comprises a deaminase domain. In some embodiments the RNA-guided DNA binding agent comprises an APOBEC3A deaminase (A3A) and an RNA-guided nickase.

In some embodiments, the composition further comprises a uracil glycosylase inhibitor (UGI). In some embodiments, the composition comprises an RNA-guided DNA binding agent that the RNA-guided DNA binding agent generates a cytosine (C) to thymine (T) conversion with the HLA-A genomic target sequence. In some embodiments, the composition comprises an RNA-guided DNA binding agent that generates an adenosine (A) to guanine (G) conversion with the HLA-A genomic target sequence.

In some embodiments, an engineered human cell produced by the methods described herein is provided. In some embodiments, the engineered human cell produced by the methods and compositions described herein is an allogeneic cell. In some embodiments, the methods produce a composition comprising an engineered human cell having reduced or eliminated HLA-A expression. In some embodiments, the engineered human cell produced by the methods disclosed herein elicits a reduced response from CD8+ T cells as compared to an unmodified cell as measured in an in vitro cell culture assay containing CD8+ T cells.

In some embodiments, the compositions disclosed herein further comprise a pharmaceutically acceptable carrier. In some embodiments, a cell produced by the compositions disclosed herein comprising a pharmaceutically acceptable carrier is provided. In some embodiments, compositions comprising the cells disclosed herein are provided.

### 1. HLA-A Guide RNAs

The methods and compositions provided herein disclose guide RNAs useful for reducing or eliminating the expression of HLA-A protein on the surface of a human cell. In some embodiments, such guide RNAs direct an RNA-guided DNA binding agent to an HLA-A genomic target sequence and may be referred to herein as “HLA-A guide RNAs.” In some embodiments, the HLA-A guide RNA directs an RNA-guided DNA binding agent to a human HLA-A genomic target sequence. In some embodiments, the HLA-A guide RNA comprises a guide sequence selected from SEQ ID NO: 1-211.

In some embodiments, a composition is provided comprising an HLA-A guide RNA described herein and an RNA-guided DNA binding agent or a nucleic acid encoding an RNA-guided DNA binding agent.

In some embodiments, a composition is provided comprising an HLA-A single-guide RNA (sgRNA) comprising a guide sequence selected from SEQ ID NO: 1-211. In some embodiments, a composition is provided comprising HLA-A sgRNA described herein and an RNA-guided DNA binding agent or a nucleic acid encoding an RNA-guided DNA binding agent.

In some embodiments, a composition is provided comprising an HLA-A dual-guide RNA (dgRNA) comprising a guide sequence selected from SEQ ID NO: 1-211. In some embodiments, a composition is provided comprising a HLA-a dgRNA described herein and an RNA-guided DNA binding agent or a nucleic acid encoding an RNA-guided DNA binding agent.

In some embodiments, the HLA-A gRNA comprises a guide sequence selected from any one of SEQ ID NOs: 1-211. Exemplary HLA-A guide sequences are shown below in Table 2 (SEQ ID NOs: 1-95 with corresponding guide RNA sequences SEQ ID NOs: 249-343 and 344-438), Table 3 (SEQ ID NOs: 96-128 with corresponding guide RNA sequences SEQ ID NOs: 439-471 and 472-504), Table 4 (SEQ ID NOs:129-182), and Table 5 (SEQ ID NOs: 183-211 with corresponding guide RNA sequences SEQ ID NOs: 505-532 and 533-560).

TABLE 2
Exemplary HLA-A guide RNAs

| | | | | Exemplary | |
|---|---|---|---|---|---|
| | | | | Guide RNA | |
| | | | | Modified | |
| | | | | Sequence | |
| | | | Exemplary | (four terminal U | |
| | | | Guide RNA | residues are | |
| | | | Full | optional and may | |
| | SEQ ID | | Sequence | include 0, 1, 2, | |
| | NO to the | | (SEQ ID | 3, 4, or more Us) | Genomic |
| Guide | Guide | Guide | NOS: 249- | (SEQ ID NOS: | Coordinates |
| ID | Sequence | Sequence | 343) | 344-438) | (hg38) |
| G018983 | 1 | UGGAGGGC | UGGAGGGC | mU*mG*mG*A | chr6:29945290- |
| | | CUGAUGUG | CUGAUGUG | GGGCCUGAUG | 29945310 |
| | | UGUU | UGUUGUUU | UGUGUUGUUU | (mismatch to |
| | | | UAGAGCUA | UAGAmGmCmU | hg38 = 2) |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018984 | 2 | GCCUGAUG | GCCUGAUG | mG*mC*mC*UG | chr6:29945296- |
| | | UGUGUUGG | UGUGUUGG | AUGUGUGUUG | 29945316 |
| | | GUGU | GUGUGUUU | GGUGUGUUUU | (mismatch to |
| | | | UAGAGCUA | AGAmGmCmU | hg38 = 2) |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018985 | 3 | CCUGAUGU | CCUGAUGU | mC*mC*mU*GA | chr6:29945297- |
| | | GUGUUGGG | GUGUUGGG | UGUGUGUUGG | 29945317 |
| | | UGUU | UGUUGUUU | GUGUUGUUUU | (mismatch to |
| | | | UAGAGCUA | AGAmGmCmU | hg38 = 1) |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018986 | 4 | CCCAACAC | CCCAACAC | mC*mC*mC*AA | chr6:29945300- |
| | | CCAACACA | CCAACACA | CACCCAACAC | 29945320 |
| | | CAUC | CAUCGUUU | ACAUCGUUUU | (mismatch to |
| | | | UAGAGCUA | AGAmGmCmU | hg38 = 1) |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018965 | 5 | UCAGGAAA | UCAGGAAA | mU*mC*mA*G | chr6:29890117- |
| | | CAUGAAGA | CAUGAAGA | GAAACAUGAA | 29890137 |
| | | AAGC | AAGCGUUU | GAAAGCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019018 | 6 | AGGCGCCU | AGGCGCCU | mA*mG*mG*C | chr6:29927058- |
| | | GGGCCUCU | GGGCCUCU | GCCUGGGCCU | 29927078 |
| | | CCCG | CCCGGUUU | CUCCCGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018937 | 7 | CGGGCUGG | CGGGCUGG | mC*mG*mG*GC | chr6:29934330- |
| | | CCUCCCAC | CCUCCCAC | UGGCCUCCCA | 29934350 |
| | | AAGG | AAGGGUUU | CAAGGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018990 | 8 | ACGGCCAU | ACGGCCAU | mA*mC*mG*GC | chr6:29942541- |
| | | CCUCGGCG | CCUCGGCG | CAUCCUCGGC | 29942561 |
| | | UCUG | UCUGGUUU | GUCUGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018991 | 9 | GACGGCCA | GACGGCCA | mG*mA*mC*G | chr6:29942542- |
| | | UCCUCGGC | UCCUCGGC | GCCAUCCUCG | 29942562 |
| | | GUCU | GUCUGUUU | GCGUCUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018992 | 10 | GACGCCGA | GACGCCGA | mG*mA*mC*GC | chr6:29942543- |
| | | GGAUGGCC | GGAUGGCC | CGAGGAUGGC | 29942563 |
| | | GUCA | GUCAGUUU | CGUCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018993 | 11 | UGACGGCC | UGACGGCC | mU*mG*mA*C | chr6:29942543- |
| | | AUCCUCGG | AUCCUCGG | GGCCAUCCUC | 29942563 |
| | | CGUC | CGUCGUUU | GGCGUCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018994 | 12 | GGCGCCAU | GGCGCCAU | mG*mG*mC*GC | chr6:29942550- |
| | | GACGGCCA | GACGGCCA | CAUGACGGCC | 29942570 |
| | | UCCU | UCCUGUUU | AUCCUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018995 | 13 | ACAGCGAC | ACAGCGAC | mA*mC*mA*GC | chr6:29942864- |
| | | GCCGCGAG | GCCGCGAG | GACGCCGCGA | 29942884 |
| | | CCAG | CCAGGUUU | GCCAGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018996 | 14 | CGACGCCG | CGACGCCG | mC*mG*mA*CG | chr6:29942868- |
| | | CGAGCCAG | CGAGCCAG | CCGCGAGCCA | 29942888 |
| | | AGGA | AGGAGUUU | GAGGAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018997 | 15 | CGAGCCAG | CGAGCCAG | mC*mG*mA*GC | chr6:29942876- |
| | | AGGAUGGA | AGGAUGGA | CAGAGGAUGG | 29942896 |
| | | GCCG | GCCGGUUU | AGCCGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018998 | 16 | CGGCUCCA | CGGCUCCA | mC*mG*mG*CU | chr6:29942876- |
| | | UCCUCUGG | UCCUCUGG | CCAUCCUCUG | 29942896 |
| | | CUCG | CUCGGUUU | GCUCGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018999 | 17 | GAGCCAGA | GAGCCAGA | mG*mA*mG*CC | chr6:29942877- |
| | | GGAUGGAG | GGAUGGAG | AGAGGAUGGA | 29942897 |
| | | CCGC | CCGCGUUU | GCCGCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019000 | 18 | GCGCCCGC | GCGCCCGC | mG*mC*mG*CC | chr6:29942883- |
| | | GGCUCCAU | GGCUCCAU | CGCGGCUCCA | 29942903 |
| | | CCUC | CCUCGUUU | UCCUCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019001 | 19 | GCCCGUCC | GCCCGUCC | mG*mC*mC*CG | chr6:29943062- |
| | | GUGGGGGA | GUGGGGGA | UCCGUGGGGG | 29943082 |
| | | UGAG | UGAGGUUU | AUGAGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019002 | 20 | UCAUCCCC | UCAUCCCC | mU*mC*mA*UC | chr6:29943063- |
| | | CACGGACG | CACGGACG | CCCCACGGAC | 29943083 |
| | | GGCC | GGCCGUUU | GGGCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019003 | 21 | AUCUCGGA | AUCUCGGA | mA*mU*mC*UC | chr6:29943092- |
| | | CCCGGAGA | CCCGGAGA | GGACCCGGAG | 29943112 |
| | | CUGU | CUGUGUUU | ACUGUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019004 | 22 | GGGGUCCC | GGGGUCCC | mG*mG*mG*G | chr6:29943115- |
| | | GCGGCUUC | GCGGCUUC | UCCCGCGGCU | 29943135 |
| | | GGGG | GGGGGUUU | UCGGGGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019005 | 23 | CUCGGGGU | CUCGGGGU | mC*mU*mC*GG | chr6:29943118- |
| | | CCCGCGGC | CCCGCGGC | GGUCCCGCGG | 29943138 |
| | | UUCG | UUCGGUUU | CUUCGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019006 | 24 | UCUCGGGG | UCUCGGGG | mU*mC*mU*CG | chr6:29943119- |
| | | UCCCGCGG | UCCCGCGG | GGGUCCCGCG | 29943139 |
| | | CUUC | CUUCGUUU | GCUUCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019007 | 25 | GUCUCGGG | GUCUCGGG | mG*mU*mC*UC | chr6:29943120- |
| | | GUCCCGCG | GUCCCGCG | GGGGUCCCGC | 29943140 |
| | | GCUU | GCUUGUUU | GGCUUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019008 | 26 | GCAAGGGU | GCAAGGGU | mG*mC*mA*A | chr6:29943126- |
| | | CUCGGGGU | CUCGGGGU | GGGUCUCGGG | 29943146 |
| | | CCCG | CCCGGUUU | GUCCCGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019009 | 27 | GGACCCCG | GGACCCCG | mG*mG*mA*CC | chr6:29943128- |
| | | AGACCCUU | AGACCCUU | CCGAGACCCU | 29943148 |
| | | GCCC | GCCCGUUU | UGCCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019010 | 28 | GACCCCGA | GACCCCGA | mG*mA*mC*CC | chr6:29943129- |
| | | GACCCUUG | GACCCUUG | CGAGACCCUU | 29943149 |
| | | CCCC | CCCCGUUU | GCCCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019011 | 29 | CGAGACCC | CGAGACCC | mC*mG*mA*G | chr6:29943134- |
| | | UUGCCCCG | UUGCCCCG | ACCCUUGCCC | 29943154 |
| | | GGAG | GGAGGUUU | CGGGAGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019012 | 30 | CUCCCGGG | CUCCCGGG | mC*mU*mC*CC | chr6:29943134- |
| | | GCAAGGGU | GCAAGGGU | GGGGCAAGGG | 29943154 |
| | | CUCG | CUCGGUUU | UCUCGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019013 | 31 | UCUCCCGG | UCUCCCGG | mU*mC*mU*CC | chr6:29943135- |
| | | GGCAAGGG | GGCAAGGG | CGGGGCAAGG | 29943155 |
| | | UCUC | UCUCGUUU | GUCUCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019014 | 32 | CUCUCCCG | CUCUCCCG | mC*mU*mC*UC | chr6:29943136- |
| | | GGGCAAGG | GGGCAAGG | CCGGGGCAAG | 29943156 |
| | | GUCU | GUCUGUUU | GGUCUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019015 | 33 | CCUUGCCC | CCUUGCCC | mC*mC*mU*UG | chr6:29943140- |
| | | CGGGAGAG | CGGGAGAG | CCCCGGGAGA | 29943160 |
| | | GCCC | GCCCGUUU | GGCCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019016 | 34 | CUGGGCCU | CUGGGCCU | mC*mU*mG*G | chr6:29943142- |
| | | CUCCCGGG | CUCCCGGG | GCCUCUCCCG | 29943162 |
| | | GCAA | GCAAGUUU | GGGCAAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019017 | 35 | CCUGGGCC | CCUGGGCC | mC*mC*mU*GG | chr6:29943143- |
| | | UCUCCCGG | UCUCCCGG | GCCUCUCCCG | 29943163 |
| | | GGCA | GGCAGUUU | GGGCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G019019 | 36 | UUUAGGCC | UUUAGGCC | mU*mU*mU*A | chr6:29943188- |
| | | AAAAAUCC | AAAAAUCC | GGCCAAAAAU | 29943208 |
| | | CCCC | CCCCGUUU | CCCCCCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021208 | 37 | CGCUGCAG | CGCUGCAG | mC*mG*mC*UG | chr6:29943528- |
| | | CGCACGGG | CGCACGGG | CAGCGCACGG | 29943548 |
| | | UACC | UACCGUUU | GUACCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021209 | 38 | GCUGCAGC | GCUGCAGC | mG*mC*mU*GC | chr6:29943529- |
| | | GCACGGGU | GCACGGGU | AGCGCACGGG | 29943549 |
| | | ACCA | ACCAGUUU | UACCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021210 | 39 | CUGCAGCG | CUGCAGCG | mC*mU*mG*CA | chr6:29943530- |
| | | CACGGGUA | CACGGGUA | GCGCACGGGU | 29943550 |
| | | CCAG | CCAGGUUU | ACCAGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018932 | 40 | CGCACGGG | CGCACGGG | mC*mG*mC*AC | chr6:29943536- |
| | | UACCAGGG | UACCAGGG | GGGUACCAGG | 29943556 |
| | | GCCA | GCCAGUUU | GGCCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018933 | 41 | GCACGGGU | GCACGGGU | mG*mC*mA*CG | chr6:29943537- |
| | | ACCAGGGG | ACCAGGGG | GGUACCAGGG | 29943557 |
| | | CCAC | CCACGUUU | GCCACGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018934 | 42 | CACGGGUA | CACGGGUA | mC*mA*mC*GG | chr6:29943538- |
| | | CCAGGGGC | CCAGGGGC | GUACCAGGGG | 29943558 |
| | | CACG | CACGGUUU | CCACGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018935 | 43 | GGGAGGCG | GGGAGGCG | mG*mG*mG*A | chr6:29943549- |
| | | CCCCGUGG | CCCCGUGG | GGCGCCCCGU | 29943569 |
| | | CCCC | CCCCGUUU | GGCCCCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018936 | 44 | GCGAUCAG | GCGAUCAG | mG*mC*mG*A | chr6:29943556- |
| | | GGAGGCGC | GGAGGCGC | UCAGGGAGGC | 29943576 |
| | | CCCG | CCCGGUUU | GCCCCGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021211 | 45 | UCCUUGUG | UCCUUGUG | mU*mC*mC*UU | chr6:29943589- |
| | | GGAGGCCA | GGAGGCCA | GUGGGAGGCC | 29943609 |
| | | GCCC | GCCCGUUU | AGCCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018938 | 46 | CUCCUUGU | CUCCUUGU | mC*mU*mC*CU | chr6:29943590- |
| | | GGGAGGCC | GGGAGGCC | UGUGGGAGGC | 29943610 |
| | | AGCC | AGCCGUUU | CAGCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018939 | 47 | GGCUGGCC | GGCUGGCC | mG*mG*mC*U | chr6:29943590- |
| | | UCCCACAA | UCCCACAA | GGCCUCCCAC | 29943610 |
| | | GGAG | GGAGGUUU | AAGGAGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018940 | 48 | UUGUCUCC | UUGUCUCC | mU*mU*mG*U | chr6:29943599- |
| | | CCUCCUUG | CCUCCUUG | CUCCCCUCCU | 29943619 |
| | | UGGG | UGGGGUUU | UGUGGGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018941 | 49 | CCACAAGG | CCACAAGG | mC*mC*mA*CA | chr6:29943600- |
| | | AGGGGAGA | AGGGGAGA | AGGAGGGGAG | 29943620 |
| | | CAAU | CAAUGUUU | ACAAUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018942 | 50 | CACAAGGA | CACAAGGA | mC*mA*mC*AA | chr6:29943601- |
| | | GGGGAGAC | GGGGAGAC | GGAGGGGAGA | 29943621 |
| | | AAUU | AAUUGUUU | CAAUUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018943 | 51 | CAAUUGUC | CAAUUGUC | mC*mA*mA*U | chr6:29943602- |
| | | UCCCCUCC | UCCCCUCC | UGUCUCCCCU | 29943622 |
| | | UUGU | UUGUGUUU | CCUUGUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018944 | 52 | CCAAUUGU | CCAAUUGU | mC*mC*mA*AU | chr6:29943603- |
| | | CUCCCCUC | CUCCCCUC | UGUCUCCCCU | 29943623 |
| | | CUUG | CUUGGUUU | CCUUGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018945 | 53 | AUCCCUCG | AUCCCUCG | mA*mU*mC*CC | chr6:29943774- |
| | | AAUACUGA | AAUACUGA | UCGAAUACUG | 29943794 |
| | | UGAG | UGAGGUUU | AUGAGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018946 | 54 | AACCACUC | AACCACUC | mA*mA*mC*CA | chr6:29943779- |
| | | AUCAGUAU | AUCAGUAU | CUCAUCAGUA | 29943799 |
| | | UCGA | UCGAGUUU | UUCGAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018947 | 55 | GAACCACU | GAACCACU | mG*mA*mA*CC | chr6:29943780- |
| | | CAUCAGUA | CAUCAGUA | ACUCAUCAGU | 29943800 |
| | | UUCG | UUCGGUUU | AUUCGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018948 | 56 | GAGGAAAA | GAGGAAAA | mG*mA*mG*G | chr6:29943822- |
| | | GUCACGGG | GUCACGGG | AAAAGUCACG | 29943842 |
| | | CCCA | CCCAGUUU | GGCCCAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018949 | 57 | GGCCCGUG | GGCCCGUG | mG*mG*mC*CC | chr6:29943824- |
| | | ACUUUUCC | ACUUUUCC | GUGACUUUUC | 29943844 |
| | | UCUC | UCUCGUUU | CUCUCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018950 | 58 | UGCUUCAC | UGCUUCAC | mU*mG*mC*U | chr6:29943857- |
| | | ACUCAAUG | ACUCAAUG | UCACACUCAA | 29943877 |
| | | UGUG | UGUGGUUU | UGUGUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018951 | 59 | GCUUCACA | GCUUCACA | mG*mC*mU*UC | chr6:29943858- |
| | | CUCAAUGU | CUCAAUGU | ACACUCAAUG | 29943878 |
| | | GUGU | GUGUGUUU | UGUGUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018952 | 60 | CUUCACAC | CUUCACAC | mC*mU*mU*CA | chr6:29943859- |
| | | UCAAUGUG | UCAAUGUG | CACUCAAUGU | 29943879 |
| | | UGUG | UGUGGUUU | GUGUGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018953 | 61 | UUCACACU | UUCACACU | mU*mU*mC*AC | chr6:29943860- |
| | | CAAUGUGU | CAAUGUGU | ACUCAAUGUG | 29943880 |
| | | GUGG | GUGGGUUU | UGUGGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018954 | 62 | UUGAGAAU | UUGAGAAU | mU*mU*mG*A | chr6:29944026- |
| | | GGACAGGA | GGACAGGA | GAAUGGACAG | 29944046 |
| | | CACC | CACCGUUU | GACACCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021205 | 63 | AGGCAUUU | AGGCAUUU | mA*mG*mG*C | chr6:29944077- |
| | | UGCAUCUG | UGCAUCUG | AUUUUGCAUC | 29944097 |
| | | UCAU | UCAUGUUU | UGUCAUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021206 | 64 | CAGGCAUU | CAGGCAUU | mC*mA*mG*GC | chr6:29944078- |
| | | UUGCAUCU | UUGCAUCU | AUUUUGCAUC | 29944098 |
| | | GUCA | GUCAGUUU | UGUCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018955 | 65 | AGGGGCCC | AGGGGCCC | mA*mG*mG*G | chr6:29944458- |
| | | UGACCCUG | UGACCCUG | GCCCUGACCC | 29944478 |
| | | CUAA | CUAAGUUU | UGCUAAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018956 | 66 | UGGGAAAA | UGGGAAAA | mU*mG*mG*G | chr6:29944478- |
| | | GAGGGGAA | GAGGGGAA | AAAAGAGGGG | 29944498 |
| | | GGUG | GGUGGUUU | AAGGUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018957 | 67 | UGGAGGAG | UGGAGGAG | mU*mG*mG*A | chr6:29944597- |
| | | GAAGAGCU | GAAGAGCU | GGAGGAAGAG | 29944617 |
| | | CAGG | CAGGGUUU | CUCAGGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018958 | 68 | UGAGAUUU | UGAGAUUU | mU*mG*mA*G | chr6:29944642- |
| | | CUUGUCUC | CUUGUCUC | AUUUCUUGUC | 29944662 |
| | | ACUG | ACUGGUUU | UCACUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018959 | 69 | GAGAUUUC | GAGAUUUC | mG*mA*mG*A | chr6:29944643- |
| | | UUGUCUCA | UUGUCUCA | UUUCUUGUCU | 29944663 |
| | | CUGA | CUGAGUUU | CACUGAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018960 | 70 | UAAAGCAC | UAAAGCAC | mU*mA*mA*A | chr6:29944772- |
| | | CUGUUAAA | CUGUUAAA | GCACCUGUUA | 29944792 |
| | | AUGA | AUGAGUUU | AAAUGAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018961 | 71 | AAUCUGUC | AAUCUGUC | mA*mA*mU*C | chr6:29944782- |
| | | CUUCAUUU | CUUCAUUU | UGUCCUUCAU | 29944802 |
| | | UAAC | UAACGUUU | UUUAACGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018962 | 72 | GUCACAGG | GUCACAGG | mG*mU*mC*AC | chr6:29944850- |
| | | GGAAGGUC | GGAAGGUC | AGGGGAAGGU | 29944870 |
| | | CCUG | CCUGGUUU | CCCUGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018964 | 73 | AAACAUGA | AAACAUGA | mA*mA*mA*C | chr6:29944907- |
| | | AGAAAGCA | AGAAAGCA | AUGAAGAAAG | 29944927 |
| | | GGUG | GGUGGUUU | CAGGUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018966 | 74 | UGUCCUGU | UGUCCUGU | mU*mG*mU*CC | chr6:29945024- |
| | | GAGAUACC | GAGAUACC | UGUGAGAUAC | 29945044 |
| | | AGAA | AGAAGUUU | CAGAAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018967 | 75 | AUGAAGGA | AUGAAGGA | mA*mU*mG*A | chr6:29945097- |
| | | GGCUGAUG | GGCUGAUG | AGGAGGCUGA | 29945117 |
| | | CCUG | CCUGGUUU | UGCCUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018968 | 76 | AGGCUGAU | AGGCUGAU | mA*mG*mG*C | chr6:29945104- |
| | | GCCUGAGG | GCCUGAGG | UGAUGCCUGA | 29945124 |
| | | UCCU | UCCUGUUU | GGUCCUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018969 | 77 | GGCUGAUG | GGCUGAUG | mG*mG*mC*U | chr6:29945105- |
| | | CCUGAGGU | CCUGAGGU | GAUGCCUGAG | 29945125 |
| | | CCUU | CCUUGUUU | GUCCUUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018970 | 78 | CACAAUAU | CACAAUAU | mC*mA*mC*AA | chr6:29945116- |
| | | CCCAAGGA | CCCAAGGA | UAUCCCAAGG | 29945136 |
| | | CCUC | CCUCGUUU | ACCUCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018971 | 79 | GGUCCUUG | GGUCCUUG | mG*mG*mU*CC | chr6:29945118- |
| | | GGAUAUUG | GGAUAUUG | UUGGGAUAUU | 29945138 |
| | | UGUU | UGUUGUUU | GUGUUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018972 | 80 | GUCCUUGG | GUCCUUGG | mG*mU*mC*CU | chr6:29945119- |
| | | GAUAUUGU | GAUAUUGU | UGGGAUAUUG | 29945139 |
| | | GUUU | GUUUGUUU | UGUUUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018973 | 81 | CUCCCAAA | CUCCCAAA | mC*mU*mC*CC | chr6:29945124- |
| | | CACAAUAU | CACAAUAU | AAACACAAUA | 29945144 |
| | | CCCA | CCCAGUUU | UCCCAGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018974 | 82 | UCCUCUAG | UCCUCUAG | mU*mC*mC*UC | chr6:29945176- |
| | | CCACAUCU | CCACAUCU | UAGCCACAUC | 29945196 |
| | | UCUG | UCUGGUUU | UUCUGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018975 | 83 | ACAGAAGA | ACAGAAGA | mA*mC*mA*G | chr6:29945177- |
| | | UGUGGCUA | UGUGGCUA | AAGAUGUGGC | 29945197 |
| | | GAGG | GAGGGUUU | UAGAGGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018976 | 84 | CCUCUAGC | CCUCUAGC | mC*mC*mU*CU | chr6:29945177- |
| | | CACAUCUU | CACAUCUU | AGCCACAUCU | 29945197 |
| | | CUGU | CUGUGUUU | UCUGUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018977 | 85 | CCCACAGA | CCCACAGA | mC*mC*mC*AC | chr6:29945180- |
| | | AGAUGUGG | AGAUGUGG | AGAAGAUGUG | 29945200 |
| | | CUAG | CUAGGUUU | GCUAGGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018978 | 86 | GUCAGAUC | GUCAGAUC | mG*mU*mC*A | chr6:29945187- |
| | | CCACAGAA | CCACAGAA | GAUCCCACAG | 29945207 |
| | | GAUG | GAUGGUUU | AAGAUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018979 | 87 | AUCUUCUG | AUCUUCUG | mA*mU*mC*U | chr6:29945188- |
| | | UGGGAUCU | UGGGAUCU | UCUGUGGGAU | 29945208 |
| | | GACC | GACCGUUU | CUGACCGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018980 | 88 | CCCAGGCA | CCCAGGCA | mC*mC*mC*AG | chr6:29945228- |
| | | GUGACAGU | GUGACAGU | GCAGUGACAG | 29945248 |
| | | GCCC | GCCCGUUU | UGCCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018981 | 89 | CUGGGCAC | CUGGGCAC | mC*mU*mG*G | chr6:29945230- |
| | | UGUCACUG | UGUCACUG | GCACUGUCAC | 29945250 |
| | | CCUG | CCUGGUUU | UGCCUGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018982 | 90 | CCUGGGCA | CCUGGGCA | mC*mC*mU*GG | chr6:29945231- |
| | | CUGUCACU | CUGUCACU | GCACUGUCAC | 29945251 |
| | | GCCU | GCCUGUUU | UGCCUGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G021207 | 91 | CCCUGGGC | CCCUGGGC | mC*mC*mC*UG | chr6:29945232- |
| | | ACUGUCAC | ACUGUCAC | GGCACUGUCA | 29945252 |
| | | UGCC | UGCCGUUU | CUGCCGUUUU | |
| | | | UAGAGCUA | AGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018987 | 92 | UUGGGUGU | UUGGGUGU | mU*mU*mG*G | chr6:29945308- |
| | | UGGGCGGA | UGGGCGGA | GUGUUGGGCG | 29945328 |
| | | ACAG | ACAGGUUU | GAACAGGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018988 | 93 | UGGAUGUA | UGGAUGUA | mU*mG*mG*A | chr6:29945361- |
| | | UUGAGCAU | UUGAGCAU | UGUAUUGAGC | 29945381 |
| | | GCGA | GCGAGUUU | AUGCGAGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018989 | 94 | GGAUGUAU | GGAUGUAU | mG*mG*mA*U | chr6:29945362- |
| | | UGAGCAUG | UGAGCAUG | GUAUUGAGCA | 29945382 |
| | | CGAU | CGAUGUUU | UGCGAUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |
| G018963 | 95 | AACAUGAA | AACAUGAA | mA*mA*mC*A | chr6:31382543- |
| | | GAAAGCAG | GAAAGCAG | UGAAGAAAGC | 31382563 |
| | | GUGU | GUGUGUUU | AGGUGUGUUU | |
| | | | UAGAGCUA | UAGAmGmCmU | |
| | | | GAAAUAGC | mAmGmAmAm | |
| | | | AAGUUAAA | AmUmAmGmC | |
| | | | AUAAGGCU | AAGUUAAAAU | |
| | | | AGUCCGUU | AAGGCUAGUC | |
| | | | AUCAACUU | CGUUAUCAmA | |
| | | | GAAAAAGU | mCmUmUmGm | |
| | | | GGCACCGA | AmAmAmAmA | |
| | | | GUCGGUGC | mGmUmGmGm | |
| | | | UUUU | CmAmCmCmGm | |
| | | | | AmGmUmCmG | |
| | | | | mGmUmGmCm | |
| | | | | U*mU*mU*mU | |

TABLE 3
Additional Exemplary S. pyogenes HLA-A guide RNAs

| | | | | Exemplary | |
|---|---|---|---|---|---|
| | | | | Guide RNA | |
| | | | Exemplary | Modified Sequence | |
| | | | Guide RNA | (four terminal | |
| | | | Full | U residues are | |
| | | | Sequence | optional and | |
| | SEQ ID | | with PAM | may include 0, | |
| | NO to the | | (SEQ ID | 1, 2, 3, 4, or | |
| Guide | Guide | Guide | NOS: | more Us) (SEQ ID | Genomic |
| ID | Sequence | Sequence | 439-471) | NOS: 472-504) | Coordinates |
| G021885 | 96 | UAGCCCAC | UAGCCCAC | mU*mA*mG* | chr6:29942815- |
| | | GGCGAUGA | GGCGAUGA | CCCACGGCG | 29942835 |
| | | AGCG | AGCGGUUU | AUGAAGCGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021886 | 97 | GUAGCCCA | GUAGCCCA | mG*mU*mA* | chr6:29942816- |
| | | CGGCGAUG | CGGCGAUG | GCCCACGGC | 29942836 |
| | | AAGC | AAGCGUUU | GAUGAAGCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021887 | 98 | CGUAGCCC | CGUAGCCC | mC*mG*mU* | chr6:29942817- |
| | | ACGGCGAU | ACGGCGAU | AGCCCACGG | 29942837 |
| | | GAAG | GAAGGUUU | CGAUGAAGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021888 | 99 | CUUCAUCG | CUUCAUCG | mC*mU*mU* | chr6:29942817- |
| | | CCGUGGGC | CCGUGGGC | CAUCGCCGU | 29942837 |
| | | UACG | UACGGUUU | GGGCUACGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021889 | 100 | CGUGUCGU | CGUGUCGU | mC*mG*mU* | chr6:29942828- |
| | | CCACGUAG | CCACGUAG | GUCGUCCAC | 29942848 |
| | | CCCA | CCCAGUUU | GUAGCCCAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021890 | 101 | UGGACGAC | UGGACGAC | mU*mG*mG* | chr6:29942837- |
| | | ACGCAGUU | ACGCAGUU | ACGACACGC | 29942857 |
| | | CGUG | CGUGGUUU | AGUUCGUGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021891 | 102 | GGAUGGAG | GGAUGGAG | mG*mG*mA* | chr6:29942885- |
| | | CCGCGGGC | CCGCGGGC | UGGAGCCGC | 29942905 |
| | | GCCG | GCCGGUUU | GGGCGCCGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021892 | 103 | GCGGGCGC | GCGGGCGC | mG*mC*mG* | chr6:29942895- |
| | | CGUGGAUA | CGUGGAUA | GGCGCCGUG | 29942915 |
| | | GAGC | GAGCGUUU | GAUAGAGCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021893 | 104 | UGCUCUAU | UGCUCUAU | mU*mG*mC* | chr6:29942896- |
| | | CCACGGCG | CCACGGCG | UCUAUCCAC | 29942916 |
| | | CCCG | CCCGGUUU | GGCGCCCGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021894 | 105 | GGCGCCGU | GGCGCCGU | mG*mG*mC* | chr6:29942898- |
| | | GGAUAGAG | GGAUAGAG | GCCGUGGAU | 29942918 |
| | | CAGG | CAGGGUUU | AGAGCAGGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021895 | 106 | GCGCCGUG | GCGCCGUG | mG*mC*mG* | chr6:29942899- |
| | | GAUAGAGC | GAUAGAGC | CCGUGGAUA | 29942919 |
| | | AGGA | AGGAGUUU | GAGCAGGAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021896 | 107 | CGCCGUGG | CGCCGUGG | mC*mG*mC*C | chr6:29942900- |
| | | AUAGAGCA | AUAGAGCA | GUGGAUAGA | 29942920 |
| | | GGAG | GGAGGUUU | GCAGGAGGU | |
| | | | UAGAGCUA | UUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021897 | 108 | GUGGAUAG | GUGGAUAG | mG*mU*mG* | chr6:29942904- |
| | | AGCAGGAG | AGCAGGAG | GAUAGAGCA | 29942924 |
| | | GGGC | GGGCGUUU | GGAGGGGCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021898 | 109 | GGCCCCUC | GGCCCCUC | mG*mG*mC* | chr6:29942905- |
| | | CUGCUCUA | CUGCUCUA | CCCUCCUGC | 29942925 |
| | | UCCA | UCCAGUUU | UCUAUCCAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021899 | 110 | AGCAGGAG | AGCAGGAG | mA*mG*mC* | chr6:29942912- |
| | | GGGCCGGA | GGGCCGGA | AGGAGGGGC | 29942932 |
| | | GUAU | GUAUGUUU | CGGAGUAUG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021900 | 111 | GCAGGAGG | GCAGGAGG | mG*mC*mA* | chr6:29942913- |
| | | GGCCGGAG | GGCCGGAG | GGAGGGGCC | 29942933 |
| | | UAUU | UAUUGUUU | GGAGUAUUG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021901 | 112 | GGAGUGGC | GGAGUGGC | mG*mG*mA* | chr6:29943490- |
| | | UCCGCAGA | UCCGCAGA | GUGGCUCCG | 29943510 |
| | | UACC | UACCGUUU | CAGAUACCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021902 | 113 | CUCCGCAG | CUCCGCAG | mC*mU*mC*C | chr6:29943497- |
| | | AUACCUGG | AUACCUGG | GCAGAUACC | 29943517 |
| | | AGAA | AGAAGUUU | UGGAGAAGU | |
| | | | UAGAGCUA | UUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021903 | 114 | UCCGCAGA | UCCGCAGA | mU*mC*mC* | chr6:29943498- |
| | | UACCUGGA | UACCUGGA | GCAGAUACC | 29943518 |
| | | GAAC | GAACGUUU | UGGAGAACG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021904 | 115 | CAGAUACC | CAGAUACC | mC*mA*mG* | chr6:29943502- |
| | | UGGAGAAC | UGGAGAAC | AUACCUGGA | 29943522 |
| | | GGGA | GGGAGUUU | GAACGGGAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021905 | 116 | UCCCGUUC | UCCCGUUC | mU*mC*mC*C | chr6:29943502- |
| | | UCCAGGUA | UCCAGGUA | GUUCUCCAG | 29943522 |
| | | UCUG | UCUGGUUU | GUAUCUGGU | |
| | | | UAGAGCUA | UUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021906 | 117 | GCGUCUCC | GCGUCUCC | mG*mC*mG* | chr6:29943511- |
| | | UUCCCGUU | UUCCCGUU | UCUCCUUCC | 29943531 |
| | | CUCC | CUCCGUUU | CGUUCUCCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021907 | 118 | GAAGGAGA | GAAGGAGA | mG*mA*mA* | chr6:29943520- |
| | | CGCUGCAG | CGCUGCAG | GGAGACGCU | 29943540 |
| | | CGCA | CGCAGUUU | GCAGCGCAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021908 | 119 | AAGGAGAC | AAGGAGAC | mA*mA*mG* | chr6:29943521- |
| | | GCUGCAGC | GCUGCAGC | GAGACGCUG | 29943541 |
| | | GCAC | GCACGUUU | CAGCGCACG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021909 | 120 | AGAUCUAC | AGAUCUAC | mA*mG*mA* | chr6:29943566- |
| | | AGGCGAUC | AGGCGAUC | UCUACAGGC | 29943586 |
| | | AGGG | AGGGGUUU | GAUCAGGGG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021910 | 121 | UGAUCGCC | UGAUCGCC | mU*mG*mA* | chr6:29943569- |
| | | UGUAGAUC | UGUAGAUC | UCGCCUGUA | 29943589 |
| | | UCCC | UCCCGUUU | GAUCUCCCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021911 | 122 | GGGAGAUC | GGGAGAUC | mG*mG*mG* | chr6:29943569- |
| | | UACAGGCG | UACAGGCG | AGAUCUACA | 29943589 |
| | | AUCA | AUCAGUUU | GGCGAUCAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021912 | 123 | CGGGAGAU | CGGGAGAU | mC*mG*mG* | chr6:29943570- |
| | | CUACAGGC | CUACAGGC | GAGAUCUAC | 29943590 |
| | | GAUC | GAUCGUUU | AGGCGAUCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021913 | 124 | CGCCUGUA | CGCCUGUA | mC*mG*mC*C | chr6:29943573- |
| | | GAUCUCCC | GAUCUCCC | UGUAGAUCU | 29943593 |
| | | GGGC | GGGCGUUU | CCCGGGCGU | |
| | | | UAGAGCUA | UUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021914 | 125 | GGCCAGCC | GGCCAGCC | mG*mG*mC* | chr6:29943578- |
| | | CGGGAGAU | CGGGAGAU | CAGCCCGGG | 29943598 |
| | | CUAC | CUACGUUU | AGAUCUACG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021915 | 126 | UCCCGGGC | UCCCGGGC | mU*mC*mC*C | chr6:29943585- |
| | | UGGCCUCC | UGGCCUCC | GGGCUGGCC | 29943605 |
| | | CACA | CACAGUUU | UCCCACAGU | |
| | | | UAGAGCUA | UUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021916 | 127 | GGGCUGGC | GGGCUGGC | mG*mG*mG* | chr6:29943589- |
| | | CUCCCACA | CUCCCACA | CUGGCCUCC | 29943609 |
| | | AGGA | AGGAGUUU | CACAAGGAG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |
| G021917 | 128 | CUGAUCGC | CUGAUCGC | mC*mU*mG* | chr6:29943568- |
| | | CUGUAGAU | CUGUAGAU | AUCGCCUGU | 29943588 |
| | | CUCC | CUCCGUUU | AGAUCUCCG | |
| | | | UAGAGCUA | UUUUAGAmG | |
| | | | GAAAUAGC | mCmUmAmG | |
| | | | AAGUUAAA | mAmAmAmU | |
| | | | AUAAGGCU | mAmGmCAA | |
| | | | AGUCCGUU | GUUAAAAUA | |
| | | | AUCAACUU | AGGCUAGUC | |
| | | | GAAAAAGU | CGUUAUCAm | |
| | | | GGCACCGA | AmCmUmUm | |
| | | | GUCGGUGC | GmAmAmAm | |
| | | | UUUU | AmAmGmUm | |
| | | | | GmGmCmAm | |
| | | | | CmCmGmAm | |
| | | | | GmUmCmGm | |
| | | | | GmUmGmCm | |
| | | | | U*mU*mU*m | |
| | | | | U | |

*The guide sequence disclosed in this Table may be unmodified, modified with the exemplary modification pattern shown in the Table, or modified with a different modification pattern disclosed herein or available in the art.

TABLE 4
Exemplary HLA-A guide sequences

| SEQ | | | | Genomic |
|---|---|---|---|---|
| ID | | | RNA-guided DNA | Coordinates |
| NO | Guide Sequence | PAM | binding agent | (hg38) |
| 129 | AGGAUGGAGCCGCGGGC | GTGG | S. aureus Cas9 | chr6:29942884- |
| | GCC | AT | | 29942904 |
| 130 | GGAAGGAGACGCUGCA | ACGG | S. aureus Cas9 | chr6:29943519- |
| | GCGC | GT | | 29943539 |
| 131 | GACAGCGACGCCGCGAG | GAGG | S. aureus Cas9 | chr6:29942863- |
| | CCA | AT | | 29942883 |
| 132 | CGGGAAGGAGACGCUGC | TTCT | CasX | chr6:29943517- |
| | AGC | | | 29943537 |
| 133 | CCGUGCGCUGCAGCGUC | TTCC | CasX | chr6:29943523- |
| | UCC | | | 29943543 |
| 134 | ACGCAGUUCGUGCGGUU | NNNN | NME2 | chr6:29942845- |
| | CGACAGC | CC | | 29942869 |
| 135 | UCGUGCGGUUCGACAGC | NNNN | NME2 | chr6:29942852- |
| | GACGCCG | CC | | 29942876 |
| 136 | CAGCGACGCCGCGAGCC | NNNN | NME2 | chr6:29942865- |
| | AGAGGAU | CC | | 29942889 |
| 137 | GCUCUAUCCACGGCGCC | NNNN | NME2 | chr6:29942891- |
| | CGCGGCU | CC | | 29942915 |
| 138 | UCCUGCUCUAUCCACGG | NNNN | NME2 | chr6:29942895- |
| | CGCCCGC | CC | | 29942919 |
| 139 | CCGGCCCCUCCUGCUCU | NNNN | NME2 | chr6:29942903- |
| | AUCCACG | CC | | 29942927 |
| 140 | UCCGGCCCCUCCUGCUC | NNNN | NME2 | chr6:29942904- |
| | UAUCCAC | CC | | 29942928 |
| 141 | GGGAAGGAGACGCUGC | NNNN | NME2 | chr6:29943518- |
| | AGCGCACG | CC | | 29943542 |
| 142 | AGACGCUGCAGCGCACG | NNNN | NME2 | chr6:29943525- |
| | GGUACCA | CC | | 29943549 |
| 143 | GCGCACGGGUACCAGGG | NNNN | NME2 | chr6:29943535- |
| | GCCACGG | CC | | 29943559 |
| 144 | CACGGGUACCAGGGGCC | NNNN | NME2 | chr6:29943538- |
| | ACGGGGC | CC | | 29943562 |
| 145 | ACGGGUACCAGGGGCCA | NNNN | NME2 | chr6:29943539- |
| | CGGGGCG | CC | | 29943563 |
| 146 | CAGGGGCCACGGGGCGC | NNNN | NME2 | chr6:29943547- |
| | CUCCCUG | CC | | 29943571 |
| 147 | CAGGGAGGCGCCCCGUG | NNNN | NME2 | chr6:29943547- |
| | GCCCCUG | CC | | 29943571 |
| 148 | UCAGGGAGGCGCCCCGU | NNNN | NME2 | chr6:29943548- |
| | GGCCCCU | CC | | 29943572 |
| 149 | CAGGCGAUCAGGGAGGC | NNNN | NME2 | chr6:29943555- |
| | GCCCCGU | CC | | 29943579 |
| 150 | ACAGGCGAUCAGGGAG | NNNN | NME2 | chr6:29943556- |
| | GCGCCCCG | CC | | 29943580 |
| 151 | UACAGGCGAUCAGGGA | NNNN | NME2 | chr6:29943557- |
| | GGCGCCCC | CC | | 29943581 |
| 152 | GGGCGCCUCCCUGAUCG | NNNN | NME2 | chr6:29943558- |
| | CCUGUAG | CC | | 29943582 |
| 153 | GGCGCCUCCCUGAUCGC | NNNN | NME2 | chr6:29943559- |
| | CUGUAGA | CC | | 29943583 |
| 154 | GAGAUCUACAGGCGAUC | NNNN | NME2 | chr6:29943563- |
| | AGGGAGG | CC | | 29943587 |
| 155 | GGAGAUCUACAGGCGA | NNNN | NME2 | chr6:29943564- |
| | UCAGGGAG | CC | | 29943588 |
| 156 | GGGAGAUCUACAGGCG | NNNN | NME2 | chr6:29943565- |
| | AUCAGGGA | CC | | 29943589 |
| 157 | CUGAUCGCCUGUAGAUC | NNNN | NME2 | chr6:29943568- |
| | UCCCGGG | CC | | 29943592 |
| 158 | AUCGCCUGUAGAUCUCC | NNNN | NME2 | chr6:29943571- |
| | CGGGCUG | CC | | 29943595 |
| 159 | UCGCCUGUAGAUCUCCC | NNNN | NME2 | chr6:29943572- |
| | GGGCUGG | CC | | 29943596 |
| 160 | UUGUCUCCCCUCCUUGU | NNNN | NME2 | chr6:29943595- |
| | GGGAGGC | CC | | 29943619 |
| 161 | AUUGUCUCCCCUCCUUG | NNNN | NME2 | chr6:29943596- |
| | UGGGAGG | CC | | 29943620 |
| 162 | CCCAAUUGUCUCCCCUC | NNNN | NME2 | chr6:29943600- |
| | CUUGUGG | CC | | 29943624 |
| 163 | GGAUGGAGCCGCGGGCG | NGG | Spy + Base_Editor | chr6:29942885- |
| | CCG | | | 29942905 |
| 164 | GCGGGCGCCGUGGAUAG | NGG | Spy + Base_Editor | chr6:29942895- |
| | AGC | | | 29942915 |
| 165 | UGCUCUAUCCACGGCGC | NGG | Spy + Base_Editor | chr6:29942896- |
| | CCG | | | 29942916 |
| 166 | GGCGCCGUGGAUAGAGC | NGG | Spy + Base_Editor | chr6:29942898- |
| | AGG | | | 29942918 |
| 167 | GCGCCGUGGAUAGAGCA | NGG | Spy + Base_Editor | chr6:29942899- |
| | GGA | | | 29942919 |
| 168 | CGCCGUGGAUAGAGCAG | NGG | Spy + Base_Editor | chr6:29942900- |
| | GAG | | | 29942920 |
| 169 | GUGGAUAGAGCAGGAG | NGG | Spy + Base_Editor | chr6:29942904- |
| | GGGC | | | 29942924 |
| 170 | GCGUCUCCUUCCCGUUC | NGG | Spy + Base_Editor | chr6:29943511- |
| | UCC | | | 29943531 |
| 171 | GAAGGAGACGCUGCAGC | NGG | Spy + Base_Editor | chr6:29943520- |
| | GCA | | | 29943540 |
| 172 | AAGGAGACGCUGCAGCG | NGG | Spy + Base_Editor | chr6:29943521- |
| | CAC | | | 29943541 |
| 173 | GCUGCAGCGCACGGGUA | NGG | Spy + Base_Editor | chr6:29943529- |
| | CCA | | | 29943549 |
| 174 | AGAUCUACAGGCGAUCA | NGG | Spy + Base_Editor | chr6:29943566- |
| | GGG | | | 29943586 |
| 175 | CUGAUCGCCUGUAGAUC | NGG | Spy + Base_Editor | chr6:29943568- |
| | UCC | | | 29943588 |
| 176 | UGAUCGCCUGUAGAUCU | NGG | Spy + Base_Editor | chr6:29943569- |
| | CCC | | | 29943589 |
| 177 | GGGAGAUCUACAGGCG | NGG | Spy + Base_Editor | chr6:29943569- |
| | AUCA | | | 29943589 |
| 178 | CGGGAGAUCUACAGGCG | NGG | Spy + Base_Editor | chr6:29943570- |
| | AUC | | | 29943590 |
| 179 | CGCCUGUAGAUCUCCCG | NGG | Spy + Base_Editor | chr6:29943573- |
| | GGC | | | 29943593 |
| 180 | GGCCAGCCCGGGAGAUC | NGG | Spy + Base_Editor | chr6:29943578- |
| | UAC | | | 29943598 |
| 181 | UCCCGGGCUGGCCUCCC | NGG | Spy + Base_Editor | chr6:29943585- |
| | ACA | | | 29943605 |
| 182 | GGGCUGGCCUCCCACAA | NGG | Spy + Base_Editor | chr6:29943589- |
| | GGA | | | 29943609 |

*The guide sequence disclosed in this Table may be unmodified, or modified with a modification pattern disclosed herein or available in the art.

TABLE 5
Additional Exemplary HLA-A guide sequences.

| | | | | Exemplary Guide | |
|---|---|---|---|---|---|
| | | | | RNA Modified | |
| | | | Exemplary | Sequence (four | |
| | | | Guide RNA Full | terminal U residues | |
| | SEQ ID | | Sequence with | are optional and | |
| | NO to the | | PAM | may include 0, 1, 2, | Genomic |
| Guide | Guide | Guide | (SEQ ID NOS: | 3, 4, or more Us) | Coordinates |
| ID | Sequence | Sequence | 505-532) | (SEQ ID NOS: 533-560) | (hg38) |
| G021857 | 183 | ACGACA | ACGACACUGA | mA*mC*mG*ACACU | chr6:29942469- |
| | | CUGAUU | UUGGCUUCUC | GAUUGGCUUCUCGU | 29942489 |
| | | GGCUUC | GUUUUAGAGC | UUUAGAmGmCmUm | |
| | | UC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021858 | 184 | ACCCCU | ACCCCUCAUC | mA*mC*mC*CCUCA | chr6:29943058- |
| | | CAUCCC | CCCCACGGAC | UCCCCCACGGACGU | 29943078 |
| | | CCACGG | GUUUUAGAGC | UUUAGAmGmCmUm | |
| | | AC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021859 | 185 | GGCCCG | GGCCCGUCCG | mG*mG*mC*CCGUC | chr6:29943063- |
| | | UCCGUG | UGGGGGAUGA | CGUGGGGGAUGAG | 29943083 |
| | | GGGGAU | GUUUUAGAGC | UUUUAGAmGmCmU | |
| | | GA | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021860 | 186 | GCCAGG | GCCAGGUCGC | mG*mC*mC*AGGUC | chr6:29943080- |
| | | UCGCCC | CCACAGUCUC | GCCCACAGUCUCGU | 29943100 |
| | | ACAGUC | GUUUUAGAGC | UUUAGAmGmCmUm | |
| | | UC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021861 | 187 | GUUUAG | GUUUAGGCCA | mG*mU*mU*UAGGC | chr6:29943187- |
| | | GCCAAA | AAAAUCCCCC | CAAAAAUCCCCCGU | 29943207 |
| | | AAUCCC | GUUUUAGAGC | UUUAGAmGmCmUm | |
| | | CC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021862 | 188 | GGCCAA | GGCCAAAAAU | mG*mG*mC*CAAAA | chr6:29943192- |
| | | AAAUCC | CCCCCCGGGU | AUCCCCCCGGGUGU | 29943212 |
| | | CCCCGG | GUUUUAGAGC | UUUAGAmGmCmUm | |
| | | GU | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021863 | 189 | GACCAA | GACCAACCCG | mG*mA*mC*CAACC | chr6:29943197- |
| | | CCCGGG | GGGGGAUUUU | CGGGGGGAUUUUG | 29943217 |
| | | GGGAUU | GUUUUAGAGC | UUUUAGAmGmCmU | |
| | | UU | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021864 | 190 | CACGGG | CACGGGCCCA | mC*mA*mC*GGGCC | chr6:29943812- |
| | | CCCAAG | AGGCUGCUGC | CAAGGCUGCUGCGU | 29943832 |
| | | GCUGCU | GUUUUAGAGC | UUUAGAmGmCmUm | |
| | | GC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021865 | 191 | ACCCUC | ACCCUCAUGC | mA*mC*mC*CUCAU | chr6:29944349- |
| | | AUGCUG | UGCACAUGGC | GCUGCACAUGGCGU | 29944369 |
| | | CACAUG | GUUUUAGAGC | UUUAGAmGmCmUm | |
| | | GC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021866 | 192 | CCUCUA | CCUCUAGGAC | mC*mC*mU*CUAGG | chr6:29944996- |
| | | GGACCU | CUUAAGGCCC | ACCUUAAGGCCCGU | 29945016 |
| | | UAAGGC | GUUUUAGAGC | UUUAGAmGmCmUm | |
| | | CC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021867 | 193 | GCUCCU | GCUCCUUUCU | mG*mC*mU*CCUUU | chr6:29945018- |
| | | UUCUGG | GGUAUCUCAC | CUGGUAUCUCACGU | 29945038 |
| | | UAUCUC | GUUUUAGAGC | UUUAGAmGmCmUm | |
| | | AC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021868 | 194 | GCUAUG | GCUAUGGGGU | mG*mC*mU*AUGGG | chr6:29945341- |
| | | GGGUUU | UUCUUUGCAU | GUUUCUUUGCAUG | 29945361 |
| | | CUUUGC | GUUUUAGAGC | UUUUAGAmGmCmU | |
| | | AU | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021869 | 195 | GCCUUU | GCCUUUGCAG | mG*mC*mC*UUUGC | chr6:29945526- |
| | | GCAGAA | AAACAAAGUC | AGAAACAAAGUCG | 29945546 |
| | | ACAAAG | GUUUUAGAGC | UUUUAGAmGmCmU | |
| | | UC | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021870 | 196 | UGGACC | UGGACCAACC | mU*mG*mG*ACCAA | chr6:29944880- |
| | | AACCGC | GCCCUCCUGA | CCGCCCUCCUGAGU | 29944900 |
| | | CCUCCU | GUUUUAGAGC | UUUAGAmGmCmUm | (mismatch to |
| | | GA | UAGAAAUAGC | AmGmAmAmAmUmA | hg38 = 2) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021871 | 197 | AGCCUC | AGCCUCUCUG | mA*mG*mC*CUCUC | Na |
| | | UCUGAC | ACCUUUAGCA | UGACCUUUAGCAGU | |
| | | CUUUAG | GUUUUAGAGC | UUUAGAmGmCmUm | |
| | | CA | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021872 | 198 | CGCCCU | CGCCCUCCUG | mC*mG*mC*CCUCC | Na |
| | | CCUGAA | AAGGUCCUCA | UGAAGGUCCUCAGU | |
| | | GGUCCU | GUUUUAGAGC | UUUAGAmGmCmUm | |
| | | CA | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021873 | 200 | CCGCCC | CCGCCCUCCU | mC*mC*mG*CCCUCC | Na |
| | | UCCUGA | GAAGGUCCUC | UGAAGGUCCUCGUU | |
| | | AGGUCC | GUUUUAGAGC | UUAGAmGmCmUmA | |
| | | UC | UAGAAAUAGC | mGmAmAmAmUmAm | |
| | | | AAGUUAAAAU | GmCAAGUUAAAAU | |
| | | | AAGGCUAGUC | AAGGCUAGUCCGUU | |
| | | | CGUUAUCAAC | AUCAmAmCmUmUm | |
| | | | UUGAAAAAGU | GmAmAmAmAmAmG | |
| | | | GGCACCGAGU | mUmGmGmCmAmCm | |
| | | | CGGUGCUUUU | CmGmAmGmUmCmG | |
| | | | | mGmUmGmCmU*mU* | |
| | | | | mU*mU | |
| G021874 | 201 | UGGUUC | UGGUUCCCUU | mU*mG*mG*UUCCC | chr6:29943794- |
| | | CCUUUG | UGACACACAC | UUUGACACACACGU | 29943814 |
| | | ACACAC | GUUUUAGAGC | UUUAGAmGmCmUm | (mismatch to |
| | | AC | UAGAAAUAGC | AmGmAmAmAmUmA | hg38 = 3) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021875 | 202 | GACCCU | GACCCUGCUA | mG*mA*mC*CCUGC | na |
| | | GCUAAA | AAGGUCAGAG | UAAAGGUCAGAGG | |
| | | GGUCAG | GUUUUAGAGC | UUUUAGAmGmCmU | |
| | | AG | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021876 | 203 | AGGACC | AGGACCUUCA | mA*mG*mG*ACCUU | na |
| | | UUCAGG | GGAGGGCGGU | CAGGAGGGCGGUG | |
| | | AGGGCG | GUUUUAGAGC | UUUUAGAmGmCmU | |
| | | GU | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021877 | 204 | GCACAC | GCACACUUCU | mG*mC*mA*CACUU | chr6:29944671- |
| | | UUCUAC | ACCUGGGUCU | CUACCUGGGUCUGU | 29944691 |
| | | CUGGGU | GUUUUAGAGC | UUUAGAmGmCmUm | (mismatch to |
| | | CU | UAGAAAUAGC | AmGmAmAmAmUmA | hg38 = 3) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021878 | 205 | GAGCCU | GAGCCUCUCU | mG*mA*mG*CCUCU | na |
| | | CUCUGA | GACCUUUAGC | CUGACCUUUAGCGU | |
| | | CCUUUA | GUUUUAGAGC | UUUAGAmGmCmUm | |
| | | GC | UAGAAAUAGC | AmGmAmAmAmUmA | |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021879 | 206 | ACACUC | ACACUCCUCC | mA*mC*mA*CUCCU | chr6:29944054- |
| | | CUCCAG | AGCACACAUG | CCAGCACACAUGGU | 29944074 |
| | | CACACA | GUUUUAGAGC | UUUAGAmGmCmUm | (mismatch to |
| | | UG | UAGAAAUAGC | AmGmAmAmAmUmA | hg38 = 2) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021880 | 207 | CUCUGA | CUCUGACCUU | mC*mU*mC*UGACC | na |
| | | CCUUUA | UAGCAGGGUC | UUUAGCAGGGUCG | |
| | | GCAGGG | GUUUUAGAGC | UUUUAGAmGmCmU | |
| | | UC | UAGAAAUAGC | mAmGmAmAmAmUm | |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021881 | 208 | CAAGAU | CAAGAUAGCC | mC*mA*mA*GAUAG | chr6:29944043- |
| | | AGCCAC | ACAUGUGUGC | CCACAUGUGUGCGU | 29944063 |
| | | AUGUGU | GUUUUAGAGC | UUUAGAmGmCmUm | (mismatch to |
| | | GC | UAGAAAUAGC | AmGmAmAmAmUmA | hg38 = 2) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |
| G021882 | 209 | UCUGAC | UCUGACCUUU | mU*mC*mU*GACCU | chr6:29944450- |
| | | CUUUAG | AGCAGGGUCA | UUAGCAGGGUCAG | 29944470 |
| | | CAGGGU | GUUUUAGAGC | UUUUAGAmGmCmU | (mismatch to |
| | | CA | UAGAAAUAGC | mAmGmAmAmAmUm | hg38 = 3) |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021883 | 210 | UGUAAA | UGUAAAGGUG | mU*mG*mU*AAAGG | chr6:29945274- |
| | | GGUGAG | AGAGCCUGGA | UGAGAGCCUGGAG | 29945294 |
| | | AGCCUG | GUUUUAGAGC | UUUUAGAmGmCmU | (mismatch to |
| | | GA | UAGAAAUAGC | mAmGmAmAmAmUm | hg38 = 1) |
| | | | AAGUUAAAAU | AmGmCAAGUUAAA | |
| | | | AAGGCUAGUC | AUAAGGCUAGUCCG | |
| | | | CGUUAUCAAC | UUAUCAmAmCmUm | |
| | | | UUGAAAAAGU | UmGmAmAmAmAmA | |
| | | | GGCACCGAGU | mGmUmGmGmCmAm | |
| | | | CGGUGCUUUU | CmCmGmAmGmUmC | |
| | | | | mGmGmUmGmCmU* | |
| | | | | mU*mU*mU | |
| G021884 | 211 | GAAGGU | GAAGGUCCCU | mG*mA*mA*GGUCC | chr6:29944859- |
| | | CCCUGA | GAGGACCUUC | CUGAGGACCUUCGU | 29944879 |
| | | GGACCU | GUUUUAGAGC | UUUAGAmGmCmUm | (mismatch to |
| | | UC | UAGAAAUAGC | AmGmAmAmAmUmA | hg38 = 3) |
| | | | AAGUUAAAAU | mGmCAAGUUAAAA | |
| | | | AAGGCUAGUC | UAAGGCUAGUCCGU | |
| | | | CGUUAUCAAC | UAUCAmAmCmUmU | |
| | | | UUGAAAAAGU | mGmAmAmAmAmAm | |
| | | | GGCACCGAGU | GmUmGmGmCmAmC | |
| | | | CGGUGCUUUU | mCmGmAmGmUmCm | |
| | | | | GmGmUmGmCmU*m | |
| | | | | U*mU*mU | |

*The guide sequence disclosed in this Table may be unmodified, modified with the exemplary modification pattern shown in the Table, or modified with a different modification pattern disclosed herein or available in the art.

In some embodiments, the HLA-A gRNA comprises a guide sequence selected from any one of SEQ ID NOs: 1-95. In some embodiments, the HLA-A gRNA comprises a guide sequence selected from any one of SEQ ID NOs: 7, 13-18, 22, 26, 31, 33, 37-41, 43, 47, 57, 59, 62, 66, 87. In some embodiments, the HLA-A gRNA comprises a guide sequence selected from any one of SEQ ID NOs: 13-18, 26, 37-39, 41, 43, 45, 62. In some embodiments, the HLA-A gRNA comprises a guide sequence selected from any one of SEQ ID NOs: 13-18. In some embodiments, the HLA-A gRNA comprises a guide sequence selected from any one of SEQ ID NOs: 13-17. n some embodiments, the HLA-A gRNA comprises a guide sequence selected from any one of SEQ ID NOs: 37-39, 41, 43, and 45. In some embodiments, the HLA-A gRNA comprises a guide sequence selected from any one of SEQ ID NOs: 37-39.

In some embodiments, the gRNA comprises a guide sequence selected from any one of SEQ ID NOs: 1-211. In some embodiments, the HLA-A guide RNA comprises a guide sequence that is at least 17, 18, 19, or 20 contiguous nucleotides of a sequence selected from SEQ ID NOs: 1-211. In some embodiments, the HLA-A guide RNA comprises a guide sequence that is at least 95%, 90%, or 85% identical to a sequence selected from SEQ ID NOs: 1-211. In some embodiments, the HLA-A guide RNA comprises a guide sequence that is at least 95% identical to a sequence selected from SEQ ID NOs: 1-211.

In some embodiments, the HLA-A guide RNA comprises a guide sequence that comprises at least 10 contiguous nucleotides±10 nucleotides of a genomic coordinate listed in Tables 2-5. As used herein, at least 10 contiguous nucleotides±10 nucleotides of a genomic coordinate means, for example, at least 10 contiguous nucleotides within the genomic coordinates wherein the genomic coordinates include 10 nucleotides in the 5′ direction and 10 nucleotides in the 3′ direction from the ranges listed in Tables 2-5. For example, an HLA-A guide RNA may comprise 10 contiguous nucleotides within the genomic coordinates chr6:29942864 to chr6:29942903 or chr6:29943528 to chr6:29943609, including the boundary nucleotides of these ranges. In some embodiments, the HLA-A guide RNA comprises a guide sequence that is at least 17, 18, 19, or 20 contiguous nucleotides of a sequence that comprises 10 contiguous nucleotides±10 nucleotides of a genomic coordinate listed in Tables 1-2 and 5, or a guide sequence that is complementary to at least 17, 18, 19, 21, 22, 23, or 24 contiguous nucleotides of a sequence that comprises 10 contiguous nucleotides±10 nucleotides of a genomic coordinate listed in Table 4. In some embodiments, the HLA-A guide RNA comprises a guide sequence that is at least 95%, 90%, or 85% identical to a sequence selected from a sequence that is 17, 18, 19, or 20 contiguous nucleotides of a sequence that comprises 10 contiguous nucleotides±10 nucleotides of a genomic coordinate listed in Tables 1-2 and 5, or a guide sequence that is complementary to at least 17, 18, 19, 20, 21, 22, 23, or 24 contiguous nucleotides of a sequence that comprises contiguous nucleotides±10 nucleotides of a genomic coordinate listed in Table 4.

In some embodiments, the Tables 2-5 guide RNA comprises a guide sequence that comprises at least 15 contiguous nucleotides±10 nucleotides of a genomic coordinate listed in Tables 2-5. In some embodiments, the HLA-A guide RNA comprises a guide sequence that comprises at least 20 contiguous nucleotides±10 nucleotides of a genomic coordinate listed in Tables 2-5.

In some embodiments, the HLA-A guide RNA comprises SEQ ID NO: 1. In some embodiments, the HLA-A guide RNA comprises SEQ ID NO: 2. In some embodiments, the HLA-A guide RNA comprises SEQ ID NO: 3. In some embodiments, the HLA-A guide RNA comprises SEQ ID NO: 4. In some embodiments, the HLA-A guide RNA comprises SEQ ID NO: 5. In some embodiments, the HLA-A guide RNA comprises SEQ ID NO: 6. In some embodiments, the HLA-A guide RNA comprises SEQ ID NO: 7. In some embodiments, the HLA-A guide RNA comprises SEQ ID NO: 8. In some embodiments, the HLA-A guide RNA comprises SEQ ID NO: 9. In some embodiments, the HLA-A guide RNA comprises SEQ ID NO: 10. In some embodiments, the HLA-A guide RNA comprises SEQ ID NO: 11. In some embodiments, the HLA-A guide RNA comprises SEQ ID NO: 12. In some embodiments, the HLA-A guide RNA comprises SEQ ID NO: 13. In some embodiments, the HLA-A guide RNA comprises SEQ ID NO: 14. In some embodiments, the HLA-A guide RNA comprises SEQ ID NO: 15. In some embodiments, the HLA-A guide RNA comprises SEQ ID NO: 16. In some embodiments, the HLA-A guide RNA comprises SEQ ID NO: 17. In some embodiments, the HLA-A guide RNA comprises SEQ ID NO: 18. In some embodiments, the HLA-A guide RNA comprises SEQ ID NO: 19. In some embodiments, the HLA-A guide RNA comprises SEQ ID NO: 20. In some embodiments, the HLA-A guide RNA comprises SEQ ID NO: 21. In some embodiments, the HLA-A guide RNA comprises SEQ ID NO: 22. In some embodiments, the HLA-A guide RNA comprises SEQ ID NO: 23. In some embodiments, the HLA-A guide RNA comprises SEQ ID NO: 24. In some embodiments, the HLA-A guide RNA comprises SEQ ID NO: 25. In some embodiments, the HLA-A guide RNA comprises SEQ ID NO: 26. In some embodiments, the HLA-A guide RNA comprises SEQ ID NO: 27. In some embodiments, the HLA-A guide RNA comprises SEQ ID NO: 28. In some embodiments, the HLA-A guide RNA comprises SEQ ID NO: 29. In some embodiments, the HLA-A guide RNA comprises SEQ ID NO: 30. In some embodiments, the HLA-A guide RNA comprises SEQ ID NO: 31. In some embodiments, the HLA-A guide RNA comprises SEQ ID NO: 32. In some embodiments, the HLA-A guide RNA comprises SEQ ID NO: 33. In some embodiments, the HLA-A guide RNA comprises SEQ ID NO: 34. In some embodiments, the HLA-A guide RNA comprises SEQ ID NO: 35. In some embodiments, the HLA-A guide RNA comprises SEQ ID NO: 36. In some embodiments, the HLA-A guide RNA comprises SEQ ID NO: 37. In some embodiments, the HLA-A guide RNA comprises SEQ ID NO: 38. In some embodiments, the HLA-A guide RNA comprises SEQ ID NO: 39. In some embodiments, the HLA-A guide RNA comprises SEQ ID NO: 40. In some embodiments, the HLA-A guide RNA comprises SEQ ID NO: 41. In some embodiments, the HLA-A guide RNA comprises SEQ ID NO: 42. In some embodiments, the HLA-A guide RNA comprises SEQ ID NO: 43. In some embodiments, the HLA-A guide RNA comprises SEQ ID NO: 44. In some embodiments, the HLA-A guide RNA comprises SEQ ID NO: 45. In some embodiments, the HLA-A guide RNA comprises SEQ ID NO: 46. In some embodiments, the HLA-A guide RNA comprises SEQ ID NO: 47. In some embodiments, the HLA-A guide RNA comprises SEQ ID NO: 48. In some embodiments, the HLA-A guide RNA comprises SEQ ID NO: 49. In some embodiments, the HLA-A guide RNA comprises SEQ ID NO: 50. In some embodiments, the HLA-A guide RNA comprises SEQ ID NO: 51. In some embodiments, the HLA-A guide RNA comprises SEQ ID NO: 52. In some embodiments, the HLA-A guide RNA comprises SEQ ID NO: 53. In some embodiments, the HLA-A guide RNA comprises SEQ ID NO: 54. In some embodiments, the HLA-A guide RNA comprises SEQ ID NO: 55. In some embodiments, the HLA-A guide RNA comprises SEQ ID NO: 56. In some embodiments, the HLA-A guide RNA comprises SEQ ID NO: 57. In some embodiments, the HLA-A guide RNA comprises SEQ ID NO: 58. In some embodiments, the HLA-A guide RNA comprises SEQ ID NO: 59. In some embodiments, the HLA-A guide RNA comprises SEQ ID NO: 60. In some embodiments, the HLA-A guide RNA comprises SEQ ID NO: 61. In some embodiments, the HLA-A guide RNA comprises SEQ ID NO: 62. In some embodiments, the HLA-A guide RNA comprises SEQ ID NO: 63. In some embodiments, the HLA-A guide RNA comprises SEQ ID NO: 64. In some embodiments, the HLA-A guide RNA comprises SEQ ID NO: 65. In some embodiments, the HLA-A guide RNA comprises SEQ ID NO: 66. In some embodiments, the HLA-A guide RNA comprises SEQ ID NO: 67. In some embodiments, the HLA-A guide RNA comprises SEQ ID NO: 68. In some embodiments, the HLA-A guide RNA comprises SEQ ID NO: 69. In some embodiments, the HLA-A guide RNA comprises SEQ ID NO: 70. In some embodiments, the HLA-A guide RNA comprises SEQ ID NO: 71. In some embodiments, the HLA-A guide RNA comprises SEQ ID NO: 72. In some embodiments, the HLA-A guide RNA comprises SEQ ID NO: 73. In some embodiments, the HLA-A guide RNA comprises SEQ ID NO: 74. In some embodiments, the HLA-A guide RNA comprises SEQ ID NO: 75. In some embodiments, the HLA-A guide RNA comprises SEQ ID NO: 76. In some embodiments, the HLA-A guide RNA comprises SEQ ID NO: 77. In some embodiments, the HLA-A guide RNA comprises SEQ ID NO: 78. In some embodiments, the HLA-A guide RNA comprises SEQ ID NO: 79. In some embodiments, the HLA-A guide RNA comprises SEQ ID NO: 80. In some embodiments, the HLA-A guide RNA comprises SEQ ID NO: 81. In some embodiments, the HLA-A guide RNA comprises SEQ ID NO: 82. In some embodiments, the HLA-A guide RNA comprises SEQ ID NO: 83. In some embodiments, the HLA-A guide RNA comprises SEQ ID NO: 84. In some embodiments, the HLA-A guide RNA comprises SEQ ID NO: 85. In some embodiments, the HLA-A guide RNA comprises SEQ ID NO: 86. In some embodiments, the HLA-A guide RNA comprises SEQ ID NO: 87. In some embodiments, the HLA-A guide RNA comprises SEQ ID NO: 88. In some embodiments, the HLA-A guide RNA comprises SEQ ID NO: 89. In some embodiments, the HLA-A guide RNA comprises SEQ ID NO: 90. In some embodiments, the HLA-A guide RNA comprises SEQ ID NO: 91. In some embodiments, the HLA-A guide RNA comprises SEQ ID NO: 92. In some embodiments, the HLA-A guide RNA comprises SEQ ID NO: 93. In some embodiments, the HLA-A guide RNA comprises SEQ ID NO: 94. In some embodiments, the HLA-A guide RNA comprises SEQ ID NO: 95. In some embodiments, the HLA-A guide RNA comprises SEQ ID NO: 96. In some embodiments, the HLA-A guide RNA comprises SEQ ID NO: 97. In some embodiments, the HLA-A guide RNA comprises SEQ ID NO: 98. In some embodiments, the HLA-A guide RNA comprises SEQ ID NO: 99. In some embodiments, the HLA-A guide RNA comprises SEQ ID NO: 100. In some embodiments, the HLA-A guide RNA comprises SEQ ID NO: 101. In some embodiments, the HLA-A guide RNA comprises SEQ ID NO: 102. In some embodiments, the HLA-A guide RNA comprises SEQ ID NO: 103. In some embodiments, the HLA-A guide RNA comprises SEQ ID NO: 104. In some embodiments, the HLA-A guide RNA comprises SEQ ID NO: 105. In some embodiments, the HLA-A guide RNA comprises SEQ ID NO: 106. In some embodiments, the HLA-A guide RNA comprises SEQ ID NO: 107. In some embodiments, the HLA-A guide RNA comprises SEQ ID NO: 108. In some embodiments, the HLA-A guide RNA comprises SEQ ID NO: 109. In some embodiments, the HLA-A guide RNA comprises SEQ ID NO: 110. In some embodiments, the HLA-A guide RNA comprises SEQ ID NO: 111. In some embodiments, the HLA-A guide RNA comprises SEQ ID NO: 112. In some embodiments, the HLA-A guide RNA comprises SEQ ID NO: 113. In some embodiments, the HLA-A guide RNA comprises SEQ ID NO: 114. In some embodiments, the HLA-A guide RNA comprises SEQ ID NO: 115. In some embodiments, the HLA-A guide RNA comprises SEQ ID NO: 116. In some embodiments, the HLA-A guide RNA comprises SEQ ID NO: 117. In some embodiments, the HLA-A guide RNA comprises SEQ ID NO: 118. In some embodiments, the HLA-A guide RNA comprises SEQ ID NO: 119. In some embodiments, the HLA-A guide RNA comprises SEQ ID NO: 120. In some embodiments, the HLA-A guide RNA comprises SEQ ID NO: 121. In some embodiments, the HLA-A guide RNA comprises SEQ ID NO: 122. In some embodiments, the HLA-A guide RNA comprises SEQ ID NO: 123. In some embodiments, the HLA-A guide RNA comprises SEQ ID NO: 124. In some embodiments, the HLA-A guide RNA comprises SEQ ID NO: 125. In some embodiments, the HLA-A guide RNA comprises SEQ ID NO: 126. In some embodiments, the HLA-A guide RNA comprises SEQ ID NO: 127. In some embodiments, the HLA-A guide RNA comprises SEQ ID NO: 128. In some embodiments, the HLA-A guide RNA comprises SEQ ID NO: 129. In some embodiments, the HLA-A guide RNA comprises SEQ ID NO: 130. In some embodiments, the HLA-A guide RNA comprises SEQ ID NO: 131. In some embodiments, the HLA-A guide RNA comprises SEQ ID NO: 132. In some embodiments, the HLA-A guide RNA comprises SEQ ID NO: 133. In some embodiments, the HLA-A guide RNA comprises SEQ ID NO: 134. In some embodiments, the HLA-A guide RNA comprises SEQ ID NO: 135. In some embodiments, the HLA-A guide RNA comprises SEQ ID NO: 136. In some embodiments, the HLA-A guide RNA comprises SEQ ID NO: 137. In some embodiments, the HLA-A guide RNA comprises SEQ ID NO: 138. In some embodiments, the HLA-A guide RNA comprises SEQ ID NO: 139. In some embodiments, the HLA-A guide RNA comprises SEQ ID NO: 140. In some embodiments, the HLA-A guide RNA comprises SEQ ID NO: 141. In some embodiments, the HLA-A guide RNA comprises SEQ ID NO: 142. In some embodiments, the HLA-A guide RNA comprises SEQ ID NO: 143. In some embodiments, the HLA-A guide RNA comprises SEQ ID NO: 144. In some embodiments, the HLA-A guide RNA comprises SEQ ID NO: 145. In some embodiments, the HLA-A guide RNA comprises SEQ ID NO: 146. In some embodiments, the HLA-A guide RNA comprises SEQ ID NO: 147. In some embodiments, the HLA-A guide RNA comprises SEQ ID NO: 148. In some embodiments, the HLA-A guide RNA comprises SEQ ID NO: 149. In some embodiments, the HLA-A guide RNA comprises SEQ ID NO: 150. In some embodiments, the HLA-A guide RNA comprises SEQ ID NO: 151. In some embodiments, the HLA-A guide RNA comprises SEQ ID NO: 152. In some embodiments, the HLA-A guide RNA comprises SEQ ID NO: 153. In some embodiments, the HLA-A guide RNA comprises SEQ ID NO: 154. In some embodiments, the HLA-A guide RNA comprises SEQ ID NO: 155. In some embodiments, the HLA-A guide RNA comprises SEQ ID NO: 156. In some embodiments, the HLA-A guide RNA comprises SEQ ID NO: 157. In some embodiments, the HLA-A guide RNA comprises SEQ ID NO: 158. In some embodiments, the HLA-A guide RNA comprises SEQ ID NO: 159. In some embodiments, the HLA-A guide RNA comprises SEQ ID NO: 160. In some embodiments, the HLA-A guide RNA comprises SEQ ID NO: 161. In some embodiments, the HLA-A guide RNA comprises SEQ ID NO: 162. In some embodiments, the HLA-A guide RNA comprises SEQ ID NO: 163. In some embodiments, the HLA-A guide RNA comprises SEQ ID NO: 164. In some embodiments, the HLA-A guide RNA comprises SEQ ID NO: 165. In some embodiments, the HLA-A guide RNA comprises SEQ ID NO: 166. In some embodiments, the HLA-A guide RNA comprises SEQ ID NO: 167. In some embodiments, the HLA-A guide RNA comprises SEQ ID NO: 168. In some embodiments, the HLA-A guide RNA comprises SEQ ID NO: 169. In some embodiments, the HLA-A guide RNA comprises SEQ ID NO: 170. In some embodiments, the HLA-A guide RNA comprises SEQ ID NO: 171. In some embodiments, the HLA-A guide RNA comprises SEQ ID NO: 172. In some embodiments, the HLA-A guide RNA comprises SEQ ID NO: 173. In some embodiments, the HLA-A guide RNA comprises SEQ ID NO: 174. In some embodiments, the HLA-A guide RNA comprises SEQ ID NO: 175. In some embodiments, the HLA-A guide RNA comprises SEQ ID NO: 176. In some embodiments, the HLA-A guide RNA comprises SEQ ID NO: 177. In some embodiments, the HLA-A guide RNA comprises SEQ ID NO: 178. In some embodiments, the HLA-A guide RNA comprises SEQ ID NO: 179. In some embodiments, the HLA-A guide RNA comprises SEQ ID NO: 180. In some embodiments, the HLA-A guide RNA comprises SEQ ID NO: 181. In some embodiments, the HLA-A guide RNA comprises SEQ ID NO: 182. In some embodiments, the HLA-A guide RNA comprises SEQ ID NO: 183. In some embodiments, the HLA-A guide RNA comprises SEQ ID NO: 184. In some embodiments, the HLA-A guide RNA comprises SEQ ID NO: 185. In some embodiments, the HLA-A guide RNA comprises SEQ ID NO: 186. In some embodiments, the HLA-A guide RNA comprises SEQ ID NO: 187. In some embodiments, the HLA-A guide RNA comprises SEQ ID NO: 188. In some embodiments, the HLA-A guide RNA comprises SEQ ID NO: 189. In some embodiments, the HLA-A guide RNA comprises SEQ ID NO: 190. In some embodiments, the HLA-A guide RNA comprises SEQ ID NO: 191. In some embodiments, the HLA-A guide RNA comprises SEQ ID NO: 192. In some embodiments, the HLA-A guide RNA comprises SEQ ID NO: 193. In some embodiments, the HLA-A guide RNA comprises SEQ ID NO: 194. In some embodiments, the HLA-A guide RNA comprises SEQ ID NO: 195. In some embodiments, the HLA-A guide RNA comprises SEQ ID NO: 196. In some embodiments, the HLA-A guide RNA comprises SEQ ID NO: 197. In some embodiments, the HLA-A guide RNA comprises SEQ ID NO: 198. In some embodiments, the HLA-A guide RNA comprises SEQ ID NO: 199. In some embodiments, the HLA-A guide RNA comprises SEQ ID NO: 200. In some embodiments, the HLA-A guide RNA comprises SEQ ID NO: 201. In some embodiments, the HLA-A guide RNA comprises SEQ ID NO: 202. In some embodiments, the HLA-A guide RNA comprises SEQ ID NO: 203. In some embodiments, the HLA-A guide RNA comprises SEQ ID NO: 204. In some embodiments, the HLA-A guide RNA comprises SEQ ID NO: 205. In some embodiments, the HLA-A guide RNA comprises SEQ ID NO: 206. In some embodiments, the HLA-A guide RNA comprises SEQ ID NO: 207. In some embodiments, the HLA-A guide RNA comprises SEQ ID NO: 208. In some embodiments, the HLA-A guide RNA comprises SEQ ID NO: 209. In some embodiments, the HLA-A guide RNA comprises SEQ ID NO: 210. In some embodiments, the HLA-A guide RNA comprises SEQ ID NO: 211.

Additional embodiments of HLA-A guide RNAs are provided herein, including e.g., exemplary modifications to the guide RNA.

### 2. Genetic Modifications to HLA-A

In some embodiments, the methods and compositions disclosed herein genetically modify at least one nucleotide in the HLA-A gene in a cell. Genetic modifications encompass the population of modifications that results from contact with a gene editing system (e.g., the population of edits that result from Cas9 and an HLA-A guide RNA, or the population of edits that result from BC22 and an HLA-A guide RNA).

In some embodiments, the genetic modification comprises at least one nucleotide within the genomic coordinates chosen from: chr6:29942854-chr6:29942913 and chr6:29943518-chr6:29943619.

In some embodiments, the genetic modification comprises at least one nucleotide within the genomic coordinates chr6:29942864-chr6:29942903.

In some embodiments, the genetic modification comprises at least one nucleotide within the genomic coordinates chr6:29943528-chr6:29943609.

In some embodiments, the genetic modification comprises at least one nucleotide within the genomic coordinates chr6:29942864-29942884; chr6:29942868-29942888; chr6:29942876-29942896; chr6:29942877-29942897; and chr6:29942883-29942903.

In some embodiments, the genetic modification comprises at least one nucleotide within the genomic coordinates chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises at least one nucleotide within the genomic coordinates chr6:29942876-29942897.

In some embodiments, the genetic modification comprises at least one nucleotide within the genomic coordinates chr6:29943528-chr629943550.

In some embodiments, the genetic modification comprises at least one nucleotide within the genomic coordinates chosen from: chr6:29942864-29942884, chr6:29942868-29942888, chr6:29942876-29942896, and chr6:29942877-29942897.

In some embodiments, the genetic modification comprises at least one nucleotide within the genomic coordinates chosen from: chr6:29943528-29943548, chr6:29943529-29943549, and chr6:29943530-29943550.

In some embodiments, the genetic modification comprises at least one nucleotide within the genomic coordinates chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870.

In some embodiments, the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

In some embodiments, the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; and chr6:29942883-29942903.

In some embodiments, the genetic modification comprises an indel, a C to T substitution, or an A to G substitution within the genomic coordinates chosen from: chr6:29943528-29943548; chr6:29943529-29943549; chr6:29943530-29943550; chr6:29943537-29943557; chr6:29943549-29943569; and chr6:29943589-29943609.

In some embodiments, the genetic modification comprises an indel, a C to T substitution, or an A to G substitution within the genomic coordinates chosen from: chr6:29890117-29890137, chr6:29927058-29927078, chr6:29934330-29934350, chr6:29942541-29942561, chr6:29942542-29942562, chr6:29942543-29942563, chr6:29942543-29942563, chr6:29942550-29942570, chr6:29942864-29942884, chr6:29942868-29942888, chr6:29942876-29942896, chr6:29942876-29942896, chr6:29942877-29942897, chr6:29942883-29942903, chr6:29943062-29943082, chr6:29943063-29943083, chr6:29943092-29943112, chr6:29943115-29943135, chr6:29943118-29943138, chr6:29943119-29943139, chr6:29943120-29943140, chr6:29943126-29943146, chr6:29943128-29943148, chr6:29943129-29943149, chr6:29943134-29943154, chr6:29943134-29943154, chr6:29943135-29943155, chr6:29943136-29943156, chr6:29943140-29943160, chr6:29943142-29943162, chr6:29943143-29943163, chr6:29943188-29943208, chr6:29943528-29943548, chr6:29943529-29943549, chr6:29943530-29943550, chr6:29943536-29943556, chr6:29943537-29943557, chr6:29943538-29943558, chr6:29943549-29943569, chr6:29943556-29943576, chr6:29943589-29943609, chr6:29943590-29943610, chr6:29943590-29943610, chr6:29943599-29943619, chr6:29943600-29943620, chr6:29943601-29943621, chr6:29943602-29943622, chr6:29943603-29943623, chr6:29943774-29943794, chr6:29943779-29943799, chr6:29943780-29943800, chr6:29943822-29943842, chr6:29943824-29943844, chr6:29943857-29943877, chr6:29943858-29943878, chr6:29943859-29943879, chr6:29943860-29943880, chr6:29944026-29944046, chr6:29944077-29944097, chr6:29944078-29944098, chr6:29944458-29944478, chr6:29944478-29944498, chr6:29944597-29944617, chr6:29944642-29944662, chr6:29944643-29944663, chr6:29944772-29944792, chr6:29944782-29944802, chr6:29944850-29944870, chr6:29944907-29944927, chr6:29945024-29945044, chr6:29945097-29945117, chr6:29945104-29945124, chr6:29945105-29945125, chr6:29945116-29945136, chr6:29945118-29945138, chr6:29945119-29945139, chr6:29945124-29945144, chr6:29945176-29945196, chr6:29945177-29945197, chr6:29945177-29945197, chr6:29945180-29945200, chr6:29945187-29945207, chr6:29945188-29945208, chr6:29945228-29945248, chr6:29945230-29945250, chr6:29945231-29945251, chr6:29945232-29945252, chr6:29945308-29945328, chr6:29945361-29945381, chr6:29945362-29945382, and chr6:31382543-31382563.

In some embodiments, the genetic modification comprises an indel, a C to T substitution, or an A to G substitution within the genomic coordinates chosen from: chr6:29942815-29942835, chr6:29942816-29942836, chr6:29942817-29942837, chr6:29942817-29942837, chr6:29942828-29942848, chr6:29942837-29942857, chr6:29942885-29942905, chr6:29942895-29942915, chr6:29942896-29942916, chr6:29942898-29942918, chr6:29942899-29942919, chr6:29942900-29942920, chr6:29942904-29942924, chr6:29942905-29942925, chr6:29942912-29942932, chr6:29942913-29942933, chr6:29943490-29943510, chr6:29943497-29943517, chr6:29943498-29943518, chr6:29943502-29943522, chr6:29943502-29943522, chr6:29943511-29943531, chr6:29943520-29943540, chr6:29943521-29943541, chr6:29943566-29943586, chr6:29943569-29943589, chr6:29943569-29943589, chr6:29943570-29943590, chr6:29943573-29943593, chr6:29943578-29943598, chr6:29943585-29943605, chr6:29943589-29943609, chr6:29943568-29943588, and chr6:29942815-29942835.

In some embodiments, the genetic modification comprises an indel, a C to T substitution, or an A to G substitution within the genomic coordinates chosen from: chr6:29942884-29942904, chr6:29943519-29943539, chr6:29942863-29942883.

In some embodiments, the genetic modification comprises an indel, a C to T substitution, or an A to G substitution within the genomic coordinates chosen from: chr6:29943517-29943537, and chr6:29943523-29943543.

In some embodiments, the genetic modification comprises an indel, a C to T substitution, or an A to G substitution within the genomic coordinates chosen from: chr6:29942845-29942869, chr6:29942852-29942876, chr6:29942865-29942889, chr6:29942891-29942915, chr6:29942895-29942919, chr6:29942903-29942927, chr6:29942904-29942928, chr6:29943518-29943542, chr6:29943525-29943549, chr6:29943535-29943559, chr6:29943538-29943562, chr6:29943539-29943563, chr6:29943547-29943571, chr6:29943547-29943571, chr6:29943548-29943572, chr6:29943555-29943579, chr6:29943556-29943580, chr6:29943557-29943581, chr6:29943558-29943582, chr6:29943559-29943583, chr6:29943563-29943587, chr6:29943564-29943588, chr6:29943565-29943589, chr6:29943568-29943592, chr6:29943571-29943595, chr6:29943572-29943596, chr6:29943595-29943619, chr6:29943596-29943620, chr6:29943600-29943624.

In some embodiments, the genetic modification comprises an indel, a C to T substitution, or an A to G substitution within the genomic coordinates chosen from: chr6:29942885-29942905, chr6:29942895-29942915, chr6:29942896-29942916, chr6:29942898-29942918, chr6:29942899-29942919, chr6:29942900-29942920, chr6:29942904-29942924, chr6:29943511-29943531, chr6:29943520-29943540, chr6:29943521-29943541, chr6:29943529-29943549, chr6:29943566-29943586, chr6:29943568-29943588, chr6:29943569-29943589, chr6:29943569-29943589, chr6:29943570-29943590, chr6:29943573-29943593, chr6:29943578-29943598, chr6:29943585-29943605, and chr6:29943589-29943609.

In some embodiments, the genetic modification comprises an indel, a C to T substitution, or an A to G substitution within the genomic coordinates chosen from: chr6:29942469-29942489, chr6:29943058-29943078, chr6:29943063-29943083, chr6:29943080-29943100, chr6:29943187-29943207, chr6:29943192-29943212, chr6:29943197-29943217, chr6:29943812-29943832, chr6:29944349-29944369, chr6:29944996-29945016, chr6:29945018-29945038, chr6:29945341-29945361, chr6:29945526-29945546.

In some embodiments, the genetic modification comprises an indel, a C to T substitution, or an A to G substitution within the genomic coordinates: chr6:29942876-29942897.

In some embodiments, the genetic modification comprises an indel, a C to T substitution, or an A to G substitution within the genomic coordinates chosen from: chr6:29942864-29942884, chr6:29942868-29942888, chr6:29942876-29942896, and chr6:29942877-29942897.

In some embodiments, the genetic modification comprises an indel, a C to T substitution, or an A to G substitution within the genomic coordinates: chr6:29943528-chr629943550.

In some embodiments, the genetic modification comprises an indel, a C to T substitution, or an A to G substitution within the genomic coordinates chosen from: chr6:29943528-29943548, chr6:29943529-29943549, and chr6:29943530-29943550.

In some embodiments, the modification to HLA-A comprises any one or more of an insertion, deletion, substitution or deamination of at least one nucleotide in a target sequence. In some embodiments, the modification to HLA-A comprises an insertion of 1, 2, 3, 4 or 5 or more nucleotides in a target sequence. In some embodiments, the modification to HLA-A comprises a deletion of 1, 2, 3, 4 or 5 or more nucleotides in a target sequence. In other embodiments, the modification to HLA-A comprises an insertion of 1, 2, 3, 4, 5, 6, 7, 8, 9, 10, 15, 20 or 25 or more nucleotides in a target sequence. In other embodiments, the modification to HLA-A comprises a deletion of 1, 2, 3, 4, 5, 6, 7, 8, 9, 10, 15, 20 or 25 or more nucleotides in a target sequence. In some embodiments, the modification to HLA-A comprises an indel, which is generally defined in the art as an insertion or deletion of less than 1000 base pairs (bp). In some embodiments, the modification to HLA-A comprises an indel which results in a frameshift mutation in a target sequence. In some embodiments, the modification to HLA-A comprises a substitution of 1, 2, 3, 4, 5, 6, 7, 8, 9, 10, 15, 20 or 25 or more nucleotides in a target sequence. In some embodiments, the modification to HLA-A comprises one or more of an insertion, deletion, or substitution of nucleotides resulting from the incorporation of a template nucleic acid. In some embodiments, the modification to HLA-A comprises an insertion of a donor nucleic acid in a target sequence. In some embodiments, the modification to HLA-A is not transient.

### 3. Efficacy of HLA-A Guide RNAs

The efficacy of an HLA-A guide RNA may be determined by techniques available in the art that assess the editing efficiency of a guide RNA, and the expression of HLA-A protein on the surface of a cell. In some embodiments, the reduction or elimination of HLA-A protein on the surface of a cell may be determined by comparison to an unmodified cell (or “relative to an unmodified cell”). An engineered cell or cell population may also be compared to a population of unmodified cells.

An “unmodified cell” (or “unmodified cells”) refers to a control cell (or cells) of the same type of cell in an experiment or test, wherein the “unmodified” control cell has not been contacted with an HLA-A guide. Therefore, an unmodified cell (or cells) may be a cell that has not been contacted with a guide RNA, or a cell that has been contacted with a guide RNA that does not target HLA-A.

In some embodiments, the efficacy of an HLA-A guide RNA is determined by measuring levels of HLA-A protein on the surface of a cell. In some embodiments, HLA-A protein levels are measured by flow cytometry (e.g., with an antibody against HLA-A2/HLA-A3). In some embodiments, the population of cells is enriched (e.g., by FACS or MACS) and is at least 65%, 70%, 80%, 90%, 91%, 92%, 93%, or 94% HLA-A negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% HLA-A negative as measured by flow cytometry relative to a population of unmodified cells. In some embodiments, the population of cells is at least 65% HLA-A negative as measured by flow cytometry relative to a population of unmodified cells. In some embodiments, the population of cells is at least 70% HLA-A negative as measured by flow cytometry relative to a population of unmodified cells. In some embodiments, the population of cells is at least 80% HLA-A negative as measured by flow cytometry relative to a population of unmodified cells. In some embodiments, the population of cells is at least 90% HLA-A negative as measured by flow cytometry relative to a population of unmodified cells. In some embodiments, the population of cells is at least 95% MHC I negative as measured by flow cytometry relative to a population of unmodified cells. In some embodiments, the population of cells is at least 100% HLA-A negative as measured by flow cytometry relative to a population of unmodified cells.

In some embodiments, an effective HLA-A guide RNA may be determined by measuring the response of immune cells in vitro or in vivo (e.g., CD8+ T cells) to the genetically modified target cell. For example, a reduced response from CD8+ T cells is indicative of an effective HLA-A guide RNA. A CD8+ T cell response may be evaluated by an assay that measures CD8+ T cell activation responses, e.g., CD8+ T cell proliferation, expression of activation markers, and/or cytokine production (IL-2, IFN-γ, TNF-α) (e.g., flow cytometry, ELISA). The CD8+ T cell response may be assessed in vitro or in vivo. In some embodiments, the CD8+ T cell response may be evaluated by co-culturing the genetically modified cell with CD8+ T cells in vitro. In some embodiments, CD8+ T cell activity may be evaluated in an in vivo model, e.g., a rodent model. In an in vivo model, e.g., genetically modified cells may be administered with CD8+ T cell; survival of the genetically modified cells is indicative of the ability to avoid CD8+ T cell lysis. In some embodiments, the methods produce a composition comprising a cell that survives in vivo in the presence of CD8+ T cells for greater than 1, 2, 3, 4, 5, or 6 weeks or more. In some embodiments, the methods produce a composition comprising a cell that survives in vivo in the presence of CD8+ T cells for at least one week to six weeks. In some embodiments, the methods produce a composition comprising a cell that survives in vivo in the presence of CD8+ T cells for at least two to four weeks. In some embodiments, the methods produce a composition comprising a cell that survives in vivo in the presence of CD8+ T cells for at least four to six weeks. In some embodiments, the methods produce a composition comprising a cell that survives in vivo in the presence of CD8+ T cells for more than six weeks.

The efficacy of an HLA-A guide RNA may also be assessed by the survival of the cell post-editing. In some embodiments, the cell survives post editing for at least one week to six weeks. In some embodiments, the cell survives post editing for at least two weeks. In some embodiments, the cell survives post editing for at least three weeks. In some embodiments, the cell survives post editing for at least four weeks. In some embodiments, the cell survives post editing for at least five weeks. In some embodiments, the cell survives post editing for at least six weeks. In some embodiments, the cell survives post editing for at least one week to twelve weeks. The viability of a genetically modified cell may be measured using standard techniques, including e.g., by measures of cell death, by flow cytometry live/dead staining, or cell proliferation.

In some embodiments, the engineered cell is assessed by the persistence of the engineered human cell which has reduced or eliminated HLA-A expression and is homozygous for HLA-B and homozygous for HLA-C. As used herein, “persistence” refers to the ability of the engineered cell to exist in an in vitro and/or in vivo environment with reactive or responding T cells and/or NK cells present, e.g., the ability to exist in vivo after transfer into a recipient. In some embodiments, the engineered human T cells are protective against NK-mediated rejection. In some embodiments, the ratio of viable engineered cells in vivo in the presence of NK cells relative to viable engineered cells in vivo in the absence of NK cells is at least 0.3:1 or greater, at least 20 days, at least 30 days, at least 40 days, at least 50 days, at least 60 days, at least 70 days, at least 80 days, or at least 90 days after transfer into a recipient, as demonstrated herein. In some embodiments, at least 90 days after transfer into a recipient, the ratio of viable engineered cells in vivo in the presence of NK cells relative to viable engineered cells in vivo in the absence of NK cells is at least 0.4:1 or greater, 0.5:1 or greater, 0.6:1 or greater, 0.7:1 or greater, 0.8:1 or greater, or 0.9:1 or greater, as demonstrated herein. In some embodiments, the engineered human T cells are protective against CD8+ T cell-mediated rejection.

In some embodiments, the engineered cells may be assessed using a mixed lymphocyte reaction (MLR). (See e.g., DeWolf et al., Transplantation 100:1639-1649 (2017). In some embodiments, engineered human cells are mixed with labeled unedited (non-engineered) responding T cells, and the MLR assay measures proliferation of responding T cells activated by allorecognition (i.e., through mismatched HLA molecules on the surface of the engineered human cell).

### D. Methods and Compositions for Reducing or Eliminating MHC Class II and Additional Modifications

In some embodiments, multiplex gene editing may be performed in a cell. In some embodiments, the methods comprise reducing or eliminating expression of HLA-A protein on the surface of a cell comprising genetically modifying the HLA-A gene comprising contacting the cell with a composition comprising a HLA-A guide RNA disclosed herein; and optionally an RNA-guided DNA binding agent or a nucleic acid encoding an RNA-guided DNA binding agent, the method further comprising contacting with one or more compositions selected from: (a) a guide RNA that directs an RNA-guided DNA binding agent to the CIITA gene; (b) a guide RNA that directs an RNA-guided DNA binding agent to a locus in the genome of the cell other than HLA-A or CIITA; and (c) a donor nucleic acid for insertion in the genome of the cell.

### 1. MHC Class II Knock Out

In some embodiments, methods for reducing or eliminating expression of HLA-A protein on the surface of a cell by genetically modifying HLA-A as disclosed herein are provided, wherein the methods and compositions further provide for reducing or eliminating expression of MHC class II protein on the surface of the cell relative to an unmodified cell. In some embodiments, MHC class II protein expression is reduced or eliminated by contacting the cell with a CIITA guide RNA. In some embodiments, the cell is an allogeneic cell. In some embodiments, the cell is homozygous for HLA-B and homozygous for HLA-C.

In some embodiments, methods are provided for reducing surface expression of MHC class II on the engineered human cell. MHC class II expression is impacted by a variety of proteins. (See e.g., Crivello et al., Journal Immunology 202:1895-1903 (2019).) For example, the CIITA protein functions as a transcriptional activator (activating the MHC class II promoter) and is essential for MHC class II protein expression. In some embodiments, MHC class II protein expression is reduced or eliminated by genetically modifying a gene selected from: CIITA, HLA-DR, HLA-DQ, HLA-DP, RFXS, RFXB/ANK, RFXAP, CREB, NF-YA, NF-YB, and NF-YC. In some embodiments, MHC class II protein expression is reduced or eliminated by genetically modifying the CIITA gene. In some embodiments, MHC class II protein expression is reduced or eliminated by genetically modifying the HLA-DR gene. In some embodiments, MHC class II protein expression is reduced or eliminated by genetically modifying the HLA-DQ gene. In some embodiments, MHC class II protein expression is reduced or eliminated by genetically modifying the HLA-DP gene. In some embodiments, MHC class II protein expression is reduced or eliminated by genetically modifying the RFX5 gene. In some embodiments, MHC class II protein expression is reduced or eliminated by genetically modifying the RFXB/ANK gene. In some embodiments, MHC class II protein expression is reduced or eliminated by genetically modifying the RFXAP gene. In some embodiments, MHC class II protein expression is reduced or eliminated by genetically modifying the CREB gene. In some embodiments, MHC class II protein expression is reduced or eliminated by genetically modifying the NK-YA gene. In some embodiments, MHC class II protein expression is reduced or eliminated by genetically modifying the NK-YB gene. In some embodiments, MHC class II protein expression is reduced or eliminated by genetically modifying the NK-YC gene.

In some embodiments, methods are provided for making an engineered human cell which has reduced or eliminated expression of HLA-A protein relative to an unmodified cell, wherein the cell is homozygous for HLA-B and homozygous for HLA-C, further comprising reducing or eliminating the surface expression of MHC class II protein in the cell relative to an unmodified cell. In some embodiments, the methods comprise contacting the cell with a CIITA guide RNA.

In some embodiments, the efficacy of a CIITA guide RNA is determined by measuring levels of CIITA protein in a cell. The levels of CIITA protein may be detected by, e.g., cell lysate and western blot with an anti-CIITA antibody. In some embodiments, the efficacy of a CIITA guide RNA is determined by measuring levels of CIITA protein in the cell nucleus. In some embodiments, the efficacy of a CIITA guide RNA is determined by measuring levels of CIITA mRNA in a cell. The levels of CIITA mRNA may be detected by e.g., RT-PCR. In some embodiments, a decrease in the levels CIITA protein and/or CIITA mRNA in the target cell as compared to an unmodified cell is indicative of an effective CIITA guide RNA.

In some embodiments, the efficacy of a CIITA guide RNA is determined by measuring the reduction or elimination of MHC class II protein expression by the target cells. The CIITA protein functions as a transactivator, activating the MHC class II promoter, and is essential for the expression of MHC class II protein. In some embodiments, MHC class II protein expression may be detected on the surface of the target cells. In some embodiments, MHC class II protein expression is measured by flow cytometry. In some embodiments, an antibody against MHC class II protein (e.g., anti-HLA-DR, -DQ, -DP) may be used to detect MHC class II protein expression e.g., by flow cytometry. In some embodiments, a reduction or elimination in MHC class II protein on the surface of a cell (or population of cells) as compared to an unmodified cell (or population of unmodified cells) is indicative of an effective CIITA guide RNA. In some embodiments, a cell (or population of cells) that has been contacted with a particular CIITA guide RNA and RNA-guided DNA binding agent that is negative for MHC class II protein by flow cytometry is indicative of an effective CIITA guide RNA.

In some embodiments, the MHC class II protein expression is reduced or eliminated in a population of cells using the methods and compositions disclosed herein. In some embodiments, the population of cells is enriched (e.g., by FACS or MACS) and is at least 65%, 70%, 80%, 90%, 91%, 92%, 93%, or 94% MHC class II negative as measured by flow cytometry relative to a population of unmodified cells. In some embodiments, the population of cells is not enriched (e.g., by FACS or MACS) and is at least 65%, 70%, 80%, 90%, 91%, 92%, 93%, or 94% MHC class II negative as measured by flow cytometry relative to a population of unmodified cells.

In some embodiments, the population of cells is at least 65% MHC II negative as measured by flow cytometry relative to a population of unmodified cells. In some embodiments, the population of cells is at least 70% MHC class II negative as measured by flow cytometry relative to a population of unmodified cells. In some embodiments, the population of cells is at least 80% MHC II negative as measured by flow cytometry relative to a population of unmodified cells. In some embodiments, the population of cells is at least 90% MHC class II negative as measured by flow cytometry relative to a population of unmodified cells. In some embodiments, the population of cells is at least 91% MHC class II negative as measured by flow cytometry relative to a population of unmodified cells. In some embodiments, the population of cells is at least 92% MHC II negative as measured by flow cytometry relative to a population of unmodified cells. In some embodiments, the population of cells is at least 93% MHC class II negative as measured by flow cytometry relative to a population of unmodified cells. In some embodiments, the population of cells is at least 94% MHC class II negative as measured by flow cytometry relative to a population of unmodified cells.

In some embodiments, the population of cells elicits a reduced response from immune cells in vitro or in vivo (e.g., CD4+ T cells). A CD4+ T cell response may be evaluated by an assay that measures the activation response of CD4+ T cells e.g., CD4+ T cell proliferation, expression of activation markers, and/or cytokine production (IL-2, IL-12, IFN-γ) (e.g., flow cytometry, ELISA). The response of CD4+ T cells may be evaluated in in vitro cell culture assays in which the genetically modified cell is co-cultured with cells comprising CD4+ T cells. For example, the engineered cell may be co-cultured e.g., with PBMCs, purified CD3+ T cells comprising CD4+ T cells, purified CD4+ T cells, or a CD4+ T cell line. The CD4+ T cell response elicited from the engineered cell may be compared to the response elicited from an unmodified cell.

In some embodiments, an engineered human cell is provided wherein the cell has reduced or eliminated expression of HLA-A and MHC class II protein on the cell surface, wherein the cell comprises a genetic modification in the HLA-A gene, wherein the cell is homozygous for HLA-B and homozygous for HLA-C, and wherein the cell comprises a modification in the CIITA gene. In some embodiments, the engineered cell elicits a reduced response from CD4+ T cells and elicits a reduced response from CD8+ T cells.

### 2. Exogenous Nucleic Acids Knock in

In some embodiments, the present disclosure provides methods and compositions for reducing or eliminating expression of HLA-A protein on the surface of a cell by genetically modifying HLA-A as disclosed herein, wherein the methods and compositions further provide for expression of a protein encoded by an exogenous nucleic acid (e.g., an antibody, chimeric antigen receptor (CAR), T cell receptor (TCR), cytokine or cytokine receptor, chemokine or chemokine receptor, enzyme, fusion protein, or other type of cell-surface bound or soluble polypeptide). In some embodiments, the exogenous nucleic acid encodes a protein that is expressed on the cell surface. For example, in some embodiments, the exogenous nucleic acid encodes a targeting receptor expressed on the cell surface (described further herein). In some embodiments, the genetically modified cell may function as a “cell factory” for the expression of a secreted polypeptide encoded by an exogenous nucleic acid, including e.g., as a source for continuous production of a polypeptide in vivo (as described further herein). In some embodiments, the cell is an allogeneic cell. In some embodiments, the cell is homozygous for HLA-B and homozygous for HLA-C.

In some embodiments, the methods comprise reducing expression of HLA-A protein on the surface of a cell comprising genetically modifying the HLA-A gene comprising contacting the cell with a composition comprising an HLA-A guide RNA disclosed herein, the method further comprising contacting the cell with an exogenous nucleic acid.

In some embodiments, the methods comprise reducing or eliminating expression of HLA-A protein on the surface of a cell, comprising genetically modifying the cell with one or more compositions comprising a HLA-A guide RNA as disclosed herein, an exogenous nucleic acid encoding a polypeptide (e.g., a targeting receptor), and an RNA-guided DNA binding agent or a nucleic acid encoding an RNA-guided DNA binding agent.

In some embodiments, the methods comprise reducing or eliminating expression of HLA-A protein and MHC class II protein on the surface of a cell, comprising genetically modifying the cell with one or more compositions comprising a HLA-A guide RNA as disclosed herein, a CIITA guide RNA, an exogenous nucleic acid encoding a polypeptide (e.g., a targeting receptor), and an RNA-guided DNA binding agent or a nucleic acid encoding an RNA-guided DNA binding agent.

In some embodiments, the exogenous nucleic acid encodes a polypeptide that is expressed on the surface of the cell. In some embodiments, the exogenous nucleic acid encodes a soluble polypeptide. As used herein, “soluble” polypeptide refers to a polypeptide that is secreted by the cell. In some embodiments, the soluble polypeptide is a therapeutic polypeptide. In some embodiments, the soluble polypeptide is an antibody. In some embodiments, the soluble polypeptide is an enzyme. In some embodiments, the soluble polypeptide is a cytokine. In some embodiments, the soluble polypeptide is a chemokine. In some embodiments, the soluble polypeptide is a fusion protein.

In some embodiments, the exogenous nucleic acid encodes an antibody. In some embodiments, the exogenous nucleic acid encodes an antibody fragment (e.g., Fab, Fab2). In some embodiments, the exogenous nucleic acid encodes is a full-length antibody. In some embodiments, the exogenous nucleic acid encodes is a single-chain antibody (e.g., scFv). In some embodiments, the antibody is an IgG, IgM, IgD, IgA, or IgE. In some embodiments, the antibody is an IgG antibody. In some embodiments, the antibody is an IgG1 antibody. In some embodiments, the antibody is an IgG4 antibody. In some embodiments, the heavy chain constant region contains mutations known to reduce effector functions. In some embodiments, the heavy chain constant region contains mutations known to enhance effector functions. In some embodiments, the antibody is a bispecific antibody. In some embodiments, the antibody is a single-domain antibody (e.g., VH domain-only antibody).

In some embodiments, the exogenous nucleic acid encodes a neutralizing antibody. A neutralizing antibody neutralizes the activity of its target antigen. In some embodiments, the antibody is a neutralizing antibody against a virus antigen. In some embodiments, the antibody neutralizes a target viral antigen, blocking the ability of the virus to infect a cell. In some embodiments, a cell-based neutralization assay may be used to measure the neutralizing activity of an antibody. The particular cells and readout will depend on the target antigen of the neutralizing antibody. The half maximal effective concentration (EC₅₀) of the antibody can be measured in a cell-based neutralization assay, wherein a lower EC₅₀ is indicative of more potent neutralizing antibody.

In some embodiments, the exogenous nucleic acid encodes an antibody that binds to an antigen associated with a disease or disorder (see e.g., diseases and disorders described in Section IV).

In some embodiments, the exogenous nucleic acid encodes a polypeptide that is expressed on the surface of the cell (i.e., a cell-surface bound protein). In some embodiments, the exogenous nucleic acid encodes a targeting receptor. A “targeting receptor” is a receptor present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a proliferation-inducing ligand (APRIL). In some embodiments, the targeting receptor is a TCR. In some embodiments, the targeting receptor is a TRuC. In some embodiments, the targeting receptor is a B cell receptor (BCR) (e.g., expressed on a B cell). In some embodiments, the targeting receptor is chemokine receptor. In some embodiments, the targeting receptor is a cytokine receptor.

In some embodiments, targeting receptors include a chimeric antigen receptor (CAR), a T-cell receptor (TCR), and a receptor for a cell surface molecule operably linked through at least a transmembrane domain in an internal signaling domain capable of activating a T cell upon binding of the extracellular receptor portion. In some embodiments, a CAR refers to an extracellular antigen recognition domain, e.g., an scFv, VHH, nanobody; operably linked to an intracellular signaling domain, which activates the T cell when an antigen is bound. CARs are composed of four regions: an antigen recognition domain, an extracellular hinge region, a transmembrane domain, and an intracellular T-cell signaling domain. Such receptors are well known in the art (see, e.g., WO2020092057, WO2019191114, WO2019147805, WO2018208837). A universal CAR (UniCAR) for recognizing various antigens (see, e.g., EP 2 990 416 A1) and a reversed universal CAR (RevCAR) that promotes binding of an immune cell to a target cell through an adaptor molecule (see, e.g., WO2019238722) are also contemplated. CARs can be targeted to any antigen to which an antibody can be developed and are typically directed to molecules displayed on the surface of a cell or tissue to be targeted. In some embodiments, the targeting receptor comprises an antigen recognition domain (e.g., a cancer antigen recognition domain and a subunit of a TCR (e.g., a TRuC). (See Baeuerle et al. Nature Communications 2087 (2019).)

In some embodiments, the exogenous nucleic acid encodes a TCR. In some embodiments, the exogenous nucleic acid encodes a genetically modified TCR. In some embodiments, the exogenous nucleic acid encodes is a genetically modified TCR with specificity for a polypeptide expressed by cancer cells. In some embodiments, the exogenous nucleic acid encodes a targeting receptor specific for Wilms' tumor gene (WT1) antigen. In some embodiments, the exogenous nucleic acid encodes the WT1-specific TCR (see e.g., WO2020/081613A1).

In some embodiments, an exogenous nucleic acid is inserted into the genome of the target cell. In some embodiments, the exogenous nucleic acid is integrated into the genome of the target cell. In some embodiments, the exogenous nucleic acid is integrated into the genome of the target cell by homologous recombination (HR). In some embodiments, the exogenous nucleic acid is integrated into the genome of the target cell by blunt end insertion. In some embodiments, the exogenous nucleic acid is integrated into the genome of the target cell by non-homologous end joining. In some embodiments, the exogenous nucleic acid is integrated into a safe harbor locus in the genome of the cell. In some embodiments, the exogenous nucleic acid is integrated into one of the TRAC locus, B2M locus, AAVS1 locus, and/or CIITA locus. In some embodiments, the exogenous nucleic acid is provided to the cell in a lipid nucleic acid assembly composition. In some embodiments, the lipid nucleic acid assembly composition is a lipid nanoparticle (LNP).

In some embodiments, the methods produce a composition comprising an engineered cell having reduced or eliminated HLA-A expression and comprising an exogenous nucleic acid. In some embodiments, the methods produce a composition comprising an engineered cell having reduced or eliminated HLA-A expression and that secretes and/or expresses a polypeptide encoded by an exogenous nucleic acid integrated into the genome of the cell. In some embodiments, the methods produce a composition comprising an engineered cell having reduced or eliminated HLA-A protein expression, and/or reduced or eliminated HLA-A levels in the cell nucleus, and having reduced MHC class II protein expression, and secreting and/or expressing a polypeptide encoded by an exogenous nucleic acid integrated into the genome of the cell. In some embodiments, the engineered cell elicits a reduced response from CD4+ T cells, and/or CD8+ T cells.

In some embodiments, an allogeneic cell is provided wherein the cell has reduced or eliminated expression of MHC class II and HLA-A protein on the cell surface, wherein the cell comprises a modification in the HLA-A gene as disclosed herein, wherein the cell comprises a modification in the CIITA gene, and wherein the cell further comprises an exogenous nucleic acid encoding a polypeptide (e.g., a targeting receptor).

In some embodiments, the present disclosure provides methods for reducing or eliminating expression of HLA-A protein on the surface of a cell by genetically modifying HLA-A as disclosed herein, wherein the methods further provide for reducing expression of one or more additional target genes (e.g., TRAC, TRBC). In some embodiments, the additional genetic modifications provide further advantages for use of the genetically modified cells for adoptive cell transfer applications. In some embodiments, the cell is an allogeneic cell. In some embodiments, the cell is homozygous for HLA-B and homozygous for HLA-C.

In some embodiments, the methods comprise reducing or eliminating expression of HLA-A protein on the surface of a cell, comprising genetically modifying the cell with one or more compositions comprising a HLA-A guide RNA as disclosed herein, a CIITA guide RNA, an exogenous nucleic acid encoding polypeptide (e.g., a targeting receptor), a guide RNA that directs an RNA-guided DNA binding agent to a target sequence located in an another gene, thereby reducing or eliminating expression of the other gene, and an RNA-guided DNA binding agent or a nucleic acid encoding an RNA-guided DNA binding agent. In some embodiments, the additional target gene is TRAC. In some embodiments, the additional target gene is TRBC.

### E. Exemplary Cell Types

In some embodiments, methods and compositions disclosed herein genetically modify a human cell. In some embodiments, the cell is an allogeneic cell. In some embodiments the genetically modified cell is referred to as an engineered cell. An engineered cell refers to a cell (or progeny of a cell) comprising an engineered genetic modification, e.g. that has been contacted with a gene editing system and genetically modified by the gene editing system. The terms “engineered cell” and “genetically modified cell” are used interchangeably throughout. The engineered human cell may be any of the exemplary cell types disclosed herein. Further, because MHC class I molecules are expressed on all nucleated cells, the engineered human cell may be any nucleated cell.

In some embodiments, when the cell is homozygous for HLA-B, the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

In some embodiments, when the cell is homozygous for HLA-C, the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C and the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

In some embodiments, the cell is homozygous for HLA-B and homozygous for HLA-C. In some embodiments, the HLA-B and HLA-C alleles of the engineered human cell are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02. In some embodiments, the HLA-B and HLA-C alleles are HLA-B*07:02 and HLA-C*07:02. In some embodiments, the HLA-B and HLA-C alleles are HLA-B*08:01 and HLA-C*07:01. In some embodiments, the HLA-B and HLA-C alleles are HLA-B*44:02 and HLA-C*05:01. In some embodiments, the HLA-B and HLA-C alleles are HLA-B*35:01 and HLA-C*04:01. In some embodiments, the HLA-B and HLA-C alleles are HLA-B*40:01 and HLA-C*03:04. In some embodiments, the HLA-B and HLA-C alleles are HLA-B*57:01 and HLA-C*06:02. In some embodiments, the HLA-B and HLA-C alleles are HLA-B*14:02 and HLA-C*08:02. In some embodiments, the HLA-B and HLA-C alleles are HLA-B*15:01 and HLA-C*03:03. In some embodiments, the HLA-B and HLA-C alleles are HLA-B*13:02 and HLA-C*06:02. In some embodiments, the HLA-B and HLA-C alleles are HLA-B*44:03 and HLA-C*16:01. In some embodiments, the HLA-B and HLA-C alleles are HLA-B*38:01 and HLA-C*12:03. In some embodiments, the HLA-B and HLA-C alleles are HLA-B*18:01 and HLA-C*07:01. In some embodiments, the HLA-B and HLA-C alleles are HLA-B*44:03 and HLA-C*04:01. In some embodiments, the HLA-B and HLA-C alleles are HLA-B*51:01 and HLA-C*15:02. In some embodiments, the HLA-B and HLA-C alleles are HLA-B*49:01 and HLA-C*07:01. In some embodiments, the HLA-B and HLA-C alleles are HLA-B*15:01 and HLA-C*03:04. In some embodiments, the HLA-B and HLA-C alleles are HLA-B*18:01 and HLA-C*12:03. In some embodiments, the HLA-B and HLA-C alleles are HLA-B*27:05 and HLA-C*02:02. In some embodiments, the HLA-B and HLA-C alleles are HLA-B*35:03 and HLA-C*04:01. In some embodiments, the HLA-B and HLA-C alleles are HLA-B*18:01 and HLA-C*05:01. In some embodiments, the HLA-B and HLA-C alleles are HLA-B*52:01 and HLA-C*12:02. In some embodiments, the HLA-B and HLA-C alleles are HLA-B*51:01 and HLA-C*14:02. In some embodiments, the HLA-B and HLA-C alleles are HLA-B*37:01 and HLA-C*06:02. In some embodiments, the HLA-B and HLA-C alleles are HLA-B*53:01 and HLA-C*04:01. In some embodiments, the HLA-B and HLA-C alleles are HLA-B*55:01 and HLA-C*03:03. In some embodiments, the HLA-B and HLA-C alleles are HLA-B*44:02 and HLA-C*07:04. In some embodiments, the HLA-B and HLA-C alleles are HLA-B*44:03 and HLA-C*07:01. In some embodiments, the HLA-B and HLA-C alleles are HLA-B*35:02 and HLA-C*04:01. In some embodiments, the HLA-B and HLA-C alleles are HLA-B*15:01 and HLA-C*04:01. In some embodiments, the HLA-B and HLA-C alleles are and HLA-B*40:02 and HLA-C*02:02.

In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3⁺, CD4⁺ and CD8⁺ T cells, regulatory T cells (Tregs), B cells, NK cells, and dendritic cells (DC). In some embodiments, the immune cell is allogeneic.

In some embodiments, the cell is a lymphocyte. In some embodiments, the cell is an adaptive immune cell. In some embodiments, the cell is a T cell. In some embodiments, the cell is a B cell. In some embodiments, the cell is a NK cell. In some embodiments, the cell is a macrophage. In some embodiments, the lymphocyte is allogeneic.

As used herein, a T cell can be defined as a cell that expresses a T cell receptor (“TCR” or “αβ TCR” or “γδ TCR”), however in some embodiments, the TCR of a T cell may be genetically modified to reduce its expression (e.g., by genetic modification to the TRAC or TRBC genes), therefore expression of the protein CD3 may be used as a marker to identify a T cell by standard flow cytometry methods. CD3 is a multi-subunit signaling complex that associates with the TCR. Thus, a T cell may be referred to as CD3+. In some embodiments, a T cell is a cell that expresses a CD3+ marker and either a CD4+ or CD8+ marker. In some embodiments, the T cell is allogeneic.

In some embodiments, the T cell expresses the glycoprotein CD8 and therefore is CD8+ by standard flow cytometry methods and may be referred to as a “cytotoxic” T cell. In some embodiments, the T cell expresses the glycoprotein CD4 and therefore is CD4+ by standard flow cytometry methods and may be referred to as a “helper” T cell. CD4+ T cells can differentiate into subsets and may be referred to as a Th1 cell, Th2 cell, Th9 cell, Th17 cell, Th22 cell, T regulatory (“Treg”) cell, or T follicular helper cells (“Tfh”). Each CD4+ subset releases specific cytokines that can have either proinflammatory or anti-inflammatory functions, survival or protective functions. A T cell may be isolated from a subject by CD4+ or CD8+ selection methods.

In some embodiments, the T cell is a memory T cell. In the body, a memory T cell has encountered antigen. A memory T cell can be located in the secondary lymphoid organs (central memory T cells) or in recently infected tissue (effector memory T cells). A memory T cell may be a CD8+ T cell. A memory T cell may be a CD4+ T cell.

As used herein, a “central memory T cell” can be defined as an antigen-experienced T cell, and for example, may expresses CD62L and CD45RO. A central memory T cell may be detected as CD62L+ and CD45RO+ by Central memory T cells also express CCR7, therefore may be detected as CCR7+ by standard flow cytometry methods.

As used herein, an “early stem-cell memory T cell” (or “Tscm”) can be defined as a T cell that expresses CD27 and CD45RA, and therefore is CD27+ and CD45RA+ by standard flow cytometry methods. A Tscm does not express the CD45 isoform CD45RO, therefore a Tscm will further be CD45RO− if stained for this isoform by standard flow cytometry methods. A CD45RO− CD27+ cell is therefore also an early stem-cell memory T cell. Tscm cells further express CD62L and CCR7, therefore may be detected as CD62L+ and CCR7+ by standard flow cytometry methods. Early stem-cell memory T cells have been shown to correlate with increased persistence and therapeutic efficacy of cell therapy products.

In some embodiments, the cell is a B cell. As used herein, a “B cell” can be defined as a cell that expresses CD19 and/or CD20, and/or B cell mature antigen (“BCMA”), and therefore a B cell is CD19+, and/or CD20+, and/or BCMA+ by standard flow cytometry methods. A B cell is further negative for CD3 and CD56 by standard flow cytometry methods. The B cell may be a plasma cell. The B cell may be a memory B cell. The B cell may be a naïve B cell. The B cell may be IgM+, or has a class-switched B cell receptor (e.g., IgG+, or IgA+). In some embodiments, the B cell is allogeneic.

In some embodiments, the cell is a mononuclear cell, such as from bone marrow or peripheral blood. In some embodiments, the cell is a peripheral blood mononuclear cell (“PBMC”). In some embodiments, the cell is a PBMC, e.g. a lymphocyte or monocyte. In some embodiments, the cell is a peripheral blood lymphocyte (“PBL”). In some embodiments, the mononuclear cell is allogeneic.

Cells used in ACT and/or tissue regenerative therapy are included, such as stem cells, progenitor cells, and primary cells. Stem cells, for example, include pluripotent stem cells (PSCs); induced pluripotent stem cells (iPSCs); embryonic stem cells (ESCs); mesenchymal stem cells (MSCs, e.g., isolated from bone marrow (BM), peripheral blood (PB), placenta, umbilical cord (UC) or adipose); hematopoietic stem cells (HSCs; e.g. isolated from BM or UC); neural stem cells (NSCs); tissue specific progenitor stem cells (TSPSCs); and limbal stem cells (LSCs). Progenitor and primary cells include mononuclear cells (MNCs, e.g., isolated from BM or PB); endothelial progenitor cells (EPCs, e.g. isolated from BM, PB, and UC); neural progenitor cells (NPCs); and tissue-specific primary cells or cells derived therefrom (TSCs) including chondrocytes, myocytes, and keratinocytes. Cells for organ or tissue transplantations such as islet cells, cardiomyocytes, thyroid cells, thymocytes, neuronal cells, skin cells, and retinal cells are also included.

In some embodiments, the human cell is isolated from a human subject. In some embodiments, the cell is isolated from human donor PBMCs or leukopaks. In some embodiments, the cell is from a subject with a condition, disorder, or disease. In some embodiments, the cell is from a human donor with Epstein Barr Virus (“EBV”).

In some embodiments, the methods are carried out ex vivo. As used herein, “ex vivo” refers to an in vitro method wherein the cell is capable of being transferred into a subject, e.g. as an ACT therapy. In some embodiments, an ex vivo method is an in vitro method involving an ACT therapy cell or cell population.

In some embodiments, the cell is from a cell line. In some embodiments, the cell line is derived from a human subject. In some embodiments, the cell line is a lymphoblastoid cell line (“LCL”). The cell may be cryopreserved and thawed. The cell may not have been previously cryopreserved.

In some embodiments, the cell is from a cell bank. In some embodiments, the cell is genetically modified and then transferred into a cell bank. In some embodiments the cell is removed from a subject, genetically modified ex vivo, and transferred into a cell bank. In some embodiments, a genetically modified population of cells is transferred into a cell bank. In some embodiments, a genetically modified population of immune cells is transferred into a cell bank. In some embodiments, a genetically modified population of immune cells comprising a first and second subpopulations, wherein the first and second sub-populations have at least one common genetic modification and at least one different genetic modification are transferred into a cell bank.

### F. Exemplary Gene Editing Systems

Various suitable gene editing systems may be used to make the engineered cells disclosed herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in gene editing and gene therapy.

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for gene editing in situ, a technique known as gene editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type IIs restriction endonuclease Fokl is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In some embodiments, the gene editing system is a CRISPR/Cas system, including e.g., a CRISPR guide RNA comprising a guide sequence and RNA-guided DNA binding agent, and described further herein.

### G. CRISPR Guide RNA

Provided herein are guide sequences useful for modifying a target sequence, e.g., using a guide RNA comprising a disclosed guide sequence with an RNA-guided DNA binding agent (e.g., a CRISPR/Cas system).

Each of the guide sequences disclosed herein may further comprise additional nucleotides to form a crRNA, e.g., with the following exemplary nucleotide sequence following the guide sequence at its 3′ end: GUUUUAGAGCUAUGCUGUUUUG (SEQ ID NO: 213) in 5′ to 3′ orientation. In the case of a sgRNA, the above guide sequences may further comprise additional nucleotides (scaffold sequence) to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 214) or GUUUUAGAGCUAGAAAUAGCAAGUUAAAAUAAGGCUAGUCCGUUAUCAACUU GAAAAAGUGGCACCGAGUCGGUGC (SEQ ID NO: 215, which is SEQ ID NO: 214 without the four terminal U's) in 5′ to 3′ orientation. In some embodiments, the four terminal U's of SEQ ID NO: 214 are not present. In some embodiments, only 1, 2, or 3 of the four terminal U's of SEQ ID NO: 214 are present.

In some embodiments, the sgRNA comprises any one of the guide sequences of SEQ ID Nos: 1-211 and additional nucleotides to form a crRNA, e.g., with the following exemplary scaffold nucleotide sequence following the guide sequence at its 3′ end: GUUUUAGAGCUAGAAAUAGCAAGUUAAAAUAAGGCUAGUCCGUUAUCAACUU GGCACCGAGUCGGUGC (SEQ ID NO: 216) in 5′ to 3′ orientation. SEQ ID NO: 216 lacks 8 nucleotides with reference to a wild-type guide RNA conserved sequence: GUUUUAGAGCUAGAAAUAGCAAGUUAAAAUAAGGCUAGUCCGUUAUCAACUU GAAAAAGUGGCACCGAGUCGGUGC (SEQ ID NO: 215). Other exemplary scaffold nucleotide sequences are provided in Table 6. In some embodiments, the sgRNA comprises any one of the guide sequences of SEQ ID NOs: 1-211 and additional guide scaffold sequences, in 5′ to 3′ orientation, in Table 6, including modified versions of the scaffold sequences, as shown.

In some embodiments, the guide RNA is a sgRNA comprising any one of the sequences shown in Table 2 (SEQ ID NOs: 249-343 and 344-438), Table 3 (SEQ ID NOs: 439-471 and 472-504), and Table 5 (SEQ ID NOs: 505-532 and 533-560). In some embodiments, the guide RNA is a chemically modified guide RNA. In some embodiments, the guide RNA is a chemically modified single guide RNA. The chemically modified guide RNAs may comprise one or more of the modifications as shown in Tables 2, 3, 5, and 6. The chemically modified guide RNAs may comprise one or more of modified nucleotides of any one of SEQ ID NOs: 1003, 1007-1009 and 1011-1014.

In some embodiments, the guide RNA is a sgRNA comprising any one of SEQ ID NOs: 249-343, 439-471, and 505-532 with at least one chemical modification disclosed herein. In some embodiments, the guide RNA is a sgRNA comprising a sequence that is at least 99%, 98%, 97%, 96%, 95%, 94%, 93%, 92%, 91%, or 90% identical to any one of SEQ ID NOs: 249-343, 439-471, and 505-532 with at least one chemical modification disclosed herein.

In some embodiments, the guide RNA is a sgRNA comprising the modification pattern shown in SEQ ID NO: 1013 or 1014. In some embodiments, the guide RNA is a sgRNA comprising a sequence that is at least 99%, 98%, 97%, 96%, 95%, 94%, 93%, 92%, 91%, or 90% identical to any of the nucleic acids of SEQ ID NOs: 344-438, 472-504, and 533-560.

In some embodiments, the guide RNA comprises a sgRNA comprising the modification pattern shown in SEQ ID NO: 1003. In some embodiments, the guide RNA comprises a sgRNA comprising the modified nucleotides of SEQ ID NO: 1003, including a guide sequence comprises a sequence selected from SEQ ID NOs: 1-211. In some embodiments, the guide RNA is a sgRNA comprising a sequence of SEQ ID NO: 1016 or a sequence that is at least 99%, 98%, 97%, 96%, 95%, 94%, 93%, 92%, 91%, or 90% identical to SEQ ID NO: 1016.

In some embodiments, the guide RNA comprises a single guide RNA comprising any one of the sequences of SEQ ID NOs: 344-438, 472-504, and 533-560, and 1016 or a sequence that is at least 99%, 98%, 97%, 96%, 95%, 94%, 93%, 92%, 91%, or 90% identical to any one of the sequences of SEQ ID NOs: 344-438, 472-504, and 533-560, and 1016.

In some embodiments, the guide RNA comprises a guide sequence comprising any one of SEQ ID NOs: 13-18, 26, 37-39, 41, 43, 45, and 62. In some embodiments, the guide RNA comprises a single guide RNA comprising any one of the sequences SEQ ID NOs: 356-361, 369, 380-382, 384, 386, 388, and 405, or a sequence that is at least 99%, 98%, 97%, 96%, 95%, 94%, 93%, 92%, 91%, or 90% identical to any one of the sequences SEQ ID NOs: 356-361, 369, 380-382, 384, 386, 388, and 405.

The guide RNA may further comprise a trRNA. In each composition and method embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond. In some embodiments, a crRNA and/or trRNA sequence may be referred to as a “scaffold” or “conserved portion” of a guide RNA.

In each of the compositions, use, and method embodiments described herein, the guide RNA may comprise two RNA molecules as a “dual guide RNA” or “dgRNA.” The dgRNA comprises a first RNA molecule comprising a crRNA comprising, e.g., a guide sequence shown in Tables 2-5, and a second RNA molecule comprising a trRNA. The first and second RNA molecules may not be covalently linked, but may form an RNA duplex via the base pairing between portions of the crRNA and the trRNA.

In each of the composition, use, and method embodiments described herein, the guide RNA may comprise a single RNA molecule as a “single guide RNA” or “sgRNA”. The sgRNA may comprise a crRNA (or a portion thereof) comprising a guide sequence shown in Tables 2-5, covalently linked to a trRNA. The sgRNA may comprise 17, 18, 19, or 20 contiguous nucleotides of a guide sequence shown in Tables 2-5. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one in Tables 2-5 is provided. In some embodiments, a composition comprising one or more guide RNAs comprising a guide sequence of any one in Tables 2-5 is provided, wherein the nucleotides of SEQ ID NO: 213-216 follow the guide sequence at its 3′ end. In some embodiments, the one or more guide RNAs comprising a guide sequence of any one in Tables 2-5, wherein the nucleotides of SEQ ID NO: 213-216 follow the guide sequence at its 3′ end, is modified according to the modification pattern of any one of SEQ ID NOs: 1003, 1007-1009, and 1011-1014.

In some embodiments, a composition comprising one or more guide RNAs comprising a guide sequence of any one in Tables 2-5 is provided. In one aspect, a composition comprising one or more gRNAs is provided, comprising a guide sequence that is at least 99%, 98%, 97%, 96%, 95%, 94%, 93%, 92%, 91%, or 90% identical to any of the nucleic acids of SEQ ID NOs: 1-211.

In other embodiments, a composition is provided that comprises at least one, e.g., at least two gRNA's comprising guide sequences selected from any two or more of the guide sequences shown in Tables 2-5. In some embodiments, the composition comprises at least two gRNA's that each comprise a guide sequence at least 99%, 98%, 97%, 96%, 95%, 94%, 93%, 92%, 91%, or 90% identical to any of the guide sequences shown in Tables 2-5.

In some embodiments, the guide RNA compositions of the present invention are designed to recognize (e.g., hybridize to) a target sequence in HLA-A. For example, the HLA-A target sequence may be recognized and cleaved by a provided Cas cleavase comprising a guide RNA. In some embodiments, an RNA-guided DNA binding agent, such as a Cas cleavase, may be directed by a guide RNA to a target sequence in HLA-A, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within HLA-A. In some embodiments, the compositions comprising one or more guide sequences comprise a guide sequence that is complementary to the corresponding genomic region shown in Tables 2-5, according to coordinates from human reference genome hg38. Guide sequences of further embodiments may be complementary to sequences in the close vicinity of the genomic coordinate listed in any of the Tables 2-5 within HLA-A. For example, guide sequences of further embodiments may be complementary to sequences that comprise 10 contiguous nucleotides±10 nucleotides of a genomic coordinate listed in Tables 2-5.

Without being bound by any particular theory, modifications (e.g., frameshift mutations resulting from indels occurring as a result of a nuclease-mediated DSB) in certain regions of the target gene may be less tolerable than mutations in other regions, thus the location of a DSB is an important factor in the amount or type of protein knockdown that may result. In some embodiments, a gRNA complementary or having complementarity to a target sequence within the target gene used to direct an RNA-guided DNA binding agent to a particular location in the target gene.

In some embodiments, the guide sequence is at least 99%, 98%, 97%, 96%, 95%, 94%, 93%, 92%, 91%, 90%, 85%, or 80% identical to a target sequence present in the target gene. In some embodiments, the guide sequence is at least 99%, 98%, 97%, 96%, 95%, 94%, 93%, 92%, 91%, 90%, 85%, or 80% identical to a target sequence present in the human HLA-A gene.

In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

### H. Modified gRNAs and mRNAs

In some embodiments, the gRNA (e.g., sgRNA, short-sgRNA, dgRNA, or crRNA) is modified. The term “modified” or “modification” in the context of a gRNA described herein includes, the modifications described above, including, for example, (a) end modifications, e.g., 5′ end modifications or 3′ end modifications, including 5′ or 3′ protective end modifications, (b) nucleobase (or “base”) modifications, including replacement or removal of bases, (c) sugar modifications, including modifications at the 2′, 3′, and/or 4′ positions, (d) internucleoside linkage modifications, and (e) backbone modifications, which can include modification or replacement of the phosphodiester linkages and/or the ribose sugar. A modification of a nucleotide at a given position includes a modification or replacement of the phosphodiester linkage immediately 3′ of the sugar of the nucleotide. Thus, for example, a nucleic acid comprising a phosphorothioate between the first and second sugars from the 5′ end is considered to comprise a modification at position 1. The term “modified gRNA” generally refers to a gRNA having a modification to the chemical structure of one or more of the base, the sugar, and the phosphodiester linkage or backbone portions, including nucleotide phosphates, all as detailed and exemplified herein.

Further description and exemplary patterns of modifications are provided in Table 1 of WO2019/237069 published Dec. 12, 2019, the entire contents of which are incorporated herein by reference.

In some embodiments, a gRNA comprises modifications at 1, 2, 3, 4, 5, 6, 7, 8, 9, 10, 11, 12, 13, 14, 15, 16, or more YA sites. In some embodiments, the pyrimidine of the YA site comprises a modification (which includes a modification altering the internucleoside linkage immediately 3′ of the sugar of the pyrimidine). In some embodiments, the adenine of the YA site comprises a modification (which includes a modification altering the internucleoside linkage immediately 3′ of the sugar of the adenine). In some embodiments, the pyrimidine and the adenine of the YA site comprise modifications, such as sugar, base, or internucleoside linkage modifications. The YA modifications can be any of the types of modifications set forth herein. In some embodiments, the YA modifications comprise one or more of phosphorothioate, 2′-OMe, or 2′-fluoro. In some embodiments, the YA modifications comprise pyrimidine modifications comprising one or more of phosphorothioate, 2′-OMe, 2′-H, inosine, or 2′-fluoro. In some embodiments, the YA modification comprises a bicyclic ribose analog (e.g., an LNA, BNA, or ENA) within an RNA duplex region that contains one or more YA sites. In some embodiments, the YA modification comprises a bicyclic ribose analog (e.g., an LNA, BNA, or ENA) within an RNA duplex region that contains a YA site, wherein the YA modification is distal to the YA site.

In some embodiments, the guide sequence (or guide region) of a gRNA comprises 1, 2, 3, 4, 5, or more YA sites (“guide region YA sites”) that may comprise YA modifications. In some embodiments, one or more YA sites located at 5-end, 6-end, 7-end, 8-end, 9-end, or 10-end from the 5′ end of the 5′ terminus (where “5-end”, etc., refers to position 5 to the 3′ end of the guide region, i.e., the most 3′ nucleotide in the guide region) comprise YA modifications.. A modified guide region YA site comprises a YA modification.

In some embodiments, a modified guide region YA site is within 20, 19, 18, 17, 16, 15, 14, 13, 12, 11, 10, or 9 nucleotides of the 3′ terminal nucleotide of the guide region. For example, if a modified guide region YA site is within 10 nucleotides of the 3′ terminal nucleotide of the guide region and the guide region is 20 nucleotides long, then the modified nucleotide of the modified guide region YA site is located at any of positions 11-20. In some embodiments, a modified guide region YA site is at or after nucleotide 4, 5, 6, 7, 8, 9, 10, or 11 from the 5′ end of the 5′ terminus.

In some embodiments, a modified guide region YA site is other than a 5′ end modification. For example, a sgRNA can comprise a 5′ end modification as described herein and further comprise a modified guide region YA site. Alternatively, a sgRNA can comprise an unmodified 5′ end and a modified guide region YA site. Alternatively, a short-sgRNA can comprise a modified 5′ end and an unmodified guide region YA site.

In some embodiments, a modified guide region YA site comprises a modification that at least one nucleotide located 5′ of the guide region YA site does not comprise. For example, if nucleotides 1-3 comprise phosphorothioates, nucleotide 4 comprises only a 2′-OMe modification, and nucleotide 5 is the pyrimidine of a YA site and comprises a phosphorothioate, then the modified guide region YA site comprises a modification (phosphorothioate) that at least one nucleotide located 5′ of the guide region YA site (nucleotide 4) does not comprise. In another example, if nucleotides 1-3 comprise phosphorothioates, and nucleotide 4 is the pyrimidine of a YA site and comprises a 2′-OMe, then the modified guide region YA site comprises a modification (2′-OMe) that at least one nucleotide located 5′ of the guide region YA site (any of nucleotides 1-3) does not comprise. This condition is also always satisfied if an unmodified nucleotide is located 5′ of the modified guide region YA site.

In some embodiments, the modified guide region YA sites comprise modifications as described for YA sites above. The guide region of a gRNA may be modified according to any embodiment comprising a modified guide region set forth herein. Any embodiments set forth elsewhere in this disclosure may be combined to the extent feasible with any of the foregoing embodiments.

In some embodiments, the 5′ and/or 3′ terminus regions of a gRNA are modified.

In some embodiments, the terminal (i.e., last) 1, 2, 3, 4, 5, 6, or 7 nucleotides in the 3′ terminus region are modified. Throughout, this modification may be referred to as a “3′ end modification”. In some embodiments, the terminal (i.e., last) 1, 2, 3, 4, 5, 6, or 7 nucleotides in the 3′ terminus region comprise more than one modification. In some embodiments, the 3′ end modification comprises or further comprises any one or more of the following: a modified nucleotide selected from 2′-O-methyl (2′-O-Me) modified nucleotide, 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or combinations thereof. In some embodiments, the 3′ end modification comprises or further comprises modifications of 1, 2, 3, 4, 5, 6, or 7 nucleotides at the 3′ end of the gRNA. In some embodiments, the 3′ end modification comprises or further comprises one PS linkage, wherein the linkage is between the last and second to last nucleotide. In some embodiments, the 3′ end modification comprises or further comprises two PS linkages between the last three nucleotides. In some embodiments, the 3′ end modification comprises or further comprises four PS linkages between the last four nucleotides. In some embodiments, the 3′ end modification comprises or further comprises PS linkages between any one or more of the last 2, 3, 4, 5, 6, or 7 nucleotides. In some embodiments, the gRNA comprising a 3′ end modification comprises or further comprises a 3′ tail, wherein the 3′ tail comprises a modification of any one or more of the nucleotides present in the 3′ tail. In some embodiments, the 3′ tail is fully modified. In some embodiments, the 3′ tail comprises 1, 2, 3, 4, 5, 6, 7, 8, 9, 10, 1-2, 1-3, 1-4, 1-5, 1-6, 1-7, 1-8, 1-9, or 1-10 nucleotides, optionally where any one or more of these nucleotides are modified. In some embodiments, a gRNA is provided comprising a 3′ protective end modification. In some embodiments, the 3′ tail comprises between 1 and about 20 nucleotides, between 1 and about 15 nucleotides, between 1 and about 10 nucleotides, between 1 and about 5 nucleotides, between 1 and about 4 nucleotides, between 1 and about 3 nucleotides, and between 1 and about 2 nucleotides. In some embodiments, the gRNA does not comprise a 3′ tail.

In some embodiments, the 5′ terminus region is modified, for example, the first 1, 2, 3, 4, 5, 6, or 7 nucleotides of the gRNA are modified. Throughout, this modification may be referred to as a “5′ end modification”. In some embodiments, the first 1, 2, 3, 4, 5, 6, or 7 nucleotides of the 5′ terminus region comprise more than one modification. In some embodiments, at least one of the terminal (i.e., first) 1, 2, 3, 4, 5, 6, or 7 nucleotides at the 5′ end are modified. In some embodiments, both the 5′ and 3′ terminus regions (e.g., ends) of the gRNA are modified. In some embodiments, only the 5′ terminus region of the gRNA is modified. In some embodiments, only the 3′ terminus region (plus or minus a 3′ tail) of the conserved portion of a gRNA is modified. In some embodiments, the gRNA comprises modifications at 1, 2, 3, 4, 5, 6, or 7 of the first 7 nucleotides at a 5′ terminus region of the gRNA. In some embodiments, the gRNA comprises modifications at 1, 2, 3, 4, 5, 6, or 7 of the 7 terminal nucleotides at a 3′ terminus region. In some embodiments, 2, 3, or 4 of the first 4 nucleotides at the 5′ terminus region, and/or 2, 3, or 4 of the terminal 4 nucleotides at the 3′ terminus region are modified. In some embodiments, 2, 3, or 4 of the first 4 nucleotides at the 5′ terminus region are linked with phosphorothioate (PS) bonds. In some embodiments, the modification to the 5′ terminus and/or 3′ terminus comprises a 2′-O-methyl (2′-O-Me) or 2′-O-(2-methoxyethyl) (2′-O-moe) modification. In some embodiments, the modification comprises a 2′-fluoro (2′-F) modification to a nucleotide. In some embodiments, the modification comprises a phosphorothioate (PS) linkage between nucleotides. In some embodiments, the modification comprises an inverted abasic nucleotide. In some embodiments, the modification comprises a protective end modification. In some embodiments, the modification comprises a more than one modification selected from protective end modification, 2′-O-Me, 2′-O-moe, 2′-fluoro (2′-F), a phosphorothioate (PS) linkage between nucleotides, and an inverted abasic nucleotide. In some embodiments, an equivalent modification is encompassed.

In some embodiments, a gRNA is provided comprising a 5′ end modification and a 3′ end modification. In some embodiments, the gRNA comprises modified nucleotides that are not at the 5′ or 3′ ends.

In some embodiments, a sgRNA is provided comprising an upper stem modification, wherein the upper stem modification comprises a modification to any one or more of US1-US12 in the upper stem region. In some embodiments, a sgRNA is provided comprising an upper stem modification, wherein the upper stem modification comprises a modification of at least 1, 2, 3, 4, 5, 6, 7, 8, 9, 10, 11, or all 12 nucleotides in the upper stem region. In some embodiments, an sgRNA is provided comprising an upper stem modification, wherein the upper stem modification comprises 1, 2, 3, 4, or 5 YA modifications in a YA site. In some embodiments, the upper stem modification comprises a 2′-OMe modified nucleotide, a 2′-O-moe modified nucleotide, a 2′-F modified nucleotide, and/or combinations thereof. Other modifications described herein, such as a 5′ end modification and/or a 3′ end modification may be combined with an upper stem modification.

In some embodiments, the sgRNA comprises a modification in the hairpin region. In some embodiments, the hairpin region modification comprises at least one modified nucleotide selected from a 2′-O-methyl (2′-OMe) modified nucleotide, a 2′-fluoro (2′-F) modified nucleotide, and/or combinations thereof. In some embodiments, the hairpin region modification is in the hairpin 1 region. In some embodiments, the hairpin region modification is in the hairpin 2 region. In some embodiments, the hairpin modification comprises 1, 2, or 3 YA modifications in a YA site. In some embodiments, the hairpin modification comprises at least 1, 2, 3, 4, 5, or 6 YA modifications. Other modifications described herein, such as an upper stem modification, a 5′ end modification, and/or a 3′ end modification may be combined with a modification in the hairpin region.

In some embodiments, a gRNA comprises a substituted and optionally shortened hairpin 1 region, wherein at least one of the following pairs of nucleotides are substituted in the substituted and optionally shortened hairpin 1 with Watson-Crick pairing nucleotides: H1-1 and H1-12, H1-2 and H1-11, H1-3 and H1-10, and/or H1-4 and H1-9. “Watson-Crick pairing nucleotides” include any pair capable of forming a Watson-Crick base pair, including A-T, A-U, T-A, U-A, C-G, and G-C pairs, and pairs including modified versions of any of the foregoing nucleotides that have the same base pairing preference. In some embodiments, the hairpin 1 region lacks any one or two of H1-5 through H1-8. In some embodiments, the hairpin 1 region lacks one, two, or three of the following pairs of nucleotides: H1-1 and H1-12, H1-2 and H1-11, H1-3 and H1-10 and/or H1-4 and H1-9. In some embodiments, the hairpin 1 region lacks 1-8 nucleotides of the hairpin 1 region. In any of the foregoing embodiments, the lacking nucleotides may be such that the one or more nucleotide pairs substituted with Watson-Crick pairing nucleotides (H1-1 and H1-12, H1-2 and H1-11, H1-3 and H1-10, and/or H1-4 and H1-9) form a base pair in the gRNA.

In some embodiments, the gRNA further comprises an upper stem region lacking at least 1 nucleotide, e.g., any of the shortened upper stem regions indicated in Table 7 of U.S. Application No. 62/946,905, the contents of which are hereby incorporated by reference in its entirety, or described elsewhere herein, which may be combined with any of the shortened or substituted hairpin 1 regions described herein.

In some embodiments, an sgRNA provided herein is a short-single guide RNAs (short-sgRNAs), e.g., comprising a conserved portion of an sgRNA comprising a hairpin region, wherein the hairpin region lacks at least 5-10 nucleotides or 6-10 nucleotides. In some embodiments, the 5-10 nucleotides or 6-10 nucleotides are consecutive.

In some embodiments, a short-sgRNA lacks at least nucleotides 54-58 (AAAAA) of the conserved portion of a spyCas9 sgRNA. In some embodiments, a short-sgRNA is a non-spyCas9 sgRNA that lacks nucleotides corresponding to nucleotides 54-58 (AAAAA) of the conserved portion of a spyCas9 as determined, for example, by pairwise or structural alignment.

In some embodiments, the short-sgRNA described herein comprises a conserved portion comprising a hairpin region, wherein the hairpin region lacks 5, 6, 7, 8, 9, 10, 11, or 12 nucleotides. In some embodiments, the lacking nucleotides are 5-10 lacking nucleotides or 6-10 lacking nucleotides. In some embodiments, the lacking nucleotides are consecutive. In some embodiments, the lacking nucleotides span at least a portion of hairpin 1 and a portion of hairpin 2. In some embodiments, the 5-10 lacking nucleotides comprise or consist of nucleotides 54-58, 54-61, or 53-60 of SEQ ID NO: 215.

In some embodiments, the short-sgRNA described herein further comprises a nexus region, wherein the nexus region lacks at least one nucleotide (e.g., 1, 2, 3, 4, 5, 6, 7, 8, 9, or 10 nucleotides in the nexus region). In some embodiments, the short-sgRNA lacks each nucleotide in the nexus region.

In some embodiments, the SpyCas9 short-sgRNA described herein comprises a sequence of

(SEQ ID NO: 1002)
NNNNNNNNNNNNNNNNNNNNGUUUUAGAGCUAGAAAUAGCAAGUUAAAA
UAAGGCUAGUCCGUUAUCACGAAAGGGCACCGAGUCGGUGCU.

In some embodiments, the short-sgRNA described herein comprises a modification pattern as shown in SEQ ID NO: 1003: mN*mN*mN*NNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 1003), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides.

In certain embodiments, using SEQ ID NO: 215 (“Exemplary SpyCas9 sgRNA-1”) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 6-8 nucleotides, preferably 6 nucleotides; and
      a. one or more of positions H1-1, H1-2, or H1-3 is deleted or substituted relative to Exemplary SpyCas9 sgRNA-1 (SEQ ID NO: 215) or
      b. one or more of positions H1-6 through H1-10 is substituted relative to Exemplary SpyCas9 sgRNA-1 (SEQ ID NO: 215); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 215); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 215); or
C. a substitution relative to Exemplary SpyCas9 sgRNA-1 (SEQ ID NO: 215) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. Exemplary SpyCas9 sgRNA-1 (SEQ ID NO: 215) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-OMe modified nucleotide.

In certain embodiments, Exemplary SpyCas9 sgRNA-1, or an sgRNA, such as an sgRNA comprising Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In some embodiments, the gRNA described herein further comprises a nexus region, wherein the nexus region lacks at least one nucleotide.

In some embodiments, the gRNA is chemically modified. A gRNA comprising one or more modified nucleosides or nucleotides is called a “modified” gRNA or “chemically modified” gRNA, to describe the presence of one or more non-naturally and/or naturally occurring components or configurations that are used instead of or in addition to the canonical A, G, C, and U residues. Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of an gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include phosphorothioate, phosphoroselenates, borano phosphates, borano phosphate esters, hydrogen phosphonates, phosphoroamidates, alkyl or aryl phosphonates and phosphotriesters.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20. In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges. In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a gRNA molecule are deoxynucleotides.

In some embodiments, the gRNAs disclosed herein comprise one of the modification patterns disclosed in WO2018/107028 A1, published Jun. 14, 2018 the contents of which are hereby incorporated by reference in their entirety.

The terms “mA,” “mC,” “mU,” or “mG” may be used to denote a nucleotide that has been modified with 2′-O-Me. The terms “fA,” “fC,” “fU,” or “fG” may be used to denote a nucleotide that has been substituted with 2′-F. A “*” may be used to depict a PS modification. The terms A*, C*, U*, or G* may be used to denote a nucleotide that is linked to the next (e.g., 3′) nucleotide with a PS bond. The terms “mA*,” “mC*,” “mU*,” or “mG*” may be used to denote a nucleotide that has been substituted with 2′-O-Me and that is linked to the next (e.g., 3′) nucleotide with a PS bond.

Exemplary spyCas9 sgRNA-1 (SEQ ID NO: 215)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| | |
|---|---|
| Nexus | H1-1 through H1-12 |

| | | | | | | | | | | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

### I. Ribonucleoprotein Complex

In some embodiments, the disclosure provides compositions comprising one or more gRNAs comprising one or more guide sequences from Tables 2-5 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see e.g., the list in the next paragraph), and modified (e.g., engineered or mutant) versions thereof. See e.g., US2016/0312198 A1; US 2016/0312199 A1. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015). In some embodiments, the RNA-guided DNA-binding agent comprises a Cas nickase. In some embodiments, the RNA-guided nickase is modified or derived from a Cas protein, such as a Class 2 Cas nuclease (which may be, e.g., a Cas nuclease of Type II, V, or VI). Class 2 Cas nuclease include, for example, Cas9, Cpf1, C2c1, C2c2, and C2c3 proteins and modifications thereof.

Non-limiting exemplary species that the Cas nuclease or Cas nickase can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the Cas nickase is derived from the Cas9 nuclease from Streptococcus pyogenes. In some embodiments, the Cas nickase is derived from the Cas9 nuclease from Streptococcus thermophilus. In some embodiments, the Cas nickase is a nickase form of the Cas9 nuclease from Neisseria meningitidis. See e.g., WO/2020081568, describing an Nme2Cas9 D16A nickase fusion protein. In some embodiments, the Cas nickase is derived from the Cas9 nuclease is from Staphylococcus aureus. In some embodiments, the Cas nickase is derived from the Cpf1 nuclease from Francisella novicida. In some embodiments, the Cas nickase is derived from the Cpf1 nuclease from Acidaminococcus sp. In some embodiments, the Cas nickase is derived from the Cpf1 nuclease from Lachnospiraceae bacterium ND2006. In further embodiments, the Cas nickase is derived from the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nickase is derived from a Cpf1 nuclease from an Acidaminococcus or Lachnospiraceae. As discussed elsewhere, a nickase may be derived from a nuclease by inactivating one of the two catalytic domains, e.g., by mutating an active site residue essential for nucleolysis, such as D10, H840, of N863 in Spy Cas9. One skilled in the art will be familiar with techniques for easily identifying corresponding residues in other Cas proteins, such as sequence alignment and structural alignment, which is discussed in detail below.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fokl. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1 FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises one or more heterologous functional domains (e.g., is or comprises a fusion polypeptide).

In some embodiments, the RNA-guided DNA binding agent comprises a APOBEC3 deaminase. In some embodiments, a APOBEC3 deaminase is a APOBEC3A (A3A). In some embodiments, the A3A is a human A3A. In some embodiments, the A3A is a wild-type A3A.

In some embodiments, the RNA-guided DNA binding agent comprises a deaminase and an RNA-guided nickase. In some embodiments, the mRNA further comprises a linker to link the sequencing encoding A3A to the sequence sequencing encoding RNA-guided nickase. In some embodiments, the linker is an organic molecule, group, polymer, or chemical moiety. In some embodiments, the linker is a peptide linker. In some embodiments, the peptide linker is any stretch of amino acids having at least 1, at least 2, at least 3, at least 4, at least 5, at least 6, at least 7, at least 8, at least 9, at least 10, at least 15, at least 20, at least 25, at least 30, at least 40, at least 50, or more amino acids. In some embodiments, the peptide linker is the 16 residue “XTEN” linker, or a variant thereof (See, e.g., the Examples; and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be fused at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two NLS sequences (e.g., SV40) fused at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be fused at the C-terminus of the RNA-guided DNA-binding agent. One or more linkers are optionally included at the fusion site.

In some embodiments, the RNA-guided DNA binding agent comprises an editor. An exemplary editor is BC22n which includes a H. sapiens APOBEC3A fused to S. pyogenes-D10A Cas9 nickase by an XTEN linker, and mRNA encoding BC22n. An mRNA encoding BC22n is provided (SEQ ID NO:806).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT10), autophagy-8 (ATG8) and −12 (ATG12), Fau ubiquitin-like protein (FUB1), membrane-anchored UBL (MUB), ubiquitin fold-modifier-1 (UFM1), and ubiquitin-like protein-5 (UBLS).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcVS, AU1, AUS, E, ECS, E2, FLAG, HA, nus, Softag 1, Softag 3, Strep, SBP, Glu-Glu, HSV, KT3, S, S1, T7, V5, VSV-G, 6xHis, 8xHis, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector such as an editor domain may modify or affect the target sequence. In some embodiments, the effector such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a Fokl nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

### J. Determination of Efficacy of Guide RNAs

In some embodiments, the efficacy of a guide RNA is determined when delivered or expressed together with other components (e.g., an RNA-guided DNA binding agent) forming an RNP. In some embodiments, the guide RNA is expressed together with an RNA-guided DNA binding agent, such as a Cas protein, e.g., Cas9. In some embodiments, the guide RNA is delivered to or expressed in a cell line that already stably expresses an RNA-guided DNA nuclease, such as a Cas nuclease or nickase, e.g., Cas9 nuclease or nickase. In some embodiments the guide RNA is delivered to a cell as part of a RNP. In some embodiments, the guide RNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to DSBs, SSBs, and/or site-specific binding that results in nucleic acid modification in the DNA or pre-mRNA which can produce errors in the form of insertion/deletion (indel) mutations upon repair by cellular machinery. Many mutations due to indels alter the reading frame, introduce premature stop codons, or induce exon skipping and, therefore, produce a non-functional protein.

In some embodiments, the efficacy of particular guide RNAs is determined based on in vitro models. In some embodiments, the in vitro model is T cell line. In some embodiments, the in vitro model is HEK293 T cells. In some embodiments, the in vitro model is HEK293 cells stably expressing Cas9 (HEK293 Cas9). In some embodiments, the in vitro model is a lymphoblastoid cell line. In some embodiments, the in vitro model is primary human T cells. In some embodiments, the in vitro model is primary human B cells. In some embodiments, the in vitro model is primary human peripheral blood lymphocytes. In some embodiments, the in vitro model is primary human peripheral blood mononuclear cells.

In some embodiments, the number of off-target sites at which a deletion or insertion occurs in an in vitro model is determined, e.g., by analyzing genomic DNA from the cells transfected in vitro with Cas9 mRNA and the guide RNA. In some embodiments, such a determination comprises analyzing genomic DNA from cells transfected in vitro with Cas9 mRNA, the guide RNA, and a donor oligonucleotide. Exemplary procedures for such determinations are provided in the working examples below.

In some embodiments, the efficacy of particular gRNAs is determined across multiple in vitro cell models for a guide RNA selection process. In some embodiments, a cell line comparison of data with selected guide RNAs is performed. In some embodiments, cross screening in multiple cell models is performed.

In some embodiments, the efficacy of a guide RNA is evaluated by on target cleavage efficiency. In some embodiments, the efficacy of a guide RNA is measured by percent editing at the target location, e.g., HLA-A, or CIITA. In some embodiments, deep sequencing may be utilized to identify the presence of modifications (e.g., insertions, deletions) introduced by gene editing. Indel percentage can be calculated from next generation sequencing “NGS.”

In some embodiments, the efficacy of a guide RNA is measured by the number and/or frequency of indels at off-target sequences within the genome of the target cell type. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., T cells or B cells), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cells or B cells). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., T cells or B cells) genome.

In some embodiments, linear amplification is used to detect gene editing events, such as the formation of insertion/deletion (“indel”) mutations, translocations, and homology directed repair (HDR) events in target DNA. For example, linear amplification with a unique sequence-tagged primer and isolating the tagged amplification products (herein after referred to as “UnIT,” or “Unique Identifier Tagmentation” method) may be used.

In some embodiments, the efficacy of a guide RNA is measured by the number of chromosomal rearrangements within the target cell type. Kromatid dGH assay may used to detect chromosomal rearrangements, including e.g., translocations, reciprocal translocations, translocations to off-target chromosomes, deletions (i.e., chromosomal rearrangements where fragments were lost during the cell replication cycle due to the editing event). In some embodiments, the target cell type has less than 10, less than 8, less than 5, less than 4, less than 3, less than 2, or less than 1 chromosomal rearrangement. In some embodiments, the target cell type has no chromosomal rearrangements.

### K. Delivery of gRNA Compositions

Lipid nanoparticles (LNP compositions) are a well-known means for delivery of nucleotide and protein cargo and may be used for delivery of the guide RNAs, compositions, or pharmaceutical formulations disclosed herein. In some embodiments, the LNP compositions deliver nucleic acid, protein, or nucleic acid together with protein.

In some embodiments, the invention comprises a method for delivering any one of the gRNAs disclosed herein to a subject, wherein the gRNA is formulated as an LNP. In some embodiments, the LNP comprises the gRNA and a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNP compositions comprise cationic lipids. In some embodiments, the LNP compositions comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO/2017/173054 and references described therein. In some embodiments, the LNP compositions comprise molar ratios of a cationic lipid amine to RNA phosphate (N:P) of about 4.5, 5.0, 5.5, 6.0, or 6.5. In some embodiments, the term cationic and ionizable in the context of LNP lipids is interchangeable, e.g., wherein ionizable lipids are cationic depending on the pH.

In some embodiments, the gRNAs disclosed herein are formulated as LNP compositions for use in preparing a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is formulated as an LNP or not formulated as an LNP. In some embodiments, the LNP comprises the gRNA and a Cas9 or an mRNA encoding Cas9.

In some embodiments, the guide RNA compositions described herein, alone or encoded on one or more vectors, are formulated in or administered via a lipid nanoparticle; see e.g., WO/2017/173054 and WO 2019/067992, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

### L. Therapeutic Methods and Uses

Any of the engineered human cells and compositions described herein can be used in a method of treating a variety of diseases and disorders, as described herein. In some embodiments, the genetically modified cell (engineered cell) and/or population of genetically modified cells (engineered cells) and compositions may be used in methods of treating a variety of diseases and disorders. In some embodiments, a method of treating any one of the diseases or disorders described herein is encompassed, comprising administering any one or more composition described herein.

In some embodiments, the methods and compositions described herein may be used to treat diseases or disorders in need of delivery of a therapeutic agent. In some embodiments, the invention provides a method of providing an immunotherapy in a subject, the method including administering to the subject an effective amount of an engineered cell (or population of engineered cells) as described herein, for example, a cell of any of the aforementioned cell aspects and embodiments.

In some embodiments, the methods comprise administering to a subject a composition comprising an engineered cell described herein as an adoptive cell transfer therapy. In some embodiments, the engineered cell is an allogeneic cell.

In some embodiments, the methods comprise administering to a subject a composition comprising an engineered cell described herein, wherein the cell produces, secretes, and/or expresses a polypeptide (e.g., a targeting receptor) useful for treatment of a disease or disorder in a subject. In some embodiments, the cell acts as a cell factory to produce a soluble polypeptide. In some embodiments, the cell acts as a cell factory to produce an antibody. In some embodiments, the cell continuously secretes the polypeptide in vivo. In some embodiments, the cell continuously secretes the polypeptide following transplantation in vivo for at least 1, 2, 3, 4, 5, or 6 weeks. In some embodiments, the cell continuously secretes the polypeptide following transplantation in vivo for more than 6 weeks. In some embodiments, the soluble polypeptide (e.g., an antibody) is produced by the cell at a concentration of at least 10², 10³, 10⁴, 10⁵, 10⁶, 10⁷, or 10⁸ copies per day. In some embodiments, the polypeptide is an antibody and is produced by the cell at a concentration of at least 10⁸ copies per day.

In some embodiments of the methods, the method includes administering a lymphodepleting agent or immunosuppressant prior to administering to the subject an effective amount of the engineered cell (or engineered cells) as described herein, for example, a cell of any of the aforementioned cell aspects and embodiments. In another aspect, the invention provides a method of preparing engineered cells (e.g., a population of engineered cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer (NK) cells, cytotoxic T lymphocytes (CTLs), T helper cells, B cells, or their progenitors such as hematopoietic stem cells (HSC) or induced pluripotent stem cells (iPSC) can be programmed to act in response to abnormal antigens expressed on the surface of tumor cells. Thus, cancer immunotherapy allows components of the immune system to destroy tumors or other cancerous cells. Cell-based immunotherapies have also been demonstrated to be effective in the treatment of autoimmune diseases or transplant rejection. Immune effector cells such as regulatory T cells (Tregs) or mesenchymal stem cells can be programmed to act in response to autoantigens or transplant antigens expressed on the surface of normal tissues.

In some embodiments, the invention provides a method of preparing engineered cells (e.g., a population of engineered cells). The population of engineered cells may be used for immunotherapy.

In some embodiments, the invention provides a method of treating a subject in need thereof that includes administering engineered cells prepared by a method of preparing cells described herein, for example, a method of any of the aforementioned aspects and embodiments of methods of preparing cells.

In some embodiments, the engineered cells can be used to treat cancer, infectious diseases, inflammatory diseases, autoimmune diseases, cardiovascular diseases, neurological diseases, ophthalmologic diseases, renal diseases, liver diseases, musculoskeletal diseases, red blood cell diseases, or transplant rejections. In some embodiments, the engineered cells can be used in cell transplant, e.g., to the heart, liver, lung, kidney, pancreas, skin, or brain. (See e.g., Deuse et al., Nature Biotechnology 37:252-258 (2019).)

In some embodiments, the engineered cells can be used as a cell therapy comprising an allogeneic stem cell therapy. In some embodiments, the cell therapy comprises induced pluripotent stem cells (iPSCs). iPSCs may be induced to differentiate into other cell types including e.g., beta islet cells, neurons, and blood cells. In some embodiments, the cell therapy comprises hematopoietic stem cells. In some embodiments, the stem cells comprise mesenchymal stem cells that can develop into bone, cartilage, muscle, and fat cells. In some embodiments, the stem cells comprise ocular stem cells. In some embodiments, the allogeneic stem cell transplant comprises allogeneic bone marrow transplant. In some embodiments, the stem cells comprise pluripotent stem cells (PSCs). In some embodiments, the stem cells comprise induced embryonic stem cells (ESCs).

The engineered human cells disclosed herein are suitable for further engineering, e.g., by introduction of further edited, or modified genes or alleles. Cells of the invention may also be suitable for further engineering by introduction of an exogenous nucleic acid encoding e.g., a targeting receptor, e.g., a TCR, CAR, UniCAR. CARs are also known as chimeric immunoreceptors, chimeric T cell receptors or artificial T cell receptors. In some embodiments, the TCR is a wild-type or variant TCR.

In some embodiments, the cell therapy is a transgenic T cell therapy. In some embodiments, the cell therapy comprises a Wilms' Tumor 1 (WT1) targeting transgenic T cell. In some embodiments, the cell therapy comprises a targeting receptor or a donor nucleic acid encoding a targeting receptor of a commercially available T cell therapy, such as a CAR T cell therapy. There are number of targeting receptors currently approved for cell therapy. The cells and methods provided herein can be used with these known constructs. Commercially approved cell products that include targeting receptor constructs for use as cell therapies include e.g., Kymriah® (tisagenlecleucel); Yescarta® (axicabtagene ciloleucel); Tecartus™ (brexucabtagene autoleucel); Tabelecleucel (Tab-cel®); Viralym-M (ALVR105); and Viralym-C.

In some embodiments, the methods provide for administering the engineered cells to a subject, wherein the administration is an injection. In some embodiments, the methods provide for administering the engineered cells to a subject, wherein the administration is an intravascular injection or infusion. In some embodiments, the methods provide for administering the engineered cells to a subject, wherein the administration is a single dose.

In some embodiments, the methods provide for reducing a sign or symptom associated of a subject's disease treated with a composition disclosed herein. In some embodiments, the subject has a response to treatment with a composition disclosed herein that lasts more than one week. In some embodiments, the subject has a response to treatment with a composition disclosed herein that lasts more than two weeks. In some embodiments, the subject has a response to treatment with a composition disclosed herein that lasts more than three weeks. In some embodiments, the subject has a response to treatment with a composition disclosed herein that lasts more than one month.

In some embodiments, the methods provide for administering the engineered cells to an subject, and wherein the subject has a response to the administered cell that comprises a reduction in a sign or symptom associated with the disease treated by the cell therapy. In some embodiments, the subject has a response that lasts more than one week. In some embodiments, the subject has a response that lasts more than one month. In some embodiments, the subject has a response that lasts for at least 1-6 weeks.

TABLE 6
ADDITIONAL SEQUENCES

| | SEQ | |
|---|---|---|
| Description | ID NO | Sequence |
| Exemplary | 230 | GAGUCCGAGCAGAAGAAGAA |
| guide | | |
| sequence for | | |
| EMX1 gene | | |
| Exemplary | 231 | GACCCCCUCCACCCCGCCUC |
| guide | | |
| sequence for | | |
| VEGFA gene | | |
| Exemplary | 232 | GACUUGUUUUCAUUGUUCUC |
| guide | | |
| sequence for | | |
| RAGIB gene | | |
| Exemplary | 233 | CUCUCAGCUGGUACACGGCA |
| guide | | |
| sequence for | | |
| TRAC gene | | |
| Exemplary | 234 | UGUGCAGACUCAGAGGUGAG |
| guide | | |
| sequence for | | |
| CIITA gene | | |
| Exemplary | 235 | GGCCACGGAGCGAGACAUCU |
| guide | | |
| sequence for | | |
| B2M gene | | |
| Exemplary | 236 | CCCCCGGACGGUUCAAGCAA |
| guide for | | |
| CIITA gene | | |
| | 237- | Not Used |
| | 239 | |
| G000644 | 240 | mG*mA*mG*UCCGAGCAGAAGAAGAAGUUUUAGAmG |
| guide RNA | | mCmUmAmGmAmAmAmUmAmGmCAAGUUAAAAUAAG |
| targeting | | GCUAGUCCGUUAUCAmAmCmUmUmGmAmAmAmAmA |
| EMX1 with | | mGmUmGmGmCmAmCmCmGmAmGmUmCmGmGmUmG |
| guide | | mCmU*mU*mU*mU |
| sequence SEQ | | |
| ID NO: 230 | | |
| G000645 | 241 | mG*mA*mC*CCCCUCCACCCCGCCUCGUUUUAGAmGm |
| guide RNA | | CmUmAmGmAmAmAmUmAmGmCAAGUUAAAAUAAGG |
| targeting | | CUAGUCCGUUAUCAmAmCmUmUmGmAmAmAmAmAm |
| VEGFA with | | GmUmGmGmCmAmCmCmGmAmGmUmCmGmGmUmGm |
| guide | | CmU*mU*mU*mU |
| sequence SEQ | | |
| ID NO: 231 | | |
| G000646 | 242 | mG*mA*mC*UUGUUUUCAUUGUUCUCGUUUUAGAmG |
| guide RNA | | mCmUmAmGmAmAmAmUmAmGmCAAGUUAAAAUAAG |
| targeting | | GCUAGUCCGUUAUCAmAmCmUmUmGmAmAmAmAmA |
| RAGIB with | | mGmUmGmGmCmAmCmCmGmAmGmUmCmGmGmUmG |
| guide | | mCmU*mU*mU*mU |
| sequence SEQ | | |
| ID NO: 232 | | |
| G013006 | 243 | mC*mU*mC*UCAGCUGGUACACGGCAGUUUUAGAmG |
| guide RNA | | mCmUmAmGmAmAmAmUmAmGmCAAGUUAAAAUAAG |
| targeting | | GCUAGUCCGUUAUCAmAmCmUmUmGmAmAmAmAmA |
| TRAC with | | mGmUmGmGmCmAmCmCmGmAmGmUmCmGmGmUmG |
| guide | | mCmU*mU*mU*mU |
| sequence SEQ | | |
| ID NO: 233 | | |
| G018091 | 244 | mU*mG*mU*GCAGACUCAGAGGUGAGGUUUUAGAmG |
| RNA | | mCmUmAmGmAmAmAmUmAmGmCAAGUUAAAAUAAG |
| targeting | | GCUAGUCCGUUAUCAmAmCmUmUmGmAmAmAmAmA |
| CIITA with | | mGmUmGmGmCmAmCmCmGmAmGmUmCmGmGmUmG |
| guide SEQ ID | | mCmU*mU*mU*mU |
| NO: 234 | | |
| G000529 | 245 | mG*mG*mC*CACGGAGCGAGACAUCUGUUUUAGAmG |
| RNA | | mCmUmAmGmAmAmAmUmAmGmCAAGUUAAAAUAAG |
| targeting | | GCUAGUCCGUUAUCAmAmCmUmUmGmAmAmAmAmA |
| B2M with | | mGmUmGmGmCmAmCmCmGmAmGmUmCmGmGmUmG |
| guide SEQ ID | | mCmU*mU*mU*mU |
| NO: 235 | | |
| G013675 | 246 | mC*mC*mC*CCGGACGGUUCAAGCAAGUUUUAGAmG |
| RNA | | mCmUmAmGmAmAmAmUmAmGmCAAGUUAAAAUAAG |
| targeting | | GCUAGUCCGUUAUCAmAmCmUmUmGmAmAmAmAmA |
| CIITA with | | mGmUmGmGmCmAmCmCmGmAmGmUmCmGmGmUmG |
| guide SEQ ID | | mCmU*mU*mU*mU |
| NO: 236 | | |
| G016239 | 247 | mG*mG*mC*CUCGGCGCUGACGAUCUGUUUUAGAmG |
| | | mCmUmAmGmAmAmAmUmAmGmCAAGUUAAAAUAAG |
| | | GCUAGUCCGUUAUCAmAmCmUmUmGmAmAmAmAmA |
| | | mGmUmGmGmCmAmCmCmGmAmGmUmCmGmGmUmG |
| | | mCmU*mU*mU*mU |
| G013676 | 248 | mU*mG*mG*UCAGGGCAAGAGCUAUUGUUUUAGAmG |
| | | mCmUmAmGmAmAmAmUmAmGmCAAGUUAAAAUAAG |
| | | GCUAGUCCGUUAUCAmAmCmUmUmGmAmAmAmAmA |
| | | mGmUmGmGmCmAmCmCmGmAmGmUmCmGmGmUmG |
| | | mCmU*mU*mU*mU |
| Recombinant | 800 | MDKKYSIGLDIGTNSVGWAVITDEYKVPSKKFKVLGNT |
| Cas9-NLS | | DRHSIKKNLIGALLFDSGETAEATRLKRTARRRYTRRKN |
| amino acid | | RICYLQEIFSNEMAKVDDSFFHRLEESFLVEEDKKHERHP |
| sequence | | IFGNIVDEVAYHEKYPTIYHLRKKLVDSTDKADLRLIYLA |
| | | LAHMIKFRGHFLIEGDLNPDNSDVDKLFIQLVQTYNQLF |
| | | EENPINASGVDAKAILSARLSKSRRLENLIAQLPGEKKNG |
| | | LFGNLIALSLGLTPNFKSNFDLAEDAKLQLSKDTYDDDL |
| | | DNLLAQIGDQYADLFLAAKNLSDAILLSDILRVNTEITKA |
| | | PLSASMIKRYDEHHQDLTLLKALVRQQLPEKYKEIFFDQ |
| | | SKNGYAGYIDGGASQEEFYKFIKPILEKMDGTEELLVKL |
| | | NREDLLRKQRTFDNGSIPHQIHLGELHAILRRQEDFYPFL |
| | | KDNREKIEKILTFRIPYYVGPLARGNSRFAWMTRKSEETI |
| | | TPWNFEEVVDKGASAQSFIERMTNFDKNLPNEKVLPKHS |
| | | LLYEYFTVYNELTKVKYVTEGMRKPAFLSGEQKKAIVD |
| | | LLFKTNRKVTVKQLKEDYFKKIECFDSVEISGVEDRFNAS |
| | | LGTYHDLLKIIKDKDFLDNEENEDILEDIVLTLTLFEDRE |
| | | MIEERLKTY AHLFDDKVMKQLKRRRYTGWGRLSRKLIN |
| | | GIRDKQSGKTILDFLKSDGFANRNFMQLIHDDSLTFKEDI |
| | | QKAQVSGQGDSLHEHIANLAGSPAIKKGILQTVKVVDEL |
| | | VKVMGRHKPENIVIEMARENQTTQKGQKNSRERMKRIE |
| | | EGIKELGSQILKEHPVENTQLQNEKLYLYYLQNGRDMY |
| | | VDQELDINRLSDYDVDHIVPQSFLKDDSIDNKVLTRSDK |
| | | NRGKSDNVPSEEVVKKMKNYWRQLLNAKLITQRKFDN |
| | | LTKAERGGLSELDKAGFIKRQLVETRQITKHVAQILDSR |
| | | MNTKYDENDKLIREVKVITLKSKLVSDFRKDFQFYKVRE |
| | | INNYHHAHDAYLNAVVGTALIKKYPKLESEFVYGDYKV |
| | | YDVRKMIAKSEQEIGKATAKYFFYSNIMNFFKTEITLAN |
| | | GEIRKRPLIETNGETGEIVWDKGRDFATVRKVLSMPQVN |
| | | IVKKTEVQTGGFSKESILPKRNSDKLIARKKDWDPKKYG |
| | | GFDSPTVAYSVLVVAKVEKGKSKKLKSVKELLGITIMER |
| | | SSFEKNPIDFLEAKGYKEVKKDLIIKLPKYSLFELENGRK |
| | | RMLASAGELQKGNELALPSKYVNFLYLASHYEKLKGSP |
| | | EDNEQKQLFVEQHKHYLDEIIEQISEFSKRVILADANLDK |
| | | VLSAYNKHRDKPIREQAENIIHLFTLTNLGAPAAFKYFDT |
| | | TIDRKRYTSTKEVLDATLIHQSITGLYETRIDLSQLGGDG |
| | | GGSPKKKRKV |
| ORF | 801 | ATGGACAAGAAGTACAGCATCGGACTGGACATCGGAA |
| encoding Sp. | | CAAACAGCGTCGGATGGGCAGTCATCACAGACGAATA |
| Cas9 | | CAAGGTCCCGAGCAAGAAGTTCAAGGTCCTGGGAAAC |
| | | ACAGACAGACACAGCATCAAGAAGAACCTGATCGGA |
| | | GCACTGCTGTTCGACAGCGGAGAAACAGCAGAAGCAA |
| | | CAAGACTGAAGAGAACAGCAAGAAGAAGATACACAA |
| | | GAAGAAAGAACAGAATCTGCTACCTGCAGGAAATCTT |
| | | CAGCAACGAAATGGCAAAGGTCGACGACAGCTTCTTC |
| | | CACAGACTGGAAGAAAGCTTCCTGGTCGAAGAAGACA |
| | | AGAAGCACGAAAGACACCCGATCTTCGGAAACATCGT |
| | | CGACGAAGTCGCATACCACGAAAAGTACCCGACAATC |
| | | TACCACCTGAGAAAGAAGCTGGTCGACAGCACAGACA |
| | | AGGCAGACCTGAGACTGATCTACCTGGCACTGGCACA |
| | | CATGATCAAGTTCAGAGGACACTTCCTGATCGAAGGA |
| | | GACCTGAACCCGGACAACAGCGACGTCGACAAGCTGT |
| | | TCATCCAGCTGGTCCAGACATACAACCAGCTGTTCGA |
| | | AGAAAACCCGATCAACGCAAGCGGAGTCGACGCAAA |
| | | GGCAATCCTGAGCGCAAGACTGAGCAAGAGCAGAAG |
| | | ACTGGAAAACCTGATCGCACAGCTGCCGGGAGAAAAG |
| | | AAGAACGGACTGTTCGGAAACCTGATCGCACTGAGCC |
| | | TGGGACTGACACCGAACTTCAAGAGCAACTTCGACCT |
| | | GGCAGAAGACGCAAAGCTGCAGCTGAGCAAGGACAC |
| | | ATACGACGACGACCTGGACAACCTGCTGGCACAGATC |
| | | GGAGACCAGTACGCAGACCTGTTCCTGGCAGCAAAGA |
| | | ACCTGAGCGACGCAATCCTGCTGAGCGACATCCTGAG |
| | | AGTCAACACAGAAATCACAAAGGCACCGCTGAGCGCA |
| | | AGCATGATCAAGAGATACGACGAACACCACCAGGACC |
| | | TGACACTGCTGAAGGCACTGGTCAGACAGCAGCTGCC |
| | | GGAAAAGTACAAGGAAATCTTCTTCGACCAGAGCAAG |
| | | AACGGATACGCAGGATACATCGACGGAGGAGCAAGC |
| | | CAGGAAGAATTCTACAAGTTCATCAAGCCGATCCTGG |
| | | AAAAGATGGACGGAACAGAAGAACTGCTGGTCAAGC |
| | | TGAACAGAGAAGACCTGCTGAGAAAGCAGAGAACAT |
| | | TCGACAACGGAAGCATCCCGCACCAGATCCACCTGGG |
| | | AGAACTGCACGCAATCCTGAGAAGACAGGAAGACTTC |
| | | TACCCGTTCCTGAAGGACAACAGAGAAAAGATCGAAA |
| | | AGATCCTGACATTCAGAATCCCGTACTACGTCGGACC |
| | | GCTGGCAAGAGGAAACAGCAGATTCGCATGGATGACA |
| | | AGAAAGAGCGAAGAAACAATCACACCGTGGAACTTC |
| | | GAAGAAGTCGTCGACAAGGGAGCAAGCGCACAGAGC |
| | | TTCATCGAAAGAATGACAAACTTCGACAAGAACCTGC |
| | | CGAACGAAAAGGTCCTGCCGAAGCACAGCCTGCTGTA |
| | | CGAATACTTCACAGTCTACAACGAACTGACAAAGGTC |
| | | AAGTACGTCACAGAAGGAATGAGAAAGCCGGCATTCC |
| | | TGAGCGGAGAACAGAAGAAGGCAATCGTCGACCTGCT |
| | | GTTCAAGACAAACAGAAAGGTCACAGTCAAGCAGCTG |
| | | AAGGAAGACTACTTCAAGAAGATCGAATGCTTCGACA |
| | | GCGTCGAAATCAGCGGAGTCGAAGACAGATTCAACGC |
| | | AAGCCTGGGAACATACCACGACCTGCTGAAGATCATC |
| | | AAGGACAAGGACTTCCTGGACAACGAAGAAAACGAA |
| | | GACATCCTGGAAGACATCGTCCTGACACTGACACTGT |
| | | TCGAAGACAGAGAAATGATCGAAGAAAGACTGAAGA |
| | | CATACGCACACCTGTTCGACGACAAGGTCATGAAGCA |
| | | GCTGAAGAGAAGAAGATACACAGGATGGGGAAGACT |
| | | GAGCAGAAAGCTGATCAACGGAATCAGAGACAAGCA |
| | | GAGCGGAAAGACAATCCTGGACTTCCTGAAGAGCGAC |
| | | GGATTCGCAAACAGAAACTTCATGCAGCTGATCCACG |
| | | ACGACAGCCTGACATTCAAGGAAGACATCCAGAAGGC |
| | | ACAGGTCAGCGGACAGGGAGACAGCCTGCACGAACA |
| | | CATCGCAAACCTGGCAGGAAGCCCGGCAATCAAGAAG |
| | | GGAATCCTGCAGACAGTCAAGGTCGTCGACGAACTGG |
| | | TCAAGGTCATGGGAAGACACAAGCCGGAAAACATCGT |
| | | CATCGAAATGGCAAGAGAAAACCAGACAACACAGAA |
| | | GGGACAGAAGAACAGCAGAGAAAGAATGAAGAGAAT |
| | | CGAAGAAGGAATCAAGGAACTGGGAAGCCAGATCCT |
| | | GAAGGAACACCCGGTCGAAAACACACAGCTGCAGAA |
| | | CGAAAAGCTGTACCTGTACTACCTGCAGAACGGAAGA |
| | | GACATGTACGTCGACCAGGAACTGGACATCAACAGAC |
| | | TGAGCGACTACGACGTCGACCACATCGTCCCGCAGAG |
| | | CTTCCTGAAGGACGACAGCATCGACAACAAGGTCCTG |
| | | ACAAGAAGCGACAAGAACAGAGGAAAGAGCGACAAC |
| | | GTCCCGAGCGAAGAAGTCGTCAAGAAGATGAAGAACT |
| | | ACTGGAGACAGCTGCTGAACGCAAAGCTGATCACACA |
| | | GAGAAAGTTCGACAACCTGACAAAGGCAGAGAGAGG |
| | | AGGACTGAGCGAACTGGACAAGGCAGGATTCATCAAG |
| | | AGACAGCTGGTCGAAACAAGACAGATCACAAAGCAC |
| | | GTCGCACAGATCCTGGACAGCAGAATGAACACAAAGT |
| | | ACGACGAAAACGACAAGCTGATCAGAGAAGTCAAGG |
| | | TCATCACACTGAAGAGCAAGCTGGTCAGCGACTTCAG |
| | | AAAGGACTTCCAGTTCTACAAGGTCAGAGAAATCAAC |
| | | AACTACCACCACGCACACGACGCATACCTGAACGCAG |
| | | TCGTCGGAACAGCACTGATCAAGAAGTACCCGAAGCT |
| | | GGAAAGCGAATTCGTCTACGGAGACTACAAGGTCTAC |
| | | GACGTCAGAAAGATGATCGCAAAGAGCGAACAGGAA |
| | | ATCGGAAAGGCAACAGCAAAGTACTTCTTCTACAGCA |
| | | ACATCATGAACTTCTTCAAGACAGAAATCACACTGGC |
| | | AAACGGAGAAATCAGAAAGAGACCGCTGATCGAAAC |
| | | AAACGGAGAAACAGGAGAAATCGTCTGGGACAAGGG |
| | | AAGAGACTTCGCAACAGTCAGAAAGGTCCTGAGCATG |
| | | CCGCAGGTCAACATCGTCAAGAAGACAGAAGTCCAGA |
| | | CAGGAGGATTCAGCAAGGAAAGCATCCTGCCGAAGA |
| | | GAAACAGCGACAAGCTGATCGCAAGAAAGAAGGACT |
| | | GGGACCCGAAGAAGTACGGAGGATTCGACAGCCCGA |
| | | CAGTCGCATACAGCGTCCTGGTCGTCGCAAAGGTCGA |
| | | AAAGGGAAAGAGCAAGAAGCTGAAGAGCGTCAAGGA |
| | | ACTGCTGGGAATCACAATCATGGAAAGAAGCAGCTTC |
| | | GAAAAGAACCCGATCGACTTCCTGGAAGCAAAGGGAT |
| | | ACAAGGAAGTCAAGAAGGACCTGATCATCAAGCTGCC |
| | | GAAGTACAGCCTGTTCGAACTGGAAAACGGAAGAAA |
| | | GAGAATGCTGGCAAGCGCAGGAGAACTGCAGAAGGG |
| | | AAACGAACTGGCACTGCCGAGCAAGTACGTCAACTTC |
| | | CTGTACCTGGCAAGCCACTACGAAAAGCTGAAGGGAA |
| | | GCCCGGAAGACAACGAACAGAAGCAGCTGTTCGTCGA |
| | | ACAGCACAAGCACTACCTGGACGAAATCATCGAACAG |
| | | ATCAGCGAATTCAGCAAGAGAGTCATCCTGGCAGACG |
| | | CAAACCTGGACAAGGTCCTGAGCGCATACAACAAGCA |
| | | CAGAGACAAGCCGATCAGAGAACAGGCAGAAAACAT |
| | | CATCCACCTGTTCACACTGACAAACCTGGGAGCACCG |
| | | GCAGCATTCAAGTACTTCGACACAACAATCGACAGAA |
| | | AGAGATACACAAGCACAAAGGAAGTCCTGGACGCAA |
| | | CACTGATCCACCAGAGCATCACAGGACTGTACGAAAC |
| | | AAGAATCGACCTGAGCCAGCTGGGAGGAGACGGAGG |
| | | AGGAAGCCCGAAGAAGAAGAGAAAGGTCTAG |
| ORF | 802 | ATGGACAAGAAGTACTCCATCGGCCTGGACATCGGCA |
| encoding Sp. | | CCAACTCCGTGGGCTGGGCCGTGATCACCGACGAGTA |
| Cas9 | | CAAGGTGCCCTCCAAGAAGTTCAAGGTGCTGGGCAAC |
| | | ACCGACCGGCACTCCATCAAGAAGAACCTGATCGGCG |
| | | CCCTGCTGTTCGACTCCGGCGAGACCGCCGAGGCCAC |
| | | CCGGCTGAAGCGGACCGCCCGGCGGCGGTACACCCGG |
| | | CGGAAGAACCGGATCTGCTACCTGCAGGAGATCTTCT |
| | | CCAACGAGATGGCCAAGGTGGACGACTCCTTCTTCCA |
| | | CCGGCTGGAGGAGTCCTTCCTGGTGGAGGAGGACAAG |
| | | AAGCACGAGCGGCACCCCATCTTCGGCAACATCGTGG |
| | | ACGAGGTGGCCTACCACGAGAAGTACCCCACCATCTA |
| | | CCACCTGCGGAAGAAGCTGGTGGACTCCACCGACAAG |
| | | GCCGACCTGCGGCTGATCTACCTGGCCCTGGCCCACAT |
| | | GATCAAGTTCCGGGGCCACTTCCTGATCGAGGGCGAC |
| | | CTGAACCCCGACAACTCCGACGTGGACAAGCTGTTCA |
| | | TCCAGCTGGTGCAGACCTACAACCAGCTGTTCGAGGA |
| | | GAACCCCATCAACGCCTCCGGCGTGGACGCCAAGGCC |
| | | ATCCTGTCCGCCCGGCTGTCCAAGTCCCGGCGGCTGG |
| | | AGAACCTGATCGCCCAGCTGCCCGGCGAGAAGAAGAA |
| | | CGGCCTGTTCGGCAACCTGATCGCCCTGTCCCTGGGCC |
| | | TGACCCCCAACTTCAAGTCCAACTTCGACCTGGCCGA |
| | | GGACGCCAAGCTGCAGCTGTCCAAGGACACCTACGAC |
| | | GACGACCTGGACAACCTGCTGGCCCAGATCGGCGACC |
| | | AGTACGCCGACCTGTTCCTGGCCGCCAAGAACCTGTC |
| | | CGACGCCATCCTGCTGTCCGACATCCTGCGGGTGAAC |
| | | ACCGAGATCACCAAGGCCCCCCTGTCCGCCTCCATGA |
| | | TCAAGCGGTACGACGAGCACCACCAGGACCTGACCCT |
| | | GCTGAAGGCCCTGGTGCGGCAGCAGCTGCCCGAGAAG |
| | | TACAAGGAGATCTTCTTCGACCAGTCCAAGAACGGCT |
| | | ACGCCGGCTACATCGACGGCGGCGCCTCCCAGGAGGA |
| | | GTTCTACAAGTTCATCAAGCCCATCCTGGAGAAGATG |
| | | GACGGCACCGAGGAGCTGCTGGTGAAGCTGAACCGGG |
| | | AGGACCTGCTGCGGAAGCAGCGGACCTTCGACAACGG |
| | | CTCCATCCCCCACCAGATCCACCTGGGCGAGCTGCAC |
| | | GCCATCCTGCGGCGGCAGGAGGACTTCTACCCCTTCCT |
| | | GAAGGACAACCGGGAGAAGATCGAGAAGATCCTGAC |
| | | CTTCCGGATCCCCTACTACGTGGGCCCCCTGGCCCGGG |
| | | GCAACTCCCGGTTCGCCTGGATGACCCGGAAGTCCGA |
| | | GGAGACCATCACCCCCTGGAACTTCGAGGAGGTGGTG |
| | | GACAAGGGCGCCTCCGCCCAGTCCTTCATCGAGCGGA |
| | | TGACCAACTTCGACAAGAACCTGCCCAACGAGAAGGT |
| | | GCTGCCCAAGCACTCCCTGCTGTACGAGTACTTCACCG |
| | | TGTACAACGAGCTGACCAAGGTGAAGTACGTGACCGA |
| | | GGGCATGCGGAAGCCCGCCTTCCTGTCCGGCGAGCAG |
| | | AAGAAGGCCATCGTGGACCTGCTGTTCAAGACCAACC |
| | | GGAAGGTGACCGTGAAGCAGCTGAAGGAGGACTACTT |
| | | CAAGAAGATCGAGTGCTTCGACTCCGTGGAGATCTCC |
| | | GGCGTGGAGGACCGGTTCAACGCCTCCCTGGGCACCT |
| | | ACCACGACCTGCTGAAGATCATCAAGGACAAGGACTT |
| | | CCTGGACAACGAGGAGAACGAGGACATCCTGGAGGA |
| | | CATCGTGCTGACCCTGACCCTGTTCGAGGACCGGGAG |
| | | ATGATCGAGGAGCGGCTGAAGACCTACGCCCACCTGT |
| | | TCGACGACAAGGTGATGAAGCAGCTGAAGCGGCGGC |
| | | GGTACACCGGCTGGGGCCGGCTGTCCCGGAAGCTGAT |
| | | CAACGGCATCCGGGACAAGCAGTCCGGCAAGACCATC |
| | | CTGGACTTCCTGAAGTCCGACGGCTTCGCCAACCGGA |
| | | ACTTCATGCAGCTGATCCACGACGACTCCCTGACCTTC |
| | | AAGGAGGACATCCAGAAGGCCCAGGTGTCCGGCCAG |
| | | GGCGACTCCCTGCACGAGCACATCGCCAACCTGGCCG |
| | | GCTCCCCCGCCATCAAGAAGGGCATCCTGCAGACCGT |
| | | GAAGGTGGTGGACGAGCTGGTGAAGGTGATGGGCCG |
| | | GCACAAGCCCGAGAACATCGTGATCGAGATGGCCCGG |
| | | GAGAACCAGACCACCCAGAAGGGCCAGAAGAACTCC |
| | | CGGGAGCGGATGAAGCGGATCGAGGAGGGCATCAAG |
| | | GAGCTGGGCTCCCAGATCCTGAAGGAGCACCCCGTGG |
| | | AGAACACCCAGCTGCAGAACGAGAAGCTGTACCTGTA |
| | | CTACCTGCAGAACGGCCGGGACATGTACGTGGACCAG |
| | | GAGCTGGACATCAACCGGCTGTCCGACTACGACGTGG |
| | | ACCACATCGTGCCCCAGTCCTTCCTGAAGGACGACTCC |
| | | ATCGACAACAAGGTGCTGACCCGGTCCGACAAGAACC |
| | | GGGGCAAGTCCGACAACGTGCCCTCCGAGGAGGTGGT |
| | | GAAGAAGATGAAGAACTACTGGCGGCAGCTGCTGAAC |
| | | GCCAAGCTGATCACCCAGCGGAAGTTCGACAACCTGA |
| | | CCAAGGCCGAGCGGGGCGGCCTGTCCGAGCTGGACAA |
| | | GGCCGGCTTCATCAAGCGGCAGCTGGTGGAGACCCGG |
| | | CAGATCACCAAGCACGTGGCCCAGATCCTGGACTCCC |
| | | GGATGAACACCAAGTACGACGAGAACGACAAGCTGA |
| | | TCCGGGAGGTGAAGGTGATCACCCTGAAGTCCAAGCT |
| | | GGTGTCCGACTTCCGGAAGGACTTCCAGTTCTACAAG |
| | | GTGCGGGAGATCAACAACTACCACCACGCCCACGACG |
| | | CCTACCTGAACGCCGTGGTGGGCACCGCCCTGATCAA |
| | | GAAGTACCCCAAGCTGGAGTCCGAGTTCGTGTACGGC |
| | | GACTACAAGGTGTACGACGTGCGGAAGATGATCGCCA |
| | | AGTCCGAGCAGGAGATCGGCAAGGCCACCGCCAAGTA |
| | | CTTCTTCTACTCCAACATCATGAACTTCTTCAAGACCG |
| | | AGATCACCCTGGCCAACGGCGAGATCCGGAAGCGGCC |
| | | CCTGATCGAGACCAACGGCGAGACCGGCGAGATCGTG |
| | | TGGGACAAGGGCCGGGACTTCGCCACCGTGCGGAAGG |
| | | TGCTGTCCATGCCCCAGGTGAACATCGTGAAGAAGAC |
| | | CGAGGTGCAGACCGGCGGCTTCTCCAAGGAGTCCATC |
| | | CTGCCCAAGCGGAACTCCGACAAGCTGATCGCCCGGA |
| | | AGAAGGACTGGGACCCCAAGAAGTACGGCGGCTTCGA |
| | | CTCCCCCACCGTGGCCTACTCCGTGCTGGTGGTGGCCA |
| | | AGGTGGAGAAGGGCAAGTCCAAGAAGCTGAAGTCCG |
| | | TGAAGGAGCTGCTGGGCATCACCATCATGGAGCGGTC |
| | | CTCCTTCGAGAAGAACCCCATCGACTTCCTGGAGGCC |
| | | AAGGGCTACAAGGAGGTGAAGAAGGACCTGATCATC |
| | | AAGCTGCCCAAGTACTCCCTGTTCGAGCTGGAGAACG |
| | | GCCGGAAGCGGATGCTGGCCTCCGCCGGCGAGCTGCA |
| | | GAAGGGCAACGAGCTGGCCCTGCCCTCCAAGTACGTG |
| | | AACTTCCTGTACCTGGCCTCCCACTACGAGAAGCTGA |
| | | AGGGCTCCCCCGAGGACAACGAGCAGAAGCAGCTGTT |
| | | CGTGGAGCAGCACAAGCACTACCTGGACGAGATCATC |
| | | GAGCAGATCTCCGAGTTCTCCAAGCGGGTGATCCTGG |
| | | CCGACGCCAACCTGGACAAGGTGCTGTCCGCCTACAA |
| | | CAAGCACCGGGACAAGCCCATCCGGGAGCAGGCCGA |
| | | GAACATCATCCACCTGTTCACCCTGACCAACCTGGGC |
| | | GCCCCCGCCGCCTTCAAGTACTTCGACACCACCATCGA |
| | | CCGGAAGCGGTACACCTCCACCAAGGAGGTGCTGGAC |
| | | GCCACCCTGATCCACCAGTCCATCACCGGCCTGTACG |
| | | AGACCCGGATCGACCTGTCCCAGCTGGGCGGCGACGG |
| | | CGGCGGCTCCCCCAAGAAGAAGCGGAAGGTGTGA |
| Open reading | 803 | AUGGACAAGAAGUACUCCAUCGGCCUGGACAUCGGC |
| frame for | | ACCAACUCCGUGGGCUGGGCCGUGAUCACCGACGAG |
| Cas9 with | | UACAAGGUGCCCUCCAAGAAGUUCAAGGUGCUGGGC |
| Hibit tag | | AACACCGACCGGCACUCCAUCAAGAAGAACCUGAUC |
| | | GGCGCCCUGCUGUUCGACUCCGGCGAGACCGCCGAG |
| | | GCCACCCGGCUGAAGCGGACCGCCCGGCGGCGGUAC |
| | | ACCCGGCGGAAGAACCGGAUCUGCUACCUGCAGGAG |
| | | AUCUUCUCCAACGAGAUGGCCAAGGUGGACGACUCC |
| | | UUCUUCCACCGGCUGGAGGAGUCCUUCCUGGUGGAG |
| | | GAGGACAAGAAGCACGAGCGGCACCCCAUCUUCGGC |
| | | AACAUCGUGGACGAGGUGGCCUACCACGAGAAGUAC |
| | | CCCACCAUCUACCACCUGCGGAAGAAGCUGGUGGAC |
| | | UCCACCGACAAGGCCGACCUGCGGCUGAUCUACCUG |
| | | GCCCUGGCCCACAUGAUCAAGUUCCGGGGCCACUUC |
| | | CUGAUCGAGGGCGACCUGAACCCCGACAACUCCGAC |
| | | GUGGACAAGCUGUUCAUCCAGCUGGUGCAGACCUAC |
| | | AACCAGCUGUUCGAGGAGAACCCCAUCAACGCCUCC |
| | | GGCGUGGACGCCAAGGCCAUCCUGUCCGCCCGGCUG |
| | | UCCAAGUCCCGGCGGCUGGAGAACCUGAUCGCCCAG |
| | | CUGCCCGGCGAGAAGAAGAACGGCCUGUUCGGCAAC |
| | | CUGAUCGCCCUGUCCCUGGGCCUGACCCCCAACUUCA |
| | | AGUCCAACUUCGACCUGGCCGAGGACGCCAAGCUGC |
| | | AGCUGUCCAAGGACACCUACGACGACGACCUGGACA |
| | | ACCUGCUGGCCCAGAUCGGCGACCAGUACGCCGACC |
| | | UGUUCCUGGCCGCCAAGAACCUGUCCGACGCCAUCC |
| | | UGCUGUCCGACAUCCUGCGGGUGAACACCGAGAUCA |
| | | CCAAGGCCCCCCUGUCCGCCUCCAUGAUCAAGCGGU |
| | | ACGACGAGCACCACCAGGACCUGACCCUGCUGAAGG |
| | | CCCUGGUGCGGCAGCAGCUGCCCGAGAAGUACAAGG |
| | | AGAUCUUCUUCGACCAGUCCAAGAACGGCUACGCCG |
| | | GCUACAUCGACGGCGGCGCCUCCCAGGAGGAGUUCU |
| | | ACAAGUUCAUCAAGCCCAUCCUGGAGAAGAUGGACG |
| | | GCACCGAGGAGCUGCUGGUGAAGCUGAACCGGGAGG |
| | | ACCUGCUGCGGAAGCAGCGGACCUUCGACAACGGCU |
| | | CCAUCCCCCACCAGAUCCACCUGGGCGAGCUGCACGC |
| | | CAUCCUGCGGCGGCAGGAGGACUUCUACCCCUUCCU |
| | | GAAGGACAACCGGGAGAAGAUCGAGAAGAUCCUGAC |
| | | CUUCCGGAUCCCCUACUACGUGGGCCCCCUGGCCCGG |
| | | GGCAACUCCCGGUUCGCCUGGAUGACCCGGAAGUCC |
| | | GAGGAGACCAUCACCCCCUGGAACUUCGAGGAGGUG |
| | | GUGGACAAGGGCGCCUCCGCCCAGUCCUUCAUCGAG |
| | | CGGAUGACCAACUUCGACAAGAACCUGCCCAACGAG |
| | | AAGGUGCUGCCCAAGCACUCCCUGCUGUACGAGUAC |
| | | UUCACCGUGUACAACGAGCUGACCAAGGUGAAGUAC |
| | | GUGACCGAGGGCAUGCGGAAGCCCGCCUUCCUGUCC |
| | | GGCGAGCAGAAGAAGGCCAUCGUGGACCUGCUGUUC |
| | | AAGACCAACCGGAAGGUGACCGUGAAGCAGCUGAAG |
| | | GAGGACUACUUCAAGAAGAUCGAGUGCUUCGACUCC |
| | | GUGGAGAUCUCCGGCGUGGAGGACCGGUUCAACGCC |
| | | UCCCUGGGCACCUACCACGACCUGCUGAAGAUCAUC |
| | | AAGGACAAGGACUUCCUGGACAACGAGGAGAACGAG |
| | | GACAUCCUGGAGGACAUCGUGCUGACCCUGACCCUG |
| | | UUCGAGGACCGGGAGAUGAUCGAGGAGCGGCUGAAG |
| | | ACCUACGCCCACCUGUUCGACGACAAGGUGAUGAAG |
| | | CAGCUGAAGCGGCGGCGGUACACCGGCUGGGGCCGG |
| | | CUGUCCCGGAAGCUGAUCAACGGCAUCCGGGACAAG |
| | | CAGUCCGGCAAGACCAUCCUGGACUUCCUGAAGUCC |
| | | GACGGCUUCGCCAACCGGAACUUCAUGCAGCUGAUC |
| | | CACGACGACUCCCUGACCUUCAAGGAGGACAUCCAG |
| | | AAGGCCCAGGUGUCCGGCCAGGGCGACUCCCUGCAC |
| | | GAGCACAUCGCCAACCUGGCCGGCUCCCCCGCCAUCA |
| | | AGAAGGGCAUCCUGCAGACCGUGAAGGUGGUGGACG |
| | | AGCUGGUGAAGGUGAUGGGCCGGCACAAGCCCGAGA |
| | | ACAUCGUGAUCGAGAUGGCCCGGGAGAACCAGACCA |
| | | CCCAGAAGGGCCAGAAGAACUCCCGGGAGCGGAUGA |
| | | AGCGGAUCGAGGAGGGCAUCAAGGAGCUGGGCUCCC |
| | | AGAUCCUGAAGGAGCACCCCGUGGAGAACACCCAGC |
| | | UGCAGAACGAGAAGCUGUACCUGUACUACCUGCAGA |
| | | ACGGCCGGGACAUGUACGUGGACCAGGAGCUGGACA |
| | | UCAACCGGCUGUCCGACUACGACGUGGACCACAUCG |
| | | UGCCCCAGUCCUUCCUGAAGGACGACUCCAUCGACA |
| | | ACAAGGUGCUGACCCGGUCCGACAAGAACCGGGGCA |
| | | AGUCCGACAACGUGCCCUCCGAGGAGGUGGUGAAGA |
| | | AGAUGAAGAACUACUGGCGGCAGCUGCUGAACGCCA |
| | | AGCUGAUCACCCAGCGGAAGUUCGACAACCUGACCA |
| | | AGGCCGAGCGGGGCGGCCUGUCCGAGCUGGACAAGG |
| | | CCGGCUUCAUCAAGCGGCAGCUGGUGGAGACCCGGC |
| | | AGAUCACCAAGCACGUGGCCCAGAUCCUGGACUCCC |
| | | GGAUGAACACCAAGUACGACGAGAACGACAAGCUGA |
| | | UCCGGGAGGUGAAGGUGAUCACCCUGAAGUCCAAGC |
| | | UGGUGUCCGACUUCCGGAAGGACUUCCAGUUCUACA |
| | | AGGUGCGGGAGAUCAACAACUACCACCACGCCCACG |
| | | ACGCCUACCUGAACGCCGUGGUGGGCACCGCCCUGA |
| | | UCAAGAAGUACCCCAAGCUGGAGUCCGAGUUCGUGU |
| | | ACGGCGACUACAAGGUGUACGACGUGCGGAAGAUGA |
| | | UCGCCAAGUCCGAGCAGGAGAUCGGCAAGGCCACCG |
| | | CCAAGUACUUCUUCUACUCCAACAUCAUGAACUUCU |
| | | UCAAGACCGAGAUCACCCUGGCCAACGGCGAGAUCC |
| | | GGAAGCGGCCCCUGAUCGAGACCAACGGCGAGACCG |
| | | GCGAGAUCGUGUGGGACAAGGGCCGGGACUUCGCCA |
| | | CCGUGCGGAAGGUGCUGUCCAUGCCCCAGGUGAACA |
| | | UCGUGAAGAAGACCGAGGUGCAGACCGGCGGCUUCU |
| | | CCAAGGAGUCCAUCCUGCCCAAGCGGAACUCCGACA |
| | | AGCUGAUCGCCCGGAAGAAGGACUGGGACCCCAAGA |
| | | AGUACGGCGGCUUCGACUCCCCCACCGUGGCCUACU |
| | | CCGUGCUGGUGGUGGCCAAGGUGGAGAAGGGCAAGU |
| | | CCAAGAAGCUGAAGUCCGUGAAGGAGCUGCUGGGCA |
| | | UCACCAUCAUGGAGCGGUCCUCCUUCGAGAAGAACC |
| | | CCAUCGACUUCCUGGAGGCCAAGGGCUACAAGGAGG |
| | | UGAAGAAGGACCUGAUCAUCAAGCUGCCCAAGUACU |
| | | CCCUGUUCGAGCUGGAGAACGGCCGGAAGCGGAUGC |
| | | UGGCCUCCGCCGGCGAGCUGCAGAAGGGCAACGAGC |
| | | UGGCCCUGCCCUCCAAGUACGUGAACUUCCUGUACC |
| | | UGGCCUCCCACUACGAGAAGCUGAAGGGCUCCCCCG |
| | | AGGACAACGAGCAGAAGCAGCUGUUCGUGGAGCAGC |
| | | ACAAGCACUACCUGGACGAGAUCAUCGAGCAGAUCU |
| | | CCGAGUUCUCCAAGCGGGUGAUCCUGGCCGACGCCA |
| | | ACCUGGACAAGGUGCUGUCCGCCUACAACAAGCACC |
| | | GGGACAAGCCCAUCCGGGAGCAGGCCGAGAACAUCA |
| | | UCCACCUGUUCACCCUGACCAACCUGGGCGCCCCCGC |
| | | CGCCUUCAAGUACUUCGACACCACCAUCGACCGGAA |
| | | GCGGUACACCUCCACCAAGGAGGUGCUGGACGCCAC |
| | | CCUGAUCCACCAGUCCAUCACCGGCCUGUACGAGAC |
| | | CCGGAUCGACCUGUCCCAGCUGGGCGGCGACGGCGG |
| | | CGGCUCCCCCAAGAAGAAGCGGAAGGUGUCCGAGUC |
| | | CGCCACCCCCGAGUCCGUGUCCGGCUGGCGGCUGUU |
| | | CAAGAAGAUCUCCUGA |
| Open Reading | 804 | AUGGAGGCCUCCCCCGCCUCCGGCCCCCGGCACCUGA |
| frame for | | UGGACCCCCACAUCUUCACCUCCAACUUCAACAACG |
| BC22n | | GCAUCGGCCGGCACAAGACCUACCUGUGCUACGAGG |
| | | UGGAGCGGCUGGACAACGGCACCUCCGUGAAGAUGG |
| | | ACCAGCACCGGGGCUUCCUGCACAACCAGGCCAAGA |
| | | ACCUGCUGUGCGGCUUCUACGGCCGGCACGCCGAGC |
| | | UGCGGUUCCUGGACCUGGUGCCCUCCCUGCAGCUGG |
| | | ACCCCGCCCAGAUCUACCGGGUGACCUGGUUCAUCU |
| | | CCUGGUCCCCCUGCUUCUCCUGGGGCUGCGCCGGCG |
| | | AGGUGCGGGCCUUCCUGCAGGAGAACACCCACGUGC |
| | | GGCUGCGGAUCUUCGCCGCCCGGAUCUACGACUACG |
| | | ACCCCCUGUACAAGGAGGCCCUGCAGAUGCUGCGGG |
| | | ACGCCGGCGCCCAGGUGUCCAUCAUGACCUACGACG |
| | | AGUUCAAGCACUGCUGGGACACCUUCGUGGACCACC |
| | | AGGGCUGCCCCUUCCAGCCCUGGGACGGCCUGGACG |
| | | AGCACUCCCAGGCCCUGUCCGGCCGGCUGCGGGCCA |
| | | UCCUGCAGAACCAGGGCAACUCCGGCUCCGAGACCC |
| | | CCGGCACCUCCGAGUCCGCCACCCCCGAGUCCGACAA |
| | | GAAGUACUCCAUCGGCCUGGCCAUCGGCACCAACUC |
| | | CGUGGGCUGGGCCGUGAUCACCGACGAGUACAAGGU |
| | | GCCCUCCAAGAAGUUCAAGGUGCUGGGCAACACCGA |
| | | CCGGCACUCCAUCAAGAAGAACCUGAUCGGCGCCCU |
| | | GCUGUUCGACUCCGGCGAGACCGCCGAGGCCACCCG |
| | | GCUGAAGCGGACCGCCCGGCGGCGGUACACCCGGCG |
| | | GAAGAACCGGAUCUGCUACCUGCAGGAGAUCUUCUC |
| | | CAACGAGAUGGCCAAGGUGGACGACUCCUUCUUCCA |
| | | CCGGCUGGAGGAGUCCUUCCUGGUGGAGGAGGACAA |
| | | GAAGCACGAGCGGCACCCCAUCUUCGGCAACAUCGU |
| | | GGACGAGGUGGCCUACCACGAGAAGUACCCCACCAU |
| | | CUACCACCUGCGGAAGAAGCUGGUGGACUCCACCGA |
| | | CAAGGCCGACCUGCGGCUGAUCUACCUGGCCCUGGC |
| | | CCACAUGAUCAAGUUCCGGGGCCACUUCCUGAUCGA |
| | | GGGCGACCUGAACCCCGACAACUCCGACGUGGACAA |
| | | GCUGUUCAUCCAGCUGGUGCAGACCUACAACCAGCU |
| | | GUUCGAGGAGAACCCCAUCAACGCCUCCGGCGUGGA |
| | | CGCCAAGGCCAUCCUGUCCGCCCGGCUGUCCAAGUCC |
| | | CGGCGGCUGGAGAACCUGAUCGCCCAGCUGCCCGGC |
| | | GAGAAGAAGAACGGCCUGUUCGGCAACCUGAUCGCC |
| | | CUGUCCCUGGGCCUGACCCCCAACUUCAAGUCCAAC |
| | | UUCGACCUGGCCGAGGACGCCAAGCUGCAGCUGUCC |
| | | AAGGACACCUACGACGACGACCUGGACAACCUGCUG |
| | | GCCCAGAUCGGCGACCAGUACGCCGACCUGUUCCUG |
| | | GCCGCCAAGAACCUGUCCGACGCCAUCCUGCUGUCC |
| | | GACAUCCUGCGGGUGAACACCGAGAUCACCAAGGCC |
| | | CCCCUGUCCGCCUCCAUGAUCAAGCGGUACGACGAG |
| | | CACCACCAGGACCUGACCCUGCUGAAGGCCCUGGUG |
| | | CGGCAGCAGCUGCCCGAGAAGUACAAGGAGAUCUUC |
| | | UUCGACCAGUCCAAGAACGGCUACGCCGGCUACAUC |
| | | GACGGCGGCGCCUCCCAGGAGGAGUUCUACAAGUUC |
| | | AUCAAGCCCAUCCUGGAGAAGAUGGACGGCACCGAG |
| | | GAGCUGCUGGUGAAGCUGAACCGGGAGGACCUGCUG |
| | | CGGAAGCAGCGGACCUUCGACAACGGCUCCAUCCCC |
| | | CACCAGAUCCACCUGGGCGAGCUGCACGCCAUCCUG |
| | | CGGCGGCAGGAGGACUUCUACCCCUUCCUGAAGGAC |
| | | AACCGGGAGAAGAUCGAGAAGAUCCUGACCUUCCGG |
| | | AUCCCCUACUACGUGGGCCCCCUGGCCCGGGGCAAC |
| | | UCCCGGUUCGCCUGGAUGACCCGGAAGUCCGAGGAG |
| | | ACCAUCACCCCCUGGAACUUCGAGGAGGUGGUGGAC |
| | | AAGGGCGCCUCCGCCCAGUCCUUCAUCGAGCGGAUG |
| | | ACCAACUUCGACAAGAACCUGCCCAACGAGAAGGUG |
| | | CUGCCCAAGCACUCCCUGCUGUACGAGUACUUCACC |
| | | GUGUACAACGAGCUGACCAAGGUGAAGUACGUGACC |
| | | GAGGGCAUGCGGAAGCCCGCCUUCCUGUCCGGCGAG |
| | | CAGAAGAAGGCCAUCGUGGACCUGCUGUUCAAGACC |
| | | AACCGGAAGGUGACCGUGAAGCAGCUGAAGGAGGAC |
| | | UACUUCAAGAAGAUCGAGUGCUUCGACUCCGUGGAG |
| | | AUCUCCGGCGUGGAGGACCGGUUCAACGCCUCCCUG |
| | | GGCACCUACCACGACCUGCUGAAGAUCAUCAAGGAC |
| | | AAGGACUUCCUGGACAACGAGGAGAACGAGGACAUC |
| | | CUGGAGGACAUCGUGCUGACCCUGACCCUGUUCGAG |
| | | GACCGGGAGAUGAUCGAGGAGCGGCUGAAGACCUAC |
| | | GCCCACCUGUUCGACGACAAGGUGAUGAAGCAGCUG |
| | | AAGCGGCGGCGGUACACCGGCUGGGGCCGGCUGUCC |
| | | CGGAAGCUGAUCAACGGCAUCCGGGACAAGCAGUCC |
| | | GGCAAGACCAUCCUGGACUUCCUGAAGUCCGACGGC |
| | | UUCGCCAACCGGAACUUCAUGCAGCUGAUCCACGAC |
| | | GACUCCCUGACCUUCAAGGAGGACAUCCAGAAGGCC |
| | | CAGGUGUCCGGCCAGGGCGACUCCCUGCACGAGCAC |
| | | AUCGCCAACCUGGCCGGCUCCCCCGCCAUCAAGAAG |
| | | GGCAUCCUGCAGACCGUGAAGGUGGUGGACGAGCUG |
| | | GUGAAGGUGAUGGGCCGGCACAAGCCCGAGAACAUC |
| | | GUGAUCGAGAUGGCCCGGGAGAACCAGACCACCCAG |
| | | AAGGGCCAGAAGAACUCCCGGGAGCGGAUGAAGCGG |
| | | AUCGAGGAGGGCAUCAAGGAGCUGGGCUCCCAGAUC |
| | | CUGAAGGAGCACCCCGUGGAGAACACCCAGCUGCAG |
| | | AACGAGAAGCUGUACCUGUACUACCUGCAGAACGGC |
| | | CGGGACAUGUACGUGGACCAGGAGCUGGACAUCAAC |
| | | CGGCUGUCCGACUACGACGUGGACCACAUCGUGCCC |
| | | CAGUCCUUCCUGAAGGACGACUCCAUCGACAACAAG |
| | | GUGCUGACCCGGUCCGACAAGAACCGGGGCAAGUCC |
| | | GACAACGUGCCCUCCGAGGAGGUGGUGAAGAAGAUG |
| | | AAGAACUACUGGCGGCAGCUGCUGAACGCCAAGCUG |
| | | AUCACCCAGCGGAAGUUCGACAACCUGACCAAGGCC |
| | | GAGCGGGGCGGCCUGUCCGAGCUGGACAAGGCCGGC |
| | | UUCAUCAAGCGGCAGCUGGUGGAGACCCGGCAGAUC |
| | | ACCAAGCACGUGGCCCAGAUCCUGGACUCCCGGAUG |
| | | AACACCAAGUACGACGAGAACGACAAGCUGAUCCGG |
| | | GAGGUGAAGGUGAUCACCCUGAAGUCCAAGCUGGUG |
| | | UCCGACUUCCGGAAGGACUUCCAGUUCUACAAGGUG |
| | | CGGGAGAUCAACAACUACCACCACGCCCACGACGCC |
| | | UACCUGAACGCCGUGGUGGGCACCGCCCUGAUCAAG |
| | | AAGUACCCCAAGCUGGAGUCCGAGUUCGUGUACGGC |
| | | GACUACAAGGUGUACGACGUGCGGAAGAUGAUCGCC |
| | | AAGUCCGAGCAGGAGAUCGGCAAGGCCACCGCCAAG |
| | | UACUUCUUCUACUCCAACAUCAUGAACUUCUUCAAG |
| | | ACCGAGAUCACCCUGGCCAACGGCGAGAUCCGGAAG |
| | | CGGCCCCUGAUCGAGACCAACGGCGAGACCGGCGAG |
| | | AUCGUGUGGGACAAGGGCCGGGACUUCGCCACCGUG |
| | | CGGAAGGUGCUGUCCAUGCCCCAGGUGAACAUCGUG |
| | | AAGAAGACCGAGGUGCAGACCGGCGGCUUCUCCAAG |
| | | GAGUCCAUCCUGCCCAAGCGGAACUCCGACAAGCUG |
| | | AUCGCCCGGAAGAAGGACUGGGACCCCAAGAAGUAC |
| | | GGCGGCUUCGACUCCCCCACCGUGGCCUACUCCGUGC |
| | | UGGUGGUGGCCAAGGUGGAGAAGGGCAAGUCCAAGA |
| | | AGCUGAAGUCCGUGAAGGAGCUGCUGGGCAUCACCA |
| | | UCAUGGAGCGGUCCUCCUUCGAGAAGAACCCCAUCG |
| | | ACUUCCUGGAGGCCAAGGGCUACAAGGAGGUGAAGA |
| | | AGGACCUGAUCAUCAAGCUGCCCAAGUACUCCCUGU |
| | | UCGAGCUGGAGAACGGCCGGAAGCGGAUGCUGGCCU |
| | | CCGCCGGCGAGCUGCAGAAGGGCAACGAGCUGGCCC |
| | | UGCCCUCCAAGUACGUGAACUUCCUGUACCUGGCCU |
| | | CCCACUACGAGAAGCUGAAGGGCUCCCCCGAGGACA |
| | | ACGAGCAGAAGCAGCUGUUCGUGGAGCAGCACAAGC |
| | | ACUACCUGGACGAGAUCAUCGAGCAGAUCUCCGAGU |
| | | UCUCCAAGCGGGUGAUCCUGGCCGACGCCAACCUGG |
| | | ACAAGGUGCUGUCCGCCUACAACAAGCACCGGGACA |
| | | AGCCCAUCCGGGAGCAGGCCGAGAACAUCAUCCACC |
| | | UGUUCACCCUGACCAACCUGGGCGCCCCCGCCGCCUU |
| | | CAAGUACUUCGACACCACCAUCGACCGGAAGCGGUA |
| | | CACCUCCACCAAGGAGGUGCUGGACGCCACCCUGAU |
| | | CCACCAGUCCAUCACCGGCCUGUACGAGACCCGGAU |
| | | CGACCUGUCCCAGCUGGGCGGCGACGGCGGCGGCUC |
| | | CCCCAAGAAGAAGCGGAAGGUGUGA |
| Open reading | 805 | AUGGAGGCCUCCCCCGCCUCCGGCCCCCGGCACCUGA |
| frame for | | UGGACCCCCACAUCUUCACCUCCAACUUCAACAACG |
| BC22n with | | GCAUCGGCCGGCACAAGACCUACCUGUGCUACGAGG |
| Hibit tag | | UGGAGCGGCUGGACAACGGCACCUCCGUGAAGAUGG |
| | | ACCAGCACCGGGGCUUCCUGCACAACCAGGCCAAGA |
| | | ACCUGCUGUGCGGCUUCUACGGCCGGCACGCCGAGC |
| | | UGCGGUUCCUGGACCUGGUGCCCUCCCUGCAGCUGG |
| | | ACCCCGCCCAGAUCUACCGGGUGACCUGGUUCAUCU |
| | | CCUGGUCCCCCUGCUUCUCCUGGGGCUGCGCCGGCG |
| | | AGGUGCGGGCCUUCCUGCAGGAGAACACCCACGUGC |
| | | GGCUGCGGAUCUUCGCCGCCCGGAUCUACGACUACG |
| | | ACCCCCUGUACAAGGAGGCCCUGCAGAUGCUGCGGG |
| | | ACGCCGGCGCCCAGGUGUCCAUCAUGACCUACGACG |
| | | AGUUCAAGCACUGCUGGGACACCUUCGUGGACCACC |
| | | AGGGCUGCCCCUUCCAGCCCUGGGACGGCCUGGACG |
| | | AGCACUCCCAGGCCCUGUCCGGCCGGCUGCGGGCCA |
| | | UCCUGCAGAACCAGGGCAACUCCGGCUCCGAGACCC |
| | | CCGGCACCUCCGAGUCCGCCACCCCCGAGUCCGACAA |
| | | GAAGUACUCCAUCGGCCUGGCCAUCGGCACCAACUC |
| | | CGUGGGCUGGGCCGUGAUCACCGACGAGUACAAGGU |
| | | GCCCUCCAAGAAGUUCAAGGUGCUGGGCAACACCGA |
| | | CCGGCACUCCAUCAAGAAGAACCUGAUCGGCGCCCU |
| | | GCUGUUCGACUCCGGCGAGACCGCCGAGGCCACCCG |
| | | GCUGAAGCGGACCGCCCGGCGGCGGUACACCCGGCG |
| | | GAAGAACCGGAUCUGCUACCUGCAGGAGAUCUUCUC |
| | | CAACGAGAUGGCCAAGGUGGACGACUCCUUCUUCCA |
| | | CCGGCUGGAGGAGUCCUUCCUGGUGGAGGAGGACAA |
| | | GAAGCACGAGCGGCACCCCAUCUUCGGCAACAUCGU |
| | | GGACGAGGUGGCCUACCACGAGAAGUACCCCACCAU |
| | | CUACCACCUGCGGAAGAAGCUGGUGGACUCCACCGA |
| | | CAAGGCCGACCUGCGGCUGAUCUACCUGGCCCUGGC |
| | | CCACAUGAUCAAGUUCCGGGGCCACUUCCUGAUCGA |
| | | GGGCGACCUGAACCCCGACAACUCCGACGUGGACAA |
| | | GCUGUUCAUCCAGCUGGUGCAGACCUACAACCAGCU |
| | | GUUCGAGGAGAACCCCAUCAACGCCUCCGGCGUGGA |
| | | CGCCAAGGCCAUCCUGUCCGCCCGGCUGUCCAAGUCC |
| | | CGGCGGCUGGAGAACCUGAUCGCCCAGCUGCCCGGC |
| | | GAGAAGAAGAACGGCCUGUUCGGCAACCUGAUCGCC |
| | | CUGUCCCUGGGCCUGACCCCCAACUUCAAGUCCAAC |
| | | UUCGACCUGGCCGAGGACGCCAAGCUGCAGCUGUCC |
| | | AAGGACACCUACGACGACGACCUGGACAACCUGCUG |
| | | GCCCAGAUCGGCGACCAGUACGCCGACCUGUUCCUG |
| | | GCCGCCAAGAACCUGUCCGACGCCAUCCUGCUGUCC |
| | | GACAUCCUGCGGGUGAACACCGAGAUCACCAAGGCC |
| | | CCCCUGUCCGCCUCCAUGAUCAAGCGGUACGACGAG |
| | | CACCACCAGGACCUGACCCUGCUGAAGGCCCUGGUG |
| | | CGGCAGCAGCUGCCCGAGAAGUACAAGGAGAUCUUC |
| | | UUCGACCAGUCCAAGAACGGCUACGCCGGCUACAUC |
| | | GACGGCGGCGCCUCCCAGGAGGAGUUCUACAAGUUC |
| | | AUCAAGCCCAUCCUGGAGAAGAUGGACGGCACCGAG |
| | | GAGCUGCUGGUGAAGCUGAACCGGGAGGACCUGCUG |
| | | CGGAAGCAGCGGACCUUCGACAACGGCUCCAUCCCC |
| | | CACCAGAUCCACCUGGGCGAGCUGCACGCCAUCCUG |
| | | CGGCGGCAGGAGGACUUCUACCCCUUCCUGAAGGAC |
| | | AACCGGGAGAAGAUCGAGAAGAUCCUGACCUUCCGG |
| | | AUCCCCUACUACGUGGGCCCCCUGGCCCGGGGCAAC |
| | | UCCCGGUUCGCCUGGAUGACCCGGAAGUCCGAGGAG |
| | | ACCAUCACCCCCUGGAACUUCGAGGAGGUGGUGGAC |
| | | AAGGGCGCCUCCGCCCAGUCCUUCAUCGAGCGGAUG |
| | | ACCAACUUCGACAAGAACCUGCCCAACGAGAAGGUG |
| | | CUGCCCAAGCACUCCCUGCUGUACGAGUACUUCACC |
| | | GUGUACAACGAGCUGACCAAGGUGAAGUACGUGACC |
| | | GAGGGCAUGCGGAAGCCCGCCUUCCUGUCCGGCGAG |
| | | CAGAAGAAGGCCAUCGUGGACCUGCUGUUCAAGACC |
| | | AACCGGAAGGUGACCGUGAAGCAGCUGAAGGAGGAC |
| | | UACUUCAAGAAGAUCGAGUGCUUCGACUCCGUGGAG |
| | | AUCUCCGGCGUGGAGGACCGGUUCAACGCCUCCCUG |
| | | GGCACCUACCACGACCUGCUGAAGAUCAUCAAGGAC |
| | | AAGGACUUCCUGGACAACGAGGAGAACGAGGACAUC |
| | | CUGGAGGACAUCGUGCUGACCCUGACCCUGUUCGAG |
| | | GACCGGGAGAUGAUCGAGGAGCGGCUGAAGACCUAC |
| | | GCCCACCUGUUCGACGACAAGGUGAUGAAGCAGCUG |
| | | AAGCGGCGGCGGUACACCGGCUGGGGCCGGCUGUCC |
| | | CGGAAGCUGAUCAACGGCAUCCGGGACAAGCAGUCC |
| | | GGCAAGACCAUCCUGGACUUCCUGAAGUCCGACGGC |
| | | UUCGCCAACCGGAACUUCAUGCAGCUGAUCCACGAC |
| | | GACUCCCUGACCUUCAAGGAGGACAUCCAGAAGGCC |
| | | CAGGUGUCCGGCCAGGGCGACUCCCUGCACGAGCAC |
| | | AUCGCCAACCUGGCCGGCUCCCCCGCCAUCAAGAAG |
| | | GGCAUCCUGCAGACCGUGAAGGUGGUGGACGAGCUG |
| | | GUGAAGGUGAUGGGCCGGCACAAGCCCGAGAACAUC |
| | | GUGAUCGAGAUGGCCCGGGAGAACCAGACCACCCAG |
| | | AAGGGCCAGAAGAACUCCCGGGAGCGGAUGAAGCGG |
| | | AUCGAGGAGGGCAUCAAGGAGCUGGGCUCCCAGAUC |
| | | CUGAAGGAGCACCCCGUGGAGAACACCCAGCUGCAG |
| | | AACGAGAAGCUGUACCUGUACUACCUGCAGAACGGC |
| | | CGGGACAUGUACGUGGACCAGGAGCUGGACAUCAAC |
| | | CGGCUGUCCGACUACGACGUGGACCACAUCGUGCCC |
| | | CAGUCCUUCCUGAAGGACGACUCCAUCGACAACAAG |
| | | GUGCUGACCCGGUCCGACAAGAACCGGGGCAAGUCC |
| | | GACAACGUGCCCUCCGAGGAGGUGGUGAAGAAGAUG |
| | | AAGAACUACUGGCGGCAGCUGCUGAACGCCAAGCUG |
| | | AUCACCCAGCGGAAGUUCGACAACCUGACCAAGGCC |
| | | GAGCGGGGCGGCCUGUCCGAGCUGGACAAGGCCGGC |
| | | UUCAUCAAGCGGCAGCUGGUGGAGACCCGGCAGAUC |
| | | ACCAAGCACGUGGCCCAGAUCCUGGACUCCCGGAUG |
| | | AACACCAAGUACGACGAGAACGACAAGCUGAUCCGG |
| | | GAGGUGAAGGUGAUCACCCUGAAGUCCAAGCUGGUG |
| | | UCCGACUUCCGGAAGGACUUCCAGUUCUACAAGGUG |
| | | CGGGAGAUCAACAACUACCACCACGCCCACGACGCC |
| | | UACCUGAACGCCGUGGUGGGCACCGCCCUGAUCAAG |
| | | AAGUACCCCAAGCUGGAGUCCGAGUUCGUGUACGGC |
| | | GACUACAAGGUGUACGACGUGCGGAAGAUGAUCGCC |
| | | AAGUCCGAGCAGGAGAUCGGCAAGGCCACCGCCAAG |
| | | UACUUCUUCUACUCCAACAUCAUGAACUUCUUCAAG |
| | | ACCGAGAUCACCCUGGCCAACGGCGAGAUCCGGAAG |
| | | CGGCCCCUGAUCGAGACCAACGGCGAGACCGGCGAG |
| | | AUCGUGUGGGACAAGGGCCGGGACUUCGCCACCGUG |
| | | CGGAAGGUGCUGUCCAUGCCCCAGGUGAACAUCGUG |
| | | AAGAAGACCGAGGUGCAGACCGGCGGCUUCUCCAAG |
| | | GAGUCCAUCCUGCCCAAGCGGAACUCCGACAAGCUG |
| | | AUCGCCCGGAAGAAGGACUGGGACCCCAAGAAGUAC |
| | | GGCGGCUUCGACUCCCCCACCGUGGCCUACUCCGUGC |
| | | UGGUGGUGGCCAAGGUGGAGAAGGGCAAGUCCAAGA |
| | | AGCUGAAGUCCGUGAAGGAGCUGCUGGGCAUCACCA |
| | | UCAUGGAGCGGUCCUCCUUCGAGAAGAACCCCAUCG |
| | | ACUUCCUGGAGGCCAAGGGCUACAAGGAGGUGAAGA |
| | | AGGACCUGAUCAUCAAGCUGCCCAAGUACUCCCUGU |
| | | UCGAGCUGGAGAACGGCCGGAAGCGGAUGCUGGCCU |
| | | CCGCCGGCGAGCUGCAGAAGGGCAACGAGCUGGCCC |
| | | UGCCCUCCAAGUACGUGAACUUCCUGUACCUGGCCU |
| | | CCCACUACGAGAAGCUGAAGGGCUCCCCCGAGGACA |
| | | ACGAGCAGAAGCAGCUGUUCGUGGAGCAGCACAAGC |
| | | ACUACCUGGACGAGAUCAUCGAGCAGAUCUCCGAGU |
| | | UCUCCAAGCGGGUGAUCCUGGCCGACGCCAACCUGG |
| | | ACAAGGUGCUGUCCGCCUACAACAAGCACCGGGACA |
| | | AGCCCAUCCGGGAGCAGGCCGAGAACAUCAUCCACC |
| | | UGUUCACCCUGACCAACCUGGGCGCCCCCGCCGCCUU |
| | | CAAGUACUUCGACACCACCAUCGACCGGAAGCGGUA |
| | | CACCUCCACCAAGGAGGUGCUGGACGCCACCCUGAU |
| | | CCACCAGUCCAUCACCGGCCUGUACGAGACCCGGAU |
| | | CGACCUGUCCCAGCUGGGCGGCGACGGCGGCGGCUC |
| | | CCCCAAGAAGAAGCGGAAGGUGUCCGAGUCCGCCAC |
| | | CCCCGAGUCCGUGUCCGGCUGGCGGCUGUUCAAGAA |
| | | GAUCUCCUGA |
| | 806 | Not used |
| Open reading | 807 | AUGGGACCGAAGAAGAAGAGAAAGGUCGGAGGAGG |
| frame for UGI | | AAGCACAAACCUGUCGGACAUCAUCGAAAAGGAAAC |
| | | AGGAAAGCAGCUGGUCAUCCAGGAAUCGAUCCUGAU |
| | | GCUGCCGGAAGAAGUCGAAGAAGUCAUCGGAAACAA |
| | | GCCGGAAUCGGACAUCCUGGUCCACACAGCAUACGA |
| | | CGAAUCGACAGACGAAAACGUCAUGCUGCUGACAUC |
| | | GGACGCACCGGAAUACAAGCCGUGGGCACUGGUCAU |
| | | CCAGGACUCGAACGGAGAAAACAAGAUCAAGAUGCU |
| | | GUGA |
| Open reading | 808 | AUGACCAACCUGUCCGACAUCAUCGAGAAGGAGACC |
| frame for UGI | | GGCAAGCAGCUGGUGAUCCAGGAGUCCAUCCUGAUG |
| | | CUGCCCGAGGAGGUGGAGGAGGUGAUCGGCAACAAG |
| | | CCCGAGUCCGACAUCCUGGUGCACACCGCCUACGAC |
| | | GAGUCCACCGACGAGAACGUGAUGCUGCUGACCUCC |
| | | GACGCCCCCGAGUACAAGCCCUGGGCCCUGGUGAUC |
| | | CAGGACUCCAACGGCGAGAACAAGAUCAAGAUGCUG |
| | | UCCGGCGGCUCCAAGCGGACCGCCGACGGCUCCGAG |
| | | UUCGAGUCCCCCAAGAAGAAGCGGAAGGUGGAGUGA |
| Amino acid | 809 | MDKKYSIGLDIGTNSVGWAVITDEYKVPSKKFKVLGNT |
| sequence for | | DRHSIKKNLIGALLFDSGETAEATRLKRTARRRYTRRKN |
| Cas9 encoded | | RICYLQEIFSNEMAKVDDSFFHRLEESFLVEEDKKHERHP |
| by SEQ ID | | IFGNIVDEVAYHEKYPTIYHLRKKLVDSTDKADLRLIYLA |
| Nos. 801-802 | | LAHMIKFRGHFLIEGDLNPDNSDVDKLFIQLVQTYNQLF |
| | | EENPINASGVDAKAILSARLSKSRRLENLIAQLPGEKKNG |
| | | LFGNLIALSLGLTPNFKSNFDLAEDAKLQLSKDTYDDDL |
| | | DNLLAQIGDQYADLFLAAKNLSDAILLSDILRVNTEITKA |
| | | PLSASMIKRYDEHHQDLTLLKALVRQQLPEKYKEIFFDQ |
| | | SKNGYAGYIDGGASQEEFYKFIKPILEKMDGTEELLVKL |
| | | NREDLLRKQRTFDNGSIPHQIHLGELHAILRRQEDFYPFL |
| | | KDNREKIEKILTFRIPYYVGPLARGNSRFAWMTRKSEETI |
| | | TPWNFEEVVDKGASAQSFIERMTNFDKNLPNEKVLPKHS |
| | | LLYEYFTVYNELTKVKYVTEGMRKPAFLSGEQKKAIVD |
| | | LLFKTNRKVTVKQLKEDYFKKIECFDSVEISGVEDRFNAS |
| | | LGTYHDLLKIIKDKDFLDNEENEDILEDIVLTLTLFEDRE |
| | | MIEERLKTYAHLFDDKVMKQLKRRRYTGWGRLSRKLIN |
| | | GIRDKQSGKTILDFLKSDGFANRNFMQLIHDDSLTFKEDI |
| | | QKAQVSGQGDSLHEHIANLAGSPAIKKGILQTVKVVDEL |
| | | VKVMGRHKPENIVIEMARENQTTQKGQKNSRERMKRIE |
| | | EGIKELGSQILKEHPVENTQLQNEKLYLYYLQNGRDMY |
| | | VDQELDINRLSDYDVDHIVPQSFLKDDSIDNKVLTRSDK |
| | | NRGKSDNVPSEEVVKKMKNYWRQLLNAKLITQRKFDN |
| | | LTKAERGGLSELDKAGFIKRQLVETRQITKHVAQILDSR |
| | | MNTKYDENDKLIREVKVITLKSKLVSDFRKDFQFYKVRE |
| | | INNYHHAHDAYLNAVVGTALIKKYPKLESEFVYGDYKV |
| | | YDVRKMIAKSEQEIGKATAKYFFYSNIMNFFKTEITLAN |
| | | GEIRKRPLIETNGETGEIVWDKGRDFATVRKVLSMPQVN |
| | | IVKKTEVQTGGFSKESILPKRNSDKLIARKKDWDPKKYG |
| | | GFDSPTVAYSVLVVAKVEKGKSKKLKSVKELLGITIMER |
| | | SSFEKNPIDFLEAKGYKEVKKDLIIKLPKYSLFELENGRK |
| | | RMLASAGELQKGNELALPSKYVNFLYLASHYEKLKGSP |
| | | EDNEQKQLFVEQHKHYLDEIIEQISEFSKRVILADANLDK |
| | | VLSAYNKHRDKPIREQAENIIHLFTLTNLGAPAAFKYFDT |
| | | TIDRKRYTSTKEVLDATLIHQSITGLYETRIDLSQLGGDG |
| | | GGSPKKKRKV |
| Amino acid | 810 | MDKKYSIGLDIGTNSVGWAVITDEYKVPSKKFKVLGNT |
| sequence for | | DRHSIKKNLIGALLFDSGETAEATRLKRTARRRYTRRKN |
| Cas9 with | | RICYLQEIFSNEMAKVDDSFFHRLEESFLVEEDKKHERHP |
| Hibit tag | | IFGNIVDEVAYHEKYPTIYHLRKKLVDSTDKADLRLIYLA |
| | | LAHMIKFRGHFLIEGDLNPDNSDVDKLFIQLVQTYNQLF |
| | | EENPINASGVDAKAILSARLSKSRRLENLIAQLPGEKKNG |
| | | LFGNLIALSLGLTPNFKSNFDLAEDAKLQLSKDTYDDDL |
| | | DNLLAQIGDQYADLFLAAKNLSDAILLSDILRVNTEITKA |
| | | PLSASMIKRYDEHHQDLTLLKALVRQQLPEKYKEIFFDQ |
| | | SKNGYAGYIDGGASQEEFYKFIKPILEKMDGTEELLVKL |
| | | NREDLLRKQRTFDNGSIPHQIHLGELHAILRRQEDFYPFL |
| | | KDNREKIEKILTFRIPYYVGPLARGNSRFAWMTRKSEETI |
| | | TPWNFEEVVDKGASAQSFIERMTNFDKNLPNEKVLPKHS |
| | | LLYEYFTVYNELTKVKYVTEGMRKPAFLSGEQKKAIVD |
| | | LLFKTNRKVTVKQLKEDYFKKIECFDSVEISGVEDRFNAS |
| | | LGTYHDLLKIIKDKDFLDNEENEDILEDIVLTLTLFEDRE |
| | | MIEERLKTYAHLFDDKVMKQLKRRRYTGWGRLSRKLIN |
| | | GIRDKQSGKTILDFLKSDGFANRNFMQLIHDDSLTFKEDI |
| | | QKAQVSGQGDSLHEHIANLAGSPAIKKGILQTVKVVDEL |
| | | VKVMGRHKPENIVIEMARENQTTQKGQKNSRERMKRIE |
| | | EGIKELGSQILKEHPVENTQLQNEKLYLYYLQNGRDMY |
| | | VDQELDINRLSDYDVDHIVPQSFLKDDSIDNKVLTRSDK |
| | | NRGKSDNVPSEEVVKKMKNYWRQLLNAKLITQRKFDN |
| | | LTKAERGGLSELDKAGFIKRQLVETRQITKHVAQILDSR |
| | | MNTKYDENDKLIREVKVITLKSKLVSDFRKDFQFYKVRE |
| | | INNYHHAHDAYLNAVVGTALIKKYPKLESEFVYGDYKV |
| | | YDVRKMIAKSEQEIGKATAKYFFYSNIMNFFKTEITLAN |
| | | GEIRKRPLIETNGETGEIVWDKGRDFATVRKVLSMPQVN |
| | | IVKKTEVQTGGFSKESILPKRNSDKLIARKKDWDPKKYG |
| | | GFDSPTVAYSVLVVAKVEKGKSKKLKSVKELLGITIMER |
| | | SSFEKNPIDFLEAKGYKEVKKDLIIKLPKYSLFELENGRK |
| | | RMLASAGELQKGNELALPSKYVNFLYLASHYEKLKGSP |
| | | EDNEQKQLFVEQHKHYLDEIIEQISEFSKRVILADANLDK |
| | | VLSAYNKHRDKPIREQAENIIHLFTLTNLGAPAAFKYFDT |
| | | TIDRKRYTSTKEVLDATLIHQSITGLYETRIDLSQLGGDG |
| | | GGSPKKKRKVSESATPESVSGWRLFKKIS |
| Amino acid | 811 | MEASPASGPRHLMDPHIFTSNFNNGIGRHKTYLCYEVER |
| sequence for | | LDNGTSVKMDQHRGFLHNQAKNLLCGFYGRHAELRFL |
| BC22n | | DLVPSLQLDPAQIYRVTWFISWSPCFSWGCAGEVRAFLQ |
| | | ENTHVRLRIFAARIYDYDPLYKEALQMLRDAGAQVSIMT |
| | | YDEFKHCWDTFVDHQGCPFQPWDGLDEHSQALSGRLR |
| | | AILQNQGNSGSETPGTSESATPESDKKYSIGLAIGTNSVG |
| | | WAVITDEYKVPSKKFKVLGNTDRHSIKKNLIGALLFDSG |
| | | ETAEATRLKRTARRRYTRRKNRICYLQEIFSNEMAKVDD |
| | | SFFHRLEESFLVEEDKKHERHPIFGNIVDEVAYHEKYPTI |
| | | YHLRKKLVDSTDKADLRLIYLALAHMIKFRGHFLIEGDL |
| | | NPDNSDVDKLFIQLVQTYNQLFEENPINASGVDAKAILSA |
| | | RLSKSRRLENLIAQLPGEKKNGLFGNLIALSLGLTPNFKS |
| | | NFDLAEDAKLQLSKDTYDDDLDNLLAQIGDQYADLFLA |
| | | AKNLSDAILLSDILRVNTEITKAPLSASMIKRYDEHHQDL |
| | | TLLKALVRQQLPEKYKEIFFDQSKNGYAGYIDGGASQEE |
| | | FYKFIKPILEKMDGTEELLVKLNREDLLRKQRTFDNGSIP |
| | | HQIHLGELHAILRRQEDFYPFLKDNREKIEKILTFRIPYYV |
| | | GPLARGNSRFAWMTRKSEETITPWNFEEVVDKGASAQS |
| | | FIERMTNFDKNLPNEKVLPKHSLLYEYFTVYNELTKVKY |
| | | VTEGMRKPAFLSGEQKKAIVDLLFKTNRKVTVKQLKED |
| | | YFKKIECFDSVEISGVEDRFNASLGTYHDLLKIIKDKDFL |
| | | DNEENEDILEDIVLTLTLFEDREMIEERLKTYAHLFDDKV |
| | | MKQLKRRRYTGWGRLSRKLINGIRDKQSGKTILDFLKSD |
| | | GFANRNFMQLIHDDSLTFKEDIQKAQVSGQGDSLHEHIA |
| | | NLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHI |
| | | VPQSFLKDDSIDNKVLTRSDKNRGKSDNVPSEEVVKKM |
| | | KNYWRQLLNAKLITQRKFDNLTKAERGGLSELDKAGFI |
| | | KRQLVETRQITKHVAQILDSRMNTKYDENDKLIREVKVI |
| | | TLKSKLVSDFRKDFQFYKVREINNYHHAHDAYLNAVVG |
| | | TALIKKYPKLESEFVYGDYKVYDVRKMIAKSEQEIGKAT |
| | | AKYFFYSNIMNFFKTEITLANGEIRKRPLIETNGETGEIVW |
| | | DKGRDFATVRKVLSMPQVNIVKKTEVQTGGFSKESILPK |
| | | RNSDKLIARKKDWDPKKYGGFDSPTVAYSVLVVAKVEK |
| | | GKSKKLKSVKELLGITIMERSSFEKNPIDFLEAKGYKEVK |
| | | KDLIIKLPKYSLFELENGRKRMLASAGELQKGNELALPS |
| | | KYVNFLYLASHYEKLKGSPEDNEQKQLFVEQHKHYLDE |
| | | IIEQISEFSKRVILADANLDKVLSAYNKHRDKPIREQAENI |
| | | IHLFTLTNLGAPAAFKYFDTTIDRKRYTSTKEVLDATLIH |
| | | QSITGLYETRIDLSQLGGDGGGSPKKKRKV* |
| Amino acid | 812 | MEASPASGPRHLMDPHIFTSNFNNGIGRHKTYLCYEVER |
| sequence for | | LDNGTSVKMDQHRGFLHNQAKNLLCGFYGRHAELRFL |
| BC22n with | | DLVPSLQLDPAQIYRVTWFISWSPCFSWGCAGEVRAFLQ |
| Hibit tag | | ENTHVRLRIFAARIYDYDPLYKEALQMLRDAGAQVSIMT |
| | | YDEFKHCWDTFVDHQGCPFQPWDGLDEHSQALSGRLR |
| | | AILQNQGNSGSETPGTSESATPESDKKYSIGLAIGTNSVG |
| | | WAVITDEYKVPSKKFKVLGNTDRHSIKKNLIGALLFDSG |
| | | ETAEATRLKRTARRRYTRRKNRICYLQEIFSNEMAKVDD |
| | | SFFHRLEESFLVEEDKKHERHPIFGNIVDEVAYHEKYPTI |
| | | YHLRKKLVDSTDKADLRLIYLALAHMIKFRGHFLIEGDL |
| | | NPDNSDVDKLFIQLVQTYNQLFEENPINASGVDAKAILSA |
| | | RLSKSRRLENLIAQLPGEKKNGLFGNLIALSLGLTPNFKS |
| | | NFDLAEDAKLQLSKDTYDDDLDNLLAQIGDQYADLFLA |
| | | AKNLSDAILLSDILRVNTEITKAPLSASMIKRYDEHHQDL |
| | | TLLKALVRQQLPEKYKEIFFDQSKNGYAGYIDGGASQEE |
| | | FYKFIKPILEKMDGTEELLVKLNREDLLRKQRTFDNGSIP |
| | | HQIHLGELHAILRRQEDFYPFLKDNREKIEKILTFRIPYYV |
| | | GPLARGNSRFAWMTRKSEETITPWNFEEVVDKGASAQS |
| | | FIERMTNFDKNLPNEKVLPKHSLLYEYFTVYNELTKVKY |
| | | VTEGMRKPAFLSGEQKKAIVDLLFKTNRKVTVKQLKED |
| | | YFKKIECFDSVEISGVEDRFNASLGTYHDLLKIIKDKDFL |
| | | DNEENEDILEDIVLTLTLFEDREMIEERLKTYAHLFDDKV |
| | | MKQLKRRRYTGWGRLSRKLINGIRDKQSGKTILDFLKSD |
| | | GFANRNFMQLIHDDSLTFKEDIQKAQVSGQGDSLHEHIA |
| | | NLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHI |
| | | VPQSFLKDDSIDNKVLTRSDKNRGKSDNVPSEEVVKKM |
| | | KNYWRQLLNAKLITQRKFDNLTKAERGGLSELDKAGFI |
| | | KRQLVETRQITKHVAQILDSRMNTKYDENDKLIREVKVI |
| | | TLKSKLVSDFRKDFQFYKVREINNYHHAHDAYLNAVVG |
| | | TALIKKYPKLESEFVYGDYKVYDVRKMIAKSEQEIGKAT |
| | | AKYFFYSNIMNFFKTEITLANGEIRKRPLIETNGETGEIVW |
| | | DKGRDFATVRKVLSMPQVNIVKKTEVQTGGFSKESILPK |
| | | RNSDKLIARKKDWDPKKYGGFDSPTVAYSVLVVAKVEK |
| | | GKSKKLKSVKELLGITIMERSSFEKNPIDFLEAKGYKEVK |
| | | KDLIIKLPKYSLFELENGRKRMLASAGELQKGNELALPS |
| | | KYVNFLYLASHYEKLKGSPEDNEQKQLFVEQHKHYLDE |
| | | IIEQISEFSKRVILADANLDKVLSAYNKHRDKPIREQAENI |
| | | IHLFTLTNLGAPAAFKYFDTTIDRKRYTSTKEVLDATLIH |
| | | QSITGLYETRIDLSQLGGDGGGSPKKKRKVSESATPESVS |
| | | GWRLFKKIS |
| | 813 | Not used |
| Amino acid | 814 | MTNLSDIIEKETGKQLVIQESILMLPEEVEEVIGNKPESDI |
| sequence for | | LVHTAYDESTDENVMLLTSDAPEYKPWALVIQDSNGEN |
| UGI | | KIKMLSGGSKRTADGSEFESPKKKRKVE |
| | 815 | Not used |
| G023519 | 816 | mA*mC*mU*CACGCUGGAUAGCCUCCGUUUUAGAmG |
| Guide RNA | | mCmUmAmGmAmAmAmUmAmGmCAAGUUAAAAUAAG |
| Targeting | | GCUAGUCCGUUAUCACGAAAGGGCACCGAGUCGGmU |
| B2M | | mGmC*mU |
| Open reading | 817 | AUGGACAAGAAGUACAGCAUCGGACUGGACAUCGGA |
| frame for | | ACAAACAGCGUCGGAUGGGCAGUCAUCACAGACGAA |
| Cas9 | | UACAAGGUCCCGAGCAAGAAGUUCAAGGUCCUGGGA |
| | | AACACAGACAGACACAGCAUCAAGAAGAACCUGAUC |
| | | GGAGCACUGCUGUUCGACAGCGGAGAAACAGCAGAA |
| | | GCAACAAGACUGAAGAGAACAGCAAGAAGAAGAUAC |
| | | ACAAGAAGAAAGAACAGAAUCUGCUACCUGCAGGAA |
| | | AUCUUCAGCAACGAAAUGGCAAAGGUCGACGACAGC |
| | | UUCUUCCACAGACUGGAAGAAAGCUUCCUGGUCGAA |
| | | GAAGACAAGAAGCACGAAAGACACCCGAUCUUCGGA |
| | | AACAUCGUCGACGAAGUCGCAUACCACGAAAAGUAC |
| | | CCGACAAUCUACCACCUGAGAAAGAAGCUGGUCGAC |
| | | AGCACAGACAAGGCAGACCUGAGACUGAUCUACCUG |
| | | GCACUGGCACACAUGAUCAAGUUCAGAGGACACUUC |
| | | CUGAUCGAAGGAGACCUGAACCCGGACAACAGCGAC |
| | | GUCGACAAGCUGUUCAUCCAGCUGGUCCAGACAUAC |
| | | AACCAGCUGUUCGAAGAAAACCCGAUCAACGCAAGC |
| | | GGAGUCGACGCAAAGGCAAUCCUGAGCGCAAGACUG |
| | | AGCAAGAGCAGAAGACUGGAAAACCUGAUCGCACAG |
| | | CUGCCGGGAGAAAAGAAGAACGGACUGUUCGGAAAC |
| | | CUGAUCGCACUGAGCCUGGGACUGACACCGAACUUC |
| | | AAGAGCAACUUCGACCUGGCAGAAGACGCAAAGCUG |
| | | CAGCUGAGCAAGGACACAUACGACGACGACCUGGAC |
| | | AACCUGCUGGCACAGAUCGGAGACCAGUACGCAGAC |
| | | CUGUUCCUGGCAGCAAAGAACCUGAGCGACGCAAUC |
| | | CUGCUGAGCGACAUCCUGAGAGUCAACACAGAAAUC |
| | | ACAAAGGCACCGCUGAGCGCAAGCAUGAUCAAGAGA |
| | | UACGACGAACACCACCAGGACCUGACACUGCUGAAG |
| | | GCACUGGUCAGACAGCAGCUGCCGGAAAAGUACAAG |
| | | GAAAUCUUCUUCGACCAGAGCAAGAACGGAUACGCA |
| | | GGAUACAUCGACGGAGGAGCAAGCCAGGAAGAAUUC |
| | | UACAAGUUCAUCAAGCCGAUCCUGGAAAAGAUGGAC |
| | | GGAACAGAAGAACUGCUGGUCAAGCUGAACAGAGAA |
| | | GACCUGCUGAGAAAGCAGAGAACAUUCGACAACGGA |
| | | AGCAUCCCGCACCAGAUCCACCUGGGAGAACUGCAC |
| | | GCAAUCCUGAGAAGACAGGAAGACUUCUACCCGUUC |
| | | CUGAAGGACAACAGAGAAAAGAUCGAAAAGAUCCUG |
| | | ACAUUCAGAAUCCCGUACUACGUCGGACCGCUGGCA |
| | | AGAGGAAACAGCAGAUUCGCAUGGAUGACAAGAAAG |
| | | AGCGAAGAAACAAUCACACCGUGGAACUUCGAAGAA |
| | | GUCGUCGACAAGGGAGCAAGCGCACAGAGCUUCAUC |
| | | GAAAGAAUGACAAACUUCGACAAGAACCUGCCGAAC |
| | | GAAAAGGUCCUGCCGAAGCACAGCCUGCUGUACGAA |
| | | UACUUCACAGUCUACAACGAACUGACAAAGGUCAAG |
| | | UACGUCACAGAAGGAAUGAGAAAGCCGGCAUUCCUG |
| | | AGCGGAGAACAGAAGAAGGCAAUCGUCGACCUGCUG |
| | | UUCAAGACAAACAGAAAGGUCACAGUCAAGCAGCUG |
| | | AAGGAAGACUACUUCAAGAAGAUCGAAUGCUUCGAC |
| | | AGCGUCGAAAUCAGCGGAGUCGAAGACAGAUUCAAC |
| | | GCAAGCCUGGGAACAUACCACGACCUGCUGAAGAUC |
| | | AUCAAGGACAAGGACUUCCUGGACAACGAAGAAAAC |
| | | GAAGACAUCCUGGAAGACAUCGUCCUGACACUGACA |
| | | CUGUUCGAAGACAGAGAAAUGAUCGAAGAAAGACUG |
| | | AAGACAUACGCACACCUGUUCGACGACAAGGUCAUG |
| | | AAGCAGCUGAAGAGAAGAAGAUACACAGGAUGGGGA |
| | | AGACUGAGCAGAAAGCUGAUCAACGGAAUCAGAGAC |
| | | AAGCAGAGCGGAAAGACAAUCCUGGACUUCCUGAAG |
| | | AGCGACGGAUUCGCAAACAGAAACUUCAUGCAGCUG |
| | | AUCCACGACGACAGCCUGACAUUCAAGGAAGACAUC |
| | | CAGAAGGCACAGGUCAGCGGACAGGGAGACAGCCUG |
| | | CACGAACACAUCGCAAACCUGGCAGGAAGCCCGGCA |
| | | AUCAAGAAGGGAAUCCUGCAGACAGUCAAGGUCGUC |
| | | GACGAACUGGUCAAGGUCAUGGGAAGACACAAGCCG |
| | | GAAAACAUCGUCAUCGAAAUGGCAAGAGAAAACCAG |
| | | ACAACACAGAAGGGACAGAAGAACAGCAGAGAAAGA |
| | | AUGAAGAGAAUCGAAGAAGGAAUCAAGGAACUGGG |
| | | AAGCCAGAUCCUGAAGGAACACCCGGUCGAAAACAC |
| | | ACAGCUGCAGAACGAAAAGCUGUACCUGUACUACCU |
| | | GCAGAACGGAAGAGACAUGUACGUCGACCAGGAACU |
| | | GGACAUCAACAGACUGAGCGACUACGACGUCGACCA |
| | | CAUCGUCCCGCAGAGCUUCCUGAAGGACGACAGCAU |
| | | CGACAACAAGGUCCUGACAAGAAGCGACAAGAACAG |
| | | AGGAAAGAGCGACAACGUCCCGAGCGAAGAAGUCGU |
| | | CAAGAAGAUGAAGAACUACUGGAGACAGCUGCUGAA |
| | | CGCAAAGCUGAUCACACAGAGAAAGUUCGACAACCU |
| | | GACAAAGGCAGAGAGAGGAGGACUGAGCGAACUGGA |
| | | CAAGGCAGGAUUCAUCAAGAGACAGCUGGUCGAAAC |
| | | AAGACAGAUCACAAAGCACGUCGCACAGAUCCUGGA |
| | | CAGCAGAAUGAACACAAAGUACGACGAAAACGACAA |
| | | GCUGAUCAGAGAAGUCAAGGUCAUCACACUGAAGAG |
| | | CAAGCUG |
| | | GUCAGCGACUUCAGAAAGGACUUCCAGUUCUACAAG |
| | | GUCAGAGAAAUCAACAACUACCACCACGCACACGAC |
| | | GCAUACCUGAACGCAGUCGUCGGAACAGCACUGAUC |
| | | AAGAAGUACCCGAAGCUGGAAAGCGAAUUCGUCUAC |
| | | GGAGACUACAAGGUCUACGACGUCAGAAAGAUGAUC |
| | | GCAAAGAGCGAACAGGAAAUCGGAAAGGCAACAGCA |
| | | AAGUACUUCUUCUACAGCAACAUCAUGAACUUCUUC |
| | | AAGACAGAAAUCACACUGGCAAACGGAGAAAUCAGA |
| | | AAGAGACCGCUGAUCGAAACAAACGGAGAAACAGGA |
| | | GAAAUCGUCUGGGACAAGGGAAGAGACUUCGCAACA |
| | | GUCAGAAAGGUCCUGAGCAUGCCGCAGGUCAACAUC |
| | | GUCAAGAAGACAGAAGUCCAGACAGGAGGAUUCAGC |
| | | AAGGAAAGCAUCCUGCCGAAGAGAAACAGCGACAAG |
| | | CUGAUCGCAAGAAAGAAGGACUGGGACCCGAAGAAG |
| | | UACGGAGGAUUCGACAGCCCGACAGUCGCAUACAGC |
| | | GUCCUGGUCGUCGCAAAGGUCGAAAAGGGAAAGAGC |
| | | AAGAAGCUGAAGAGCGUCAAGGAACUGCUGGGAAUC |
| | | ACAAUCAUGGAAAGAAGCAGCUUCGAAAAGAACCCG |
| | | AUCGACUUCCUGGAAGCAAAGGGAUACAAGGAAGUC |
| | | AAGAAGGACCUGAUCAUCAAGCUGCCGAAGUACAGC |
| | | CUGUUCGAACUGGAAAACGGAAGAAAGAGAAUGCUG |
| | | GCAAGCGCAGGAGAACUGCAGAAGGGAAACGAACUG |
| | | GCACUGCCGAGCAAGUACGUCAACUUCCUGUACCUG |
| | | GCAAGCCACUACGAAAAGCUGAAGGGAAGCCCGGAA |
| | | GACAACGAACAGAAGCAGCUGUUCGUCGAACAGCAC |
| | | AAGCACUACCUGGACGAAAUCAUCGAACAGAUCAGC |
| | | GAAUUCAGCAAGAGAGUCAUCCUGGCAGACGCAAAC |
| | | CUGGACAAGGUCCUGAGCGCAUACAACAAGCACAGA |
| | | GACAAGCCGAUCAGAGAACAGGCAGAAAACAUCAUC |
| | | CACCUGUUCACACUGACAAACCUGGGAGCACCGGCA |
| | | GCAUUCAAGUACUUCGACACAACAAUCGACAGAAAG |
| | | AGAUACACAAGCACAAAGGAAGUCCUGGACGCAACA |
| | | CUGAUCCACCAGAGCAUCACAGGACUGUACGAAACA |
| | | AGAAUCGACCUGAGCCAGCUGGGAGGAGACGGAGGA |
| | | GGAAGCCCGAAGAAGAAGAGAAAGGUCUAG |
| Open reading | 818 | AUGGAAGCAAGCCCGGCAAGCGGACCGAGACACCUG |
| frame for | | AUGGACCCGCACAUCUUCACAAGCAACUUCAACAAC |
| BC22 | | GGAAUCGGAAGACACAAGACAUACCUGUGCUACGAA |
| | | GUCGAAAGACUGGACAACGGAACAAGCGUCAAGAUG |
| | | GACCAGCACAGAGGAUUCCUGCACAACCAGGCAAAG |
| | | AACCUGCUGUGCGGAUUCUACGGAAGACACGCAGAA |
| | | CUGAGAUUCCUGGACCUGGUCCCGAGCCUGCAGCUG |
| | | GACCCGGCACAGAUCUACAGAGUCACAUGGUUCAUC |
| | | AGCUGGAGCCCGUGCUUCAGCUGGGGAUGCGCAGGA |
| | | GAAGUCAGAGCAUUUCUGCAGGAAAACACACACGUC |
| | | AGACUGAGAAUCUUCGCAGCAAGAAUCUAC |
| | | GACUACGACCCGCUGUACAAGGAAGCACUGCAGAUG |
| | | CUGAGAGACGCAGGAGCACAGGUCAGCAUCAUGACA |
| | | UACGACGAAUUCAAGCACUGCUGGGACACAUUCGUC |
| | | GACCACCAGGGAUGCCCGUUCCAGCCGUGGGACGGA |
| | | CUGGACGAACACAGCCAGGCACUGAGCGGAAGACUG |
| | | AGAGCAAUCCUGCAGAACCAGGGAAACAGCGGAAGC |
| | | GAAACACCGGGAACAAGCGAAAGCGCAACACCGGAA |
| | | AGCGACAAGAAGUACAGCAUCGGACUGGCCAUCGGA |
| | | ACAAACAGCGUCGGAUGGGCAGUCAUCACAGACGAA |
| | | UACAAGGUCCCGAGCAAGAAGUUCAAGGUCCUGGGA |
| | | AACACAGACAGACACAGCAUCAAGAAGAACCUGAUC |
| | | GGAGCACUGCUGUUCGACAGCGGAGAAACAGCAGAA |
| | | GCAACAAGACUGAAGAGAACAGCAAGAAGAAGAUAC |
| | | ACAAGAAGAAAGAACAGAAUCUGCUACCUGCAGGAA |
| | | AUCUUCAGCAACGAAAUGGCAAAGGUCGACGACAGC |
| | | UUCUUCCACAGACUGGAAGAAAGCUUCCUGGUCGAA |
| | | GAAGACAAGAAGCACGAAAGACACCCGAUCUUCGGA |
| | | AACAUCGUCGACGAAGUCGCAUACCACGAAAAGUAC |
| | | CCGACAAUCUACCACCUGAGAAAGAAGCUGGUCGAC |
| | | AGCACAGACAAGGCAGACCUGAGACUGAUCUACCUG |
| | | GCACUGGCACACAUGAUCAAGUUCAGAGGACACUUC |
| | | CUGAUCGAAGGAGACCUGAACCCGGACAACAGCGAC |
| | | GUCGACAAGCUGUUCAUCCAGCUGGUCCAGACAUAC |
| | | AACCAGCUGUUCGAAGAAAACCCGAUCAACGCAAGC |
| | | GGAGUCGACGCAAAGGCAAUCCUGAGCGCAAGACUG |
| | | AGCAAGAGCAGAAGACUGGAAAACCUGAUCGCACAG |
| | | CUGCCGGGAGAAAAGAAGAACGGACUGUUCGGAAAC |
| | | CUGAUCGCACUGAGCCUGGGACUGACACCGAACUUC |
| | | AAGAGCAACUUCGACCUGGCAGAAGACGCAAAGCUG |
| | | CAGCUGAGCAAGGACACAUACGACGACGACCUGGAC |
| | | AACCUGCUGGCACAGAUCGGAGACCAGUACGCAGAC |
| | | CUGUUCCUGGCAGCAAAGAACCUGAGCGACGCAAUC |
| | | CUGCUGAGCGACAUCCUGAGAGUCAACACAGAAAUC |
| | | ACAAAGGCACCGCUGAGCGCAAGCAUGAUCAAGAGA |
| | | UACGACGAACACCACCAGGACCUGACACUGCUGAAG |
| | | GCACUGGUCAGACAGCAGCUGCCGGAAAAGUACAAG |
| | | GAAAUCUUCUUCGACCAGAGCAAGAACGGAUACGCA |
| | | GGAUACAUCGACGGAGGAGCAAGCCAGGAAGAAUUC |
| | | UACAAGUUCAUCAAGCCGAUCCUGGAAAAGAUGGAC |
| | | GGAACAGAAGAACUGCUGGUCAAGCUGAACAGAGAA |
| | | GACCUGCUGAGAAAGCAGAGAACAUUCGACAACGGA |
| | | AGCAUCCCGCACCAGAUCCACCUGGGAGAACUGCAC |
| | | GCAAUCCUGAGAAGACAGGAAGACUUCUACCCGUUC |
| | | CUGAAGGACAACAGAGAAAAGAUCGAAAAGAUCCUG |
| | | ACAUUCAGAAUCCCGUACUACGUCGGACCGCUGGCA |
| | | AGAGGAAACAGCAGAUUCGCAUGGAUGACAAGAAAG |
| | | AGCGAAGAAACAAUCACACCGUGGAACUUCGAAGAA |
| | | GUCGUCGACAAGGGAGCAAGCGCACAGAGCUUCAUC |
| | | GAAAGAAUGACAAACUUCGACAAGAACCUGCCGAAC |
| | | GAAAAGGUCCUGCCGAAGCACAGCCUGCUGUACGAA |
| | | UACUUCACAGUCUACAACGAACUGACAAAGGUCAAG |
| | | UACGUCACAGAAGGAAUGAGAAAGCCGGCAUUCCUG |
| | | AGCGGAGAACAGAAGAAGGCAAUCGUCGACCUGCUG |
| | | UUCAAGACAAACAGAAAGGUCACAGUCAAGCAGCUG |
| | | AAGGAAGACUACUUCAAGAAGAUCGAAUGCUUCGAC |
| | | AGCGUCGAAAUCAGCGGAGUCGAAGACAGAUUCAAC |
| | | GCAAGCCUGGGAACAUACCACGACCUGCUGAAGAUC |
| | | AUCAAGGACAAGGACUUCCUGGACAACGAAGAAAAC |
| | | GAAGACAUCCUGGAAGACAUCGUCCUGACACUGACA |
| | | CUGUUCGAAGACAGAGAAAUGAUCGAAGAAAGACUG |
| | | AAGACAUACGCACACCUGUUCGACGACAAGGUCAUG |
| | | AAGCAGCUGAAGAGAAGAAGAUACACAGGAUGGGGA |
| | | AGACUGAGCAGAAAGCUGAUCAACGGAAUCAGAGAC |
| | | AAGCAGAGCGGAAAGACAAUCCUGGACUUCCUGAAG |
| | | AGCGACGGAUUCGCAAACAGAAACUUCAUGCAGCUG |
| | | AUCCACGACGACAGCCUGACAUUCAAGGAAGACAUC |
| | | CAGAAGGCACAGGUCAGCGGACAGGGAGACAGCCUG |
| | | CACGAACACAUCGCAAACCUGGCAGGAAGCCCGGCA |
| | | AUCAAGAAGGGAAUCCUGCAGACAGUCAAGGUCGUC |
| | | GACGAACUGGUCAAGGUCAUGGGAAGACACAAGCCG |
| | | GAAAACAUCGUCAUCGAAAUGGCAAGAGAAAACCAG |
| | | ACAACACAGAAGGGACAGAAGAACAGCAGAGAAAGA |
| | | AUGAAGAGAAUCGAAGAAGGAAUCAAGGAACUGGG |
| | | AAGCCAGAUCCUGAAGGAACACCCGGUCGAAAACAC |
| | | ACAGCUGCAGAACGAAAAGCUGUACCUGUACUACCU |
| | | GCAGAACGGAAGAGACAUGUACGUCGACCAGGAACU |
| | | GGACAUCAACAGACUGAGCGACUACGACGUCGACCA |
| | | CAUCGUCCCGCAGAGCUUCCUGAAGGACGACAGCAU |
| | | C |
| | | GACAACAAGGUCCUGACAAGAAGCGACAAGAACAGA |
| | | GGAAAGAGCGACAACGUCCCGAGCGAAGAAGUCGUC |
| | | AAGAAGAUGAAGAACUACUGGAGACAGCUGCUGAAC |
| | | GCAAAGCUGAUCACACAGAGAAAGUUCGACAACCUG |
| | | ACAAAGGCAGAGAGAGGAGGACUGAGCGAACUGGAC |
| | | AAGGCAGGAUUCAUCAAGAGACAGCUGGUCGAAACA |
| | | AGACAGAUCACAAAGCACGUCGCACAGAUCCUGGAC |
| | | AGCAGAAUGAACACAAAGUACGACGAAAACGACAAG |
| | | CUGAUCAGAGAAGUCAAGGUCAUCACACUGAAGAGC |
| | | AAGCUGGUCAGCGACUUCAGAAAGGACUUCCAGUUC |
| | | UACAAGGUCAGAGAAAUCAACAACUACCACCACGCA |
| | | CACGACGCAUACCUGAACGCAGUCGUCGGAACAGCA |
| | | CUGAUCAAGAAGUACCCGAAGCUGGAAAGCGAAUUC |
| | | GUCUACGGAGACUACAAGGUCUACGACGUCAGAAAG |
| | | AUGAUCGCAAAGAGCGAACAGGAAAUCGGAAAGGCA |
| | | ACAGCAAAGUACUUCUUCUACAGCAACAUCAUGAAC |
| | | UUCUUCAAGACAGAAAUCACACUGGCAAACGGAGAA |
| | | AUCAGAAAGAGACCGCUGAUCGAAACAAACGGAGAA |
| | | ACAGGAGAAAUCGUCUGGGACAAGGGAAGAGACUUC |
| | | GCAACAGUCAGAAAGGUCCUGAGCAUGCCGCAGGUC |
| | | AACAUCGUCAAGAAGACAGAAGUCCAGACAGGAGGA |
| | | UUCAGCAAGGAAAGCAUCCUGCCGAAGAGAAACAGC |
| | | GACAAGCUGAUCGCAAGAAAGAAGGACUGGGACCCG |
| | | AAGAAGUACGGAGGAUUCGACAGCCCGACAGUCGCA |
| | | UACAGCGUCCUGGUCGUCGCAAAGGUCGAAAAGGGA |
| | | AAGAGCAAGAAGCUGAAGAGCGUCAAGGAACUGCUG |
| | | GGAAUCACAAUCAUGGAAAGAAGCAGCUUCGAAAAG |
| | | AACCCGAUCGACUUCCUGGAAGCAAAGGGAUACAAG |
| | | GAAGUCAAGAAGGACCUGAUCAUCAAGCUGCCGAAG |
| | | UACAGCCUGUUCGAACUGGAAAACGGAAGAAAGAGA |
| | | AUGCUGGCAAGCGCAGGAGAACUGCAGAAGGGAAAC |
| | | GAACUGGCACUGCCGAGCAAGUACGUCAACUUCCUG |
| | | UACCUGGCAAGCCACUACGAAAAGCUGAAGGGAAGC |
| | | CCGGAAGACAACGAACAGAAGCAGCUGUUCGUCGAA |
| | | CAGCACAAGCACUACCUGGACGAAAUCAUCGAACAG |
| | | AUCAGCGAAUUCAGCAAGAGAGUCAUCCUGGCAGAC |
| | | GCAAACCUGGACAAGGUCCUGAGCGCAUACAACAAG |
| | | CACAGAGACAAGCCGAUCAGAGAACAGGCAGAAAAC |
| | | AUCAUCCACCUGUUCACACUGACAAACCUGGGAGCA |
| | | CCGGCAGCAUUCAAGUACUUCGACACAACAAUCGAC |
| | | AGAAAGAGAUACACAAGCACAAAGGAAGUCCUGGAC |
| | | GCAACACUGAUCCACCAGAGCAUCACAGGACUGUAC |
| | | GAAACAAGAAUCGAUCUGAGCCAGCUGGGAGGAGAC |
| | | AGCGGAGGAAGCACAAACCUGAGCGACAUCAUCGAA |
| | | AAGGAAACAGGAAAGCAGCUGGUCAUCCAGGAAAGC |
| | | AUCCUGAUGCUGCCGGAAGAAGUCGAAGAAGUCAUC |
| | | GGAAACAAGCCGGAAAGCGACAUCCUGGUCCACACA |
| | | GCAUACGACGAAAGCACAGACGAAAACGUCAUGCUG |
| | | CUGACAAGCGACGCACCGGAAUACAAGCCGUGGGCA |
| | | CUGGUCAUCCAGGACAGCAACGGAGAAAACAAGAUC |
| | | AAGAUGCUGAGCGGAGGAAGCCCGAAGAAGAAGAGA |
| | | AAGGUCUAA |
| Open reading | 819 | AUGGGACCGAAGAAGAAGAGAAAGGUCGGAGGAGG |
| frame for UGI | | AAGCACAAACCUGUCGGACAUCAUCGAAAAGGAAAC |
| | | AGGAAAGCAGCUGGUCAUCCAGGAAUCGAUCCUGAU |
| | | GCUGCCGGAAGAAGUCGAAGAAGUCAUCGGAAACAA |
| | | GCCGGAAUCGGACAUCCUGGUCCACACAGCAUACGA |
| | | CGAAUCGACAGACGAAAACGUCAUGCUGCUGACAUC |
| | | GGACGCACCGGAAUACAAGCCGUGGGCACUGGUCAU |
| | | CCAGGACUCGAACGGAGAAAACAAGAUCAAGAUGCU |
| | | GUGA |
| | 820- | Not used |
| | 899, | |
| | 903- | |
| | 971 | |
| mRNA | 972 | GGGAAGCUCAGAAUAAACGCUCAACUUUGGCCGGAU |
| encoding | | CUGCCACCAUGGAGGCCUCCCCCGCCUCCGGCCCCCG |
| BC22n | | GCACCUGAUGGACCCCCACAUCUUCACCUCCAACUUC |
| | | AACAACGGCAUCGGCCGGCACAAGACCUACCUGUGC |
| | | UACGAGGUGGAGCGGCUGGACAACGGCACCUCCGUG |
| | | AAGAUGGACCAGCACCGGGGCUUCCUGCACAACCAG |
| | | GCCAAGAACCUGCUGUGCGGCUUCUACGGCCGGCAC |
| | | GCCGAGCUGCGGUUCCUGGACCUGGUGCCCUCCCUG |
| | | CAGCUGGACCCCGCCCAGAUCUACCGGGUGACCUGG |
| | | UUCAUCUCCUGGUCCCCCUGCUUCUCCUGGGGCUGC |
| | | GCCGGCGAGGUGCGGGCCUUCCUGCAGGAGAACACC |
| | | CACGUGCGGCUGCGGAUCUUCGCCGCCCGGAUCUAC |
| | | GACUACGACCCCCUGUACAAGGAGGCCCUGCAGAUG |
| | | CUGCGGGACGCCGGCGCCCAGGUGUCCAUCAUGACC |
| | | UACGACGAGUUCAAGCACUGCUGGGACACCUUCGUG |
| | | GACCACCAGGGCUGCCCCUUCCAGCCCUGGGACGGCC |
| | | UGGACGAGCACUCCCAGGCCCUGUCCGGCCGGCUGC |
| | | GGGCCAUCCUGCAGAACCAGGGCAACUCCGGCUCCG |
| | | AGACCCCCGGCACCUCCGAGUCCGCCACCCCCGAGUC |
| | | CGACAAGAAGUACUCCAUCGGCCUGGCCAUCGGCAC |
| | | CAACUCCGUGGGCUGGGCCGUGAUCACCGACGAGUA |
| | | CAAGGUGCCCUCCAAGAAGUUCAAGGUGCUGGGCAA |
| | | CACCGACCGGCACUCCAUCAAGAAGAACCUGAUCGG |
| | | CGCCCUGCUGUUCGACUCCGGCGAGACCGCCGAGGC |
| | | CACCCGGCUGAAGCGGACCGCCCGGCGGCGGUACAC |
| | | CCGGCGGAAGAACCGGAUCUGCUACCUGCAGGAGAU |
| | | CUUCUCCAACGAGAUGGCCAAGGUGGACGACUCCUU |
| | | CUUCCACCGGCUGGAGGAGUCCUUCCUGGUGGAGGA |
| | | GGACAAGAAGCACGAGCGGCACCCCAUCUUCGGCAA |
| | | CAUCGUGGACGAGGUGGCCUACCACGAGAAGUACCC |
| | | CACCAUCUACCACCUGCGGAAGAAGCUGGUGGACUC |
| | | CACCGACAAGGCCGACCUGCGGCUGAUCUACCUGGC |
| | | CCUGGCCCACAUGAUCAAGUUCCGGGGCCACUUCCU |
| | | GAUCGAGGGCGACCUGAACCCCGACAACUCCGACGU |
| | | GGACAAGCUGUUCAUCCAGCUGGUGCAGACCUACAA |
| | | CCAGCUGUUCGAGGAGAACCCCAUCAACGCCUCCGG |
| | | CGUGGACGCCAAGGCCAUCCUGUCCGCCCGGCUGUC |
| | | CAAGUCCCGGCGGCUGGAGAACCUGAUCGCCCAGCU |
| | | GCCCGGCGAGAAGAAGAACGGCCUGUUCGGCAACCU |
| | | GAUCGCCCUGUCCCUGGGCCUGACCCCCAACUUCAA |
| | | GUCCAACUUCGACCUGGCCGAGGACGCCAAGCUGCA |
| | | GCUGUCCAAGGACACCUACGACGACGACCUGGACAA |
| | | CCUGCUGGCCCAGAUCGGCGACCAGUACGCCGACCU |
| | | GUUCCUGGCCGCCAAGAACCUGUCCGACGCCAUCCU |
| | | GCUGUCCGACAUCCUGCGGGUGAACACCGAGAUCAC |
| | | CAAGGCCCCCCUGUCCGCCUCCAUGAUCAAGCGGUA |
| | | CGACGAGCACCACCAGGACCUGACCCUGCUGAAGGC |
| | | CCUGGUGCGGCAGCAGCUGCCCGAGAAGUACAAGGA |
| | | GAUCUUCUUCGACCAGUCCAAGAACGGCUACGCCGG |
| | | CUACAUCGACGGCGGCGCCUCCCAGGAGGAGUUCUA |
| | | CAAGUUCAUCAAGCCCAUCCUGGAGAAGAUGGACGG |
| | | CACCGAGGAGCUGCUGGUGAAGCUGAACCGGGAGGA |
| | | CCUGCUGCGGAAGCAGCGGACCUUCGACAACGGCUC |
| | | CAUCCCCCACCAGAUCCACCUGGGCGAGCUGCACGCC |
| | | AUCCUGCGGCGGCAGGAGGACUUCUACCCCUUCCUG |
| | | AAGGACAACCGGGAGAAGAUCGAGAAGAUCCUGACC |
| | | UUCCGGAUCCCCUACUACGUGGGCCCCCUGGCCCGG |
| | | GGCAACUCCCGGUUCGCCUGGAUGACCCGGAAGUCC |
| | | GAGGAGACCAUCACCCCCUGGAACUUCGAGGAGGUG |
| | | GUGGACAAGGGCGCCUCCGCCCAGUCCUUCAUCGAG |
| | | CGGAUGACCAACUUCGACAAGAACCUGCCCAACGAG |
| | | AAGGUGCUGCCCAAGCACUCCCUGCUGUACGAGUAC |
| | | UUCACCGUGUACAACGAGCUGACCAAGGUGAAGUAC |
| | | GUGACCGAGGGCAUGCGGAAGCCCGCCUUCCUGUCC |
| | | GGCGAGCAGAAGAAGGCCAUCGUGGACCUGCUGUUC |
| | | AAGACCAACCGGAAGGUGACCGUGAAGCAGCUGAAG |
| | | GAGGACUACUUCAAGAAGAUCGAGUGCUUCGACUCC |
| | | GUGGAGAUCUCCGGCGUGGAGGACCGGUUCAACGCC |
| | | UCCCUGGGCACCUACCACGACCUGCUGAAGAUCAUC |
| | | AAGGACAAGGACUUCCUGGACAACGAGGAGAACGAG |
| | | GACAUCCUGGAGGACAUCGUGCUGACCCUGACCCUG |
| | | UUCGAGGACCGGGAGAUGAUCGAGGAGCGGCUGAAG |
| | | ACCUACGCCCACCUGUUCGACGACAAGGUGAUGAAG |
| | | CAGCUGAAGCGGCGGCGGUACACCGGCUGGGGCCGG |
| | | CUGUCCCGGAAGCUGAUCAACGGCAUCCGGGACAAG |
| | | CAGUCCGGCAAGACCAUCCUGGACUUCCUGAAGUCC |
| | | GACGGCUUCGCCAACCGGAACUUCAUGCAGCUGAUC |
| | | CACGACGACUCCCUGACCUUCAAGGAGGACAUCCAG |
| | | AAGGCCCAGGUGUCCGGCCAGGGCGACUCCCUGCAC |
| | | GAGCACAUCGCCAACCUGGCCGGCUCCCCCGCCAUCA |
| | | AGAAGGGCAUCCUGCAGACCGUGAAGGUGGUGGACG |
| | | AGCUGGUGAAGGUGAUGGGCCGGCACAAGCCCGAGA |
| | | ACAUCGUGAUCGAGAUGGCCCGGGAGAACCAGACCA |
| | | CCCAGAAGGGCCAGAAGAACUCCCGGGAGCGGAUGA |
| | | AGCGGAUCGAGGAGGGCAUCAAGGAGCUGGGCUCCC |
| | | AGAUCCUGAAGGAGCACCCCGUGGAGAACACCCAGC |
| | | UGCAGAACGAGAAGCUGUACCUGUACUACCUGCAGA |
| | | ACGGCCGGGACAUGUACGUGGACCAGGAGCUGGACA |
| | | UCAACCGGCUGUCCGACUACGACGUGGACCACAUCG |
| | | UGCCCCAGUCCUUCCUGAAGGACGACUCCAUCGACA |
| | | ACAAGGUGCUGACCCGGUCCGACAAGAACCGGGGCA |
| | | AGUCCGACAACGUGCCCUCCGAGGAGGUGGUGAAGA |
| | | AGAUGAAGAACUACUGGCGGCAGCUGCUGAACGCCA |
| | | AGCUGAUCACCCAGCGGAAGUUCGACAACCUGACCA |
| | | AGGCCGAGCGGGGCGGCCUGUCCGAGCUGGACAAGG |
| | | CCGGCUUCAUCAAGCGGCAGCUGGUGGAGACCCGGC |
| | | AGAUCACCAAGCACGUGGCCCAGAUCCUGGACUCCC |
| | | GGAUGAACACCAAGUACGACGAGAACGACAAGCUGA |
| | | UCCGGGAGGUGAAGGUGAUCACCCUGAAGUCCAAGC |
| | | UGGUGUCCGACUUCCGGAAGGACUUCCAGUUCUACA |
| | | AGGUGCGGGAGAUCAACAACUACCACCACGCCCACG |
| | | ACGCCUACCUGAACGCCGUGGUGGGCACCGCCCUGA |
| | | UCAAGAAGUACCCCAAGCUGGAGUCCGAGUUCGUGU |
| | | ACGGCGACUACAAGGUGUACGACGUGCGGAAGAUGA |
| | | UCGCCAAGUCCGAGCAGGAGAUCGGCAAGGCCACCG |
| | | CCAAGUACUUCUUCUACUCCAACAUCAUGAACUUCU |
| | | UCAAGACCGAGAUCACCCUGGCCAACGGCGAGAUCC |
| | | GGAAGCGGCCCCUGAUCGAGACCAACGGCGAGACCG |
| | | GCGAGAUCGUGUGGGACAAGGGCCGGGACUUCGCCA |
| | | CCGUGCGGAAGGUGCUGUCCAUGCCCCAGGUGAACA |
| | | UCGUGAAGAAGACCGAGGUGCAGACCGGCGGCUUCU |
| | | CCAAGGAGUCCAUCCUGCCCAAGCGGAACUCCGACA |
| | | AGCUGAUCGCCCGGAAGAAGGACUGGGACCCCAAGA |
| | | AGUACGGCGGCUUCGACUCCCCCACCGUGGCCUACU |
| | | CCGUGCUGGUGGUGGCCAAGGUGGAGAAGGGCAAGU |
| | | CCAAGAAGCUGAAGUCCGUGAAGGAGCUGCUGGGCA |
| | | UCACCAUCAUGGAGCGGUCCUCCUUCGAGAAGAACC |
| | | CCAUCGACUUCCUGGAGGCCAAGGGCUACAAGGAGG |
| | | UGAAGAAGGACCUGAUCAUCAAGCUGCCCAAGUACU |
| | | CCCUGUUCGAGCUGGAGAACGGCCGGAAGCGGAUGC |
| | | UGGCCUCCGCCGGCGAGCUGCAGAAGGGCAACGAGC |
| | | UGGCCCUGCCCUCCAAGUACGUGAACUUCCUGUACC |
| | | UGGCCUCCCACUACGAGAAGCUGAAGGGCUCCCCCG |
| | | AGGACAACGAGCAGAAGCAGCUGUUCGUGGAGCAGC |
| | | ACAAGCACUACCUGGACGAGAUCAUCGAGCAGAUCU |
| | | CCGAGUUCUCCAAGCGGGUGAUCCUGGCCGACGCCA |
| | | ACCUGGACAAGGUGCUGUCCGCCUACAACAAGCACC |
| | | GGGACAAGCCCAUCCGGGAGCAGGCCGAGAACAUCA |
| | | UCCACCUGUUCACCCUGACCAACCUGGGCGCCCCCGC |
| | | CGCCUUCAAGUACUUCGACACCACCAUCGACCGGAA |
| | | GCGGUACACCUCCACCAAGGAGGUGCUGGACGCCAC |
| | | CCUGAUCCACCAGUCCAUCACCGGCCUGUACGAGAC |
| | | CCGGAUCGACCUGUCCCAGCUGGGCGGCGACGGCGG |
| | | CGGCUCCCCCAAGAAGAAGCGGAAGGUGUGACUAGC |
| | | ACCAGCCUCAAGAACACCCGAAUGGAGUCUCUAAGC |
| | | UACAUAAUACCAACUUACACUUUACAAAAUGUUGUC |
| | | CCCCAAAAUGUAGCCAUUCGUAUCUGCUCCUAAUAA |
| | | AAAGAAAGUUUCUUCACAUUCUCUCGAGAAAAAAAA |
| | | AAAAUGGAAAAAAAAAAAACGGAAAAAAAAAAAAG |
| | | GUAAAAAAAAAAAAUAUAAAAAAAAAAAACAUAAA |
| | | AAAAAAAAACGAAAAAAAAAAAACGUAAAAAAAAA |
| | | AAACUCAAAAAAAAAAAAGAUAAAAAAAAAAAACCU |
| | | AAAAAAAAAAAAUGUAAAAAAAAAAAAGGGAAAAA |
| | | AAAAAAACGCAAAAAAAAAAAACACAAAAAAAAAAA |
| | | AUGCAAAAAAAAAAAAUCGAAAAAAAAAAAAUCUA |
| | | AAAAAAAAAAACGAAAAAAAAAAAACCCAAAAAAAA |
| | | AAAAGACAAAAAAAAAAAAUAGAAAAAAAAAAAAG |
| | | UUAAAAAAAAAAAACUGAAAAAAAAAAAAUUUAAA |
| | | AAAAAAAAAUCUAG |
| mRNA | 973 | GGGAAGCUCAGAAUAAACGCUCAACUUUGGCCGGAU |
| encoding | | CUGCCACCAUGGAGGCCUCCCCCGCCUCCGGCCCCCG |
| BC22n with | | GCACCUGAUGGACCCCCACAUCUUCACCUCCAACUUC |
| HiBit tag | | AACAACGGCAUCGGCCGGCACAAGACCUACCUGUGC |
| | | UACGAGGUGGAGCGGCUGGACAACGGCACCUCCGUG |
| | | AAGAUGGACCAGCACCGGGGCUUCCUGCACAACCAG |
| | | GCCAAGAACCUGCUGUGCGGCUUCUACGGCCGGCAC |
| | | GCCGAGCUGCGGUUCCUGGACCUGGUGCCCUCCCUG |
| | | CAGCUGGACCCCGCCCAGAUCUACCGGGUGACCUGG |
| | | UUCAUCUCCUGGUCCCCCUGCUUCUCCUGGGGCUGC |
| | | GCCGGCGAGGUGCGGGCCUUCCUGCAGGAGAACACC |
| | | CACGUGCGGCUGCGGAUCUUCGCCGCCCGGAUCUAC |
| | | GACUACGACCCCCUGUACAAGGAGGCCCUGCAGAUG |
| | | CUGCGGGACGCCGGCGCCCAGGUGUCCAUCAUGACC |
| | | UACGACGAGUUCAAGCACUGCUGGGACACCUUCGUG |
| | | GACCACCAGGGCUGCCCCUUCCAGCCCUGGGACGGCC |
| | | UGGACGAGCACUCCCAGGCCCUGUCCGGCCGGCUGC |
| | | GGGCCAUCCUGCAGAACCAGGGCAACUCCGGCUCCG |
| | | AGACCCCCGGCACCUCCGAGUCCGCCACCCCCGAGUC |
| | | CGACAAGAAGUACUCCAUCGGCCUGGCCAUCGGCAC |
| | | CAACUCCGUGGGCUGGGCCGUGAUCACCGACGAGUA |
| | | CAAGGUGCCCUCCAAGAAGUUCAAGGUGCUGGGCAA |
| | | CACCGACCGGCACUCCAUCAAGAAGAACCUGAUCGG |
| | | CGCCCUGCUGUUCGACUCCGGCGAGACCGCCGAGGC |
| | | CACCCGGCUGAAGCGGACCGCCCGGCGGCGGUACAC |
| | | CCGGCGGAAGAACCGGAUCUGCUACCUGCAGGAGAU |
| | | CUUCUCCAACGAGAUGGCCAAGGUGGACGACUCCUU |
| | | CUUCCACCGGCUGGAGGAGUCCUUCCUGGUGGAGGA |
| | | GGACAAGAAGCACGAGCGGCACCCCAUCUUCGGCAA |
| | | CAUCGUGGACGAGGUGGCCUACCACGAGAAGUACCC |
| | | CACCAUCUACCACCUGCGGAAGAAGCUGGUGGACUC |
| | | CACCGACAAGGCCGACCUGCGGCUGAUCUACCUGGC |
| | | CCUGGCCCACAUGAUCAAGUUCCGGGGCCACUUCCU |
| | | GAUCGAGGGCGACCUGAACCCCGACAACUCCGACGU |
| | | GGACAAGCUGUUCAUCCAGCUGGUGCAGACCUACAA |
| | | CCAGCUGUUCGAGGAGAACCCCAUCAACGCCUCCGG |
| | | CGUGGACGCCAAGGCCAUCCUGUCCGCCCGGCUGUC |
| | | CAAGUCCCGGCGGCUGGAGAACCUGAUCGCCCAGCU |
| | | GCCCGGCGAGAAGAAGAACGGCCUGUUCGGCAACCU |
| | | GAUCGCCCUGUCCCUGGGCCUGACCCCCAACUUCAA |
| | | GUCCAACUUCGACCUGGCCGAGGACGCCAAGCUGCA |
| | | GCUGUCCAAGGACACCUACGACGACGACCUGGACAA |
| | | CCUGCUGGCCCAGAUCGGCGACCAGUACGCCGACCU |
| | | GUUCCUGGCCGCCAAGAACCUGUCCGACGCCAUCCU |
| | | GCUGUCCGACAUCCUGCGGGUGAACACCGAGAUCAC |
| | | CAAGGCCCCCCUGUCCGCCUCCAUGAUCAAGCGGUA |
| | | CGACGAGCACCACCAGGACCUGACCCUGCUGAAGGC |
| | | CCUGGUGCGGCAGCAGCUGCCCGAGAAGUACAAGGA |
| | | GAUCUUCUUCGACCAGUCCAAGAACGGCUACGCCGG |
| | | CUACAUCGACGGCGGCGCCUCCCAGGAGGAGUUCUA |
| | | CAAGUUCAUCAAGCCCAUCCUGGAGAAGAUGGACGG |
| | | CACCGAGGAGCUGCUGGUGAAGCUGAACCGGGAGGA |
| | | CCUGCUGCGGAAGCAGCGGACCUUCGACAACGGCUC |
| | | CAUCCCCCACCAGAUCCACCUGGGCGAGCUGCACGCC |
| | | AUCCUGCGGCGGCAGGAGGACUUCUACCCCUUCCUG |
| | | AAGGACAACCGGGAGAAGAUCGAGAAGAUCCUGACC |
| | | UUCCGGAUCCCCUACUACGUGGGCCCCCUGGCCCGG |
| | | GGCAACUCCCGGUUCGCCUGGAUGACCCGGAAGUCC |
| | | GAGGAGACCAUCACCCCCUGGAACUUCGAGGAGGUG |
| | | GUGGACAAGGGCGCCUCCGCCCAGUCCUUCAUCGAG |
| | | CGGAUGACCAACUUCGACAAGAACCUGCCCAACGAG |
| | | AAGGUGCUGCCCAAGCACUCCCUGCUGUACGAGUAC |
| | | UUCACCGUGUACAACGAGCUGACCAAGGUGAAGUAC |
| | | GUGACCGAGGGCAUGCGGAAGCCCGCCUUCCUGUCC |
| | | GGCGAGCAGAAGAAGGCCAUCGUGGACCUGCUGUUC |
| | | AAGACCAACCGGAAGGUGACCGUGAAGCAGCUGAAG |
| | | GAGGACUACUUCAAGAAGAUCGAGUGCUUCGACUCC |
| | | GUGGAGAUCUCCGGCGUGGAGGACCGGUUCAACGCC |
| | | UCCCUGGGCACCUACCACGACCUGCUGAAGAUCAUC |
| | | AAGGACAAGGACUUCCUGGACAACGAGGAGAACGAG |
| | | GACAUCCUGGAGGACAUCGUGCUGACCCUGACCCUG |
| | | UUCGAGGACCGGGAGAUGAUCGAGGAGCGGCUGAAG |
| | | ACCUACGCCCACCUGUUCGACGACAAGGUGAUGAAG |
| | | CAGCUGAAGCGGCGGCGGUACACCGGCUGGGGCCGG |
| | | CUGUCCCGGAAGCUGAUCAACGGCAUCCGGGACAAG |
| | | CAGUCCGGCAAGACCAUCCUGGACUUCCUGAAGUCC |
| | | GACGGCUUCGCCAACCGGAACUUCAUGCAGCUGAUC |
| | | CACGACGACUCCCUGACCUUCAAGGAGGACAUCCAG |
| | | AAGGCCCAGGUGUCCGGCCAGGGCGACUCCCUGCAC |
| | | GAGCACAUCGCCAACCUGGCCGGCUCCCCCGCCAUCA |
| | | AGAAGGGCAUCCUGCAGACCGUGAAGGUGGUGGACG |
| | | AGCUGGUGAAGGUGAUGGGCCGGCACAAGCCCGAGA |
| | | ACAUCGUGAUCGAGAUGGCCCGGGAGAACCAGACCA |
| | | CCCAGAAGGGCCAGAAGAACUCCCGGGAGCGGAUGA |
| | | AGCGGAUCGAGGAGGGCAUCAAGGAGCUGGGCUCCC |
| | | AGAUCCUGAAGGAGCACCCCGUGGAGAACACCCAGC |
| | | UGCAGAACGAGAAGCUGUACCUGUACUACCUGCAGA |
| | | ACGGCCGGGACAUGUACGUGGACCAGGAGCUGGACA |
| | | UCAACCGGCUGUCCGACUACGACGUGGACCACAUCG |
| | | UGCCCCAGUCCUUCCUGAAGGACGACUCCAUCGACA |
| | | ACAAGGUGCUGACCCGGUCCGACAAGAACCGGGGCA |
| | | AGUCCGACAACGUGCCCUCCGAGGAGGUGGUGAAGA |
| | | AGAUGAAGAACUACUGGCGGCAGCUGCUGAACGCCA |
| | | AGCUGAUCACCCAGCGGAAGUUCGACAACCUGACCA |
| | | AGGCCGAGCGGGGCGGCCUGUCCGAGCUGGACAAGG |
| | | CCGGCUUCAUCAAGCGGCAGCUGGUGGAGACCCGGC |
| | | AGAUCACCAAGCACGUGGCCCAGAUCCUGGACUCCC |
| | | GGAUGAACACCAAGUACGACGAGAACGACAAGCUGA |
| | | UCCGGGAGGUGAAGGUGAUCACCCUGAAGUCCAAGC |
| | | UGGUGUCCGACUUCCGGAAGGACUUCCAGUUCUACA |
| | | AGGUGCGGGAGAUCAACAACUACCACCACGCCCACG |
| | | ACGCCUACCUGAACGCCGUGGUGGGCACCGCCCUGA |
| | | UCAAGAAGUACCCCAAGCUGGAGUCCGAGUUCGUGU |
| | | ACGGCGACUACAAGGUGUACGACGUGCGGAAGAUGA |
| | | UCGCCAAGUCCGAGCAGGAGAUCGGCAAGGCCACCG |
| | | CCAAGUACUUCUUCUACUCCAACAUCAUGAACUUCU |
| | | UCAAGACCGAGAUCACCCUGGCCAACGGCGAGAUCC |
| | | GGAAGCGGCCCCUGAUCGAGACCAACGGCGAGACCG |
| | | GCGAGAUCGUGUGGGACAAGGGCCGGGACUUCGCCA |
| | | CCGUGCGGAAGGUGCUGUCCAUGCCCCAGGUGAACA |
| | | UCGUGAAGAAGACCGAGGUGCAGACCGGCGGCUUCU |
| | | CCAAGGAGUCCAUCCUGCCCAAGCGGAACUCCGACA |
| | | AGCUGAUCGCCCGGAAGAAGGACUGGGACCCCAAGA |
| | | AGUACGGCGGCUUCGACUCCCCCACCGUGGCCUACU |
| | | CCGUGCUGGUGGUGGCCAAGGUGGAGAAGGGCAAGU |
| | | CCAAGAAGCUGAAGUCCGUGAAGGAGCUGCUGGGCA |
| | | UCACCAUCAUGGAGCGGUCCUCCUUCGAGAAGAACC |
| | | CCAUCGACUUCCUGGAGGCCAAGGGCUACAAGGAGG |
| | | UGAAGAAGGACCUGAUCAUCAAGCUGCCCAAGUACU |
| | | CCCUGUUCGAGCUGGAGAACGGCCGGAAGCGGAUGC |
| | | UGGCCUCCGCCGGCGAGCUGCAGAAGGGCAACGAGC |
| | | UGGCCCUGCCCUCCAAGUACGUGAACUUCCUGUACC |
| | | UGGCCUCCCACUACGAGAAGCUGAAGGGCUCCCCCG |
| | | AGGACAACGAGCAGAAGCAGCUGUUCGUGGAGCAGC |
| | | ACAAGCACUACCUGGACGAGAUCAUCGAGCAGAUCU |
| | | CCGAGUUCUCCAAGCGGGUGAUCCUGGCCGACGCCA |
| | | ACCUGGACAAGGUGCUGUCCGCCUACAACAAGCACC |
| | | GGGACAAGCCCAUCCGGGAGCAGGCCGAGAACAUCA |
| | | UCCACCUGUUCACCCUGACCAACCUGGGCGCCCCCGC |
| | | CGCCUUCAAGUACUUCGACACCACCAUCGACCGGAA |
| | | GCGGUACACCUCCACCAAGGAGGUGCUGGACGCCAC |
| | | CCUGAUCCACCAGUCCAUCACCGGCCUGUACGAGAC |
| | | CCGGAUCGACCUGUCCCAGCUGGGCGGCGACGGCGG |
| | | CGGCUCCCCCAAGAAGAAGCGGAAGGUGUCCGAGUC |
| | | CGCCACCCCCGAGUCCGUGUCCGGCUGGCGGCUGUU |
| | | CAAGAAGAUCUCCUGACUAGCACCAGCCUCAAGAAC |
| | | ACCCGAAUGGAGUCUCUAAGCUACAUAAUACCAACU |
| | | UACACUUUACAAAAUGUUGUCCCCCAAAAUGUAGCC |
| | | AUUCGUAUCUGCUCCUAAUAAAAAGAAAGUUUCUUC |
| | | ACAUUCUCUCGAGAAAAAAAAAAAAUGGAAAAAAAA |
| | | AAAACGGAAAAAAAAAAAAGGUAAAAAAAAAAAAU |
| | | AUAAAAAAAAAAAACAUAAAAAAAAAAAACGAAAA |
| | | AAAAAAAACGUAAAAAAAAAAAACUCAAAAAAAAA |
| | | AAAGAUAAAAAAAAAAAACCUAAAAAAAAAAAAUG |
| | | UAAAAAAAAAAAAGGGAAAAAAAAAAAACGCAAAA |
| | | AAAAAAAACACAAAAAAAAAAAAUGCAAAAAAAAA |
| | | AAAUCGAAAAAAAAAAAAUCUAAAAAAAAAAAACG |
| | | AAAAAAAAAAAACCCAAAAAAAAAAAAGACAAAAAA |
| | | AAAAAAUAGAAAAAAAAAAAAGUUAAAAAAAAAAA |
| | | ACUGAAAAAAAAAAAAUUUAAAAAAAAAAAAUCUA |
| | | G |
| | 974 | Not used |
| mRNA | 975 | GGGAGACCCAAGCUGGCUAGCUCCCGCAGUCGGCGU |
| encoding UGI | | CCAGCGGCUCUGCUUGUUCGUGUGUGUGUCGUUGCA |
| | | GGCCUUAUUCGGAUCCGCCACCAUGGGACCGAAGAA |
| | | GAAGAGAAAGGUCGGAGGAGGAAGCACAAACCUGUC |
| | | GGACAUCAUCGAAAAGGAAACAGGAAAGCAGCUGGU |
| | | CAUCCAGGAAUCGAUCCUGAUGCUGCCGGAAGAAGU |
| | | CGAAGAAGUCAUCGGAAACAAGCCGGAAUCGGACAU |
| | | CCUGGUCCACACAGCAUACGACGAAUCGACAGACGA |
| | | AAACGUCAUGCUGCUGACAUCGGACGCACCGGAAUA |
| | | CAAGCCGUGGGCACUGGUCAUCCAGGACUCGAACGG |
| | | AGAAAACAAGAUCAAGAUGCUGUGAUAGUCUAGACA |
| | | UCACAUUUAAAAGCAUCUCAGCCUACCAUGAGAAUA |
| | | AGAGAAAGAAAAUGAAGAUCAAUAGCUUAUUCAUCU |
| | | CUUUUUCUUUUUCGUUGGUGUAAAGCCAACACCCUG |
| | | UCUAAAAAACAUAAAUUUCUUUAAUCAUUUUGCCUC |
| | | UUUUCUCUGUGCUUCAAUUAAUAAAAAAUGGAAAGA |
| | | ACCUCGAGUCUAG |
| | 976- | Not used |
| | 999 | |
| Lentiviral | 1000 | gcgatcgcagtaatcaattacggggtcattagttcatagcccatatatggagttccgcgttac |
| genome | | ataacttacggtaaatggcccgcctggctgaccgcccaacgacccccgcccattgacgtc |
| encoding | | aataatgacgtatgttcccatagtaacgccaatagggactttccattgacgtcaatgggtgg |
| HLA-E | | agtatttacggtaaactgcccacttggcagtacatcaagtgtatcatatgccaagtacgccc |
| expressed by | | cctattgacgtcaatgacggtaaatggcccgcctggcattatgcccagtacatgaccttatg |
| an EF1a | | ggactttcctacttggcagtacatctacgtattagtcatcgctattaccatgGTGATGC |
| promoter | | GGTTTTGGCAGTACATCAATGGGCGTGGATAGCGGTT |
| | | TGACTCACGGGGATTTCCAAGTCTCCACCCCATTGACG |
| | | TCAATGGGAGTTTGTTTTGGCACCAAAATCAACGGGA |
| | | CTTTCCAAAATGTCGTAACAACTCCGCCCCATTGACGC |
| | | AAATGGGCGGTAGGCGTGTACGGTGGGAGGTCTATAT |
| | | AAGCAGAGCTcgtttagtgaaccggggtctctctggttagaccagatctgagcct |
| | | gggagctctctggctaactagggaacccactgcttaagcctcaataaagcttgccttgagt |
| | | gcttcaagtagtgtgtgcccgtctgttgtgtgactctggtaactagagatccctcagacccttt |
| | | tagtcagtgtggaaaatctctagcagtggcgcccgaacagggacctgaaagcgaaaggg |
| | | aaaccagagctctctcgacgcaggactcggcttgctgaagcgcgcacggcaagaggcg |
| | | aggggcggcgactggtgagtacgccaaaaattttgactagcggaggctagaaggagag |
| | | agatgggtgcgagagcgtcagtattaagcgggggagaattagatcgcgatgggaaaaaa |
| | | ttcggttaaggccagggggaaagaaaaaatataaattaaaacatatagtatgggcaagca |
| | | gggagctagaacgattcgcagttaatcctggcctgttagaaacatcagaaggctgtagac |
| | | aaatactgggacagctacaaccatcccttcagacaggatcagaagaacttagatcattatat |
| | | aatacagtagcaaccctctattgtgtgcatcaaaggatagagataaaagacaccaaggaa |
| | | gctttagacaagatagaggaagagcaaaacaaaagtaagaccaccgcacagcaagcgg |
| | | ccgctgatcttcagacctggaggaggagatatgagggacaattggagaagtgaattatata |
| | | aatataaagtagtaaaaattgaaccattaggagtagcacccaccaaggcaaagagaaga |
| | | gtggtgcagagagaaaaaagagcagtgggaataggagctttgttccttgggttcttggga |
| | | gcagcaggaagcactatgggcgcagcctcaatgacgctgacggtacaggccagacaat |
| | | tattgtctggtatagtgcagcagcagaacaatttgctgagggctattgaggcgcaacagca |
| | | tctgttgcaactcacagtctggggcatcaagcagctccaggcaagaatcctggctgtgga |
| | | aagatacctaaaggatcaacagctcctggggatttggggttgctctggaaaactcatttgca |
| | | ccactgctgtgccttggaatgctagttggagtaataaatctctggaacagatttggaatcaca |
| | | cgacctggatggagtgggacagagaaattaacaattacacaagcttaatacactccttaatt |
| | | gaagaatcgcaaaaccagcaagaaaagaatgaacaagaattattggaattagataaatgg |
| | | gcaagtttgtggaattggtttaacataacaaattggctgtggtatataaaattattcataatgat |
| | | agtaggaggcttggtaggtttaagaatagtttttgctgtactttctatagtgaatagagttagg |
| | | cagggatattcaccattatcgtttcagacccacctcccaaccccgaggggacccgacagg |
| | | cccgaaggaatagaagaagaaggtggagagagagacagagacagatccattcgattag |
| | | tgaacggatctcgacggtatcggttaacttttaaaagaaaaggggggattggggggtaca |
| | | gtgcaggggaaagaatagtagacataatagcaacagacatacaaactaaagaattacaaa |
| | | aacaaattacaaaaattcaaaattttggctcccgatcgttgcgttacacacacaattactgct |
| | | gatcgagtgtagccttcccacagtccccgagaagttggggggaggggtcggcaattgaa |
| | | ccggtgcctagagaaggtggcgcggggtaaactgggaaagtgatgtcgtgtactggctc |
| | | cgcctttttcccgaggggggggagaaccgtatataagtgcagtagtcgccgtgaacgttc |
| | | tttttcgcaacgggtttgccgccagaacacaggtaagtgccgtgtgtggttcccgcgggcc |
| | | tggcctctttacgggttatggcccttgcgtgccttgaattacttccacgcccctggctgcagt |
| | | acgtgattcttgatcccgagcttcgggttggaagtggggggagagttcgaggccttgcgc |
| | | ttaaggagccccttcgcctcgtgcttgagttgaggcctggcttgggcgctggggccgccg |
| | | cgtgcgaatctggtggcaccttcgcgcctgtctcgctgctttcgataagtctctagccattta |
| | | aaatttttgatgacctgctgcgacgctttttttctggcaagatagtcttgtaaatgcgggccaa |
| | | gatgtgcacactggtatttcggtttttggggccgcgggcggcgacggggcccgtgcgtcc |
| | | cagcgcacatgttcggcgaggcggggcctgcgagcgcggccaccgagaatcggacgg |
| | | gggtagtctcaagctggccggcctgctctggtgcctggcctcgcgccgccgtgtatcgcc |
| | | ccgccctgggcggcaaggctggcccggtcggcaccagttgcgtgagcggaaagatgg |
| | | ccgcttcccggccctgctgcagggagctcaaaatggaggacgcggcgctcgggagagc |
| | | ggggggtgagtcacccacacaaaggaaaagggcctttccgtcctcagccgtcgcttcat |
| | | gtgactccacggagtaccgggcgccgtccaggcacctcgattagttctcgagcttttggag |
| | | tacgtcgtctttaggttggggggaggggttttatgcgatggagtttccccacactgagtggg |
| | | tggagactgaagttaggccagcttggcacttgatgtaattctccttggaatttgccctttttga |
| | | gtttggatcttggttcattctcaagcctcagacagtggttcaaagtttttttCTTCCATT |
| | | TCAGGTGTCGTGAtctagacgccaccATGTCTCGCTCCGTGGC |
| | | CTTAGCTGTGCTCGCGCTACTCTCTCTTTCTGGCCTAG |
| | | AGGCTGTTATGGCTCCGCGGACTTTAATTTTAGGTGGT |
| | | GGCGGATCCGGTGGAGGCGGTTCTGGTGGAGGCGGCT |
| | | CCATCCAGCGTACGCCAAAGATTCAGGTTTACTCACGT |
| | | CATCCAGCAGAGAATGGAAAGTCAAATTTCCTGAATT |
| | | GCTATGTGTCTGGGTTTCATCCATCCGACATTGAAGTT |
| | | GACTTACTGAAGAATGGAGAGAGAATTGAAAAAGTG |
| | | GAGCATTCAGACTTGTCTTTCAGCAAGGACTGGTCTTT |
| | | CTATCTCTTGTACTACACTGAATTCACCCCCACTGAAA |
| | | AAGATGAGTATGCCTGCCGTGTGAACCATGTGACTTT |
| | | GTCACAGCCCAAGATAGTTAAGTGGGATCGCGACATG |
| | | GGTGGTGGCGGTTCTGGTGGTGGCGGTAGTGGCGGCG |
| | | GAGGAAGCGGTGGTGGCGGTTCCGGATCTCACTCCTT |
| | | GAAGTATTTCCACACTTCCGTGTCCCGGCCCGGCCGCG |
| | | GGGAGCCCCGCTTCATCTCTGTGGGCTACGTGGACGA |
| | | CACCCAGTTCGTGCGCTTCGACAACGACGCCGCGAGT |
| | | CCGAGGATGGTGCCGCGGGCGCCGTGGATGGAGCAGG |
| | | AGGGGTCAGAGTATTGGGACCGGGAGACACGGAGCG |
| | | CCAGGGACACCGCACAGATTTTCCGAGTGAACCTGCG |
| | | GACGCTGCGCGGCTACTACAATCAGAGCGAGGCCGGG |
| | | TCTCACACCCTGCAGTGGATGCATGGCTGCGAGCTGG |
| | | GGCCCGACAGGCGCTTCCTCCGCGGGTATGAACAGTT |
| | | CGCCTACGACGGCAAGGATTATCTCACCCTGAATGAG |
| | | GACCTGCGCTCCTGGACCGCGGTGGACACGGCGGCTC |
| | | AGATCTCCGAGCAAAAGTCAAATGATGCCTCTGAGGC |
| | | GGAGCACCAGAGAGCCTACCTGGAAGACACATGCGTG |
| | | GAGTGGCTCCACAAATACCTGGAGAAGGGGAAGGAG |
| | | ACGCTGCTTCACCTGGAGCCCCCAAAGACACACGTGA |
| | | CTCACCACCCCATCTCTGACCATGAGGCCACCCTGAG |
| | | GTGCTGGGCTCTGGGCTTCTACCCTGCGGAGATCACAC |
| | | TGACCTGGCAGCAGGATGGGGAGGGCCATACCCAGGA |
| | | CACGGAGCTCGTGGAGACCAGGCCTGCTGGGGATGGA |
| | | ACCTTCCAGAAGTGGGCAGCTGTGGTGGTGCCTTCTG |
| | | GAGAGGAGCAGAGATACACGTGCCATGTGCAGCATGA |
| | | GGGGCTACCCGAGCCCGTCACCCTGAGATGGAAGCCG |
| | | GCTTCCCAGCCCACCATCCCCATCGTGGGCATCATTGC |
| | | TGGCCTGGTTCTCCTTGGATCTGTGGTCTCTGGAGCTG |
| | | TGGTTGCTGCTGTGATATGGAGGAAGAAGAGCTCAGG |
| | | TGGAAAAGGAGGGAGCTACTATAAGGCTGAGTGGAG |
| | | CGACAGTGCCCAGGGGTCTGAGTCTCACAGCTTGTAAa |
| | | agtagaagttgtctcctcctgcactgactgactgatacaatcgatttctggatccgcaggcct |
| | | ctgctagaagttgtctcctcctgcactgactgactgatacaatcgatttctggatccgcaggc |
| | | ctctgctagcttgactgactgagtcgacAATCAACCTCTGGATTACAA |
| | | AATTTGTGAAAGATTGACTGGTATTCTTAACTATGTTG |
| | | CTCCTTTTACGCTATGTGGATACGCTGCTTTAATGCCT |
| | | TTGTATCATGCTATTGCTTCCCGTATGGCTTTCATTTTC |
| | | TCCTCCTTGTATAAATCCTGGTTGCTGTCTCTTTATGAG |
| | | GAGTTGTGGCCCGTTGTCAGGCAACGTGGCGTGGTGT |
| | | GCACTGTGTTTGCTGACGCAACCCCCACTGGTTGGGGC |
| | | ATTGCCACCACCTGTCAGCTCCTTTCCGGGACTTTCGC |
| | | TTTCCCCCTCCCTATTGCCACGGCGGAACTCATCGCCG |
| | | CCTGCCTTGCCCGCTGCTGGACAGGGGCTCGGCTGTTG |
| | | GGCACTGACAATTCCGTGGTGTTGTCGGGGAAGCTGA |
| | | CGTCCTTTCCATGGCTGCTCGCCTGTGTTGCCACCTGG |
| | | ATTCTGCGCGGGACGTCCTTCTGCTACGTCCCTTCGGC |
| | | CCTCAATCCAGCGGACCTTCCTTCCCGCGGCCTGCTGC |
| | | CGGCTCTGCGGCCTCTTCCGCGTCTTCGCCTTCGCCCT |
| | | CAGACGAGTCGGATCTCCCTTTGGGCcgcctccccgcctggaatt |
| | | cgagctcggtacctttaagaccaatgacttacaaggcagctgtagatcttagccactttttaa |
| | | aagaaaaggggggactggaagggctaattcactcccaacgaagacaagatctgctttttg |
| | | cttgtactgggtctctctggttagaccagatctgagcctgggagctctctggctaactaggg |
| | | aacccactgcttaagcctcaataaagcttgccttgagtgcttcaagtagtgtgtgcccgtctg |
| | | ttgtgtgactctggtaactagagatccctcagacccttttagtcagtgtggaaaatctctagc |
| | | agtcctggccaacgtgagcaccgtgctgacctccaaatatcgttaagctggagcctggga |
| | | gccggcctggccctccgccccccccacccccgcagcccacccctggtctttgaataaagt |
| | | ctgagtgagtggccgacagtgcccgtggagttctcgtgacctgaggtgcagggccggcg |
| | | ctagggacacgtccgtgcacgtgccgaggccccctgtgcagctgcaagggacaggcct |
| | | agccctgcaggcctaactccgcccatcccgcccctaactccgcccagttccgcccattctc |
| | | cgcctcatggctgactaattttttttatttatgcagaggccgaggccgcctcggcctctgagc |
| | | tattccagaagtagtgaggacgcttttttggaggccgaggcttttgcaaagatcgaacaag |
| | | agacaggacctgcaggttaattaaatttaaatcatgtgagcaaaaggccagcaaaaggcc |
| | | aggaaccgtaaaaaggccgcgttgctggcgtttttccataggctccgcccccctgacgag |
| | | catcacaaaaatcgacgctcaagtcagaggtggcgaaacccgacaggactataaagata |
| | | ccaggcgtttccccctggaagctccctcgtgcgctctcctgttccgaccctgccgcttaccg |
| | | gatacctgtccgcctttctcccttcgggaagcgtggcgctttctcatagctcacgctgtaggt |
| | | atctcagttcggtgtaggtcgttcgctccaagctgggctgtgtgcacgaaccccccgttca |
| | | gcccgaccgctgcgccttatccggtaactatcgtcttgagtccaacccggtaagacacga |
| | | cttatcgccactggcagcagccactggtaacaggattagcagagcgaggtatgtaggcg |
| | | gtgctacagagttcttgaagtggtggcctaactacggctacactagaagaacagtatttggt |
| | | atctgcgctctgctgaagccagttaccttcggaaaaagagttggtagctcttgatccggcaa |
| | | acaaaccaccgctggtagcggtggtttttttgtttgcaagcagcagattacgcgcagaaaa |
| | | aaaggatctcaagaagatcctttgatcttttctacggggtctgacgctcagtggaacgaaaa |
| | | ctcacgttaagggattttggtcatgagattatcaaaaaggatcttcacctagatccttttaaatt |
| | | aaaaatgaagttttaaatcaatctaaagtatatatgagtaaacttggtctgacagttaccaatg |
| | | cttaatcagtgaggcacctatctcagcgatctgtctatttcgttcatccatagttgcatttaaat |
| | | ggccggcctggcgcgccgtttaaacctagatattgatagtctgatcggtcaacgtataatcg |
| | | agtcctagcttttgcaaacatctatcaagagacaggatcagcaggaggctttcgcatgagt |
| | | attcaacatttccgtgtcgcccttattcccttttttgcggcattttgccttcctgtttttgctc |
| | | acccagaaacgctggtgaaagtaaaagatgctgaagatcagttgggtgcgcgagtgggttacat |
| | | cgaactggatctcaacagcggtaagatccttgagagttttcgccccgaagaacgctttcca |
| | | atgatgagcacttttaaagttctgctatgtggcgcggtattatcccgtattgacgccgggcaa |
| | | gagcaactcggtcgccgcatacactattctcagaatgacttggttgagtattcaccagtcac |
| | | agaaaagcatcttacggatggcatgacagtaagagaattatgcagtgctgccataaccatg |
| | | agtgataacactgcggccaacttacttctgacaacgattggaggaccgaaggagctaacc |
| | | gcttttttgcacaacatgggggatcatgtaactcgccttgatcgttgggaaccggagctgaa |
| | | tgaagccataccaaacgacgagcgtgacaccacgatgcctgtagcaatggcaacaacct |
| | | tgcgtaaactattaactggcgaactacttactctagcttcccggcaacagttgatagactgg |
| | | atggaggcggataaagttgcaggaccacttctgcgctcggcccttccggctggctggttta |
| | | ttgctgataaatctggagccggtgagcgtgggtctcgcggtatcattgcagcactggggcc |
| | | agatggtaagccctcccgtatcgtagttatctacacgacggggagtcaggcaactatggat |
| | | gaacgaaatagacagatcgctgagataggtgcctcactgattaagcattggtaaccgattc |
| | | taggtgcattggcgcagaaaaaaatgcctgatgcgacgctgcgcgtcttatactcccacat |
| | | atgccagattcagcaacggatacggcttccccaacttgcccacttccatacgtgtcctcctt |
| | | accagaaatttatccttaagatcccgaatcgtttaaac |
| HD1 TCR | 1001 | ttggccactccctctctgcgcgctcgctcgctcactgaggccgggcgaccaaaggtcgcc |
| insertion | | cgacgcccgggctttgcccgggcggcctcagtgagcgagcgagcgcgcagagaggg |
| including | | agtggccaactccatcactaggggttcctagatcttgccaacataccataaacctcccattct |
| ITRs | | gctaatgcccagcctaagttggggagaccactccagattccaagatgtacagtttgctttgc |
| | | tgggcctttttcccatgcctgcctttactctgccagagttatattgctggggttttgaagaagat |
| | | cctattaaataaaagaataagcagtattattaagtagccctgcatttcaggtttccttgagtgg |
| | | caggccaggcctggccgtgaacgttcactgaaatcatggcctcttggccaagattgatag |
| | | cttgtgcctgtccctgagtcccagtccatcacgagcagctggtttctaagatgctatttcccg |
| | | tataaagcatgagaccgtgacttgccagccccacagagccccgcccttgtccatcactgg |
| | | catctggactccagcctgggttggggcaaagagggaaatgagatcatgtcctaaccctga |
| | | tcctcttgtcccacagatatccagaaccctgaccctgcggctccggtgcccgtcagtgggc |
| | | agagcgcacatcgcccacagtccccgagaagttggggggaggggtcggcaattgaacc |
| | | ggtgcctagagaaggtggcgcggggtaaactgggaaagtgatgtcgtgtactggctccg |
| | | cctttttcccgaggggggggagaaccgtatataagtgcagtagtcgccgtgaacgttcttt |
| | | ttcgcaacgggtttgccgccagaacacaggtaagtgccgtgtgtggttcccgcgggcctg |
| | | gcctctttacgggttatggcccttgcgtgccttgaattacttccacgcccctggctgcagtac |
| | | gtgattcttgatcccgagcttcgggttggaagtgggtgggagagttcgaggccttgcgctt |
| | | aaggagccccttcgcctcgtgcttgagttgaggcctggcttgggcgctggggccgccgc |
| | | gtgcgaatctggtggcaccttcgcgcctgtctcgctgctttcgataagtctctagccatttaa |
| | | aatttttgatgacctgctgcgacgctttttttctggcaagatagtcttgtaaatgcgggccaag |
| | | atgtgcacactggtatttcggtttttggggccgcgggcggcgacggggcccgtgcgtccc |
| | | agcgcacatgttcggcgaggcggggcctgcgagcgcggccaccgagaatcggacgg |
| | | gggtagtctcaagctggccggcctgctctggtgcctggcctcgcgccgccgtgtatcgcc |
| | | ccgccctgggcggcaaggctggcccggtcggcaccagttgcgtgagcggaaagatgg |
| | | ccgcttcccggccctgctgcagggagctcaaaatggaggacgcggcgctcgggagagc |
| | | gggcgggtgagtcacccacacaaaggaaaagggcctttccgtcctcagccgtcgcttcat |
| | | gtgactccacggagtaccgggcgccgtccaggcacctcgattagttctcgagcttttggag |
| | | tacgtcgtctttaggttggggggaggggttttatgcgatggagtttccccacactgagtggg |
| | | tggagactgaagttaggccagcttggcacttgatgtaattctccttggaatttgccctttttga |
| | | gtttggatcttggttcattctcaagcctcagacagtggttcaaagtttttttcttccatttcagg |
| | | tgtcgtgatgcggccgccaccatgggatcttggacactgtgttgcgtgtccctgtgcatcctg |
| | | gtggccaagcacacagatgccggcgtgatccagtctcctagacacgaagtgaccgagat |
| | | gggccaagaagtgaccctgcgctgcaagcctatcagcggccacgattacctgttctggta |
| | | cagacagaccatgatgagaggcctggaactgctgatctacttcaacaacaacgtgcccat |
| | | cgacgacagcggcatgcccgaggatagattcagcgccaagatgcccaacgccagcttc |
| | | agcaccctgaagatccagcctagcgagcccagagatagcgccgtgtacttctgcgccag |
| | | cagaaagacaggcggctacagcaatcagccccagcactttggagatggcacccggctg |
| | | agcatcctggaagatctgaagaacgtgttcccacctgaggtggccgtgttcgagccttctg |
| | | aggccgagatcagccacacacagaaagccacactcgtgtgtctggccaccggcttctatc |
| | | ccgatcacgtggaactgtcttggtgggtcaacggcaaagaggtgcacagcggcgtcagc |
| | | accgatcctcagcctctgaaagagcagcccgctctgaacgacagcagatactgcctgag |
| | | cagcagactgagagtgtccgccaccttctggcagaaccccagaaaccacttcagatgcc |
| | | aggtgcagttctacggcctgagcgagaacgatgagtggacccaggatagagccaagcct |
| | | gtgacacagatcgtgtctgccgaagcctggggcagagccgattgtggctttaccagcgag |
| | | agctaccagcagggcgtgctgtctgccacaatcctgtacgagatcctgctgggcaaagcc |
| | | actctgtacgccgtgctggtgtctgccctggtgctgatggccatggtcaagcggaaggata |
| | | gcaggggcggctccggtgccacaaacttctccctgctcaagcaggccggagatgtggaa |
| | | gagaaccctggccctatggaaaccctgctgaaggtgctgagcggcacactgctgtggca |
| | | gctgacatgggtccgatctcagcagcctgtgcagtctcctcaggccgtgattctgagagaa |
| | | ggcgaggacgccgtgatcaactgcagcagctctaaggccctgtacagcgtgcactggta |
| | | cagacagaagcacggcgaggcccctgtgttcctgatgatcctgctgaaaggcggcgagc |
| | | agaagggccacgagaagatcagcgccagcttcaacgagaagaagcagcagtccagcc |
| | | tgtacctgacagccagccagctgagctacagcggcacctacttttgtggcaccgcctggat |
| | | caacgactacaagctgtctttcggagccggcaccacagtgacagtgcgggccaatattca |
| | | gaaccccgatcctgccgtgtaccagctgagagacagcaagagcagcgacaagagcgtg |
| | | tgcctgttcaccgacttcgacagccagaccaacgtgtcccagagcaaggacagcgacgt |
| | | gtacatcaccgataagactgtgctggacatgcggagcatggacttcaagagcaacagcg |
| | | ccgtggcctggtccaacaagagcgatttcgcctgcgccaacgccttcaacaacagcattat |
| | | ccccgaggacacattcttcccaagtcctgagagcagctgcgacgtgaagctggtggaaa |
| | | agagcttcgagacagacaccaacctgaacttccagaacctgagcgtgatcggcttcagaa |
| | | tcctgctgctcaaggtggccggcttcaacctgctgatgaccctgagactgtggtccagcta |
| | | acctCGACTGTGCCTTCTAGTTGCCAGCCATCTGTTGTTT |
| | | GCCCCTCCCCCGTGCCTTCCTTGACCCTGGAAGGTGCC |
| | | ACTCCCACTGTCCTTTCCTAATAAAATGAGGAAATTGC |
| | | ATCGCATTGTCTGAGTAGGTGTCATTCTATTCTGGGGG |
| | | GTGGGGTGGGGCAGGACAGCAAGGGGGAGGATTGGG |
| | | AAGACAATAGCAGGCATGCTGGGGATGCGGTGGGCTC |
| | | TATGGcttctgaggcggaaagaaccagctggggctctagggggtatccccactagtc |
| | | gtgtaccagctgagagactctaaatccagtgacaagtctgtctgcctattcaccgattttgatt |
| | | ctcaaacaaatgtgtcacaaagtaaggattctgatgtgtatatcacagacaaaactgtgcta |
| | | gacatgaggtctatggacttcaagagcaacagtgctgtggcctggagcaacaaatctgac |
| | | tttgcatgtgcaaacgccttcaacaacagcattattccagaagacaccttcttccccagccc |
| | | aggtaagggcagctttggtgccttcgcaggctgtttccttgcttcaggaatggccaggttct |
| | | gcccagagctctggtcaatgatgtctaaaactcctctgattggtggtctcggccttatccatt |
| | | gccaccaaaaccctctttttactaagaaacagtgagccttgttctggcagtccagagaatga |
| | | cacgggaaaaaagcagatgaagagaaggtggcaggagagggcacgtggcccagcct |
| | | cagtctctagatctaggaacccctagtgatggagttggccactccctctctgcgcgctcgct |
| | | cgctcactgaggccgcccgggcaaagcccgggcgtcgggcgacctttggtcgcccggc |
| | | ctcagtgagcgagcgagcgcgcagagagggagtggccaa |
| Guide | 1002 | NNNNNNNNNNNNNNNNNNNNGUUUUAGAGCUAGAA |
| Scaffold | | AUAGCAAGUUAAAAUAAGGCUAGUCCGUUAUCACGA |
| | | AAGGGCACCGAGUCGGUGCU |
| Guide | 1003 | mN*mN*mN*NNNNNNNNNNNNNNNNNGUUUUAGAmG |
| scaffold | | mCmUmAmGmAmAmAmUmAmGmCAAGUUAAAAUAAG |
| | | GCUAGUCCGUUAUCACGAAAGGGCACCGAGUCGGmU |
| | | mGmC*mU |
| | 1004 | Not Used |
| mRNA | 1005 | GGGAAGCUCAGAAUAAACGCUCAACUUUGGCCGGAU |
| sequence | | CUGCCACCAUGACCAACCUGUCCGACAUCAUCGAGA |
| encoding UGI | | AGGAGACCGGCAAGCAGCUGGUGAUCCAGGAGUCCA |
| | | UCCUGAUGCUGCCCGAGGAGGUGGAGGAGGUGAUCG |
| | | GCAACAAGCCCGAGUCCGACAUCCUGGUGCACACCG |
| | | CCUACGACGAGUCCACCGACGAGAACGUGAUGCUGC |
| | | UGACCUCCGACGCCCCCGAGUACAAGCCCUGGGCCCU |
| | | GGUGAUCCAGGACUCCAACGGCGAGAACAAGAUCAA |
| | | GAUGCUGUCCGGCGGCUCCAAGCGGACCGCCGACGG |
| | | CUCCGAGUUCGAGUCCCCCAAGAAGAAGCGGAAGGU |
| | | GGAGUGAUAGCUAGCACCAGCCUCAAGAACACCCGA |
| | | AUGGAGUCUCUAAGCUACAUAAUACCAACUUACACU |
| | | UUACAAAAUGUUGUCCCCCAAAAUGUAGCCAUUCGU |
| | | AUCUGCUCCUAAUAAAAAGAAAGUUUCUUCACAUUC |
| | | UCUCGAGAAAAAAAAAAAAUGGAAAAAAAAAAAAC |
| | | GGAAAAAAAAAAAAGGUAAAAAAAAAAAAUAUAAA |
| | | AAAAAAAAACAUAAAAAAAAAAAACGAAAAAAAAA |
| | | AAACGUAAAAAAAAAAAACUCAAAAAAAAAAAAGA |
| | | UAAAAAAAAAAAACCUAAAAAAAAAAAAUGUAAAA |
| | | AAAAAAAAGGGAAAAAAAAAAAACGCAAAAAAAAA |
| | | AAACACAAAAAAAAAAAAUGCAAAAAAAAAAAAUCG |
| | | AAAAAAAAAAAAUCUAAAAAAAAAAAACGAAAAAA |
| | | AAAAAACCCAAAAAAAAAAAAGACAAAAAAAAAAAA |
| | | UAGAAAAAAAAAAAAGUUAAAAAAAAAAAACUGAA |
| | | AAAAAAAAAAUUUAAAAAAAAAAAAUCUAG |
| Guide | 1006 | GUUUUAGAGCUAGAAAUAGCAAGUUAAAAUAAGGC |
| scaffold 90- | | UAGUCCGUUAUCACGAAAGGGCACCGAGUCGGUGC |
| mer | | |
| Guide | 1007 | GUUUUAGAmGmCmUmAmGmAmAmAmUmAmGmCAA |
| scaffold 90- | | GUUAAAAUAAGGCUAGUCCGUUAUCACGAAAGGGCA |
| mer with | | CCGAGUCGG*mU*mG*mC |
| modification | | |
| Guide | 1008 | GUUUUAGAmGmCmUmAmGmAmAmAmUmAmGmCAA |
| scaffold 90- | | GUUAAAAUAAGGCUAGUCCGUUAUCAmCmGmAmAm |
| mer with | | AmGmGmGmCmAmCmCmGmAmGmUmCmGmG*mU*mG |
| modification | | *mC |
| Guide | 1009 | GUUUUAGAmGmCmUmAmGmAmAmAmUmAmGmCAA |
| scaffold 88- | | GUUAAAAUAAGGCUAGUCCGUUAUCAACUUGGCACC |
| mer with | | GAGUCGG*mU*mG*mC |
| modification | | |
| Guide | 1010 | GUUUUAGAGCUAGAAAUAGCAAGUUAAAAUAAGGC |
| scaffold 88- | | UAGUCCGUUAUCAAAAUGGCACCGAGUCGGUGC |
| mer | | |
| Guide | 1011 | GUUUUAGAmGmCmUmAmGmAmAmAmUmAmGmCAA |
| scaffold 88- | | GUUAAAAUAAGGCUAGUCCGUUAUCAAAAUGGCACC |
| mer with | | GAGUCGG*mU*mG*mC |
| modification | | |
| Guide | 1012 | GUUUUAGAmGmCmUmAmGmAmAmAmUmAmGmCAA |
| scaffold 88- | | GUUAAAAUAAGGCUAGUCCGUUAUCAmAmAmAmUm |
| mer with | | GmGmCmAmCmCmGmAmGmUmCmGmG*mU*mG*mC |
| modification | | |
| Guide | 1013 | GUUUUAGAmGmCmUmAmGmAmAmAmUmAmGmCAA |
| scaffold | | GUUAAAAUAAGGCUAGUCCGUUAUCAmAmCmUmUm |
| | | GmAmAmAmAmAmGmUmGmGmCmAmCmCmGmAmGm |
| | | UmCmGmGmUmGmCmU*mU*mU*mU |
| Guide | 1014 | mN*mN*mN*NNNNNNNNNNNNNNNNNGUUUUAGAmG |
| scaffold | | mCmUmAmGmAmAmAmUmAmGmCAAGUUAAAAUAAG |
| | | GCUAGUCCGUUAUCAmAmCmUmUmGmAmAmAmAmA |
| | | mGmUmGmGmCmAmCmCmGmAmGmUmCmGmGmUmG |
| | | mCmU*mU*mU*mU |
| G023523 | 1015 | GCUGCAGCGCACGGGUACCAGUUUUAGAGCUAGAAA |
| Exemplary | | UAGCAAGUUAAAAUAAGGCUAGUCCGUUAUCACGAA |
| 91-mer full | | AGGGCACCGAGUCGGUGCU |
| sequence | | |
| G023523 | 1016 | mG*mC*mU*GCAGCGCACGGGUACCAGUUUUAGAmG |
| Exemplary | | mCmUmAmGmAmAmAmUmAmGmCAAGUUAAAAUAAG |
| 91-mer | | GCUAGUCCGUUAUCACGAAAGGGCACCGAGUCGGmU |
| modified | | mGmC*mU |
| sequence | | |

*The guide sequence disclosed in this Table may be unmodified, modified with the exemplary modification pattern shown in the Table, or modified with a different modification pattern disclosed herein or available in the art.

### IV. EXAMPLES

The following examples are provided to illustrate certain disclosed embodiments and are not to be construed as limiting the scope of this disclosure in any way.

### Example 1: General Methods

### 1.1. Next-Generation Sequencing (“NGS”) and Analysis for On-Target Cleavage Efficiency.

Genomic DNA was extracted using QuickExtract™ DNA Extraction Solution (Lucigen, Cat. No. QE09050) according to manufacturer's protocol.

To quantitatively determine the efficiency of editing at the target location in the genome, deep sequencing was utilized to identify the presence of insertions, deletions, and substitution introduced by gene editing. PCR primers were designed around the target site within the gene of interest (e.g., HLA-A) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### 1.2. T Cell Culture Media Preparation.

T cell culture media compositions used below are described here. “X-VIVO Base Media” consists of X-VIVO™ 15 Media, 1% Penstrep, 50 μM Beta-Mercaptoethanol, 10 mM NAC. In addition to above mentioned components, other variable media components used were: 1. Serum (Fetal Bovine Serum (FBS)); and 2. Cytokines (IL-2, IL-7, IL-15).

### 1.3. Preparation of Lipid Nanoparticles.

The lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A 49Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG in a 50:38:9:3 molar ratio, respectively. The lipid nucleic acid assemblies were formulated with a lipid amine to RNA phosphate (N:P) molar ratio of about 6, and a ratio of gRNA to mRNA of 1:1 or 1:2 by weight.

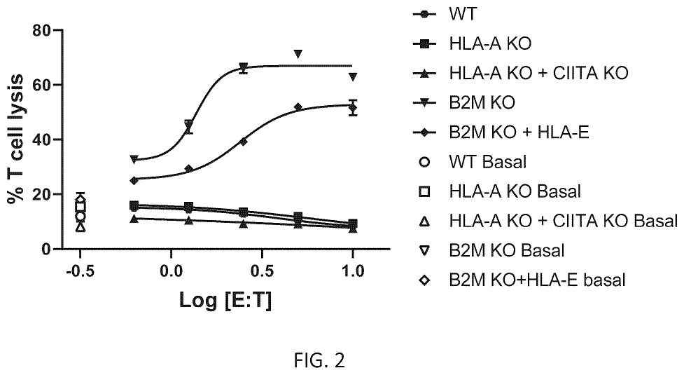

Lipid nanoparticles (LNP compositions) were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNP compositions were held for 1 hour at room temperature (RT), and further diluted with water (approximately 1:1 v/v). LNP compositions were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.4. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation sequence was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanlayzer (Agilent).

Streptococcus pyogenes (“Spy”) Cas9 mRNA was generated from plasmid DNA encoding an open reading frame according to SEQ ID NOs: 801-803 (see sequences in Table 6). BC22n mRNA was generated from plasmid DNA encoding an open reading frame according to SEQ ID NOs: 804-805. UGI mRNA was generated from plasmid DNA encoding an open reading frame according to SEQ ID NOs: 807-808. When SEQ ID NOs: 801-808 are referred to below with respect to RNAs, it is understood that Ts should be replaced with Us (which were N1-methyl pseudouridines as described above). Messenger RNAs used in the Examples include a cap and a 3′ polyadenylation region, e.g., up to 100 nts, and are identified by the SEQ ID NOs: 801-808 in Table 6.

### Example 2: Screening of HLA-A Guide RNAs with Cas9

Eighty-eight sgRNAs designed for the disruption of the HLA-A gene were screened for efficacy in T cells by assessing loss of two allelic versions of the MHC I surface protein, HLA-A2 and HLA-A3. The donor had an HLA-A phenotype of A*02:01:01G and 03:01:01G. The percentage of T cells double negative for HLA-A2 and A3 (“% A2−/A3−”) was determined by flow cytometry following editing at the HLA-A locus by electroporation with Cas9 ribonucleoprotein (RNP) and each test guide. Generally, unless otherwise indicated, guide RNAs used throughout the Examples identified as “G” refer to 100-nt modified sgRNA format, unless indicated otherwise, such as those shown in the Tables provided herein.

### 2.1. RNP Electroporation of T Cells

Cas9 editing activity was assessed using electroporation of Cas9 ribonucleoprotein (RNP). Upon thaw, Pan CD3+ T cells (StemCell, HLA-A*02.01/A*03.01) were plated at a density of 0.5×10\hat{ }⁶ cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 μM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec). Cells were expanded in T cell RPMI media for 72 hours prior to RNP transfection.

HLA-A targeting sgRNAs were removed from their storage plates and denatured for 2 minutes at 95° C. before cooling at room temperature for 10 minutes. RNP mixture of 20 μM sgRNA and 10 μM Cas9-NLS protein (SEQ ID NO: 800) was prepared and incubated at for 10 minutes. Five μL of RNP mixture was combined with 100,000 cells in 20 μL P3 electroporation Buffer (Lonza). 22 μL of RNP/cell mix was transferred to the corresponding wells of a Lonza shuttle 96-well electroporation plate. Cells were electroporated in duplicate with the manufacturer's pulse code. T cell RPMI media was added to the cells immediately post electroporation. Electroporated T cells were subsequently cultured and collected for NGS sequencing as described in Example 1 at 2 days post edit.

### 2.2. Flow Cytometry

On day 7 post-edit, T cells were phenotyped by flow cytometry to determine HLA-A protein expression following editing at the HLA-A locus. Briefly, T cells were incubated in a cocktail of antibodies targeting two allelic versions of the MHC I surface protein corresponding the cells donor's genotype HLA-A2, (eBioscience Cat. No. 17-9876-42) and HLA-A3 (eBioscience Cat. No. 12-5754-42). Cells were subsequently washed, processed on a Cytoflex flow cytometer (Beckman Coulter) and analyzed using the FlowJo software package. T cells were gated based on size, shape, viability, and HLA-A2 and HLA-A3 expression. Table 7 shows the mean percentage of cells double negative for HLA-A2 and HLA-A3 following editing at the HLA-A locus.

TABLE 7
Mean percentage of T cells HLA-A negative (double negative
for HLA-A2 and HLA-A3) following editing at the HLA-A locus

| | Mean % | SD % |
|---|---|---|
| Guide ID | A2−/A3− | A2−/A3− |
| G018932 | 39.30 | 1.56 |
| G018933 | 68.45 | 4.03 |
| G018934 | 34.40 | 0.57 |
| G018935 | 62.25 | 0.92 |
| G018936 | 7.62 | 0.28 |
| G018937 | 18.85 | 1.34 |
| G018938 | 0.05 | 0.04 |
| G018939 | 24.30 | 0.14 |
| G018940 | 3.99 | 0.06 |
| G018941 | 0.02 | 0.02 |
| G018942 | 1.97 | 0.19 |
| G018943 | 10.80 | 0.57 |
| G018944 | 1.78 | 0.16 |
| G018945 | 8.85 | 0.03 |
| G018946 | 8.08 | 0.44 |
| G018947 | 8.53 | 0.50 |
| G018948 | 8.57 | 0.59 |
| G018949 | 51.95 | 0.92 |
| G018950 | 1.80 | 0.08 |
| G018951 | 40.25 | 0.21 |
| G018952 | 3.40 | 0.30 |
| G018953 | 23.35 | 0.64 |
| G018954 | 57.50 | 1.41 |
| G018955 | 5.65 | 0.59 |
| G018956 | 40.45 | 0.21 |
| G018957 | 33.65 | 2.47 |
| G018958 | 1.52 | 0.00 |
| G018959 | 4.69 | 0.16 |
| G018960 | 0.13 | 0.00 |
| G018961 | 0.88 | 0.05 |
| G018962 | 0.78 | 0.01 |
| G018963 | 37.50 | 1.56 |
| G018964 | 12.75 | 0.64 |
| G018965 | 1.26 | 0.09 |
| G018966 | 0.28 | 0.06 |
| G018967 | 0.31 | 0.17 |
| G018968 | 0.34 | 0.07 |
| G018969 | 0.52 | 0.28 |
| G018970 | 0.55 | 0.13 |
| G018971 | 0.36 | 0.13 |
| G018972 | 17.15 | 0.78 |
| G018973 | 2.04 | 0.28 |
| G018974 | 1.26 | 0.03 |
| G018975 | 7.52 | 1.15 |
| G018976 | 3.75 | 0.22 |
| G018977 | 22.45 | 0.64 |
| G018978 | 7.79 | 0.64 |
| G018979 | 45.80 | 0.71 |
| G018980 | 35.70 | 1.98 |
| G018981 | 1.74 | 0.16 |
| G018982 | 3.31 | 0.22 |
| G018983 | 0.03 | 0.02 |
| G018984 | 0.78 | 0.04 |
| G018985 | 0.01 | 0.00 |
| G018986 | 0.01 | 0.00 |
| G018987 | 1.55 | 0.21 |
| G018988 | 1.72 | 0.08 |
| G018989 | 6.92 | 0.06 |
| G018990 | 13.70 | 0.99 |
| G018991 | 19.35 | 0.49 |
| G018992 | 21.70 | 2.26 |
| G018993 | 14.40 | 0.28 |
| G018994 | 25.35 | 0.64 |
| G018995 | 89.70 | 0.28 |
| G018996 | 92.35 | 0.07 |
| G018997 | 94.90 | 1.84 |
| G018998 | 90.50 | 0.42 |
| G018999 | 96.40 | 0.28 |
| G019000 | 94.95 | 0.21 |
| G019001 | 3.36 | 0.28 |
| G019002 | 0.02 | 0.00 |
| G019003 | 7.32 | 0.08 |
| G019004 | 52.70 | 2.40 |
| G019005 | 1.33 | 0.06 |
| G019006 | 8.18 | 0.98 |
| G019007 | 15.05 | 1.77 |
| G019008 | 58.65 | 2.19 |
| G019009 | 26.95 | 5.87 |
| G019010 | 4.69 | 0.04 |
| G019011 | 3.88 | 0.07 |
| G019012 | 23.75 | 1.91 |
| G019013 | 40.40 | 0.85 |
| G019014 | 26.55 | 0.07 |
| G019015 | 27.40 | 2.40 |
| G019016 | 20.20 | 0.00 |
| G019017 | 3.53 | 0.15 |
| G019018 | 18.60 | 0.28 |
| G019019 | 0.91 | 0.06 |

### Example 3: Screening of HLA-A Guides with BC22n and Cas9

HLA-A guide RNAs were screened for efficacy in T cells by assessing loss of HLA-A cell surface expression. The percentage of T cells negative for HLA-A protein in an HLA-A2 background (“% HLA-A2-”) was assayed by flow cytometry following HLA-A editing by mRNA delivery.

3.1. mRNA Electroporation of T Cells

Cas9 and BC22n editing activity was assessed using electroporation of mRNA encoding Cas9 (SEQ ID NO:802), mRNA encoding BC22n (SEQ ID NO:806), or mRNA encoding UGI (SEQ ID NO:807), as provided below. Upon thaw, Pan CD3+ T cells (StemCell, HLA-A*02.01/A*02.01) were plated at a density of 1× 10\hat{ }⁶ cells/mL in TCGM composed of CTS OpTmizer T Cell Expansion SFM (Thermofisher, Cat. A3705001) supplemented with 5% human AB serum (Gemini, Cat. 100-512), 1× GlutaMAX (Thermofisher, Cat.35050061), 10 mM HEPES (Thermofisher, Cat. 15630080), 1× of Penicillin-Streptomycin, further supplemented with 200 U/mL IL-2 (Peprotech, Cat. 200-02), ng/ml IL-7 (Peprotech, Cat. 200-07), 10 ng/ml IL-15 (Peprotech, Cat. 200-15). T cells were activated with TransAct™ (1:100 dilution, Miltenyi Biotec). Cells were expanded in T cell RPMI media for 72 hours at 37° C. prior to mRNA electroporation.

HLA-A sgRNAs were removed from their storage plates and denatured for 2 minutes at 95° C. before incubating at room temperature for 5 minutes. BC22n electroporation mix was prepared with 100,000 T cells in P3 buffer (Lonza), 200 ng of mRNA encoding UGI, 200 ng of mRNA encoding BC22n and 20 pmoles of sgRNA. Cas9 electroporation mix was prepared with 100,000 T cells in P3 buffer (Lonza), 200 ng of mRNA encoding UGI, 200 ng ofmRNA encoding Cas9 and 20 pmoles of sgRNA. Each mix was transferred to the corresponding wells of a Lonza shuttle 96-well electroporation plate. Cells were electroporated in duplicate using Lonza shuttle 96w using manufacturer's pulse code. Immediately post electroporation, cells were recovered in pre-warmed TCGM without cytokines and incubated at 37° C. for 15 minutes. Electroporated T cells were subsequently cultured in TCGM with further supplemented with 200 U/mL IL-2 (Peprotech, Cat. 200-02), 10 ng/ml IL-7 (Peprotech, Cat. 200-07), 10 ng/ml IL-15 (Peprotech, Cat. 200-15) and collected for flow cytometry 8 days post edit.

### 3.2. Flow Cytometry

On day 8 post-edit, T cells were phenotyped by flow cytometry to determine HLA-A protein expression. Briefly, T cells were incubated with antibodies targeting HLA-A2, (eBioscience Cat. No. 17-9876-42). Cells were subsequently washed, processed on a Cytoflex flow cytometer (Beckman Coulter) and analyzed using the FlowJo software package. T cells were gated based on size, shape, viability, and HLA-A2 expression. Table 8 shows the percentage of cells negative for HLA-A surface proteins following genomic editing of HLA-A with BC22n or Cas9.

TABLE 8
Percentage of cells negative for HLA-A surface protein
following genomic editing of HLA-A with BC22n or Cas9.

| | BC22n | | Cas9 | |
|---|---|---|---|---|
| Intellia ID | Mean % A2− | SD % A2− | Mean % A2− | SD % A2− |
| G018932 | 20.15 | 2.76 | 43.30 | 1.70 |
| G018933 | 10.35 | 1.20 | 74.00 | 0.57 |
| G018934 | 0.50 | 0.14 | 15.30 | 1.56 |
| G018935 | 0.00 | 0.00 | 69.30 | 0.28 |
| G018936 | 0.10 | 0.00 | 29.65 | 2.62 |
| G018937 | 0.15 | 0.07 | 50.50 | 0.71 |
| G018938 | 0.00 | 0.00 | 0.00 | 0.00 |
| G018939 | 0.00 | 0.00 | 44.90 | 1.27 |
| G018940 | 0.00 | 0.00 | 12.00 | 0.42 |
| G018941 | 0.00 | 0.00 | 2.65 | 0.35 |
| G018942 | 0.10 | 0.00 | 2.15 | 0.07 |
| G018943 | 0.00 | 0.00 | 16.20 | 0.42 |
| G018944 | 0.00 | 0.00 | 3.00 | 0.28 |
| G018945 | 0.05 | 0.07 | 3.20 | 0.42 |
| G018946 | 0.00 | 0.00 | 2.30 | 0.14 |
| G018947 | 0.00 | 0.00 | 1.55 | 0.49 |
| G018949 | 0.00 | 0.00 | 47.10 | 0.57 |
| G018950 | 0.00 | 0.00 | 0.30 | 0.00 |
| G018951 | 0.00 | 0.00 | 13.30 | 0.28 |
| G018952 | 0.00 | 0.00 | 0.50 | 0.00 |
| G018953 | 0.00 | 0.00 | 3.65 | 0.64 |
| G018955 | 0.20 | 0.14 | 5.20 | 0.28 |
| G018958 | 0.00 | 0.00 | 1.30 | 0.28 |
| G018959 | 0.00 | 0.00 | 3.70 | 0.14 |
| G018960 | 0.00 | 0.00 | 0.35 | 0.07 |
| G018961 | 0.00 | 0.00 | 0.40 | 0.00 |
| G018962 | 0.00 | 0.00 | 2.90 | 0.42 |
| G018963 | 0.00 | 0.00 | 12.50 | 0.14 |
| G018964 | 0.00 | 0.00 | 6.45 | 0.64 |
| G018965 | 0.00 | 0.00 | 0.90 | 0.00 |
| G018966 | 0.00 | 0.00 | 1.30 | 0.14 |
| G018968 | 0.10 | 0.00 | 0.10 | 0.00 |
| G018969 | 0.00 | 0.00 | 0.80 | 0.14 |
| G018970 | 0.00 | 0.00 | 0.95 | 0.07 |
| G018971 | 0.00 | 0.00 | 0.10 | 0.00 |
| G018972 | 0.05 | 0.07 | 3.40 | 0.28 |
| G018973 | 0.00 | 0.00 | 1.35 | 0.07 |
| G018974 | 0.00 | 0.00 | 0.45 | 0.07 |
| G018976 | 0.05 | 0.07 | 2.45 | 0.07 |
| G018977 | 0.00 | 0.00 | 12.45 | 1.06 |
| G018978 | 0.00 | 0.00 | 1.75 | 0.07 |
| G018979 | 0.05 | 0.07 | 37.40 | 0.71 |
| G018980 | 0.05 | 0.07 | 32.40 | 2.40 |
| G018981 | 0.00 | 0.00 | 17.45 | 0.35 |
| G018982 | 0.00 | 0.00 | 26.35 | 0.92 |
| G018983 | 0.00 | 0.00 | 0.25 | 0.07 |
| G018984 | 0.00 | 0.00 | 0.65 | 0.07 |
| G018986 | 0.00 | 0.00 | 1.85 | 0.21 |
| G018987 | 0.00 | 0.00 | 2.25 | 0.07 |
| G018988 | 0.00 | 0.00 | 0.15 | 0.07 |
| G018989 | 0.00 | 0.00 | 1.85 | 0.07 |
| G018990 | 0.25 | 0.07 | 17.45 | 1.06 |
| G018991 | 0.20 | 0.00 | 23.15 | 0.92 |
| G018992 | 0.20 | 0.14 | 38.15 | 0.07 |
| G018993 | 0.15 | 0.07 | 12.15 | 1.34 |
| G018994 | 4.35 | 0.35 | 23.75 | 0.49 |
| G018995 | 0.55 | 0.07 | 94.27 | 0.30 |
| G018996 | 0.85 | 0.07 | 92.39 | 0.83 |
| G018997 | 97.80 | 0.08 | 95.03 | 1.87 |
| G018998 | 74.75 | 7.71 | 93.33 | 0.18 |
| G018999 | 98.26 | 0.30 | 96.05 | 2.27 |
| G019000 | 9.05 | 0.35 | 94.67 | 0.74 |
| G019001 | 0.05 | 0.07 | 4.05 | 0.64 |
| G019002 | 0.00 | 0.00 | 0.05 | 0.07 |
| G019003 | 0.00 | 0.00 | 11.10 | 0.00 |
| G019004 | 0.00 | 0.00 | 30.70 | 0.00 |
| G019005 | 0.00 | 0.00 | 1.65 | 0.35 |
| G019006 | 0.00 | 0.00 | 4.75 | 0.49 |
| G019007 | 0.00 | 0.00 | 5.35 | 0.78 |
| G019008 | 0.00 | 0.00 | 55.20 | 3.54 |
| G019009 | 0.00 | 0.00 | 19.55 | 2.19 |
| G019010 | 0.05 | 0.07 | 5.40 | 0.14 |
| G019011 | 0.00 | 0.00 | 4.40 | 0.85 |
| G019012 | 0.05 | 0.07 | 22.90 | 2.55 |
| G019013 | 0.00 | 0.00 | 30.60 | 2.40 |
| G019014 | 0.05 | 0.07 | 14.65 | 0.49 |
| G019015 | 0.00 | 0.00 | 44.70 | 1.70 |
| G019016 | 0.00 | 0.00 | 13.95 | 0.35 |
| G019017 | 0.00 | 0.00 | 2.35 | 0.35 |
| G019018 | 0.00 | 0.00 | 19.90 | 0.00 |
| G019019 | 0.00 | 0.00 | 3.20 | 0.14 |
| G021205 | 0.00 | 0.00 | 0.00 | 0.00 |
| G021206 | 0.00 | 0.00 | 4.10 | 0.28 |
| G021207 | 0.00 | 0.00 | 2.80 | 0.28 |
| G021208 | 84.75 | 2.05 | 58.50 | 0.28 |
| G021209 | 97.96 | 0.16 | 83.35 | 1.77 |
| G021210 | 71.45 | 2.90 | 75.20 | 1.70 |
| G021211 | 0.10 | 0.00 | 67.80 | 1.70 |

### Example 4: NK Cell Functional Killing Assays

T cells edited in various combinations to disrupt CIITA, HLA-A, or B2M or to overexpress HLA-E were tested for their ability to resist natural killer (NK) cell mediated killing.

### 4.1. Engineering T Cells and Purification

Upon thaw, Pan CD3+ T cells (StemCell, HLA-A*02.01/A*03.01) were plated at a density of 0.5×10\hat{ }⁶ cells/mL in T cell RPMI media composed of RPMI 1640 (Invitrogen, Cat. 22400-089) containing 5% (v/v) of fetal bovine serum, 1× Glutamax (Gibco, Cat. 35050-061), 50 μM of 2-Mercaptoethanol, 100 μM non-essential amino acids (Invitrogen, Cat. 11140-050), 1 mM sodium pyruvate, 10 mM HEPES buffer, 1% of Penicillin-Streptomycin, and 100 U/mL of recombinant human interleukin-2 (Peprotech, Cat. 200-02). T cells were activated with TransAct™ (1:100 dilution, Miltenyi Biotec).

As described in Table 9, one day following activation, T cells were edited with to disrupt the B2M gene. Briefly, LNP compositions containing Cas9 mRNA and sgRNA G000529 (SEQ ID NO: 245) targeting B2M were formulated as described in Example 1. LNP compositions were incubated in RPMI-based media with cytokines as described above supplemented with 1 ug/ml recombinant human ApoE3 (Peprotech, Cat. 350-02) for 15 minutes at 37° C. LNP mix was added to two million activated T cells to yield a final concentration of 2.5 ug total LNP/mL.

TABLE 9
Order of sequential editing and viral transduction

| Condition | Day 1 | Day 2 | Day 3 |
|---|---|---|---|
| Unedited | | | |
| B2M⁻ | B2M LNP | | |
| B2M⁻ + HLA-E | B2M LNP | | HLA-E lentivirus |
| HLA-A⁻ MHC II⁻ | | CIITA LNP | HLA-A LNP |
| HLA-A⁻ | | | HLA-A LNP |

Two days post activation, additional T cells were edited with LNP compositions to disrupt the CIITA gene. This was performed as described for B2M editing using LNP compositions containing Cas9 mRNA and sgRNA G013675 (SEQ ID NO: 246) targeting CIITA. LNP compositions used in this step were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight.

Three days post activation, all edited and unedited cells were resuspended in fresh media without TransAct. A B2M-edited T cell sample was transduced by centrifugation at 1000 g at 37 C for 1 hour with lentivirus expressing HLA-E from an EF1a promoter (SEQ ID NO. 1000) at an MOI of 10. A CIITA-edited T cell sample was further edited with LNP compositions to disrupt the HLA-A gene. Editing was performed as described for B2M editing above using LNP compositions containing Cas9 mRNA and sgRNA 6019000 targeting HLA-A formulated with lipid A, cholesterol, DSPC, and PEG2k-DMG in a molar ratio, respectively. The lipid nucleic acid assemblies were formulated with a lipid amine to RNA phosphate (N:P) molar ratio of about 6, and a ratio of gRNA to mRNA of 1:2 by weight. Four days post activation, all cells were transferred to GREX plate (Wilson Wolf, Cat. 80240M) for expansion.

Seven days post activation, HLA-E infected T cells were selected for HLA-E expression using Biotinylated Anti-HLA-E Antibody (Biolegend). and Anti-Biotin microbeads (Miltenyi Biotec, Cat #130-090-485) and a magnetic LS Column (Miltenyi Biotec, Cat #130-042-401) according to manufacturer's protocols.

Similarly, nine days post activation CIITA edited T cells were negatively selected for lack of MHC II expression. using Biotinylated Anti-HLA-Class II Antibody (Miltenyi, Cat. 130-104-823), Anti-Biotin microbeads (Miltenyi Biotec, Cat. 130-090-485) and a magnetic LS Column (Miltenyi Biotec, Cat. 130-042-401) according to manufacturer's protocols.

### 4.2 Flow Cytometry

NK cell mediated cytotoxicity towards engineered T cells was assayed. For this the T cells were co-cultured with the HLA-B/C matched CTV labelled NK cells at effector to target ratios (E:T) of 10:1, 5:1, 2.5:1, 1.25:1 and 0.625:1 for 21 hours. The cells were stained with 7AAD (BD Pharmingen, Cat. 559925), processed on a Cytoflex flow cytometer (Beckman Coulter) and analyzed using the FlowJo software package. T cells were gated based on CTV negativity, size, and shape and viability. Table 10 and FIG. 2 show the percentage of T cell lysis following NK cell challenge.

TABLE 10
Percentage T cell lysis following NK cell challenge to engineered T cells

| | | | | | HLA-A⁻ | | | | B2M⁻ + | | |
|---|---|---|---|---|---|---|---|---|---|---|---|
| | Unedited | | HLA-A⁻ | | MHC II⁻ | | B2M⁻ | | HLA-E | | |
| Log(E:T) | Mean | SD | Mean | SD | Mean | SD | Mean | SD | Mean | SD | n |
| Basal | 12.0 | 1.9 | 15.5 | 0.2 | 8.2 | 0.4 | 11.1 | 0.1 | 18.1 | 2.5 | 2 |
| −0.20 | 15.1 | 0.0 | 16.0 | 0.5 | 11.2 | 0.8 | 32.6 | 1.6 | 25.0 | 0.9 | 2 |
| 0.10 | 14.5 | 0.2 | 15.6 | 0.4 | 10.6 | 0.1 | 44.7 | 2.3 | 29.4 | 0.1 | 2 |
| 0.40 | 12.8 | 0.6 | 13.6 | 0.4 | 9.3 | 0.1 | 66.0 | 1.8 | 39.3 | 0.1 | 2 |
| 0.70 | 10.4 | 0.4 | 11.9 | 0.2 | 9.2 | 0.4 | 71.2 | 1.3 | 51.9 | 1.6 | 2 |
| 1.00 | 8.4 | 0.1 | 9.4 | 0.6 | 7.6 | 0.1 | 62.8 | 0.6 | 51.7 | 2.8 | 2 |

### Example 5: LNP Dose Response Curves for Top HLA-A Guides

### 5.1 T Cell Preparation

Cryopreserved CD8/CD4+ selected T-cells isolated from a leukopak (Hemacare) were thawed and rested overnight at 1.5×10\hat{ }⁶ cells/ml in T cell growth media (TCGM) composed of CTS OpTmizer T Cell Expansion SFM (Thermofisher, Cat. A3705001) supplemented with 5% human AB serum (Gemini, Cat. 100-512), 1× GlutaMAX (Thermofisher, Cat.35050061), 10 mM HEPES (Thermofisher, Cat. 15630080), 200 U/mL IL-2 (Peprotech, Cat. 200-02), 5 ng/ml IL-7 (Peprotech, Cat. 200-07), 5 ng/ml IL-15 (Peprotech, Cat. 200-15).

T cells were activated using T cell TransAct′ (Miltenyi, Cat. 130-111-160) at 1:50 dilution and incubated in 37° C. incubator for 48 hours. After the incubation, the cells were counted on Vi-cell and resuspended in TCGM as described above but with 2.5% serum to a final concentration of 0.5×10\hat{ }⁶ cells/ml. After 24 hours, the cells were counted on Vi-cell, resuspended in 5% serum TCGM and transferred to a 96-well plate. Meanwhile, APOE (Peprotech, Cat. 350-02) was added into serum-free TCGM at a final concentration of 10 μg/ml and incubated with different HLA-A LNP compositions (see Table 11) at titrated LNP total RNA concentrations (10 μg/mL, 5 μg/ml, 2.5 μg/ml, 1.25 μg/ml, 0.625 μg/ml, 0.3125 μg/ml, 0.15625 μg/ml, and 0.078125 μg/ml) for 15 minutes. LNP compositions were contain mRNA encoding a Cas9 (SEQ ID NO:802) and guides as specified in Table 11 and were formulated with lipid A, cholesterol, DSPC, and PEG2k-DMG in a 50:39.5:9:1.5 molar ratio, respectively. The lipid nucleic acid assemblies were formulated with a lipid amine to RNA phosphate (N:P) molar ratio of about 6, and a ratio of gRNA to mRNA of 1:2 by weight. After the incubation with APOE, LNP suspension was added to T cells at 1:1 ratio and incubated at 37° C. for 24 hours. After 24 hours, the cells were counted on Vi-cell and split at 1:5 ratio and cultured for 96 hours. After incubation, an aliquot of 0.1-0.5×10\hat{ }⁶ cells was taken for flow cytometry analysis.

### 5.2 Flow Cytometry

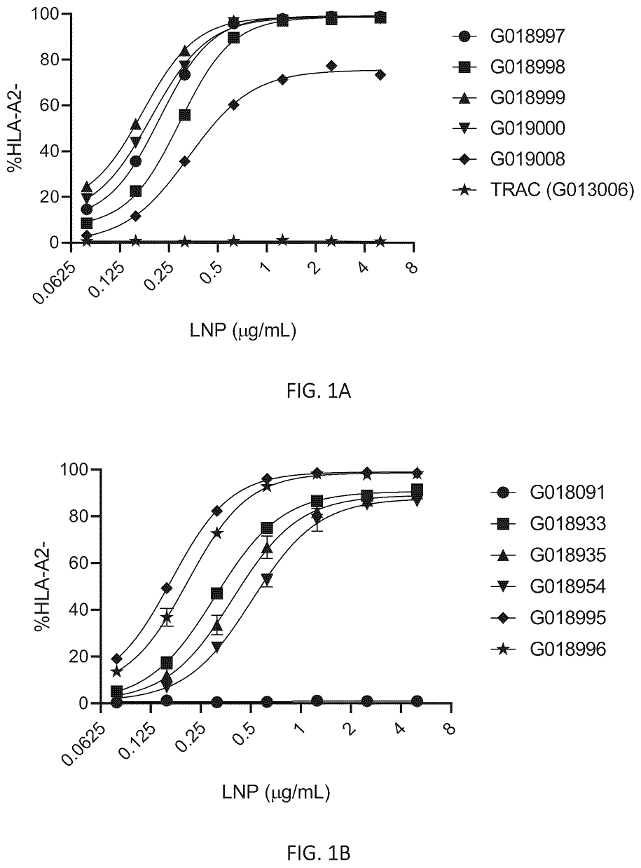

For flow cytometric analysis, cells were washed in FACS buffer (PBS+2% FBS+2 mM EDTA) and incubated with APC-conjugated anti-human HLA-A2 antibody (Biolegend®, 343308) and PC5.5-conjugated CD3 antibody (Biolegend®, Cat. 317336) at 1:200 dilution for 30 mins at 4° C. After the incubation, the cells were washed, resuspended in FACS buffer and processed by flow cytometry, for example using a Beckman Coulter CytoflexS, and analyzed using the FlowJo software package. Table 12 and FIGS. 1A-1B show the percent editing at each LNP dose.

TABLE 11
Maximum indel % and EC50 for HLA-A targeting guides

| sgRNA | Max | EC50 |
|---|---|---|
| G018933 | 90.71 | 0.3043 |
| G018935 | 89.04 | 0.3906 |
| G018954 | 87.68 | 0.5089 |
| G018995 | 98.99 | 0.1665 |
| G018996 | 98.61 | 0.2085 |
| G018997 | 99.12 | 0.2196 |
| G018998 | 98.64 | 0.2914 |
| G018999 | 98.74 | 0.1724 |
| G019000 | 98.61 | 0.1945 |
| G019008 | 75.53 | 0.3322 |
| G013006 TRAC | | |
| G018091 CIITA | 1.017 | 0.8941 |

TABLE 12
Percentage of HLA-A− cells after editing with various guides.

| | LNP | | | |
|---|---|---|---|---|
| | Concentration | | | |
| | (ug total | Mean % | | |
| sgRNA | RNA/ml) | HLA-A− | SD | n |
| G018933 | 5 | 91.45 | 0.35 | 2 |
| G018933 | 2.5 | 88.8 | 1.27 | 2 |
| G018933 | 1.25 | 86.55 | 0.35 | 2 |
| G018933 | 0.63 | 75 | 0.14 | 2 |
| G018933 | 0.31 | 47 | 0.00 | 2 |
| G018933 | 0.16 | 17.55 | 0.35 | 2 |
| G018933 | 0.08 | 5.115 | 0.28 | 2 |
| G018935 | 5 | 89.75 | 1.34 | 2 |
| G018935 | 2.5 | 86.8 | 0.28 | 2 |
| G018935 | 1.25 | 81.8 | 0.99 | 2 |
| G018935 | 0.63 | 66.8 | 4.81 | 2 |
| G018935 | 0.31 | 33.55 | 4.17 | 2 |
| G018935 | 0.16 | 11.91 | 2.96 | 2 |
| G018935 | 0.08 | 3.01 | 1.09 | 2 |
| G018954 | 5 | 86.5 | 86.4 | 2 |
| G018954 | 2.5 | 86 | 84 | 2 |
| G018954 | 1.25 | 82 | 75 | 2 |
| G018954 | 0.63 | 50.5 | 54.5 | 2 |
| G018954 | 0.31 | 24.8 | 23 | 2 |
| G018954 | 0.16 | 7.31 | 6.2 | 2 |
| G018954 | 0.08 | 2.09 | 1.78 | 2 |
| G018995 | 5 | 98.5 | 0.3 | 2 |
| G018995 | 2.5 | 98.8 | 0.1 | 2 |
| G018995 | 1.25 | 98.55 | 0.35 | 2 |
| G018995 | 0.63 | 96 | 0 | 2 |
| G018995 | 0.31 | 82.25 | 1.25 | 2 |
| G018995 | 0.16 | 49.25 | 0.55 | 2 |
| G018995 | 0.08 | 19 | 0.3 | 2 |
| G018996 | 5 | 98.25 | 0.21 | 2 |
| G018996 | 2.5 | 97.75 | 0.64 | 2 |
| G018996 | 1.25 | 98.2 | 0.71 | 2 |
| G018996 | 0.63 | 92.75 | 0.49 | 2 |
| G018996 | 0.31 | 72.7 | 1.41 | 2 |
| G018996 | 0.16 | 36.8 | 3.82 | 2 |
| G018996 | 0.08 | 13.5 | 1.13 | 2 |
| G018997 | 5 | 98.8 | 0.1 | 2 |
| G018997 | 2.5 | 98.75 | 0.05 | 2 |
| G018997 | 1.25 | 97.8 | 0.3 | 2 |
| G018997 | 0.63 | 95.8 | 1.6 | 2 |
| G018997 | 0.31 | 73.45 | 0.15 | 2 |
| G018997 | 0.16 | 35.65 | 0.25 | 2 |
| G018997 | 0.08 | 14.65 | 0.15 | 2 |
| G018998 | 5 | 98.35 | 0.15 | 2 |
| G018998 | 2.5 | 97.65 | 0.15 | 2 |
| G018998 | 1.25 | 97.05 | 0.45 | 2 |
| G018998 | 0.63 | 89.6 | 1.4 | 2 |
| G018998 | 0.31 | 55.8 | 0.4 | 2 |
| G018998 | 0.16 | 22.6 | 0.8 | 2 |
| G018998 | 0.08 | 8.55 | 0.09 | 2 |
| G018999 | 5 | 98.45 | 0.35 | 2 |
| G018999 | 2.5 | 98.5 | 0.3 | 2 |
| G018999 | 1.25 | 98.05 | 0.55 | 2 |
| G018999 | 0.63 | 97.1 | 0.1 | 2 |
| G018999 | 0.31 | 84 | 0.4 | 2 |
| G018999 | 0.16 | 51.95 | 0.25 | 2 |
| G018999 | 0.08 | 24.7 | 0.4 | 2 |
| G019000 | 5 | 97.9 | 0 | 2 |
| G019000 | 2.5 | 98.5 | 0.1 | 2 |
| G019000 | 1.25 | 97.2 | 0.6 | 2 |
| G019000 | 0.63 | 96.05 | 0.35 | 2 |
| G019000 | 0.31 | 77 | 0.6 | 2 |
| G019000 | 0.16 | 43.7 | 1.1 | 2 |
| G019000 | 0.08 | 19.1 | 0.2 | 2 |
| G019008 | 5 | 73.35 | 1.20 | 2 |
| G019008 | 2.5 | 77.35 | 0.78 | 2 |
| G019008 | 1.25 | 71.25 | 2.19 | 2 |
| G019008 | 0.63 | 60.3 | 1.84 | 2 |
| G019008 | 0.31 | 35.65 | 2.19 | 2 |
| G019008 | 0.16 | 11.6 | 0.71 | 2 |
| G019008 | 0.08 | 3.17 | 0.41 | 2 |
| G018091 | 5 | 0.99 | 0.29 | 2 |
| G018091 | 2.5 | 1.00 | 0.52 | 2 |
| G018091 | 1.25 | 1.12 | 1.10 | 2 |
| G018091 | 0.63 | 0.64 | 0.02 | 2 |
| G018091 | 0.31 | 0.44 | 0.02 | 2 |
| G018091 | 0.16 | 1.22 | 0.52 | 2 |
| G018091 | 0.08 | 0.35 | 0.16 | 2 |
| G013006 | 5 | 0.51 | 0.28 | 2 |
| G013006 | 2.5 | 0.71 | 0.1 | 2 |
| G013006 | 1.25 | 1.13 | 0.315 | 2 |
| G013006 | 0.63 | 0.69 | 0.02 | 2 |
| G013006 | 0.31 | 0.36 | 0.015 | 2 |
| G013006 | 0.16 | 0.82 | 0.19 | 2 |
| G013006 | 0.08 | 0.7 | 0.02 | 2 |

### Example 6: Multi-Editing WT1 T Cells with Sequential LNP Delivery

T cells were engineered with a series of gene disruptions and insertions. Healthy donor cells were treated sequentially with four LNP compositions, each LNP co-formulated with mRNA encoding Cas9 and a sgRNA targeting either TRAC (G013006) (SEQ ID NO: 243), TRBC (G016239) (SEQ ID NO: 247), CIITA (G013676) (SEQ ID NO: 248), or HLA-A (G018995) (sgRNA comprising SEQ ID NO: 13, as shown in Table 2). LNP compositions were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. A transgenic T cell receptor targeting Wilm's tumor antigen (WT1 TCR) (SEQ ID NO: 1001) was integrated into the TRAC cut site by delivering a homology directed repair template using AAV.

### 6.1. T Cell Preparation

T cells were isolated from the leukapheresis products of three healthy HLA-A2+ donors (STEMCELL Technologies). T cells were isolated using EasySep Human T cell Isolation kit (STEMCELL Technologies, Cat. 17951) following manufacturers protocol and cryopreserved using Cryostor CS10 (STEMCELL Technologies, Cat. 07930). The day before initiating T cell editing, cells were thawed and rested overnight in T cell activation media (TCAM): CTS OpTmizer (Thermofisher, Cat. A3705001) supplemented with 2.5% human AB serum (Gemini, Cat. 100-512), 1× GlutaMAX (Thermofisher, Cat.35050061), 10 mM HEPES (Thermofisher, Cat. 15630080), 200 U/mL IL-2 (Peprotech, Cat. 200-02), IL-7 (Peprotech, Cat. 200-07), IL-15 (Peprotech, Cat. 200-15).

### 6.2. LNP Treatment and Expansion of T Cells

LNP compositions were prepared each day in ApoE containing media and delivered to T cells as described in Table 13 and below.

TABLE 13
Order of editing for T cell engineering

| Group | Day 1 | Day 2 | Day 3 | Day 4 |
|---|---|---|---|---|
| 1 | Unedited | Unedited | Unedited | Unedited |
| 2 | TRBC | CIITA | TRAC | HLA-A |
| 3 | TRBC | HLA-A | TRAC | CIITA |
| 4 | TRBC | | TRAC | |

On day 1, LNP compositions as indicated in Table 13 were incubated at a concentration of 5 ug/mL in TCAM containing 5 ug/mL rhApoE3 (Peprotech, Cat. 350-02). Meanwhile, T cells were harvested, washed, and resuspended at a density of 2×10\hat{ }⁶ cells/mL in TCAM with a 1:50 dilution of T Cell TransAct, human reagent (Miltenyi, Cat. 130-111-160). T cells and LNP-ApoE media were mixed at a 1:1 ratio and T cells plated in culture flasks overnight.

On day 1, LNP compositions as indicated in Table 13 were incubated at a concentration of 25 ug/mL in TCAM containing 20 ug/mL rhApoE3 (Peprotech, Cat. 350-02). LNP-ApoE solution was then added to the appropriate culture at a 1:10 ratio.

On day 3, TRAC-LNP compositions was incubated at a concentration of 5 ug/mL in TCAM containing 10 ug/mL rhApoE3 (Peprotech, Cat. 350-02). T cells were harvested, washed, and resuspended at a density of 1×10\hat{ }⁶ cells/mL in TCAM. T cells and LNP-ApoE media were mixed at a 1:1 ratio and T cells plated in culture flasks. WT1 AAV was then added to each group at a MOI of 3×10\hat{ }⁵ genome copies/cell.

On day 4, LNP compositions as indicated in Table 13 were incubated at a concentration of 5 ug/mL in TCAM containing 5 ug/mL rhApoE3 (Peprotech, Cat. 350-02). LNP-ApoE solution was then added to the appropriate culture at a 1:1 ratio.

On days 5-11, T cells were transferred to a 24-well GREX plate (Wilson Wolf, Cat. 80192) in T cell expansion media (TCEM): CTS OpTmizer (Thermofisher, Cat. A3705001) supplemented with 5% CTS Immune Cell Serum Replacement (Thermofisher, Cat. A2596101), 1× GlutaMAX (Thermofisher, Cat. 35050061), 10 mM HEPES (Thermofisher, Cat. 15630080), 200 U/mL IL-2 (Peprotech, Cat. 200-02), IL-7 (Peprotech, Cat. 200-07), and IL-15 (Peprotech, Cat. 200-15)). Cells were expanded per manufacturers protocols. T-cells were expanded for 6-days, with media exchanges every other day. Cells were counted using a Vi-CELL cell counter (Beckman Coulter) and fold expansion was calculated by dividing cell yield by the starting material as shown in Table 14.

TABLE 14
Fold expansion following multi-edit T cell engineering

| Group | Donor A | Donor B | Donor C | Mean | SD |
|---|---|---|---|---|---|
| 1 | 331.40 | 362.24 | 533.18 | 408.94 | 108.69 |
| 2 | 61.82 | 72.15 | 116.13 | 83.37 | 28.84 |
| 3 | 64.08 | 76.29 | 157.75 | 99.37 | 50.92 |
| 4 | No data | 146.78 | 331.67 | 239.22 | 130.74 |

### 6.3. Quantification of T Cell Editing by Flow Cytometry and NGS

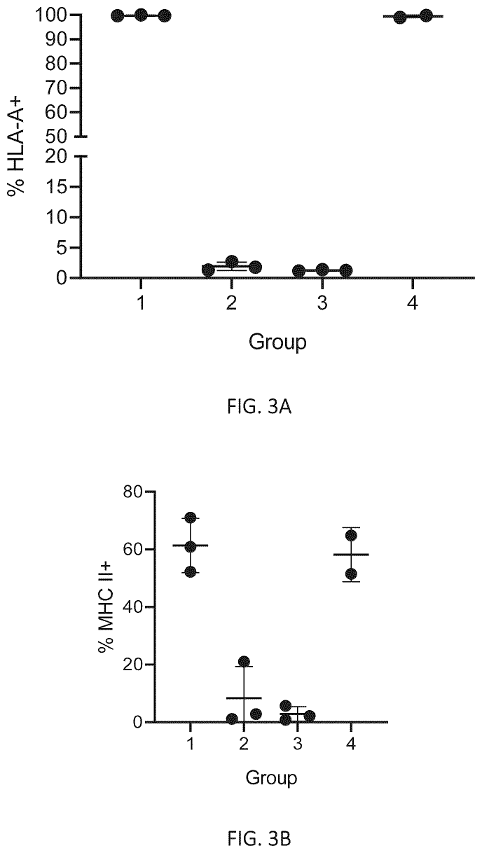

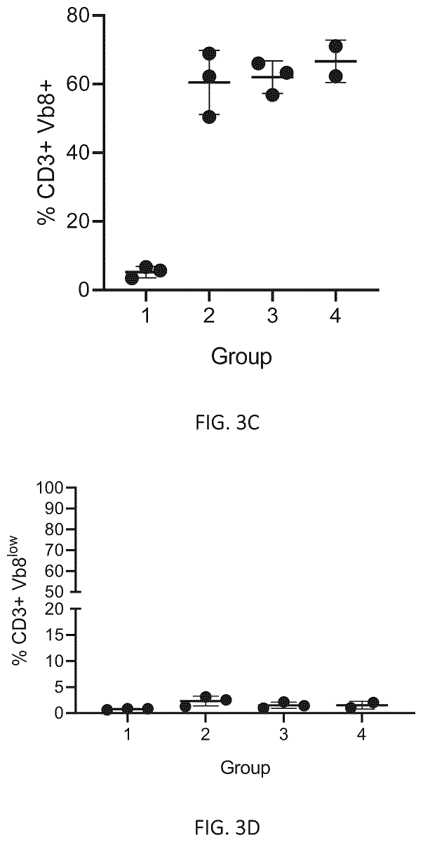

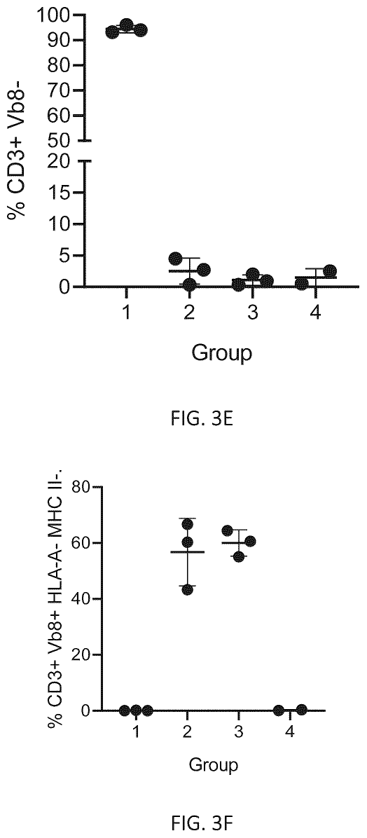

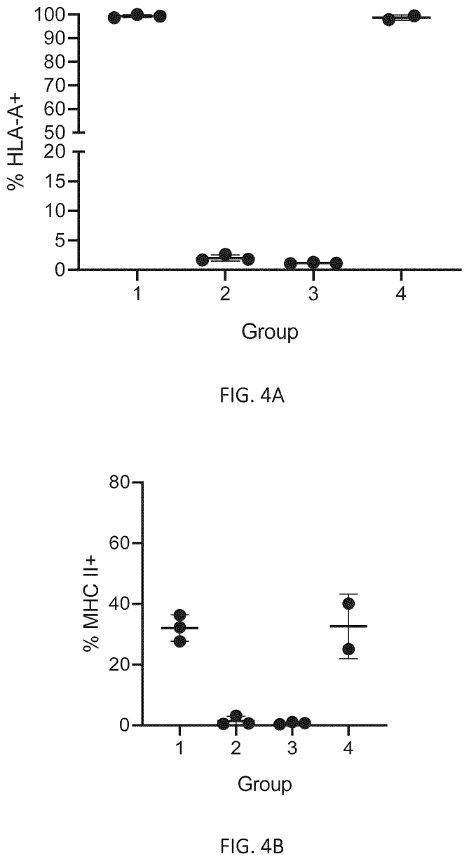

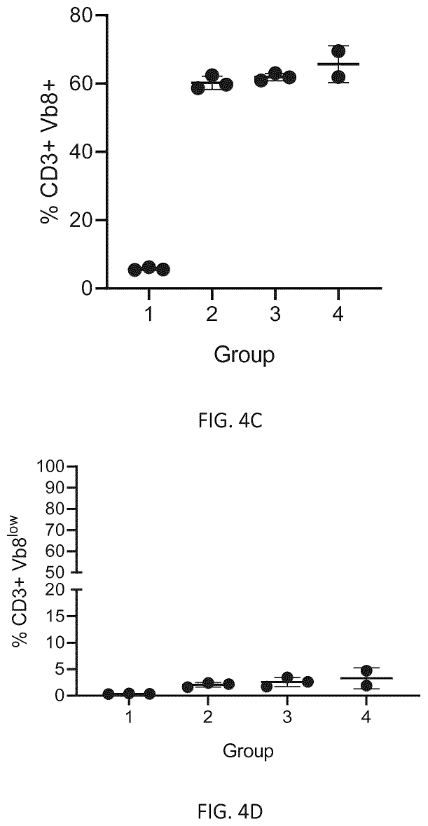

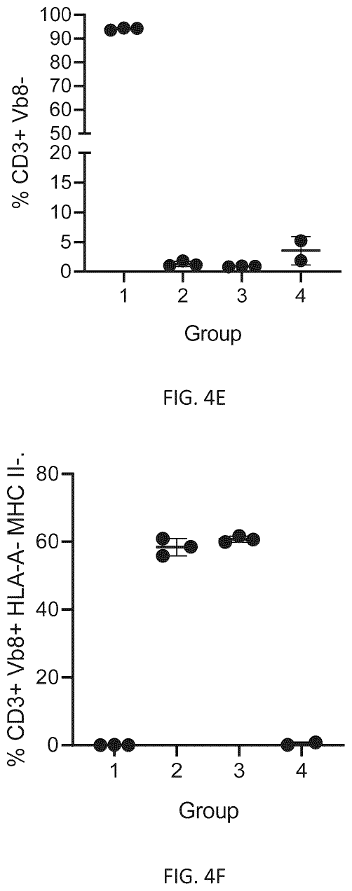

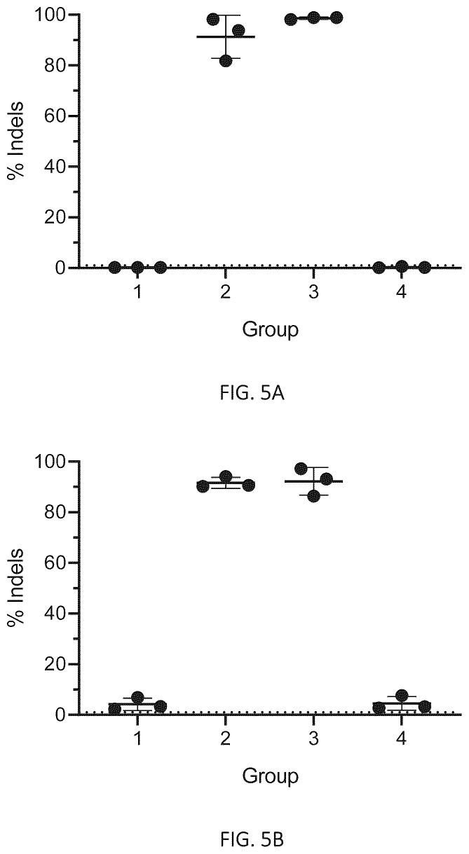

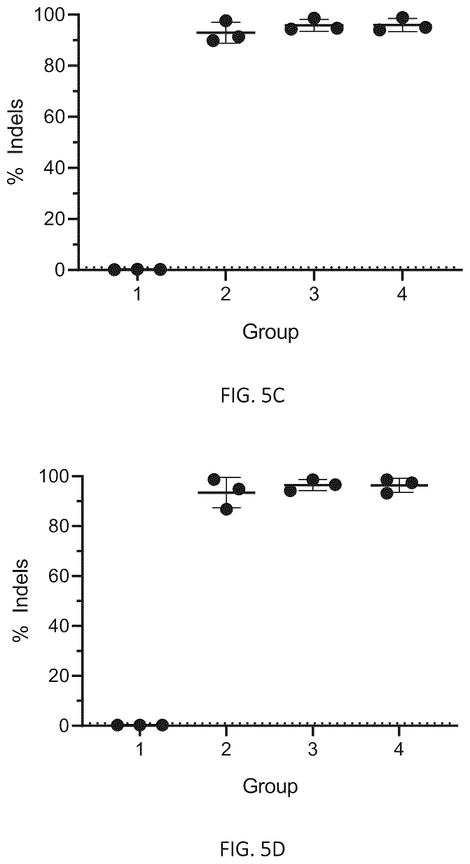

Post expansion, edited T cells were assayed by flow cytometry to determine HLA-A2 expression (HLA-A+), HLA-DR-DP-DQ expression (MHC II+) following knockdown CIITA, WT1-TCR expression (CD3+ Vb8+), and the expression of residual endogenous TCRs (CD3+ Vb8−) or mispaired TCRs (CD3+ Vb8low). T cells were incubated with an antibody cocktail targeting the following molecules: CD4 (Biolegend, Cat. 300524), CD8 (Biolegend, Cat. 301045), Vb8 (Biolegend, Cat. 348106), CD3 (Biolegend, Cat. 300327), HLA-A2 (Biolegend, Cat. 343306), HLA-DRDPDQ (Biolegend, Cat 361706), CD62L (Biolegend, Cat. 304844), CD45RO (Biolegend, Cat. 304230). Cells were subsequently washed, analyzed on a Cytoflex LX instrument (Beckman Coulter) using the FlowJo software package. T cells were gated on size and CD4/CD8 status, before expression of editing and insertion markers was determined. The percentage of cells expressing relevant cell surface proteins following sequential T cell engineering are shown in Table 15 and FIGS. 3A-F for CD8+ T cells and Table 16 and FIGS. 4A-F for CD4+ T cells. The percent of fully edited CD4+ or CD8+ T cells was gated as % CD3+ Vb8+ HLA-A− MHC II−. High levels of HLA-A and MHC II knockdown, as well as WT1-TCR insertion and endogenous TCR KO are observed in edited samples. In addition to flow cytometry analysis, genomic DNA was prepared and NGS analysis performed as described in Example 1 to determine editing rates at each target site. Table 17 and FIGS. 5A-D show results for percent editing at the CIITA, HLA-A, and TRBC1/2 loci, with patterns across the groups consistent with what was identified by flow cytometry. TRBC1/2 loci were edited to >90-95% in all groups.

TABLE 15
Percentage of CD8⁺ cell with cell surface phenotype following sequential T cell engineering

| | | | | | | | % Fully edited |
|---|---|---|---|---|---|---|---|
| | | | % | | % | % Residual | CD3⁺ Vb8⁺ |
| | | % | MHC II⁺ | % WT1 | Mispaired | endogenous | HLA-A2⁻ |
| | | HLA-A I⁺ | HLA-DR- | TCR | TCR | TCR | HLA-DR- |
| Donor | Group | HLA-A2⁺ | DP-DQ⁺ | CD3⁺ Vb8⁺ | CD3⁺ Vb8^{low} | CD3⁺ Vb8⁻ | DP-DQ⁻ |
| A | 1 | 100.0 | 60.9 | 6.7 | 0.8 | 93.2 | 0.0 |
| B | Unedited | 99.7 | 71.0 | 3.4 | 0.6 | 96.1 | 0.2 |
| C | | 99.7 | 52.2 | 5.7 | 0.8 | 94.0 | 0.0 |
| A | 2 | 2.7 | 1.2 | 68.9 | 1.3 | 0.4 | 66.7 |
| B | | 1.3 | 21.0 | 50.4 | 3.1 | 4.5 | 43.3 |
| C | | 1.8 | 2.9 | 62.2 | 2.6 | 2.7 | 60.3 |
| A | 3 | 1.3 | 0.8 | 66.0 | 1.4 | 0.3 | 64.4 |
| B | | 1.4 | 2.2 | 56.8 | 2.2 | 2.0 | 55.1 |
| C | | 1.2 | 5.7 | 63.3 | 1.0 | 0.9 | 60.6 |
| B | 4 | 99.8 | 64.8 | 62.3 | 2.0 | 2.5 | 0.1 |
| C | | 99.0 | 51.5 | 71.0 | 1.0 | 0.5 | 0.4 |

TABLE 16
Percentage of CD4+ cells with cell surface phenotype following sequential T cell engineering

| | | | | | | | % Fully edited |
|---|---|---|---|---|---|---|---|
| | | | % | | % | % Residual | CD3⁺ Vb8⁺ |
| | | % | MHC II⁺ | % WT1 | Mispaired | endogenous | HLA-A2⁻ |
| | | HLA-A I⁺ | HLA-DR- | TCR | TCR | TCR | HLA-DR- |
| Donor | Group | HLA-A2⁺ | DP-DQ⁺ | CD3⁺ Vb8⁺ | CD3⁺ Vb8^{low} | CD3⁺ Vb8⁻ | DP-DQ⁻ |
| A | 1 | 100.0 | 36.3 | 5.4 | 0.4 | 94.5 | 0.0 |
| B | Unedited | 98.7 | 27.6 | 5.6 | 0.4 | 94.3 | 0.0 |
| C | | 99.3 | 32.3 | 6.2 | 0.3 | 93.6 | 0.1 |
| A | 2 | 2.6 | 0.7 | 62.4 | 2.4 | 1.1 | 60.9 |
| B | | 1.8 | 0.5 | 59.7 | 2.2 | 1.0 | 58.5 |
| C | | 1.7 | 3.2 | 58.6 | 1.6 | 1.8 | 55.8 |
| A | 3 | 1.3 | 0.8 | 63.0 | 3.4 | 0.8 | 61.7 |
| B | | 1.1 | 1.1 | 61.8 | 2.6 | 0.9 | 60.6 |
| C | | 1.1 | 0.4 | 60.9 | 1.7 | 1.0 | 59.9 |
| B | 4 | 99.5 | 25.1 | 61.9 | 1.9 | 5.2 | 0.1 |
| C | | 97.9 | 40.1 | 69.5 | 4.7 | 1.9 | 0.8 |

TABLE 17
Percent indels at CIITA, HLA-A, TRBC1 and TRBC2 following sequential T cell editing

| | CIITA (G013676) | | | HLA-A (G018995) | | | TRBC1 (G016239) | | | TRBC2 (G016239) | | |
|---|---|---|---|---|---|---|---|---|---|---|---|---|
| | Donor | Donor | Donor | Donor | Donor | Donor | Donor | Donor | Donor | Donor | Donor | Donor |
| Group | A | B | C | A | B | C | A | B | C | A | B | C |
| 1 | 0.2 | 0.2 | 0.2 | 6.9 | 3.3 | 2.3 | 0.1 | 0.3 | 0.2 | 0.3 | 0.3 | 0.3 |
| 2 | 98.2 | 81.8 | 93.8 | 94.1 | 90.2 | 90.6 | 97.6 | 89.9 | 91.4 | 98.7 | 86.8 | 94.9 |
| 3 | 98.9 | 98.1 | 98.9 | 97.2 | 86.4 | 93.1 | 98.6 | 94.4 | 94.7 | 98.6 | 94.2 | 96.6 |
| 4 | 0.1 | 0.2 | 0.6 | 7.6 | 2.7 | 3.2 | 98.9 | 94 | 95 | 98.6 | 93.2 | 97.4 |

### Example 7: Off-Target Analysis of HLA-A Human Guides

Screening for potential off-target genomic sites cleaved by Cas9 targeting HLA-A was performed. (See, e.g., Cameron et al., Nature Methods. 6, 600-606; 2017). In this experiment, 10 sgRNA targeting human HLA-A and three control guides targeting EMX1, VEGFA, and RAG1B with known off-target profiles were screened using purified genomic DNA from lymphoblast cell line NA24385 (Coriell Institute). The number of potential off-target sites were detected using a sgRNA as shown in Table 18 at a concentration of 192 nM sgRNA and 64 nM RNP in the biochemical assay. The assay identified potential off-target sites for the sgRNAs tested.

TABLE 18
Off-Target Analysis

| | | Guide Sequence | Off-Target |
|---|---|---|---|
| gRNA ID | Target | (SEQ ID NO:) | Site Count |
| G018995 | HLA-A | ACAGCGACGCCGCGAGCCAG | 17 |
| | | (SEQ ID NO: 13) | |
| G018996 | HLA-A | CGACGCCGCGAGCCAGAGGA | 48 |
| | | (SEQ ID NO: 14) | |
| G018997 | HLA-A | CGAGCCAGAGGAUGGAGCCG | 1299 |
| | | (SEQ ID NO: 15) | |
| G018998 | HLA-A | CGGCUCCAUCCUCUGGCUCG | 250 |
| | | (SEQ ID NO: 16) | |
| G018999 | HLA-A | GAGCCAGAGGAUGGAGCCGC | 733 |
| | | (SEQ ID NO: 17) | |
| G019000 | HLA-A | GCGCCCGCGGCUCCAUCCUC | 386 |
| | | (SEQ ID NO: 18) | |
| G018933 | HLA-A | GCACGGGUACCAGGGGCCAC | 865 |
| | | (SEQ ID NO: 41) | |
| G018935 | HLA-A | GGGAGGCGCCCCGUGGCCCC | 258 |
| | | (SEQ ID NO: 43) | |
| G019008 | HLA-A | GCAAGGGUCUCGGGGUCCCG | 324 |
| | | (SEQ ID NO: 26) | |
| G018954 | HLA-A | UUGAGAAUGGACAGGACACC | 227 |
| | | (SEQ ID NO: 62) | |
| G000644 | EMX1 | GAGUCCGAGCAGAAGAAGAA | 253 |
| | | (SEQ ID NO: 230) | |
| G000645 | VEGFA | GACCCCCUCCACCCCGCCUC | 3856 |
| | | (SEQ ID NO: 231) | |
| G000646 | RAG1B | GACUUGUUUUCAUUGUUCUC | 62 |
| | | (SEQ ID NO: 232) | |

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

### Example 8: HLA-A and CIITA Partial-Matching in an NK Cell In Vivo Killing Mouse Model

Female NOG-hIL-15 mice were engrafted with 1.5×10\hat{ }⁶ primary NK cells followed by the injection of engineered T cells containing luciferase+/−HLA-A, CIITA, or HLA-A/CIITA KO 4 weeks later in order to determine 1) whether engrafted NK cells can readily lyse control T cells (B2M^{-/-}), and 2) whether the addition of a partial-matching edit (HLA-A or CIITA) provides a protective effect for T cells from NK cell lysis in vivo.

### 8.1. Preparation of T Cells Containing Luciferase+/−HLA-A, CIITA, or HLA-A/CIITA KO

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1×10\hat{ }⁶ cells/ml into T cell growth media (TCGM) composed of OpTmizer TCGM as described in Example 3 further supplemented with with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were activated using T cell TransAct™ (Miltenyi Biotec, Cat. 130-111-160) at 1:100 dilution at 37° C. for 24 hours.

Twenty-four hours post activation, 1×10\hat{ }⁶ T cells in 500 μl fresh TCGM without cytokines were transduced by centrifugation 1000×G for 60 minutes at 37° C. with 150 μl of luciferase lentivirus (Imanis Life Sciences, Cat #LV050L). Transduced cells were expanded in 24-well G-Rex plate (Wilson Wolf, Cat. 80192M) in TCGM with cytokines at 3TC for 24 hours.

Forty-eight hours post activation, luciferase LV infected T cells were edited to disrupt the B2M or HLA-A genes. Briefly, LNP compositions containing mRNA encoding cas9 (SEQ ID NO:802) and sgRNA 6019000 (SEQ ID NO: 18) targeting HLA-A were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions containing the Cas9 mRNA and sgRNA G000529 (SEQ ID NO: 245) targeting B2M were formulated as described in Example 1. LNP compositions were incubated in Optmizer TCGM without serum or cytokines further supplemented with 1 ug/ml recombinant human ApoE3 (Peprotech, Cat. 350-02) for 15 minutes at 37° C. T cells were washed and suspended in TCGM with cytokines. Pre-incubated LNP and T cells were mixed to yield final concentrations of 0.5×10\hat{ }⁶ T cells/ml and 2.5 ug total RNA/mL of LNP in TCGM with 5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). An additional group of cells were mock edited with media containing ApoE3 but no LNP compositions. All cells were incubated at 37° C. for 24 hours.

Seventy-two hours post activation, the cells were edited to disrupt CIITA, and LNP were administered either on luciferase and HLA-A edited cells or luciferase cells alone. Briefly, cells were transduced with LNP compositions containing the Cas9 mRNA and sgRNA G013675 (SEQ ID NO: 246) as described for HLA-A editing. LNP compositions targeting CIITA were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. Ninety-six hours post activation, cells were washed and transferred to a 24-well G-Rex. Media with fresh cytokines was replaced every 2 days. On day 15 post activation, edited T cells were sorted on GFP⁺ cells using BD FACS Aria Flow Sorter to enrich for luciferase-expressing cells. For B2M KO luciferase group, cells were sorted on GFP⁺ and MHC-I⁻. Sorted cells were rested overnight in TCGM media with cytokines in a 37° C. incubator. The next day, T cells were re-stimulated with T-cell TrasnAct™ at 1:100 dilution for 24 hours. Twenty-four hours after restimulation, TransAct was washed out and T cells were cultured and maintained in G-Rex plate for 15 days with regular changes in media and cytokines.

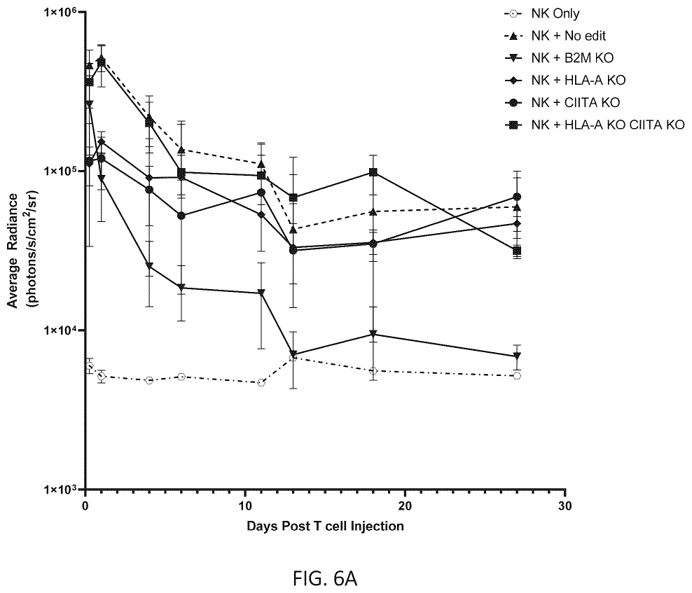

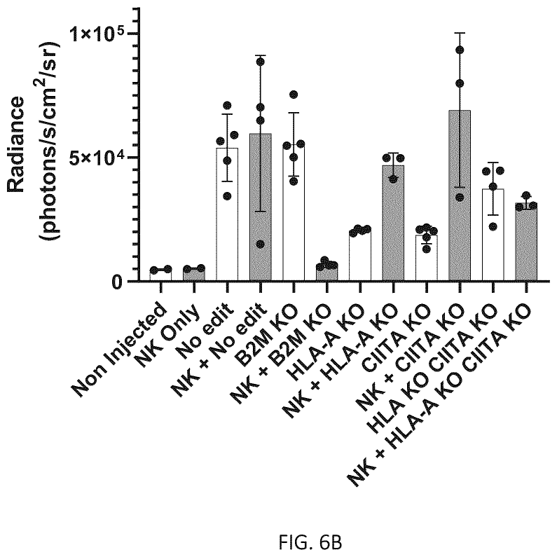

Fifteen days after restimulation, NK cell mediated cytotoxicity towards engineered T cells was assayed in vitro as in Example 4 with the following exceptions. Assays were performed using OpTmizer TCGM with 100 μl/ml IL-2. T cells were co-cultured overnight with the HLA-B/C matched CTV labelled NK cells at effector to target ratios (E:T) of 10:1, 5:1, 2.5:1, 1.25:1 and 0.625:1. The cells were incubated with BrightGlo Luciferase reagents (Promega, Cat. E2620) and processed on the CellTiter Glo Program in ClarioStar to determine lysis of T cells by NK cells based on luciferase signal. Table 19 and FIG. 6A show the percentage of T cell lysis following NK cell challenge. In vitro, B2M edited cells showed sensitivity to NK killing, while HLA-A edited, CIITA edited and HLA-A, CIITA double edited cells showed protection from NK mediated lysis.

TABLE 19
Percentage of lysis of luciferase transduced T cell following NK cell challenge

| | | | | | | | HLA-A KO, | | | | |
|---|---|---|---|---|---|---|---|---|---|---|---|
| | No edit | | HLA-A KO | | CIITA KO | | CIITA KO | | B2M KO | | |
| E:T | Mean | SD | Mean | SD | Mean | SD | Mean | SD | Mean | SD | n |
| 10 | 19.22 | 3.16 | 28.55 | 1.02 | 22.96 | 3.59 | 22.22 | 3.15 | 68.09 | 0.11 | 2 |
| 5 | 13.04 | 1.71 | 27.18 | 4.35 | 22.85 | 6.93 | 13.78 | 4.55 | 53.87 | 3.30 | 2 |
| 2.5 | 1.56 | 1.35 | 26.56 | 3.75 | 26.59 | 2.44 | 21.32 | 0.72 | 39.46 | 7.05 | 2 |
| 1.25 | −0.26 | 1.94 | 19.78 | 3.24 | 19.91 | 5.38 | 12.86 | 0.54 | 25.79 | 7.96 | 2 |
| 0.625 | 8.67 | 6.81 | 25.44 | 0.23 | 18.32 | 4.28 | 19.80 | 7.20 | 29.31 | 2.67 | 2 |
| 0.3125 | 2.96 | 7.66 | 22.40 | 0.83 | 19.13 | 1.34 | 13.34 | 2.48 | 9.32 | 0.84 | 2 |

8.2. HLA-A and CIITA Double Knockout T Cells are Protected from NK Killing

For the in vivo study, NK cells isolated from a leukopak by methods known in the art were washed with HBSS (Gibco, Cat. No. 14025-092) and resuspended at 10×10\hat{ }⁶ cells/mL for injection in 150 μL HBSS. Twenty-two female NOG-hIL-15 mice (Taconic) were dosed by tail vein injection with 1.5×10\hat{ }⁶ isolated NK cells. An addition 27 female NOG-hIL-15 served NK-non-injected controls.

Twenty-eight days after NK cell injection, mice were injected with unedited or engineered T cells as described in Table 19. Briefly, engineered T cells were injected 16 days post second activation after washing in PBS and resuspending in HBSS solution at a concentration of 6×10\hat{ }⁶ cells/150 μL.

IVIS imaging of live mice was performed to identify luciferase-positive T cells by IVIS spectrum. IVIS imaging was done at 6 hours, 24 hours, 48 hours, 8 days, 13 days, 18 days, and 27 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 20 and FIG. 6A shows radiance (photons/s/cm2/sr) from luciferase expressing T cells present at the various time points after injection. FIG. 6B shows radiance (photons/s/cm2/sr) from luciferase expressing T cells present in the various mice groups after 27 days. In vivo, B2M edited cells showed sensitivity to NK killing, while HLA-A edited, CIITA edited and HLA-A, CIITA double edited cells showed protection from NK mediated lysis. Unexpectedly, even after a reduction in one of the three highly polymorphic MHC class I proteins (HLA-A) the cells are protected against NK-mediated rejection.

TABLE 20
Radiance (photons/s/cm2/sr) from luciferase expressing T cells in
treated mice at intervals after T cell injection.

| | Timepoint | No NK cell injection | | | NK cell injection | | |
|---|---|---|---|---|---|---|---|
| T cell injection | (days) | Mean | SD | n | Mean | SD | n |
| No T cells | 0.25 | 5,065 | 474 | 2 | 6,010 | 651 | 2 |
| | 1 | 5,225 | 431 | 2 | 5,150 | 467 | 2 |
| | 4 | 4,715 | 403 | 2 | 4,860 | 57 | 2 |
| | 6 | 5,145 | 884 | 2 | 5,110 | 226 | 2 |
| | 11 | 5,230 | 382 | 2 | 4,700 | 99 | 2 |
| | 13 | 6,920 | 948 | 2 | 6,735 | 35 | 2 |
| | 18 | 5,055 | 148 | 2 | 5,570 | 28 | 2 |
| | 27 | 4,740 | 311 | 2 | 5,185 | 290 | 2 |
| No edit | 0.25 | 477,200 | 51,237 | 5 | 464,000 | 112,493 | 4 |
| | 1 | 547,600 | 59,315 | 5 | 517,500 | 95,710 | 4 |
| | 4 | 285,600 | 43,328 | 5 | 219,750 | 77,298 | 4 |
| | 6 | 249,400 | 58,748 | 5 | 137,000 | 69,190 | 4 |
| | 11 | 131,500 | 28,671 | 5 | 111,150 | 36,287 | 4 |
| | 13 | 147,000 | 15,732 | 5 | 43,168 | 52,128 | 4 |
| | 18 | 112,100 | 20,768 | 5 | 55,825 | 47,391 | 4 |
| | 27 | 53,960 | 13,546 | 5 | 59,700 | 31,479 | 4 |
| B2M KO | 0.25 | 662,600 | 193,865 | 5 | 261,850 | 135,636 | 4 |
| | 1 | 555,200 | 122,508 | 5 | 89,400 | 41,151 | 4 |
| | 4 | 266,200 | 68,845 | 5 | 25,175 | 11,072 | 4 |
| | 6 | 202,600 | 41,825 | 5 | 18,500 | 7,048 | 4 |
| | 11 | 106,320 | 14,377 | 5 | 17,100 | 9,440 | 4 |
| | 13 | 57,714 | 45,535 | 5 | 7,048 | 2,735 | 4 |
| | 18 | 77,080 | 7,792 | 5 | 9,453 | 4,592 | 4 |
| | 27 | 55,240 | 12,780 | 5 | 6,860 | 1,207 | 4 |
| HLA-A KO | 0.25 | 160,000 | 30,315 | 5 | 111,500 | 30,533 | 4 |
| | 1 | 206,800 | 38,493 | 5 | 153,000 | 24,427 | 4 |
| | 4 | 120,200 | 23,488 | 5 | 91,025 | 69,091 | 4 |
| | 6 | 81,100 | 16,903 | 5 | 91,408 | 106,141 | 4 |
| | 11 | 55,520 | 6,843 | 5 | 53,367 | 21,985 | 3 |
| | 13 | 30,716 | 23,658 | 5 | 33,233 | 13,615 | 3 |
| | 18 | 21,802 | 10,911 | 5 | 35,667 | 5,601 | 3 |
| | 27 | 20,600 | 808 | 4 | 46,900 | 4,937 | 3 |
| CIITA KO | 0.25 | 121,400 | 19,680 | 5 | 116,350 | 82,606 | 4 |
| | 1 | 168,200 | 32,760 | 5 | 120,225 | 43,535 | 4 |
| | 4 | 93,600 | 23,187 | 5 | 76,450 | 31,056 | 4 |
| | 6 | 71,298 | 40,161 | 5 | 52,500 | 35,590 | 4 |
| | 11 | 59,100 | 13,805 | 5 | 73,500 | 77,242 | 4 |
| | 13 | 43,870 | 22,810 | 5 | 31,760 | 30,831 | 4 |
| | 18 | 28,422 | 14,019 | 5 | 35,000 | 7,902 | 3 |
| | 27 | 18,780 | 3,505 | 5 | 69,067 | 31,194 | 3 |
| HLA-A KO | 0.25 | 259,250 | 59,824 | 4 | 363,000 | 113,731 | 4 |
| CIITA KO | 1 | 456,750 | 69,188 | 4 | 481,500 | 142,778 | 4 |
| | 4 | 170,500 | 26,665 | 4 | 200,750 | 70,415 | 4 |
| | 6 | 108,950 | 11,046 | 4 | 98,633 | 27,450 | 3 |
| | 11 | 97,350 | 19,982 | 4 | 93,867 | 32,173 | 3 |
| | 13 | 85,708 | 58,720 | 4 | 68,357 | 54,428 | 3 |
| | 18 | 20,923 | 22,172 | 4 | 98,633 | 27,450 | 3 |
| | 27 | 37,375 | 10,602 | 4 | 31,733 | 2,593 | 3 |

### Example 9: HLA-A and CIITA Partial-Matching in an NK Cell In Vivo Killing Mouse Model

Female NOG-hIL-15 mice were engrafted with 1.5×10\hat{ }⁶ primary NK cells followed by the injection of engineered T cells containing luciferase+/−HLA-A/CIITA KO with HD1 TCR 4 weeks later in order to determine 1) whether engrafted NK cells can readily lyse control T cells (B2M^{-/-}), and 2) whether the addition of a partial-matching edit (HLA-A & CIITA) provides a protective effect for T cells with the exogenous HD1 TCR from NK cell lysis in vivo.

### 9.1. Preparation of T Cells Containing Luciferase+/−HLA-A/CIITA KO and HD1 TCR

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride red blood cell lysis buffer (Stemcell Technologies; Cat. 07800) for 15 minutes to lyse red blood cells. Peripheral blood mononuclear cell (PBMC) count was determined post lysis, and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10\hat{ }⁶ cells/ml into T cell activation media (TCAM) composed of OpTmizer TCGM as described in Example 3 and further supplemented with 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). Cells were rested at 37° C. for 24 hours.

Twenty-four hours post thawing, T cells were counted and resuspended at 2×10\hat{ }⁶ cells/ml in TCAM media and 1:50 of Transact was added. Cells were mixed and incubated for 20-30 mins at 37° C. LNP compositions containing mRNA encoding Cas9 (SEQ ID NO:802) and sgRNA G013675 (SEQ ID NO: 246), targeting CIITA were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with ug/ml recombinant human ApoE3 (Peprotech, Cat. 350-02) for 15 minutes at 37° C. Pre-incubated LNP compositions and T cells with Transact were mixed to yield final concentrations of 1×10\hat{ }⁶ T cells/ml and 2.5 μg total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15). An additional group of cells were mock-edited with media containing ApoE3 but no LNP compositions. All cells were incubated at 37° C. for 24 hours.

After 48 hours post activation, all groups were transduced with EFla-GFP-Luc lentivirus. Lentivirus was removed from −80° C. and thawed on ice. Cells were collected as per groups and centrifuged at 500×g for 5 mins to wash off the LNP compositions and media. Cells were resuspended, individually according to their groups, at 2×10\hat{ }⁶ cells/ml in TCAM media. 500 ul of the cell suspension was then transferred to a sterile Eppendorf tube (total 1×10\hat{ }⁶ cells), and 100 ul of lentivirus was added. Cells were centrifuged at 1000×G for minutes at 37° C. After centrifugation, the cells were combined according to their groups and resuspended at 1×10\hat{ }⁶ cells/ml of TCAM media containing final concentration of 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15) followed by incubating at 37° C. for 24 hours.

Seventy-two hours post activation, luciferase-transduced T cells were treated with LNP compositions to disrupt TRAC genes and further treated with HD1 AAV to insert the HD1 TCR at the TRAC locus. Cells were collected as per groups and centrifuged at 500×g for 5 mins to wash off the lentivirus and media. The cells were then resuspended in TCAM media at 1×10\hat{ }⁶ cells/ml in TCAM media. LNP compositions containing mRNA encoding Cas9 (SEQ ID NO:802) and sgRNA G013006 (SEQ ID NO: 243), targeting TRAC were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with 5 ug/ml recombinant human ApoE3 (Peprotech, Cat. 350-02) for 15 minutes at 37° C. Pre-incubated LNP compositions and T cells with Transact were mixed to yield final concentrations of 1×10\hat{ }⁶ T cells/ml and 2.5 μg total RNA/mL of LNP in TCAM with 2.5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15). A vial of EF1α-HD1 AAV was thawed on benchtop and added to the TRAC LNP treated cells at 3×10\hat{ }⁵ GC/cell. Cells were then incubated at 37° C. for 24 hours.

Ninety-six hours post activation cells were then treated for a final round of editing either with TRBC LNP alone or in combination with HLA-A LNP. The B2M KO group was treated with B2M LNP. Cells were collected as per groups and centrifuged at 500×g for 5 mins to wash off the LNP compositions and media. The cells were then resuspended in TCAM media at 1×10\hat{ }⁶ cells/ml in TCAM media. Briefly, LNP compositions containing mRNA encoding Cas9 (SEQ ID NO:802) and sgRNA G018995 (sgRNA comprising SEQ ID NO: 13, as shown in Table 2) targeting HLA-A were formulated as described in Example 1. LNP compositions containing the Cas9 mRNA and sgRNA G000529 (SEQ ID NO: 245) targeting B2M and LNP compositions containing the Cas9 mRNA and sgRNA G016239 (SEQ ID NO: 247) targeting TRBC were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM and further supplemented with 5 ug/ml recombinant human ApoE3 (Peprotech, Cat. 350-02) for 15 minutes at 37° C. Pre-incubated LNP compositions and T cells with Transact were mixed to yield final concentrations of 1×10\hat{ }⁶ T cells/ml and 2.5 μg total RNA/mL of LNP in TCAM with 2.5% human AB serum, 100 U/mL of recombinant human interleukin-2 (Peprotech, Cat. 200-02), 5 ng/ml IL-7 (Peprotech, Cat. 200-07), and IL-15 (Peprotech, Cat. 200-15). For simultaneous TRBC and HLA-A editing, LNP and ApoE3 were formulated at 4× the final concentration followed by adding TRBC LNP first to the T cells and incubating at 37° C. for 15 mins. After incubation preformulated HLA-A LNP compositions were added, the cells were incubated for 24 hours.

After the final round of editing, the cells were washed by spinning at 500XG for 5 mins and resuspended in TCGM media containing with 5% human AB serum, 100 U/mL of recombinant human interleukin-2 (Peprotech, Cat. 200-02), 5 ng/ml IL-7 (Peprotech, Cat. 200-07), and 5 ng/ml IL-15 (Peprotech, Cat. 200-15).

On day 5 post activation, edited T cells were sorted on GFP⁺ cells using a BD FACS Aria Flow Sorter to enrich for luciferase-expressing cells. Sorted cells were rested overnight in TCGM media with cytokines in a 37° C. incubator. The next day, T cells were re-stimulated with T-cell TransAct™ at 1:100 dilution for 24 hours. Twenty-four hours after restimulation, TransAct™ was washed out and T cells were cultured and maintained in G-Rex plate for 15 days with regular changes in media and cytokines.

Fifteen days after first restimulation, editing levels were confirmed via flow cytometry, and cells were washed and resuspend in HBSS buffer for injections.

9.2. HLA-A and CIITA Double Knockout T Cells Show Protection from NK Killing

For the in vivo study, NK cells isolated from a leukopak by methods known in the art were washed with HBSS (Gibco, Cat. No. 14025-092) and resuspended at 10×10\hat{ }⁶ cells/mL for injection in 150 μL HBSS. Thirty female NOG-hIL-15 mice (Taconic) were dosed by tail vein injection with 1.5×10\hat{ }⁶ isolated NK cells. An addition 25 female NOG-hIL-15 served as NK-non-injected controls.

Twenty-eight days after NK cell injection, mice were injected with unedited or engineered T cells as described in Table 21. Briefly, 0.2×10\hat{ }⁶ engineered T cells were injected 16 days post second activation after washing in PBS and resuspending in HBSS solution at a concentration of 6.0×10\hat{ }⁶ cells/150 μL.

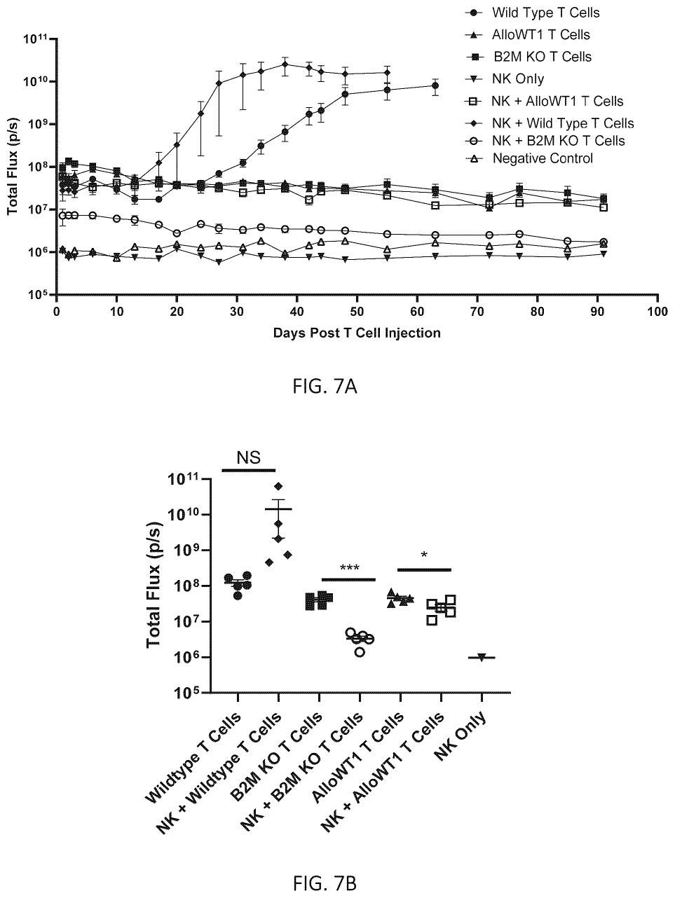

IVIS imaging of live mice was performed to identify luciferase-positive T cells by IVIS spectrum. IVIS imaging was done at 24 hours, 48 hours, 72 hours, 6 days, 10 days, 13 days, 17 days, 20 days, 24 days, 27 days, 31 days, 34 days, 38 days, 42 days, 44 days, 48 days, 55 days, 63 days, 72 days, 77 days, 85 days, and 91 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 22 and FIG. 7A shows radiance (photons/s/cm2/sr) from luciferase expressing T cells present at the various time points after injection out to 91 days. FIG. 7B shows radiance (photons/s/cm²/sr) from luciferase expressing T cells present in the various mice groups after 31 days. In vivo, B2M edited cells showed sensitivity to NK killing, while the HLA-A, CIITA double edited cells showed protection from NK mediated lysis.

TABLE 21
T-Cell Engineering

| | | | | | | | | | Day |
|---|---|---|---|---|---|---|---|---|---|
| Group | Day 0 | Day 1 | Day2 | Day3 | Day4 | Day6 | Day 7 | Day 8 | 16 |
| HLA-A | Thaw | CIITA | GFP- | TRAC + AAV | TRBC, | Flow | Re-stim | Expand | Wash |
| CIITA | | | Luc | | HLA-A | & | | in G-Rex | & |
| KO | | | LV | | | Sort | | | Inject |
| B2M | Thaw | B2M | GFP- | TRAC + AAV | TRBC | Flow | Re-stim | Expand | Wash |
| Control | | | Luc | | | & | | in G-Rex | & |
| | | | LV | | | Sort | | | Inject |
| No Edit | Thaw | — | GFP- | — | — | Flow | Re-stim | Expand | Wash |
| | | | Luc | | | & | | in G-Rex | & |
| | | | LV | | | Sort | | | Inject |

TABLE 22
Total Flux (photons/s) from luciferase expressing T cells in treated mice at intervals after
T cell injection.

| T cell | Timepoint | No NK cell injection | | | NK cell injection | | |
|---|---|---|---|---|---|---|---|
| injection | (days) | Mean | SD | n | Mean | SD | n |
| No T cells | 1 | 1170000 | 0 | 1 | 1060000 | 0 | 1 |
| | 2 | 884000 | 0 | 1 | 728000 | 0 | 1 |
| | 3 | 1090000 | 0 | 1 | 771000 | 0 | 1 |
| | 6 | 1040000 | 0 | 1 | 888000 | 0 | 1 |
| | 10 | 741000 | 0 | 1 | 799000 | 0 | 1 |
| | 13 | 1350000 | 0 | 1 | 751000 | 0 | 1 |
| | 17 | 1210000 | 0 | 1 | 709000 | 0 | 1 |
| | 20 | 1530000 | 0 | 1 | 1190000 | 0 | 1 |
| | 24 | 1280000 | 0 | 1 | 823000 | 0 | 1 |
| | 27 | 1430000 | 0 | 1 | 577000 | 0 | 1 |
| | 31 | 1310000 | 0 | 1 | 970000 | 0 | 1 |
| | 34 | 1840000 | 0 | 1 | 800000 | 0 | 1 |
| | 38 | 937000 | 0 | 1 | 750000 | 0 | 1 |
| | 42 | 1450000 | 0 | 1 | 757000 | 0 | 1 |
| | 44 | 1770000 | 0 | 1 | 797000 | 0 | 1 |
| | 48 | 1850000 | 0 | 1 | 666000 | 0 | 1 |
| | 55 | 1170000 | 0 | 1 | 723000 | 0 | 1 |
| | 63 | 1680000 | 0 | 1 | 799000 | 0 | 1 |
| | 72 | 1400000 | 0 | 1 | 840000 | 0 | 1 |
| | 77 | 1570000 | 0 | 1 | 801000 | 0 | 1 |
| | 85 | 1220000 | 0 | 1 | 770000 | 0 | 1 |
| | 91 | 1580000 | 0 | 1 | 905000 | 0 | 1 |
| No edit | 1 | 37560000 | 34014482.9 | 5 | 27882000 | 27141262.31 | 5 |
| | 2 | 40698000 | 22307084.5 | 5 | 28640000 | 14568047.23 | 5 |
| | 3 | 34210000 | 18847559.5 | 5 | 25692000 | 14362636.25 | 5 |
| | 6 | 51440000 | 10855551.6 | 5 | 37700000 | 34510288.32 | 5 |
| | 10 | 29460000 | 5028220.36 | 5 | 34060000 | 24420544.63 | 5 |
| | 13 | 17350000 | 8731122.49 | 5 | 42864000 | 47552123.82 | 5 |
| | 17 | 17380000 | 4065956.22 | 5 | 124180000 | 217126534.5 | 5 |
| | 20 | 35860000 | 9912012.91 | 5 | 329720000 | 644006666.9 | 5 |
| | 24 | 41400000 | 6393355.93 | 5 | 1784780000 | 3583692731 | 5 |
| | 27 | 70500000 | 28116809.9 | 5 | 9112600000 | 19172106869 | 5 |
| | 31 | 124260000 | 57196923 | 5 | 14383000000 | 27254468202 | 5 |
| | 34 | 313000000 | 256943574 | 5 | 17450000000 | 24859612829 | 5 |
| | 38 | 667800000 | 614512978 | 5 | 25316000000 | 26111305597 | 5 |
| | 42 | 1727400000 | 1703225998 | 5 | 21084000000 | 16956611690 | 5 |
| | 44 | 2101400000 | 2213844349 | 5 | 16975000000 | 13721121188 | 4 |
| | 48 | 5068000000 | 4995313854 | 5 | 15106666667 | 11613532337 | 3 |
| | 55 | 6386750000 | 5350377767 | 4 | 16303333333 | 11913187371 | 3 |
| | 63 | 8105750000 | 6722716632 | 4 | | | |
| | 72 | | | | | | |
| | 77 | | | | | | |
| | 85 | | | | | | |
| | 91 | | | | | | |
| B2M KO | 1 | 96334000 | 62882587.3 | 5 | 7192000 | 6901425.215 | 5 |
| | 2 | 138300000 | 57619007.3 | 5 | 7296000 | 2213194.524 | 5 |
| | 3 | 117980000 | 43943736.8 | 5 | 7342000 | 2837475.991 | 5 |
| | 6 | 104240000 | 34772230.3 | 5 | 7276000 | 2743998.907 | 5 |
| | 10 | 81120000 | 19876921.3 | 5 | 6124000 | 1967035.841 | 5 |
| | 13 | 45386000 | 24729233.3 | 5 | 5748000 | 3248448.861 | 5 |
| | 17 | 50600000 | 19718899.6 | 5 | 4390000 | 902607.3343 | 5 |
| | 20 | 38200000 | 12211470 | 5 | 2772000 | 947507.2559 | 5 |
| | 24 | 32180000 | 17561520.4 | 5 | 4566000 | 1182742.576 | 5 |
| | 27 | 35840000 | 15497354.6 | 5 | 3626000 | 1995903.304 | 5 |
| | 31 | 41380000 | 12243243 | 5 | 3344000 | 1295812.486 | 5 |
| | 34 | 40740000 | 13481394.6 | 5 | 3864000 | 506635.964 | 5 |
| | 38 | 33980000 | 15116117.2 | 5 | 3468000 | 1330139.09 | 5 |
| | 42 | 38840000 | 15452605 | 5 | 3504000 | 688534.676 | 5 |
| | 44 | 35280000 | 19116929.7 | 5 | 3266000 | 910291.1622 | 5 |
| | 48 | 31600000 | 17624982.3 | 5 | 3196000 | 726691.1311 | 5 |
| | 55 | 38920000 | 30824779 | 5 | 2654000 | 475794.0731 | 5 |
| | 63 | 29300000 | 22330584.4 | 5 | 2530000 | 274135.0032 | 5 |
| | 72 | 19070000 | 13309188.6 | 5 | 2522000 | 437344.258 | 5 |
| | 77 | 30680000 | 24960508.8 | 5 | 2650000 | 531554.3246 | 5 |
| | 85 | 24738000 | 22937833.8 | 5 | 1816000 | 410524.0553 | 5 |
| | 91 | 18234000 | 10913394.5 | 5 | 1736000 | 297707.9105 | 5 |
| HLA-A KO | 1 | 63960000 | 33085918.5 | 5 | 59320000 | 32265414.92 | 5 |
| CIITA KO | 2 | 55412000 | 31461432.3 | 5 | 49560000 | 9862707.539 | 5 |
| | 3 | 64686000 | 39918742.2 | 5 | 41264000 | 22521777.9 | 5 |
| | 6 | 88440000 | 22053865.9 | 5 | 33442000 | 18099663.53 | 5 |
| | 10 | 68320000 | 18250397.3 | 5 | 42040000 | 4585084.514 | 5 |
| | 13 | 57880000 | 8452041.17 | 5 | 37028000 | 20443236.53 | 5 |
| | 17 | 39320000 | 11283040.4 | 5 | 41400000 | 10968135.67 | 5 |
| | 20 | 40480000 | 12259363.8 | 5 | 37540000 | 8371260.359 | 5 |
| | 24 | 39900000 | 18287017.3 | 5 | 37740000 | 9070446.516 | 5 |
| | 27 | 37800000 | 14406422.2 | 5 | 31840000 | 11387185.78 | 5 |
| | 31 | 46160000 | 13751836.2 | 5 | 25020000 | 11377477.75 | 5 |
| | 34 | 39820000 | 8990383.75 | 5 | 28980000 | 5348551.206 | 5 |
| | 38 | 42620000 | 8249363.61 | 5 | 31000000 | 7146677.55 | 5 |
| | 42 | 30740000 | 10083798.9 | 5 | 16928000 | 9138868.639 | 5 |
| | 44 | 31740000 | 9619667.35 | 5 | 26580000 | 7343500.528 | 5 |
| | 48 | 30740000 | 9147021.37 | 5 | 28620000 | 3141178.123 | 5 |
| | 55 | 27600000 | 5482244.07 | 5 | 21340000 | 3673281.911 | 5 |
| | 63 | 24820000 | 6599015.08 | 5 | 12428000 | 3646082.83 | 5 |
| | 72 | 10918000 | 3813609.84 | 5 | 13094000 | 3349355.162 | 5 |
| | 77 | 24840000 | 4728953.37 | 5 | 14200000 | 3801973.172 | 5 |
| | 85 | 15520000 | 4283923.44 | 5 | 14580000 | 2920102.738 | 5 |
| | 91 | 17260000 | 5452797.45 | 5 | 11256000 | 2456141.283 | 5 |

### Example 10: MHCI and MHCII KO In-Vivo Efficacy of HD1 T Cells

Female NOG-hIL-15 mice were engrafted with 0.2×10\hat{ }⁶ human acute lymphoblastic leukemia (ALL) cell line 697-Luc2, followed by the injection of 10×10\hat{ }⁶ engineered T cells with various edits in order to determine whether the edits provide a specific anti-tumor effect. Groups of T cells studied include: a control group of T cells with no edits (697 only); T cells with edits in TRAC and TRBC (TCR KO); T cells with edits in TRAC and TRBC and insertion of HD1 (TCR KO/WT1 insert); T cells with edits in TRAC and TRBC, insertion of HD1, and disruption in HLA-A (HLA-A KO); T cells with edits in TRAC and TRBC, insertion of HD1, and edits in HLA-A and in CIITA (AlloWT1); and T cells with edits in TRAC and TRBC and insertion of HD1 in the presence of a DNA PKi compound, and edits in HLA-A and in CIITA (AlloWT1+PKi Compound 1).

### 10.1. T Cell Preparation

T cells from HLA-A2+ donor (110046967) were isolated from the leuokopheresis products of healthy donor (STEMCELL Technologies). T cells were isolated using EasySep Human T cell isolation kit (STEMCELL Technologies, Cat #17951) following manufacturer's protocol and cryopreserved using Cryostor CS10 (STEMCELL Technologies, Cat #07930). The day before initiating T cell editing, cells were thawed and rested overnight in T cell activation media TCAM: CTS OpTmizer (Thermofisher #A3705001) supplemented with 2.5% human AB serum (Gemini #100-512), 1× GlutaMAX (Thermofisher #35050061), HEPES (Thermofisher #15630080), 200 U/mL IL-2 (Peprotech #200-02), IL-7 (Peprotech #200-07), IL-15 (Peprotech #200-15).

10.2 Multi-Editing T Cells with Sequential LNP Delivery

T cells were prepared by treating healthy donor cells sequentially with four LNP compositions co-formulated with Cas9 mRNA and sgRNA targeting either TRAC, TRBC, CIITA, and HLA-A. The lipid portion of the LNP compositions included Lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. A transgenic WT1-targeting TCR was site-specifically integrated into the TRAC cut site by delivering a homology-directed repair template using AAV indicated in Table 24, in combination with the small molecule inhibitor of DNA-dependent protein kinase to boost the tgTCR insertion rate. The inhibitor, referred to hereinafter as “DNAPKI Compound 1” is 9-(4,4-difluorocyclohexyl)-7-methyl-2-((7-methyl-[1,2,4]triazolo[1,5-a]pyridin-6-yl)amino)-7,9-dihydro-8H-purin-8-one, also depicted as:

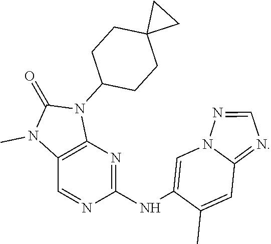 [SMILES: Cc1cc2ncnn2cc1Nc1ncc2c(n1)n(C1CCC3(CC1)CC3)c(=O)n2C]

DNAPKI Compound 1 was prepared as follows:

General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl3 at ambient temperature. Chemical shifts are reported in parts per million (ppm) relative to CDCl3 (7.26). Data for 1H NMR are reported as follows: chemical shift, multiplicity (br=broad, s=singlet, d=doublet, t=triplet, q=quartet, dd=doublet of doublets, dt=doublet of triplets m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Example 1—Compound 1

Intermediate 1a: (E)-N,N-dimethyl-N′-(4-methyl-5-nitropyridin-2-yl)formimidamide

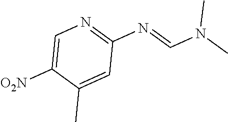

To a solution of 4-methyl-5-nitro-pyridin-2-amine (5 g, 1.0 equiv.) in toluene (0.3 M) was added DMF-DMA (3.0 equiv.). The mixture was stirred at 110° C. for 2 h. The reaction mixture was concentrated under reduced pressure to give a residue and purified by column chromatography to afford product as a yellow solid (59%). ¹H NMR (400 MHz, (CD₃)₂SO) δ 8.82 (s, 1H), 8.63 (s, 1H), 6.74 (s, 1H), 3.21 (m, 6H).

Intermediate 1b: (E)-N-hydroxy-N′-(4-methyl-5-nitropyridin-2-yl)formimidamide

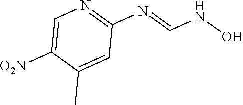

To a solution of Intermediate 1a (4 g, 1.0 equiv.) in MeOH (0.2 M) was added NH₂OH·HCl (2.0 equiv.). The reaction mixture was stirred at 80° C. for 1 h. The reaction mixture was filtered, and the filtrate was concentrated under reduced pressure to give a residue. The residue was partitioned between H₂O and EtOAc, followed by 2× extraction with EtOAc. The organic phases were concentrated under reduced pressure to give a residue and purified by column chromatography to afford product as a white solid (66%). 1H NMR (400 MHz, (CD₃)₂SO) δ 10.52 (d, J=3.8 Hz, 1H), 10.08 (dd, J=9.9, 3.7 Hz, 1H), 8.84 (d, J=3.8 Hz, 1H), 7.85 (dd, J=9.7, 3.8 Hz, 1H), 7.01 (d, J=3.9 Hz, 1H), 3.36 (s, 3H).

Intermediate 1c: 7-methyl-6-nitro-[1,2,4]triazolo[1,5-a]pyridine

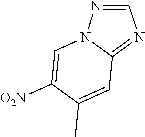

To a solution of Intermediate 1b (2.5 g, 1.0 equiv.) in THF (0.4 M) was added trifluoroacetic anhydride (1.0 equiv.) at 0° C. The mixture was stirred at 25° C. for 18 h. The reaction mixture was filtered, and the filtrate was concentrated under reduced pressure to give a residue. The residue was purified by column chromatography to afford product as a white solid (44%). ¹H NMR (400 MHz, CDCl₃) δ 9.53 (s, 1H), 8.49 (s, 1H), 7.69 (s, 1H), 2.78 (d, J=1.0 Hz, 3H).

Intermediate 1d: 7-methyl-[1,2,4]triazolo[1,5-a]pyridin-6-amine

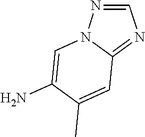

To a mixture of Pd/C (10% w/w, 0.2 equiv.) in EtOH (0.1 M) was added Intermediate 1c (1.0 equiv. and ammonium formate (5.0 equiv.). The mixture was heated at 105° C. for 2 h. The reaction mixture was filtered, and the filtrate was concentrated under reduced pressure to give a residue. The residue was purified by column chromatography to afford product as a pale brown solid. ¹H NMR (400 MHz, (CD₃)₂SO) δ 8.41 (s, 2H), 8.07 (d, J=9.0 Hz, 2H), 7.43 (s, 1H), 2.22 (s, 3H).

Intermediate 1e: 8-methylene-1,4-dioxaspiro[4.5]decane

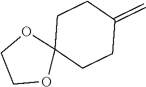

To a solution of methyl(triphenyl)phosphonium bromide (1.15 equiv.) in THF (0.6 M) was added n-BuLi (1.1 equiv.) at −78° C. dropwise, and the mixture was stirred at 0° C. for 1 h. Then, 1,4-dioxaspiro[4.5]decan-8-one (50 g, 1.0 equiv.) was added to the reaction mixture. The mixture was stirred at 25° C. for 12 h. The reaction mixture was poured into aq. NH₄Cl at 0° C., diluted with H₂O, and extracted 3× with EtOAc. The combined organic layers were concentrated under reduced pressure to give a residue and purified by column chromatography to afford product as a colorless oil (51%). ¹H NMR (400 MHz, CDCl₃) δ 4.67 (s, 1H), 3.96 (s, 4H), 2.82 (t, J=6.4 Hz, 4H), 1.70 (t, J=6.4 Hz, 4H).

Intermediate 1 f: 7,10-dioxadispiro[2.2.4⁶.2³]dodecane

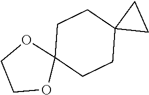 [SMILES: C1COC2(CCC3(CC3)CC2)O1]

To a solution of Intermediate 4a (5 g, 1.0 equiv.) in toluene (3 M) was added ZnEt₂ (2.57 equiv.) dropwise at −40° C. and the mixture was stirred at −40° C. for 1 h. Then diiodomethane (6.0 equiv.) was added dropwise to the mixture at −40° C. under N₂. The mixture was then stirred at 20° C. for 17 h under N₂ atmosphere. The reaction mixture was poured into aq. NH₄Cl at 0° C. and extracted 2× with EtOAc. The combined organic phases were washed with brine (20 mL), dried with anhydrous Na₂SO₄, filtered, and the filtrate was concentrated in vacuum. The residue was purified by column chromatography to afford product as a pale yellow oil (73%).

Intermediate 1g: spiro[2.5]octan-6-one

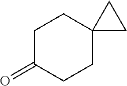

To a solution of Intermediate 4b (4 g, 1.0 equiv.) in 1:1 THF/H₂O (1.0 M) was added TFA (3.0 equiv.). The mixture was stirred at 20° C. for 2 h under N₂ atmosphere. The reaction mixture was concentrated under reduced pressure to remove THF, and the residue adjusted pH to 7 with 2 M NaOH (aq.). The mixture was poured into water and 3× extracted with EtOAc. The combined organic phase was washed with brine, dried with anhydrous Na₂SO₄, filtered, and the filtrate was concentrated in vacuum. The residue was purified by column chromatography to afford product as a pale yellow oil (68%). ¹H NMR (400 MHz, CDCl₃) δ 2.35 (t, J=6.6 Hz, 4H), 1.62 (t, J=6.6 Hz, 4H), 0.42 (s, 4H).

Intermediate 1h: N-(4-methoxy benzyl)spiro[2.5]octan-6-amine

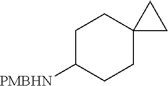

To a mixture of Intermediate 4c (2 g, 1.0 equiv.) and (4-methoxyphenyl)methanamine (1.1 equiv.) in DCM (0.3 M) was added AcOH (1.3 equiv.). The mixture was stirred at 20° C. for 1 h under N₂ atmosphere. Then, NaBH(OAc)₃ (3.3 equiv.) was added to the mixture at 0° C., and the mixture was stirred at 20° C. for 17 h under N₂ atmosphere. The reaction mixture was concentrated under reduced pressure to remove DCM, and the resulting residue was diluted with H₂O and extracted 3× with EtOAc. The combined organic layers were washed with brine, dried over Na₂SO₄, filtered, and the filtrate was concentrated under reduced pressure to give a residue. The residue was purified by column chromatography to afford product as a gray solid (51%). ¹H NMR (400 MHz, (CD₃)₂SO) δ 7.15-7.07 (m, 2H), 6.77-6.68 (m, 2H), 3.58 (s, 3H), 3.54 (s, 2H), 2.30 (ddt, J=10.1, 7.3, 3.7 Hz, 1H), 1.69-1.62 (m, 2H), 1.37 (td, J=12.6, 3.5 Hz, 2H), 1.12-1.02 (m, 2H), 0.87-0.78 (m, 2H), 0.13-0.04 (m, 2H).

Intermediate 1i: spiro[2.5]octan-6-amine

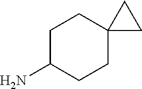

To a suspension of Pd/C (10% w/w, 1.0 equiv.) in MeOH (0.25 M) was added Intermediate 4d (2 g, 1.0 equiv.) and the mixture was stirred at 80° C. at 50 Psi for 24 h under Hz atmosphere. The reaction mixture was filtered, and the filtrate was concentrated under reduced pressure to give a residue that was purified by column chromatography to afford product as a white solid. ¹H NMR (400 MHz, (CD₃)₂SO) δ 2.61 (tt, J=10.8, 3.9 Hz, 1H), 1.63 (ddd, J=9.6, 5.1, 2.2 Hz, 2H), 1.47 (td, J=12.8, 3.5 Hz, 2H), 1.21-1.06 (m, 2H), 0.82-0.72 (m, 2H), 0.14-0.05 (m, 2H).

Intermediate 1j: ethyl 2-chloro-4-(spiro[2.5]octan-6-ylamino)pyrimidine-5-carboxylate

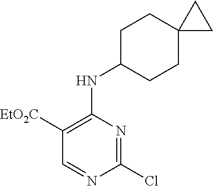 [SMILES: CCOC(=O)c1cnc(Cl)nc1NC1CCC2(CC1)CC2]

To a mixture of ethyl 2,4-dichloropyrimidine-5-carboxylate (2.7 g, 1.0 equiv.) and Intermediate 1i (1.0 equiv.) in ACN (0.5-0.6 M) was added K₂CO₃ (2.5 equiv.) in one portion under N₂. The mixture was stirred at 20° C. for 12 h. The reaction mixture was filtered, and the filtrate was concentrated under reduced pressure to give a residue. The residue was purified by column chromatography to afford product as a white solid (54%). ¹H NMR (400 MHz, (CD₃)₂SO) δ 8.64 (s, 1H), 8.41 (d, J=7.9 Hz, 1H), 4.33 (q, J=7.1 Hz, 2H), 4.08 (d, J=9.8 Hz, 1H), 1.90 (dd, J=12.7, 4.8 Hz, 2H), 1.64 (t, J=12.3 Hz, 2H), 1.52 (q, J=10.7, 9.1 Hz, 2H), 1.33 (t, J=7.1 Hz, 3H), 1.12 (d, J=13.0 Hz, 2H), 0.40-0.21 (m, 4H).

Intermediate 1k: 2-chloro-4-(spiro[2.5]octan-6-ylamino)pyrimidine-5-carboxylic acid

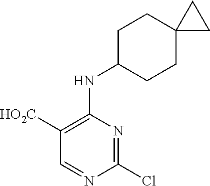 [SMILES: O=C(O)c1cnc(Cl)nc1NC1CCC2(CC1)CC2]

To a solution of Intermediate 1j (2 g, 1.0 equiv.) in 1:1 THF/H₂O (0.3 M) was added LiOH (2.0 equiv.). The mixture was stirred at 20° C. for 12 h. The reaction mixture was filtered, and the filtrate was concentrated under reduced pressure to give a residue. The residue was adjusted to pH 2 with 2 M HCl, and the precipitate was collected by filtration, washed with water, and tried under vacuum. Product was used directly in the next step without additional purification (82%). ¹H NMR (400 MHz, (CD₃)₂SO) δ 13.54 (s, 1H), 8.38 (d, J=8.0 Hz, 1H), 8.35 (s, 1H), 3.82 (qt, J=8.2, 3.7 Hz, 1H), 1.66 (dq, J=12.8, 4.1 Hz, 2H), 1.47-1.34 (m, 2H), 1.33-1.20 (m, 2H), 0.86 (dt, J=13.6, 4.2 Hz, 2H), 0.08 (dd, J=8.3, 4.8 Hz, 4H).

Intermediate 1l: 2-chloro-9-(spiro[2.5]octan-6-yl)-7,9-dihydro-8H-purin-8-one

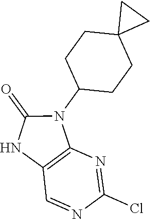 [SMILES: O=c1[nH]c2cnc(Cl)nc2n1C1CCC2(CC1)CC2]

To a mixture of Intermediate 1k (1.5 g, 1.0 equiv.) and Et3N (1.0 equiv.) in DMF (0.3 M) was added DPPA (1.0 equiv.). The mixture was stirred at 120° C. for 8 h under N₂ atmosphere. The reaction mixture was poured into water. The precipitate was collected by filtration, washed with water, and dried under vacuum to give a residue that was used directly in the next step without additional purification (67%). ¹H NMR (400 MHz, (CD₃)₂SO) δ 11.68 (s, 1H), 8.18 (s, 1H), 4.26 (ddt, J=12.3, 7.5, 3.7 Hz, 1H), 2.42 (qd, J=12.6, 3.7 Hz, 2H), 1.95 (td, J=13.3, 3.5 Hz, 2H), 1.82-1.69 (m, 2H), 1.08-0.95 (m, 2H), 0.39 (tdq, J=11.6, 8.7, 4.2, 3.5 Hz, 4H).

Intermediate 1m: 2-chloro-7-methyl-9-(spiro[2.5]octan-6-yl)-7,9-dihydro-8H-purin-8-one

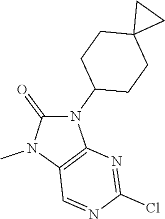 [SMILES: Cn1c(=O)n(C2CCC3(CC2)CC3)c2nc(Cl)ncc21]

To a mixture of Intermediate 11 (1.0 g, 1.0 equiv.) and NaOH (5.0 equiv.) in 1:1 THF/H₂O (0.3-0.5 M) was added MeI (2.0 equiv.). The mixture was stirred at 20° C. for 12 h under N₂ atmosphere. The reaction mixture was concentrated under reduced pressure to afford a residue that was purified by column chromatography to afford product as a pale yellow solid (67%). ¹H NMR (400 MHz, CDCl₃) δ 7.57 (s, 1H), 4.03 (tt, J=12.5, 3.9 Hz, 1H), 3.03 (s, 3H), 2.17 (qd, J=12.6, 3.8 Hz, 2H), 1.60 (td, J=13.4, 3.6 Hz, 2H), 1.47-1.34 (m, 2H), 1.07 (s, 1H), 0.63 (dp, J=14.0, 2.5 Hz, 2H), −0.05 (s, 4H).

Compound 1: 7-methyl-2-((7-methyl-[1,2,4]triazolo[1,5-a]pyridin-6-yl)amino)-9-(spiro[2.5]octan-6-yl)-7,9-dihydro-8H-purin-8-one

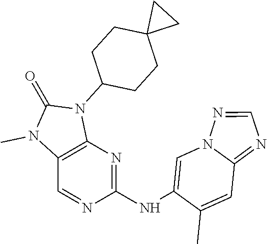 [SMILES: Cc1cc2ncnn2cc1Nc1ncc2c(n1)n(C1CCC3(CC1)CC3)c(=O)n2C]

To a mixture of Intermediate 1m (1.0 equiv.) and Intermediate 1d (1.0 equiv.), Pd(dppf)Cl₂ (0.2 equiv.), XantPhos (0.4 equiv.), and C_{S2}CO₃ (2.0 equiv.) in DMF (0.2-0.3 M) was degassed and purged 3× with N₂, and the mixture was stirred at 130° C. for 12 h under N₂ atmosphere. The mixture was then poured into water and extracted 3× with DCM. The combined organic phase was washed with brine, dried over Na₂SO₄, filtered, and the filtrate was concentrated in vacuum. The residue was purified by column chromatography to afford product as an off-white solid. ¹H NMR (400 MHz, (CD₃)₂SO) δ 9.09 (s, 1H), 8.73 (s, 1H), 8.44 (s, 1H), 8.16 (s, 1H), 7.78 (s, 1H), 4.21 (t, J=12.5 Hz, 1H), 3.36 (s, 3H), 2.43 (s, 3H), 2.34 (dt, J=13.0, 6.5 Hz, 2H), 1.93-1.77 (m, 2H), 1.77-1.62 (m, 2H), 0.91 (d, J=13.2 Hz, 2H), 0.31 (t, J=7.1 Hz, 2H). MS: 405.5 m/z [M+H].

The sequential edits occurred for each group as illustrated in Table 23.

TABLE 23
T cell engineering

| Group Name | Day 1 | Day 2 | Day 3 | Day 4 |
|---|---|---|---|---|
| TCR KO | TRBC | | TRAC | |
| TCR KO/WT1 | TRBC | | TRAC/AAV | |
| Insert | | | | |
| WT1/HLA-A | | HLA-A | TRAC/AAV | TRBC |
| AlloWT1 | CIITA | HLA-A | TRAC/AAV | TRBC |
| AlloWT1 + DNA | CIITA | HLA-A | TRAC/AAV + | TRBC |
| PKi Compound 1 | | | Compound 1 | |
| | | | (0.25 uM) | |

### 10.3. LNP Treatment and Expansion of T Cells

LNP compositions were formulated in ApoE-containing media and delivered to T cells as follows: on day 1, LNP compositions as indicated in Table 24 were incubated at a concentration of 5 ug/mL in TCAM containing 5 ug/mL rhApoE3 (Peprotech 350-02). Meanwhile, T cells were harvested, washed, and resuspended at a density of 2×10\hat{ }⁶ cells/mL in TCAM with a 1:50 dilution of T Cell TransAct, human reagent (Miltenyi, 130-111-160). T cells and LNP-ApoE media were mixed at a 1:1 ratio and T cells plated in culture flasks overnight.

On day 2, LNP compositions as indicated in Table 23 were incubated at a concentration of 25 ug/mL in TCAM containing 20 ug/mL rhApoE3 (Peprotech 350-02). LNP-ApoE solution was then added to the appropriate culture at a 1:10 ratio.

On day 3, TRAC-LNP compositions (Table 23) were incubated at a concentration of 5 ug/mL in TCAM containing 10 ug/mL rhApoE3 (Peprotech 350-02). Meanwhile, T cells were harvested, washed, and resuspended at a density of 1×10\hat{ }⁶ cells/mL in TCAM. T cells and LNP-ApoE media were mixed at a 1:1 ratio, and T cells were plated in culture flasks. WT1 AAV was then added to the relevant groups at an MOI of 3×10\hat{ }⁵ GC/cell. Compound 1 was added to the relevant groups at a final concentration of 0.25 uM.

On day 4, LNP compositions as indicated in Table 23 were incubated at a concentration of 5 ug/mL in TCAM containing 5 ug/mL rhApoE3 (Peprotech 350-02). T cells were washed by centrifugation and resuspended at a density of 1×10\hat{ }⁶ cells/mL LNP-ApoE solution was then added to the appropriate cultures at a 1:1 ratio.

On days 5 through 11, T cells were transferred to a GREX plate (Wilson Wolf) in T cell expansion media (TCEM: CTS OpTmizer (Thermofisher #A3705001) supplemented with 5% CTS Immune Cell Serum Replacement (Thermofisher #A2596101), 1× GlutaMAX (Thermofisher #35050061), 10 mM HEPES (Thermofisher #15630080), 200 U/mL IL-2 (Peprotech #200-02), IL-7 (Peprotech #200-07), IL-15 (Peprotech #200-15) and expanded. Briefly, T-cells were expanded for 6-days, with fresh cytokine supplementation every other day. Cells were counted using a Vi-CELL cell counter (Beckman Coulter) and fold expansion was calculated by dividing cell yield by the starting material.

### 10.4. Quantification of T Cell Editing by Flow Cytometry and NGS

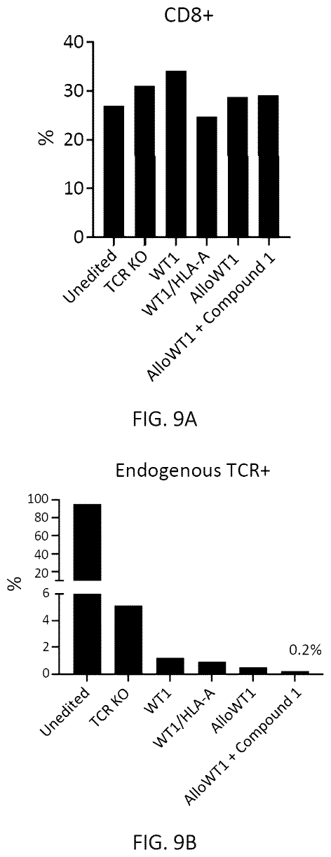

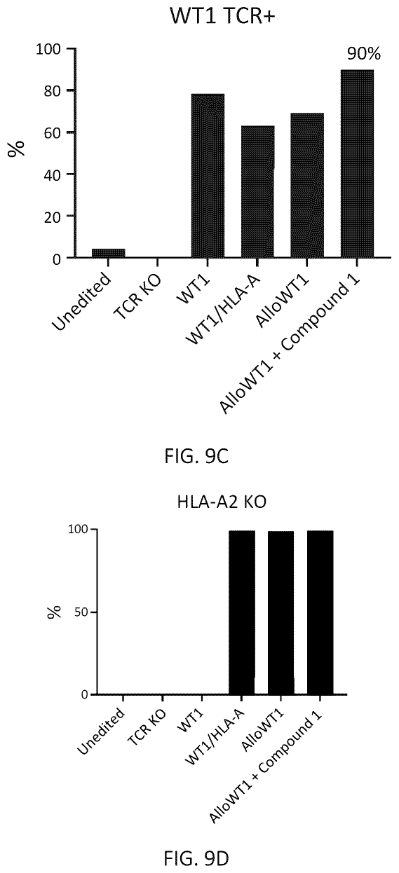

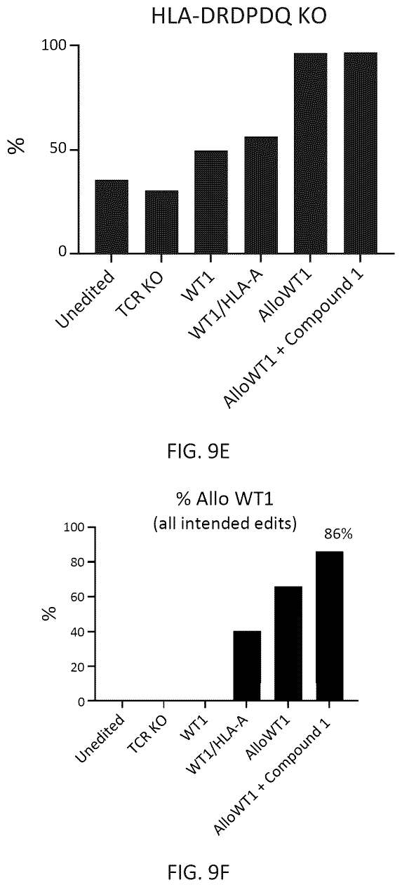

Post expansion, edited T cells were stained in an antibody cocktail to determine HLA-A2 knockout (HLA-A2⁻), HLA-DR-DP-DQ knockdown via CIITA knockout (HLA-DRDPDQ⁻), WT1-TCR insertion (CD₃⁺Vb8⁺), and the percentage of cells expressing residual endogenous (CD₃⁺Vb8⁻). Cells were subsequently washed, analyzed on a Cytoflex LX instrument (Beckman Coulter) using the FlowJo software package. T cells were gated on size and CD₈+ status, before editing and insertion rates were determined. Editing and insertion rates can be found in Table 24 and FIGS. 9A-9F. The percent of fully edited AlloWT1-T cells expressing the WT1-TCR with knockout of HLA-A and CIITA was gated as % CD₃⁺Vb8⁺HLA-A⁻FILA-DRDPDQ⁻. High levels of HLA-A and CIITA knockout, as well as WT1-TCR insertion and endogenous TCR KO were observed in edited samples. Notably, T cells receiving DNA PK inhibitor Compound 1 showed improved editing efficiencies

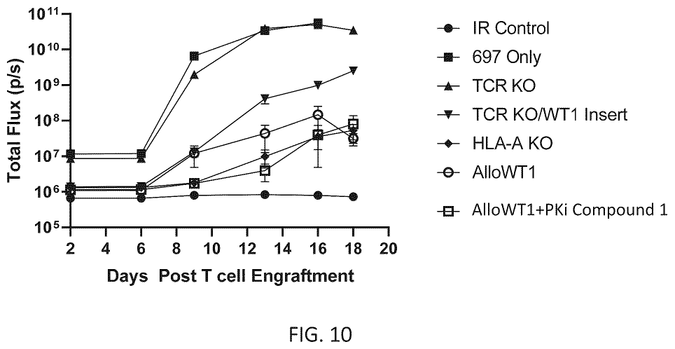

IVIS imaging of live mice was performed to identify luciferase-positive tumor cells by IVIS spectrum. IVIS imaging was done at 2 days, 6 days, 9 days, 13 days, 16 days, and 18 days after T cell injection. Mice were prepared for imaging with an injection of D-luciferin i.p. at 10 μL/g body weight per the manufacturer's recommendation, about 150 μL per animal. Animals were anesthetized and then placed in the IVIS imaging unit. The visualization was performed with the exposure time set to auto, field of view D, medium binning, and F/stop set to 1. Table 25 and FIG. 10 show radiance (photons/s/cm2/sr) from luciferase expressing T cells present at the various time points after injection out to 18 days.

TABLE 24
T cell editing efficiency

| | | Endogenous | WT1 | HLA- | HLA- | |
|---|---|---|---|---|---|---|
| | CD8+ | TCR+ | TCR+ | A2- | DRDPDQ- | AlloWT1+ |
| Unedited | 26.9 | 95.4 | 4.39 | 0.66 | 35.7 | 0.00292 |
| TCR KO | 31.1 | 5.12 | 0.5 | 0.62 | 30.8 | 0.23 |
| WT1 | 34.2 | 1.2 | 78.5 | 0.47 | 49.7 | 0.03 |
| WT1/HLA-A | 24.8 | 0.93 | 63.3 | 99.1 | 56.4 | 40.5 |
| AlloWT1 | 28.8 | 0.51 | 69.3 | 98.7 | 96.2 | 66.1 |
| AlloWT1 + | 29.2 | 0.23 | 89.8 | 99 | 96.5 | 86 |
| Compound 1 | | | | | | |

TABLE 25
Total Flux (photons/s) from luciferase-expressing target cells
in treated mice at intervals after T cell injection.

| | | Mean | SD | n |
|---|---|---|---|---|
| IR Control | 2 | 668000 | 0 | 1 |
| | 6 | 662000 | 0 | 1 |
| | 9 | 802000 | 0 | 1 |
| | 13 | 834000 | 0 | 1 |
| | 16 | 799000 | 0 | 1 |
| | 18 | 727000 | 0 | 1 |
| 697 Only | 2 | 11695000 | 6766940.65 | 8 |
| | 6 | 11756250 | 6759771.63 | 8 |
| | 9 | 6542375000 | 4097940177 | 8 |
| | 13 | 34156125000 | 19588932739 | 8 |
| | 16 | 56000000000 | 14890936841 | 8 |
| | 18 | | | |
| TCR KO | 2 | 8696250 | 3615004.20 | 8 |
| | 6 | 8755000 | 3659211.47 | 8 |
| | 9 | 1985750000 | 1311102671 | 8 |
| | 13 | 39295000000 | 18556359711 | 8 |
| | 16 | 50442857143 | 12082474518 | 7 |
| | 18 | 35000000000 | 0 | 1 |
| TCR KO/WT1 | 2 | 1395750 | 651356.99 | 8 |
| Insert | 6 | 1418625 | 660585.66 | 8 |
| | 9 | 13293750 | 10040193.42 | 8 |
| | 13 | 416762500 | 340405656.90 | 8 |
| | 16 | 987625000 | 637380114.80 | 8 |
| | 18 | 2523750000 | 1518542699 | 8 |
| HLA-A KO | 2 | 1306375 | 514478.92 | 8 |
| | 6 | 1323750 | 504219.55 | 8 |
| | 9 | 1785000 | 691416.77 | 8 |
| | 13 | 9851428.57 | 13794971.82 | 7 |
| | 16 | 35832857.14 | 53937852.11 | 7 |
| | 18 | 53608571.43 | 65167479.22 | 7 |
| AlloWT1 | 2 | 1085625 | 137185.94 | 8 |
| | 6 | 1100250 | 136031.25 | 8 |
| | 9 | 12085000 | 20455051.77 | 8 |
| | 13 | 43676250 | 87426018.67 | 8 |
| | 16 | 146917500 | 310795920.60 | 8 |
| | 18 | 31418750 | 33596200.65 | 8 |
| AlloWT1 + | 2 | 1138000 | 429877.06 | 8 |
| DNAPki | 6 | 1152750 | 420860.26 | 8 |
| | 9 | 1720000 | 654391.77 | 8 |
| | 13 | 3976250 | 5828721.83 | 8 |
| | 16 | 39420000 | 97704137.36 | 8 |
| | 18 | 80597500 | 162813409.10 | 8 |

### 10.5. Engineered T Cell Cytokine Release

Engineered T cells prepared as described in Example 10.1 and 10.2 were assayed for their cytokine release profiles. In vitro OCI-AML3 tumor cell killing assays were separately performed (data not shown) using the engineered T cells. The supernatants from the tumor cell killing assays were used to evaluate each engineered T cell's cytokine release profile.

Briefly, TCR KO T cells, Autologous WT1 T cells (TCR KO+WT1 TCR insertion), and Allogeneic WT1 T cells (as indicated in Table 24) were thawed and rested overnight in TCGM supplemented with IL-2, IL-7, and IL-15. The following day, a coculture assay was set up where each group of engineered T cells was co-cultured with OCI-AML3 target tumor. First, OCI-AML3 target tumor cells were pulsed with VLD peptide at different concentrations (500, 50, 0.5, 0.05, and 0.005 nM) for 1 hr. Next, T cells from each group were counted and resuspended in TCGM media without cytokines and co-cultured with pulsed OCI-AML3 at 1:1 E:T ratio. The T cell numbers in the co-culture were normalized to the insertion rates to keep the E:T consistent among different groups. After 24 hours of co-culture, the supernatant from each co-culture sample was diluted 5× in Diluent 2 from the U-PLEX Immuno-Oncology Group 1 (hu) Assays kit (MSD, Cat No. K151AEL-2). 50 μL of diluted samples from each group were loaded onto the meso scale discovery (MSD) plate and incubated for 1 hour.

For each of the cytokines measured, biotinylated capture antibody from the U-PLEX Immuno-Oncology Group 1 (hu) Assays (MSD, Cat No. K151AEL-2) was added to the assigned linker according to the kit's protocol. The antibody-linker mixtures were vortexed and incubated at room temperature for 30 minutes. Post incubation, the plate was washed, sealed, and stored overnight.

The following day, calibrators containing standards for each of the cytokines (IL-2 and IFN-γ) to be assayed were reconstituted as per the manufacturer's instructions and diluted to create a 4-fold standard curve.

The plates were washed, and 50 μL of the detection antibody solution (prepared according to kit instructions) was added to each well of the MSD plate. The plate was incubated for 1 hour.

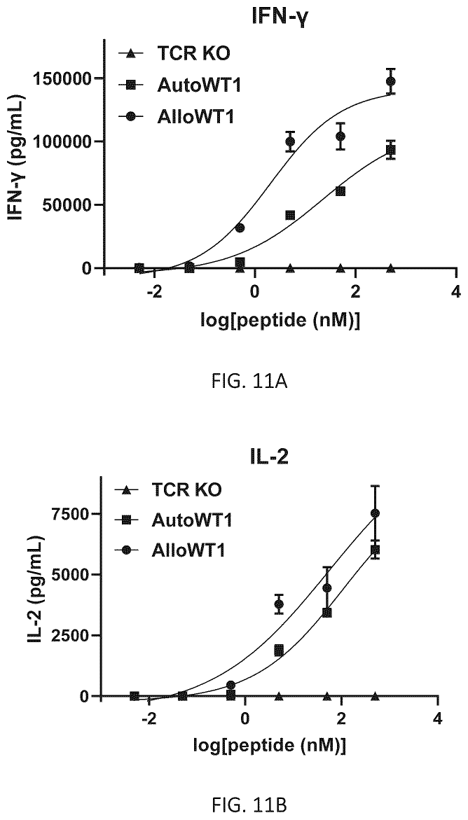

After incubation, the plate was washed and read immediately on the MSD instrument. Cytokine release is shown in Tables 26-27 and FIGS. 11A-11B.

TABLE 26
IFN- γ

| Log[peptide | | | | | | |
|---|---|---|---|---|---|---|
| (nM)] | TCR KO | | AutoWT1 | | AlloWT1 | |
| 2.70 | 122.55 | 25.96 | 93417.51 | 7094.06 | 147620.65 | 9709.50 |
| 1.70 | 134.20 | 16.97 | 60680.24 | 2770.37 | 104018.15 | 10358.48 |
| 0.70 | 144.94 | 24.90 | 41863.52 | 1759.74 | 99896.25 | 7700.60 |
| −0.30 | 146.14 | 58.09 | 4812.67 | 175.51 | 31820.97 | 1331.50 |
| −1.30 | 155.20 | 11.49 | 77.72 | 23.65 | 1592.76 | 131.04 |
| −2.30 | 110.63 | 22.03 | 69.41 | 3.27 | 351.29 | 23.17 |

TABLE 27
IL-2

| Log[peptide | | | | | | |
|---|---|---|---|---|---|---|
| (nM)] | TCR KO | | AutoWT1 | | AlloWT1 | |
| 2.70 | 4.21 | 0.63 | 6031.67 | 373.56 | 7525.26 | 1116.85 |
| 1.70 | 4.17 | 0.76 | 3419.94 | 97.86 | 4450.71 | 861.82 |
| 0.70 | 5.28 | 0.25 | 1882.55 | 204.86 | 3780.66 | 381.75 |
| −0.30 | 6.62 | 2.96 | 69.51 | 6.86 | 452.94 | 20.13 |
| −1.30 | 5.87 | 1.47 | 4.88 | 1.07 | 10.91 | 2.80 |
| −2.30 | 6.55 | 2.18 | 5.19 | 1.32 | 4.94 | 2.17 |

### Example 11: Mixed Lymphocyte Reaction Assay

T cells were isolated from peripheral blood of a healthy human donor with the following MHC I phenotype: HLA-A*02:01:01G, 03:01:01G, HLA-B*07:02:01G, HLA-C*07:02:01G. Briefly, a leukapheresis pack (Stemcell Technologies) was treated in ammonium chloride RBC lysis buffer (Stemcell Technologies; Cat. 07800) for 15 minutes to lyse red blood cells. Peripheral blood mononuclear cell (PBMC) count was determined post lysis and T cell isolation was performed using EasySep Human T cell isolation kit (Stemcell Technologies, Cat. 17951) according to manufacturer's protocol. Isolated CD3+ T cells were re-suspended in Cryostor CS10 media (Stemcell Technologies, Cat. 07930) and frozen down in liquid nitrogen until further use.

Frozen T cells were thawed at a cell concentration of 1.5×10\hat{ }⁶ cells/ml into T cell activation media (TCAM) composed of OpTmizer TCGM as described in Example 3 further supplemented with 100 U/mL of recombinant human interleukin-2 (Peprotech, Cat. 200-02), ng/ml IL-7 (Peprotech, Cat. 200-07), 5 ng/ml IL-15 (Peprotech, Cat. 200-15). Cells were rested at 37° C. for 24 hours.

Twenty-four hours post thawing T cells were counted and resuspended at 2×10\hat{ }⁶ cells/ml in TCAM media and 1:50 v/v of TransAct (Miltenyi Biotec Cat. 30-111-160) was added. 1×10\hat{ }⁶ cells were added to each well of a 24-well tissue culture plate, keeping 2 wells for each group to be engineered and 2 wells as unedited controls (Groups engineered: Unedited or WT, B2M KO (also indicated as HLA-I or HLA class I), CIITA (also indicated as HLA class II or HLA-II) KO, B2M+CIITA DKO, HLA-A KO, HLA-A+CIITA DKO). The plate was transferred to a 37° C. incubator. LNP compositions containing mRNA encoding cas9 (SEQ ID NO:802) and sgRNA G013675 (SEQ ID NO: 236), targeting CIITA were formulated with lipid A, cholesterol, DSPC, and PEG2k-DMG in a 50:38.5:10:1.5 molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 5 ug/ml were incubated in OpTmizer TCAM, further supplemented with 5 ug/ml recombinant human ApoE3 (Peprotech, Cat. 350-02) for 15 minutes at 37° C. In 6 out of the 12 wells, pre-incubated LNP and T cells with Transact were mixed to yield final concentrations of 1×10\hat{ }⁶ T cells/ml and 2.5 ug total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15) (These would be 2 wells for the CIITA KO group, 2 wells for HLA-A+CIITA DKO group and 2 wells for the B2M+CIITA DKO group). All the additional wells were mock edited with media containing ApoE3 but no LNP compositions. All cells were incubated at 37° C. for 24 hours.

24 hours post activation, 2 previously untreated wells and 2 CIITA LNP containing wells were treated with LNP compositions for B2M (for B2M KO and B2M+CIITA DKO groups); and 2 previously untreated wells and 2 CIITA LNP containing wells were treated with LNP compositions for HLA-A (for HLA-A KO and HLA-A+CIITA DKO groups). LNP compositions containing the Cas9 mRNA and sgRNA G000529 (SEQ ID NO: 245) targeting B2M, and LNP compositions containing mRNA encoding cas9 (SEQ ID NO:802) and sgRNA G018995 (sgRNA comprising SEQ ID NO: 13, as shown in Table 2) targeting HLA-A were formulated lipid A, cholesterol 1, DSPC, and PEG2k-DMG in a molar ratio, respectively. The lipid nucleic acid assemblies were formulated with a lipid amine to RNA phosphate (N:P) molar ratio of about 6, and a ratio of gRNA to mRNA of 1:2 by weight. LNP compositions at 25 ug/ml were incubated in OpTmizer TCAM, further supplemented with 20 ug/ml recombinant human ApoE3 (Peprotech, Cat. 350-02) for minutes at 37° C. The B2M and HLA-A LNP compositions, were added to the appropriate wells of the 24 well plate, as mentioned above, to yield final concentrations of 2.5 μg total RNA/mL of LNP in TCAM media with 2.5% human AB serum, 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15). An additional group of cells were mock edited with media containing ApoE3 but no LNP compositions, to serve as the unedited or WT control. All cells were incubated at 37° C. for 24 hours.

24 hours post the second round of editing, cells were washed by spinning at 500XG for 5 mins and resuspended in TCEM media containing with 5% CTS™ Immune Cell SR (Gibco Cat. A2596101), 100 U/mL of recombinant human interleukin-2 (Peprotech, Cat. 200-02), 5 ng/ml IL-7 (Peprotech, Cat. 200-07), 5 ng/ml IL-15 (Peprotech, Cat. 200-15. The cells were cultured and maintained in G-Rex plate for 7 days with regular changes in media and cytokines, after which they were re-suspended in Cryostor CS10 media (Stemcell Technologies, Cat. 07930) and frozen down in liquid nitrogen until further use.

Six groups of donor T cells (wildtype unedited, B2M KO, HLA-A KO, CIITA KO, HLA-A+CIITA DKO, B2M+CIITA DKO) were thawed and resuspended in TCGM at 1×10\hat{ }⁶/mL+100 U/ml IL-2, 0.5 ng/mL IL-7 & IL-15 (Donor and Host HLA-genotypes are shown below in Table 28). Peripheral blood mononuclear cells (PBMCs) from 3 hosts (Autologous host, Allogeneic host (HLA-B and C matched host), and Positive control host (HLA-A, HLA-B and HLA-C mismatched) were thawed, resuspended in TCGM at 1×10\hat{ }⁶/mL+100 U/ml IL-2, 0.5 ng/mL IL-7 & IL-15. Donor and host cells were rested overnight in a 37° C. incubator. The following day, donor cell flasks were irradiated at 4000 rad and spun down, and each group was resuspended at 1×10\hat{ }⁶/mL in TCGM without cytokines. Host PBMCs from the two hosts were depleted of CD56⁺ cells using the CD56 MicroBeads (Miltenyi Biotec, Cat. No. 130-050-401). About 1×10\hat{ }⁶ cells from each host were saved in 15 mL tubes for unlabeled flow controls. To label 18×10\hat{ }⁶ cells of each host, a vial of Cell Trace Violet (Thermo Fisher, Cat. No. C34571) was brought to room temperature and reconstituted using 20 μL DMSO to generate a stock of 5 mM CTV. Host cells were resuspended at −1×10\hat{ }⁶/mL in phosphate buffered saline (Corning, Cat. No. 21-040-CV) and transferred to another 50 mL conical tube. After adding 18 μL CTV into the tubes to stain host cells, the tubes were transferred to a 37° C. incubator for 15 minutes. Following that, the tubes were topped up to 40 mL with TCGM without cytokines to absorb any unbound dye. The labelled host cells were then spun down at 500×g for 5 minutes and resuspended in TCGM without cytokines at 1×10\hat{ }⁶/mL. 50,000 cells per 50 μL per well of host PBMCs were plated per well from appropriate hosts. In the wells requiring 4× host cells (control samples to normalize the data), 200,000 host cells were plated per 200 μL per well. In the host cells labelled “host+TransAct” (proliferation positive control), 50,000 cells per 50 μL per well of host PBMCs were seeded followed by the addition of 1 μL of T Cell TransAct™, human (Miltenyi Biotec, Cat. No. 130-111-160), and the volume of these wells was made up to 200 μL with cytokine free TCGM. The irradiated donor cells were plated according to the plate layout at 150,000 cells per 150 μL per well. For flow controls, 50,000 cells from one donor and host each were plated together. The volume in all wells was filled to 200 μL with TCGM without cytokines.

On day 5 post co-culture, half the media (˜100 μL) from each well was replaced with fresh media (TCGM without cytokines).

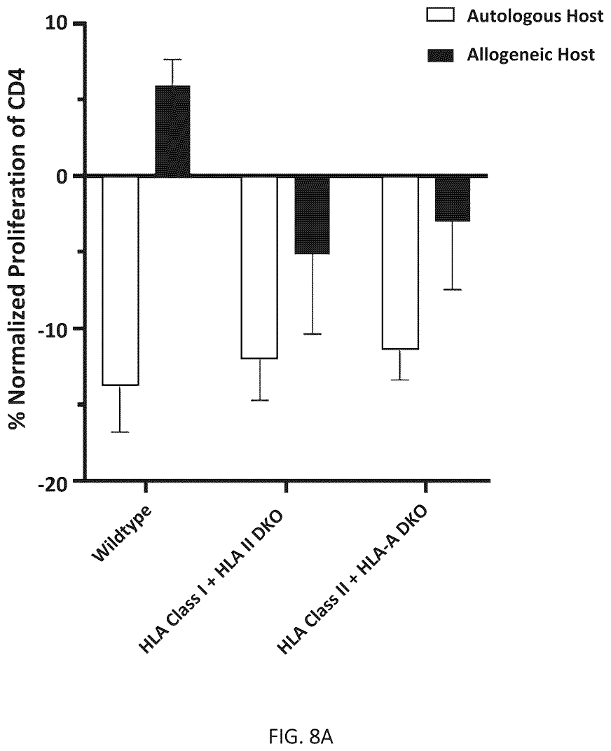

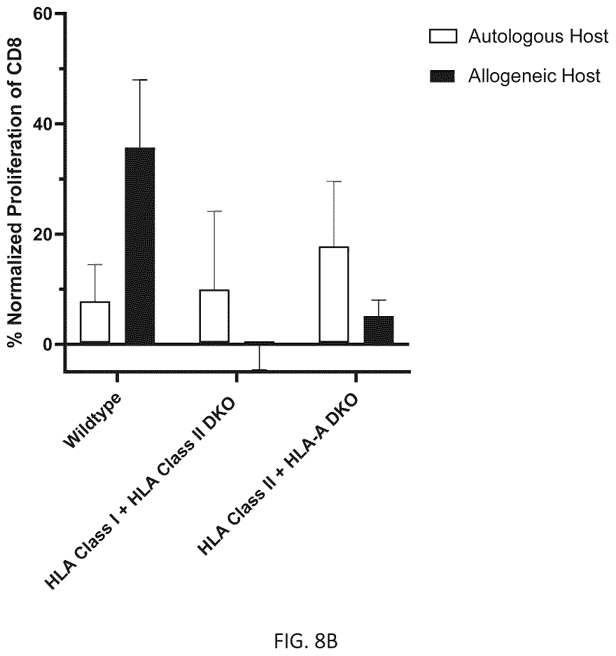

On day 8 post co-culture, the assay plate was stained and analyzed by flow cytometry. For the purpose of staining, the plate was spun at 600×g for 3 minutes, flicked to remove media, and 100 μL of a 1:100 v/v solution of Fc blocker (Biolegend, Cat #422302) in FACS buffer was added to each well. Cells were resuspended in the Fc blocker, and the plate was incubated at room temperature for 5 minutes. An antibody cocktail was prepared such that each antibody was present at a 1:100 v/v dilution, and 100 μL of this antibody mixture was added to each sample well. The plate was protected from light by covering with an aluminum foil and incubated at 2-8° C. for 20-30 minutes. After staining, the plate was spun at 600×g for 3 minutes, flicked to remove media and washed with 200 μL of FACS buffer. The plate was washed again, and the cell pellets were resuspended in 70 μL of a 1:200 v/v solution of the viability dye 7-AAD (BD Pharmingen, Cat #51-68981E). Unstained wells were resuspended in 70 μL of FACS buffer. The plate was run on fast mode (60 seconds per well) on Cytoflex flow cytometer. The results, shown in Tables 29A and 29B and FIGS. 8A and 8B (figures show a subset of data for Wildtype, B2M KO, and HLA-A+CIITA DKO), demonstrate that the HLA-A+CIITA DKO cells elicit minimal CD4 and CD8 responses in the allogeneic host (HLA-B and C matched), which were comparable to the response elicited by B2M+CIITA DKO cells. Results for each group have been normalized to that of the proliferation of the 4× host group, for the respective host.

TABLE 28
Genotypes of T cell donor and PBMC Hosts

| | HLA-A | HLA-B | HLA-C | HLA-DR | HLA-DQ | HLA-DP |
|---|---|---|---|---|---|---|
| T cell | A*02:01:01G, | B*07:02:01G | C*07:02:01G | DRB1*15:01:01G, | DQA1*01:02:01G, | DPA1*01:03:01G, |
| Donor | 03:01:01G | | | DRB5*01:01:01G | DQB1*06:02:01G | 02:07:01G, |
| and | | | | | | DPB1*04:01:01G, |
| Autologous | | | | | | 19:01:01G |
| Host | | | | | | |
| B, C | A*02:01:01G | B*07:02:01G, | C*05:01:01G, | DRB1*13:01:01G, | DQB1*06:02:01G, | DPB1*02:01:02G, |
| matched | | 44:02:01G | 07:02:01G | 15:01:01G, | 06:03:01G, | 04:02:01G, |
| Host | | | | DRB3*01:01:02G, | DQA1*01:02:01G, | DPA1*01:03:01G |
| | | | | DRB5*01:01:01 | 01:03:01G | |
| HLA | A*11:01:01G, | B*40:01:01G | C*03:04:01G | DRB1*08:01:01G, | DQB1*04:02:01G, | DPB1*03:01: |
| mis- | 24:02:01G | | | 13:02:01G, | 06:04:01G | 01G, |
| matched | | | | DRB3*03:01:01G | | 05:01:01G |
| Host | | | | | | |

TABLE 29A
Proliferation of Host CD4+ T Cells

| | Autologous Host | | Allogeneic Host | | Positive Control Host | |
|---|---|---|---|---|---|---|
| | Average % | SD % | Average % | SD % | Average % | SD % |
| | Normalized | Normalized | Normalized | Normalized | Normalized | Normalized |
| Group | Proliferation | Proliferation | Proliferation | Proliferation | Proliferation | Proliferation |
| WT | −13.76 | 3.05 | 5.93 | 1.72 | 39.07 | 3.68 |
| B2M KO | −13.50 | 2.66 | −3.22 | 5.10 | 42.47 | 3.20 |
| CIITA KO | −12.62 | 4.27 | −7.00 | 5.54 | −8.83 | 14.93 |
| B2M + | −11.98 | 2.76 | −5.15 | 5.21 | −14.20 | 4.64 |
| CIITA KO | | | | | | |
| HLA-A KO | −9.14 | 7.96 | 7.67 | 12.41 | 41.83 | 5.01 |
| HLA-A + | −11.33 | 2.03 | −3.00 | 4.47 | −3.97 | 6.57 |
| CIITA KO | | | | | | |

TABLE 29B
Proliferation of Host CD8+ T Cells

| | Autologous Host | | Allogeneic Host | | Positive Control Host | |
|---|---|---|---|---|---|---|
| | Average % | SD % | Average % | SD % | Average % | SD % |
| | Normalized | Normalized | Normalized | Normalized | Normalized | Normalized |
| Group | Proliferation | Proliferation | Proliferation | Proliferation | Proliferation | Proliferation |
| WT | 7.53 | 6.95 | 35.71 | 12.28 | 74.00 | 1.42 |
| B2M KO | −8.87 | 3.75 | 20.41 | 0.95 | 31.97 | 11.70 |
| CIITA KO | 1.43 | 5.24 | 6.17 | 4.89 | 56.07 | 8.53 |
| B2M + | 9.63 | 14.50 | −0.05 | 4.59 | 0.47 | 5.23 |
| CIITA KO | | | | | | |
| HLA-A | 22.40 | 23.65 | 25.31 | 16.59 | 71.83 | 2.25 |
| KO | | | | | | |
| HLA-A + | 17.57 | 12.00 | 5.14 | 2.88 | 58.13 | 7.02 |
| CIITA KO | | | | | | |

### Example 12: Sequential Delivery of Multiple LNP Compositions for Multiple Gene Disruptions and Insertions

T cells were engineered with a series of gene disruptions and insertions. Healthy donor cells were treated sequentially with four LNP compositions, each LNP composition co-formulated with mRNA encoding Cas9 (SEQ ID NO: 802) and sgRNA targeting either TRAC (G013006) (SEQ ID NO: 243), TRBC (G016239) (SEQ ID NO: 247), CIITA (G013675) (SEQ ID NO: 246), or HLA-A (G018995) (sgRNA comprising SEQ ID NO: 13, as shown in Table 2). LNP compositions were formulated according to the Groups indicated in Table 30 with either lipid A, cholesterol, DSPC, and PEG2k-DMG in a 35:47.5:15:2.5 molar ratio (Groups 1 and 2), respectively or lipid A, cholesterol, DSPC, and PEG2k-DMG in a 50:35:10:1.5 molar ratio (Group 3), respectively at the indicated doses. Groups 1 and 2 differ in LNP concentration. The lipid nucleic acid assemblies were formulated with a lipid amine to RNA phosphate (N:P) molar ratio of about 6, and a ratio of gRNA to mRNA of 1:2 by weight. A transgenic WT1 targeting TCR was site-specifically integrated into the TRAC cut site by delivering a homology directed repair template using AAV. LNP compositions were prepared each day and delivered to T cells as described in Table 30.

### 12.1 T Cell Preparation

T cells from three HLA-A*02:01+ serotypes were isolated from the leukopheresis products of two healthy donors (STEMCELL Technologies). T cells were isolated using EasySep Human T cell isolation kit (STEMCELL Technologies, Cat #17951) following manufacturer's protocol and cryopreserved using Cryostor CS10 (STEMCELL Technologies, Cat #07930). The day before initiating T cell editing, cells were thawed and rested overnight in T cell activation media (TCAM: CTS OpTmizer, Thermofisher #A3705001) supplemented with 2.5% human AB serum (Gemini #100-512), 1× GlutaMAX (Thermofisher #35050061), mM HEPES (Thermofisher #15630080), 200 U/mL IL-2 (Peprotech #200-02), IL-7 (Peprotech #200-07), and IL-15 (Peprotech #200-15).

### 12.2 LNP Treatment and Expansion of T Cells

LNP compositions were thawed and diluted on each day in ApoE containing media and delivered to T cells as follows.

TABLE 30
Order of Editing for T Cell Engineering

| | Day 1 Edit | Day 2 Edit | Day 3 Edit | Day 4 Edit |
|---|---|---|---|---|
| | (LNP | (LNP | (LNP | (LNP |
| | formulation & | formulation & | formulation & | formulation & |
| | final | final | final | final |
| Group | concentration) | concentration) | concentration) | concentration) |
| Group 1 | CIITA KO | HLA-A KO | TRAC KI | TRBC KO |
| | (Lipid A: | (Lipid A: | (Lipid A: | (Lipid A: |
| | 35:47.5:15:2.5, | 35:47.5:15:2.5, | 35:47.5:15:2.5, | 35:47.5:15:2.5, |
| | 0.65 μg/mL) | 0.65 μg/mL) | 0.65 μg/mL) | 0.65 μg/mL) |
| Group 2 | CIITA KO | HLA-A KO | TRAC KI | TRBC KO |
| | (Lipid A: | (Lipid A: | (Lipid A: | (Lipid A: |
| | 35:47.5:15:2.5, | 35:47.5:15:2.5, | 35:47.5:15:2.5, | 35:47.5:15:2.5, |
| | 2.5 μg/mL) | 2.5 μg/mL) | 2.5 μg/mL) | 2.5 μg/mL) |
| Group 3 | CIITA KO | HLA-A KO | TRAC KI | TRBC KO |
| | (Lipid A: | (Lipid A: | (Lipid A: | (Lipid A: |
| | 50:35.5:10:1.5, | 50:35.5:10:1.5, | 50:35.5:10:1.5, | 50:35.5:10:1.5, |
| | 2.5 μg/mL) | 2.5 μg/mL) | 2.5 μg/mL) | 2.5 μg/mL) |
| Unedited | None | None | None | None |

On day 1, LNP compositions as indicated in Table 30 were incubated in TCAM containing 5 μg/mL rhApoE3 (Peprotech 350-02). Meanwhile, T cells were harvested, washed, and resuspended at a density of 2×10\hat{ }⁶ cells/mL in TCAM with a 1:50 dilution of T Cell TransAct, human reagent (Miltenyi, 130-111-160). T cells and LNP-ApoE media were mixed at a 1:1 ratio and T cells plated in culture flasks overnight.

On day 2, LNP compositions as indicated in Table 30 were incubated at a concentration of 25 μg/mL in TCAM containing 20 μg/mL rhApoE3 (Peprotech 350-02). LNP-ApoE solution was then added to the appropriate culture at a 10:1 ratio.

On day 3, as indicated in Table 30 TRAC-LNP compositions were incubated in TCAM containing 5 μg/mL rhApoE3 (Peprotech 350-02). Meanwhile, T cells were harvested, washed, and resuspended at a density of 1×10\hat{ }⁶ cells/mL in TCAM. T cells and LNP-ApoE media were mixed at a 1:1 ratio, and T cells were plated in culture flasks. WT1 AAV was then added to each group at a MOI of 3×10\hat{ }⁵ GC/cell. The DNA-PK inhibitor “Compound 1” was added to each group at a concentration of 0.25 μM

On day 4, LNP compositions as indicated in Table 30 were incubated in TCAM containing 5 μg/mL rhApoE3 (Peprotech 350-02). Meanwhile, T cells were harvested, washed, and resuspended at a density of 1×10\hat{ }⁶ cells/mL in TCAM. T cells and LNP-ApoE media were mixed at a 1:1 ratio and T cells plated in culture flasks.

On days 5-13, T cells were transferred to a 24-well GREX plate (Wilson Wolf, 80192) in T cell expansion media (TCEM: CTS OpTmizer, Thermofisher #A3705001) supplemented with 5% human AB serum (Gemini #100-5121, 1× GlutaMAX (Thermofisher #35050061], 10 mM HEPES (Thermofisher #15630080), 200 U/mL IL-2 (Peprotech #200-02), IL-7 (Peprotech #200-07), IL-15 (Peprotech #200-15) and expanded per manufacturers' protocols. Briefly, T-cells were expanded for 8-days, with media exchanges every 2-3 days.

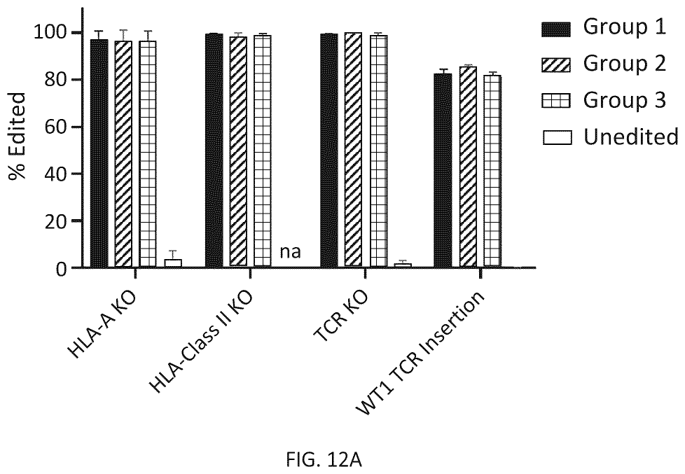

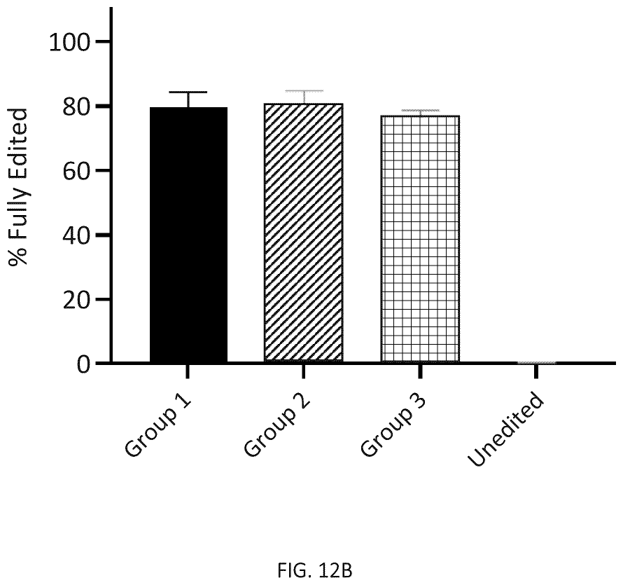

Post expansion, edited T cells were assayed by flow cytometry to determine HLA-A*02:01 knockout, HLA-DR-DP-DQ knockdown via CIITA knockout, WT1-TCR insertion (CD3⁺Vb8⁺), and the percentage of cells expressing residual endogenous (CD3⁺Vb8⁻). T Cells were incubated with an antibody cocktail targeting the following molecules: Vb8 (Biolegend, Cat. 348104), HLA-A2 (Biolegend, Cat. 343320), HLA-DRDPDQ (Biolegend, Cat. 361712), CD4 (Biolegend, Cat. 300538), CD8 (Biolegend, Cat. 301046), CD3 (Biolegend, Cat. 317336), CCR7 (Biolegend, Cat. 353214), CD62L (Biolegend, Cat. 304820), CD45RA (Biolegend, Cat. 304134), CD45RO (Biolegend, Cat. 304230), CD56 (Biolegend, Cat. 318328), and Viakrome (Beckman Coulter, Cat. C36628). Cells were subsequently washed, processed on a Cytoflex LX instrument (Beckman Coulter) and analyzed using the FlowJo software package. T cells were gated on size and CD4/CD8 status, before editing and insertion rates were determined. The percentage of cells expressing relevant cell surface proteins following sequential T cell engineering are shown in Table 31 and FIG. 12A for CD8+ T cells respectively. The percent of T cells with all intended edits (insertion of the WT1-TCR, combined with knockout of HLA-A and CIITA) was gated as % CD3⁺Vb8⁺ HLA-A⁻HLA-DRDPDQ⁻ and is shown in FIG. 12B. High levels of HLA-A and CIITA knockout, as well as WT1-TCR insertion were observed in edited samples from all groups yielding >75% of fully edited CD8+ T cells. The lower dosage (0.65 μg/mL) used with Lipid A 35:15:47.5:2.5 composition showed similar potency in editing T cells across all targets as the Lipid A 50:10:35.5:1.5 formulation at a higher dose (2.5 μg/mL).

TABLE 31
Editing rates in CD8+ T cells

| | Group 1 | | | Group 2 | | | Group 3 | | | Unedited | | |
|---|---|---|---|---|---|---|---|---|---|---|---|---|
| Edit | Mean | SD | N | Mean | SD | N | Mean | SD | N | Mean | SD | N |
| Fully Edited | 79.6 | 4.7 | 3.0 | 80.5 | 4.2 | 3.0 | 76.8 | 1.9 | 3.0 | 0.2 | 0.2 | 3.0 |
| (Vb8+, CD3+, HLA- | | | | | | | | | | | | |
| DRPDPDQ-, HLA- | | | | | | | | | | | | |
| A*02:01-) | | | | | | | | | | | | |
| HLA-A KO (HLA- | 97.1 | 3.6 | 3.0 | 96.4 | 4.7 | 3.0 | 96.4 | 4.4 | 3.0 | 3.6 | 3.8 | 3.0 |
| A*02:01-) | | | | | | | | | | | | |
| CIITA KO (HLA- | 99.3 | 0.4 | 3.0 | 97.7 | 2.1 | 3.0 | 98.7 | 0.9 | 3.0 | na | na | na |
| DRDPDQ-) | | | | | | | | | | | | |
| TCR KO (CD3-) | 99.3 | 0.1 | 3.0 | 99.7 | 0.1 | 3.0 | 98.7 | 1.1 | 3.0 | 1.8 | 1.4 | 3.0 |
| WT1 TCR Insertion | 82.6 | 2.0 | 3.0 | 85.6 | 0.8 | 3.0 | 81.1 | 2.1 | 3.0 | 0.2 | 0.2 | 3.0 |
| (Vb8+) | | | | | | | | | | | | |

### Example 13: Cytotoxic Susceptibility of Engineered T Cells

Engineered T cells were assayed for cytotoxic susceptibility when targeted by natural killer (NK) cells.

NK cells (Stemcell Technologies) were thawed and resuspended at a cell concentration of 1×10\hat{ }⁶ cells/ml into T cell growth media (TCGM) composed of OpTmizer TCGM and further supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15). Cells were incubated at 37° C. for 24 hours.

Twenty-four hours post thaw, the NK cells were labelled with 0.5 NM Cell Trace Violet (CTV) as follows: a vial of CTV (CellTrace™ Violet Cell Proliferation Kit, for flow cytometry, Cat. C34571) was reconstituted in DMSO from the kit to give a 5 mM stock concentration. Two μL of CTV stock was diluted with 18 μL Phosphate-Buffered Saline (PBS) (Corning, Cat. 21-040-CV) to obtain a concentration of 0.5 mM. NK cells were centrifuged at 500×g for 5 minutes, the media was aspirated, and cells were resuspended in PBS at a concentration of 1×10\hat{ }⁶ cells/mL such that the final concentration of CTV dye was 0.5 μM. The cells were mixed with CTV dye solution incubated at 37° C. for 20 minutes. Unbound dye was quenched by the addition of TCGM and incubated for 5 minutes. The cells were centrifuged at 500×g for 5 minutes. Cells are resuspended in TCGM supplemented with 100 U/mL of recombinant human interleukin-2 (Peprotech, Cat. 200-02), 5 ng/mL IL-7 (Peprotech, Cat. 200-07), 5 ng/mL IL-15 (Peprotech, Cat. 200-15) at a concentration of 2×10\hat{ }⁶ cells/mL. To test a range of effector:target (E:T) ratios, CTV-labelled NK cells were aliquoted in 100 μL of media in a 6-point, 2-fold serial dilution with the highest number of cells being 2×10\hat{ }⁵ cells. Media-only samples were included as negative controls.

T cells were engineered using BC22n and UGI mRNA using G023523 (SEQ ID NO: 1016) targeting HLA-A as a test sample and with G023519 (SEQ ID NO: 816) targeting B2M as a positive control for NK killing.

T cells were prepared from a leukopak using the EasySep Human T Cell Isolation Kit (Stem Cell Technology, Cat. 17951) following the manufacturers protocol. T cells were cryopreserved in Cryostor CS10 freezing media (Cat. 07930) for future use. Upon thaw, T cells were plated at a density of 1.0×10\hat{ }⁶ cells/mL in T cell R10 media composed of RPMI 1640 (Corning, Cat. 10-040-CV) containing 10% (v/v) of fetal bovine serum, 2 mM Glutamax (Gibco, Cat. 35050-061), 22 μM of 2-Mercaptoethanol, 100 uM non-essential amino acids (Corning, Cat. 25-025-Cl), 1 mM sodium pyruvate, 10 mM HEPES buffer, 1% of Penicillin-Streptomycin, plus 100 U/mL of recombinant human interleukin-2 (Peprotech, Cat. 200-02). T cells were activated with Dynabeads® Human T-Activator CD3/CD28 (Gibco, Cat. 11141D). Cells were expanded in T cell media for 72 hours prior to mRNA transfection.

Solutions containing mRNA encoding BC22n (SEQ ID NO: 972) or UGI (SEQ ID NO: 1005) were prepared in sterile water. 50 NM targeting sgRNAs were removed from their storage plates and denatured for 2 minutes at 95° C. before cooling on ice. Seventy-two hours post activation, T cells were harvested, centrifuged, and resuspended at a concentration of 12.5×10\hat{ }⁶ T cells/mL in P3 electroporation buffer (Lonza). For each well to be electroporated, 1×10\hat{ }⁵ T cells were mixed with 200 ng of editor mRNA (BC22n), 200 ng of UGI mRNA, and 20 pmols of sgRNA in a final volume of 20 μL of P3 electroporation buffer. This mix was electroporated using the manufacturer's pulse code.

Unedited T cells were assayed as a negative control for NK killing. Other controls for flow cytometry included CTV-labelled NK cells without T cells; a “unstained” sample combining unlabelled NK cells and T cells; and a 1:1 mix of unlabeled heat killed and non-heat killed NK cells and T cells stained with 7AAD. T cells were resuspended at a density of 2×10\hat{ }⁵ cells in TCGM composed of OpTmizer TCGM and further supplemented with 100 U/mL of recombinant human interleukin-2 (Peprotech, Cat. 200-02), 5 ng/mL IL-7 (Peprotech, Cat. 200-07), and 5 ng/mL IL-15 (Peprotech, Cat. 200-15). Twenty thousand T cells were added to each well of NK cells and media controls. Cells were incubated at 37° C. for 24 hours.

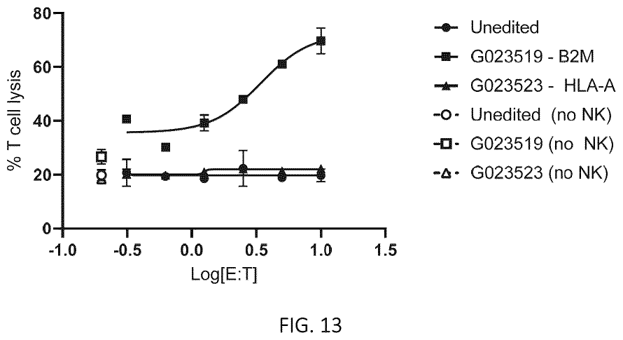

At 24 hours, half of the volume of the cells from the LD heat killed well were heat killed and transferred back to the same well in the assay plate. Cells were centrifuged and resuspended in 80 μL of a 1:200 v/v solution of 7-AAD (BD Biosciences, Cat. 559925) in FACS buffer (PBS+2% FBS (Gibco, Cat. A31605-02)+2 mM EDTA (Invitrogen, Cat. 15-575-020)). Data for specific lysis of T cells were acquired by flow cytometry using a Cytoflex LX instrument (Beckman Coulter) and analyzed using the FlowJo software package. Gates were first drawn on the CTV negative population to gate out the NK cells, followed by gating on singlets after which a gate was drawn on the 7-AAD negative population to gate for the live T cells. The percent lysis of T cells was calculated by subtracting the live cell percentage from 100. T cells edited using BC22n and HLA-A guide G023523 (SEQ ID NO: 1016) were protected from NK cell mediated cytotoxicity as shown in Table 32 and FIG. 13.

TABLE 32
Mean percentage lysis of engineered T cells exposed to HLA-B
and C matched NK cells

| | Unedited | | | G023519 B2M | | | G023523 HLA-A | | |
|---|---|---|---|---|---|---|---|---|---|
| E:T | Mean | SD | n | Mean | SD | n | Mean | SD | n |
| 10 | 19.65 | 2.33 | 2 | 69.60 | 4.81 | 2 | 22.23 | 1.10 | 3 |
| 5 | 18.80 | 1.59 | 3 | 61.10 | 0.85 | 2 | 21.35 | 0.49 | 2 |
| 2.5 | 22.27 | 6.62 | 3 | 47.95 | 0.49 | 2 | 22.10 | 1.27 | 2 |
| 1.25 | 18.47 | 1.27 | 3 | 39.20 | 2.98 | 3 | 21.00 | 0.81 | 3 |
| 0.63 | 19.30 | 0.66 | 3 | 30.20 | NA | 1 | 19.75 | 0.35 | 2 |
| 0.31 | 20.70 | 5.02 | 3 | 40.60 | NA | 1 | 20.27 | 1.67 | 3 |
| 0 | 19.77 | 2.01 | 3 | 26.57 | 2.73 | 3 | 18.30 | 1.41 | 3 |

### Example 14: Editing Human T Cells with BC22n, UGI and 91-Mer sgRNAs

The base editing efficacy of 91-mer sgRNA as assessed by receptor knockout was compared to that of a 100-mer sgRNA format with the same guide sequence.

The tested 91-mer sgRNA include a 20-nucleotide guide sequence (as represented by N) and a guide scaffold as follows: mN*mN*mN*NNGUUUUAGAmGmCmUmAmGmAmAmAmU mAmGmCAAGUUAAAAUAAGGCUAGUCCGUUAUCACGAAAGGGCACCGAGUCG GmUmGmC*mU (SEQ ID NO: 1003), where A, C, G, U, and N are adenine, cytosine, guanine, uracil, and any ribonucleotide, respectively, unless otherwise indicated. An m is indicative of a 2′O-methyl modification, and an * is indicative of a phosphorothioate linkage between the nucleotides. Unmodified and modified versions of the guide is provided in Table 6 (Sequence Table).

### Example 14.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10\hat{ }⁶ cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatments.

### Example 14.2. T Cell LNP Treatment and Expansion

Forty-eight hours post-activation, T cells were harvested, centrifuged at 500 g for min, and resuspended at a concentration of 1×10\hat{ }⁶ T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). 50 μL of T cells in TCPM (5×10\hat{ }⁴ T cells) were added per well to be treated in flat-bottom 96-well plates.

LNPs were prepared as described in Example 1 at a ratio of 35:47.5:15:2.5 (Lipid A/cholesterol/DSPC/PEG2k-DMG). The LNPs were formulated with a lipid amine to RNA phosphate (N:P) molar ratio of about 6. LNPs encapsulated a single RNA species, either a sgRNA as described in Table 34, BC22n mRNA (SEQ ID No: 972), or UGI mRNA (SEQ ID No. 1005).

TABLE 33
100-mer and 91-mer sgRNAs.

| Gene target | 100-mer | 91-mer |
|---|---|---|
| HLA-A | G021209 | G023523 |
| | (SEQ ID | (SEQ ID |
| | NO: 381) | NO: 1016) |

Prior to T cell treatment, LNPs encapsulating a sgRNA were diluted to 6.64 μg/mL in T cell treatment media (TCTM): a version of TCGM containing 20 ug/mL rhApoE3 in the absence of interleukins 2, 5 or 7. These LNPs were incubated at 37° C. for 15 minutes and serially diluted 1:4 using TCTM, which resulted in an 8-point dilution series ranging from 6.64 μg/mL to zero. Similarly, single-cargo LNPs with BC22n mRNA (SEQ ID NO: 972) or UGI mRNA (SEQ ID NO: 1005) were diluted in TCTM to 3.32 and 1.67 μg/mL, respectively, incubated at 37° C. for 15 minutes, and mixed 1:1 by volume with sgRNA LNPs serially diluted in the previous step. Last, 50 μL, from the resulting mix was added to T cells in 96-well plates at a 1:1 ratio by volume. T cells were incubated at 37° C. for 24 hours, at which time they were harvested, centrifuged at 500 g for 5 min, resuspended in 200 μL, of TCGM and returned to the incubator.

### Example 14.4. Evaluation of Receptor Knockout by Flow Cytometry

The set of sgRNAs targeting the HLA-A gene were evaluated by flow cytometry instead of NGS due to the hyperpolymorphic nature of the HLA-A locus.

Seven days post LNP treatment, T cells were assayed by flow cytometry to evaluate receptor knockout. T cells were incubated with a fixable viability dye (Beckman Coulter, Cat. C36628) and an antibody cocktail targeting HLA-A2 (Biolegend, Cat. 343304). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

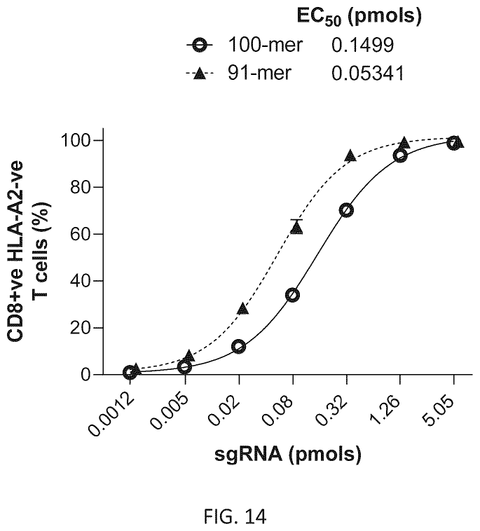

As shown in Tables 34 and 35 and FIG. 14, the 91-mer sgRNA tested outperformed the 100-mer version. Targets with a lower potency (i.e., higher EC50) in the 100-mer format (HLA-A) seem to benefit the most from usage of 91-mer sgRNAs.

TABLE 34
Mean percentage of CD8+ T cells that are negative
for HLA-A2 surface receptors following treatment sgRNA
targeting HLA-A, in the 100-mer or 91-mer formats.

| | HLA-A (HLA-A2−) | |
|---|---|---|
| sgRNA | 100-mer | 91-mer |

| (ng) | Mean | SD | Mean | SD |
|---|---|---|---|---|
| 166.00 | 98.8 | 0.1 | 99.6 | 0.2 |
| 41.50 | 93.6 | 0.8 | 99.2 | 0.4 |
| 10.38 | 70.2 | 1.0 | 93.8 | 1.4 |
| 2.59 | 34.0 | 2.1 | 63.2 | 3.0 |
| 0.65 | 12.1 | 1.3 | 28.5 | 1.2 |
| 0.16 | 3.3 | 0.2 | 8.3 | 0.6 |
| 0.04 | 0.9 | 0.3 | 2.6 | 0.5 |
| 0.00 | 0.1 | 0.0 | 0.3 | 0.2 |

TABLE 35
Amount (pmol) of sgRNA that lead to a 50% loss of receptor expression
in the surface of CD8+ T cells (EC50s). The far right column
shows the fold-increase in potency achieved by 91-mer sgRNA when
compared to the 100-mer with the same guide sequence.

| | | | | | EC50 |
|---|---|---|---|---|---|
| | 100-mer | | 91-mer | | shift |
| Gene | sgRNA | EC50 | sgRNA | EC50 | (100-mer/ |
| target | ID | (pmols) | ID | (pmols) | 91-mer) |
| HLA-A | G021209 | 0.150 | G023523 | 0.053 | 2.81 |

### Example 15: Correlation Between HLA-A Editing by NGS and Protein KO by Flow Cytometry

Frozen T cells from three T cell donors, the first heterozygous for HLA-A*02:01:01G, 03:01:01G, the second homozygous for HLA-A*02:01:01G, and the third homozygous for HLA-A*03:01:01G, were thawed at a cell concentration of 1.5×10\hat{ }⁶ cells/mL into T cell growth media (TCGM) composed of CTS OpTmizer media (Gibco, Cat. #A10485-01) with 2.5 percent GemCell Plus Human AB Serum (Gemini, Cat. #100-512), and 10 mL each of GlutaMAX 100× (Gibco, Cat. #35050061), HEPES (Gibco, Cat. #15630080) and Pen/Strep (Gibco, Cat. #15140-122), further supplemented with 100 U/mL of recombinant human interleukin-2 (Peprotech, Cat. #200-02), 5 ng/mL IL-7 (Peprotech, Cat. #200-07), 5 ng/mL IL-15 (Peprotech, Cat. #200-15), and rested overnight in a 37° C. incubator.

Twenty-four (24) hours post thaw, cells were activated using T cell TransAct™ (Miltenyi Biotec, Cat. #130-111-160) at 1:100 dilution at 37° C. for 24 hours. Cells were plated at 1×10\hat{ }⁵ cells per 100 μL per well and then transfected with a serial dilution of LNP-formulated guides, starting from 5 μg/mL as the highest dose and down to 0.04 μg/mL.

On Day 5 post transfection, cells from each donor were spun and collected for NGS assay. Genomic DNA was extracted using QuickExtract DNA extraction solution. PCR1 was performed to amplify the gene-specific sequences, while PCR2 was performed to amplify the common adaptor for sequencing (NEB Cat. #N0494). PCR samples were cleaned using AMPure XP Beads (Beckman Coulter Cat. #A63881) before sequencing by NGS.

On Day 8 post transfection, the assay plate was stained and analyzed by flow cytometry. For the purpose of staining, the plate was spun at 500×g for 5 minutes, flicked to remove media, and 100 μL of a 1:100 v/v solution of Fc blocker (Biolegend, Cat. #422302) in FACS buffer was added to each well. Cells were resuspended in the Fc blocker, and the plate was incubated at room temperature for 5 minutes. An antibody cocktail was prepared such that each antibody (HLA-A2 Monoclonal Antibody (BB7.2), APC, eBioscience, Cat. #17-9876-42 and HLA-A3 Monoclonal Antibody (GAP.A3), PE, eBioscience, Cat. #12-5754-42) was present at a 1:100 v/v dilution, and 100 μL of this antibody mixture was added to each sample well. The plate was protected from light by covering with an aluminum foil and incubated at 2-8° C. for 20-30 minutes. After staining, the plate was spun at 600×g for 3 minutes, flicked to remove media, and washed with 200 μL of FACS buffer. The plate was washed again, and the cell pellets were resuspended in 100 μL of FACS buffer. The plate was run on fast mode (60 seconds per well) on a Cytoflex flow cytometer. Data analysis was conducted on FlowJo.

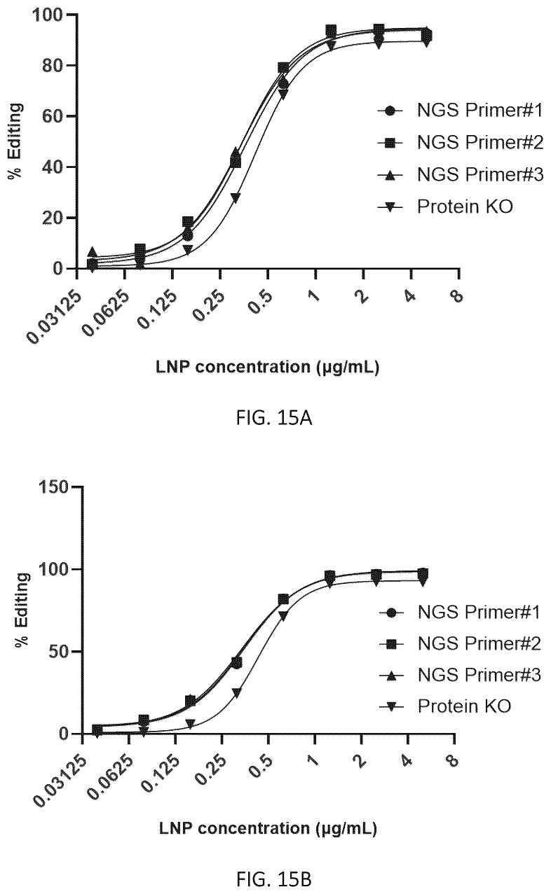

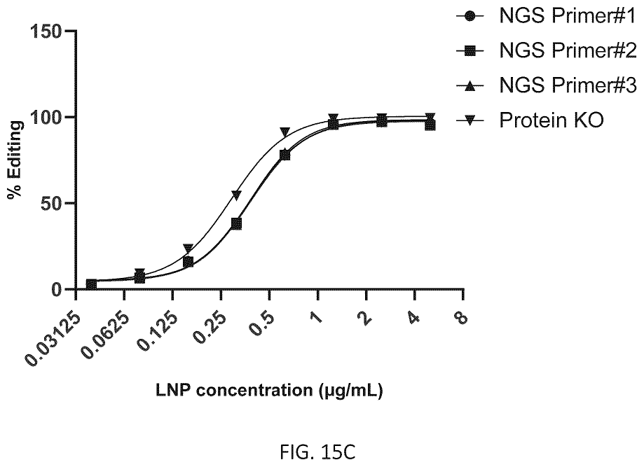

High correlation between protein knockout and editing was observed in all three donors, and for three unique primer sets, as shown in Tables 36-38 and FIGS. 15A-15C.

TABLE 36
HLA-A gene editing correlation to protein knockout in Donor A

| | NGS | NGS | NGS | |
|---|---|---|---|---|
| LNP | Primer 1 | Primer 2 | Primer 3 | Protein |
| Concentration | (% Edit) | (% Edit) | (% Edit) | KO |
| 5 | 92.7 | 91.9 | 93.5 | 89.15 |
| 2.5 | 93.6 | 94.4 | 92.7 | 88.35 |
| 1.25 | 93.2 | 94 | 92.8 | 87.55 |
| 0.63 | 72.9 | 79.3 | 74.3 | 68.45 |
| 0.31 | 41.8 | 41.8 | 46.1 | 27.6 |
| 0.17 | 12.9 | 18.5 | 15.8 | 7.23 |
| 0.08 | 4.7 | 7.8 | 1.9 | 1.44 |
| 0.04 | 2 | 1.7 | 6.8 | 0.30 |

TABLE 37
HLA-A gene editing correlation to protein knockout in Donor B

| | NGS | NGS | NGS | |
|---|---|---|---|---|
| LNP | Primer 1 | Primer 2 | Primer 3 | Protein |
| Concentration | (% Edit) | (% Edit) | (% Edit) | KO |
| 5 | 97.9 | 97.5 | 97.9 | 92.3 |
| 2.5 | 97.2 | 96.9 | 97.2 | 92.6 |
| 1.25 | 96.4 | 96.1 | 96.5 | 91.25 |
| 0.63 | 82.1 | 81.9 | 82 | 71.35 |
| 0.31 | 42.4 | 43.6 | 44.7 | 24.5 |
| 0.17 | 20.3 | 20.2 | 21.2 | 5.65 |
| 0.08 | 7.4 | 8.6 | 8.4 | 0.94 |
| 0.04 | 2.1 | 2.7 | 2.3 | 0.15 |

TABLE 38
HLA-A gene editing correlation to protein knockout in Donor C

| | NGS | NGS | NGS | |
|---|---|---|---|---|
| LNP | Primer 1 | Primer 2 | Primer 3 | Protein |
| Concentration | (% Edit) | (% Edit) | (% Edit) | KO |
| 5 | 96.6 | 95.3 | 96.6 | 99.295 |
| 2.5 | 97.3 | 97.4 | 97.3 | 99.165 |
| 1.25 | 95.7 | 95.8 | 97.4 | 98.9 |
| 0.63 | 77.9 | 78.1 | 79.4 | 91 |
| 0.31 | 37.7 | 38.5 | 37.7 | 54.25 |
| 0.17 | 16.3 | 16 | 16.7 | 23.35 |
| 0.08 | 7 | 6.8 | 6.5 | 9.22 |
| 0.04 | 3.1 | 2.5 | 2.6 | 3.108 |

### Example 16. Additional Embodiments

The following numbered embodiments provide additional support for and descriptions of the embodiments herein.

Embodiment 1 is an engineered human cell, which has reduced or eliminated surface expression of HLA-A relative to an unmodified cell, comprising a genetic modification in the HLA-A gene, wherein the cell is homozygous for HLA-B and homozygous for HLA-C.

Embodiment 2 is an engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: (a) chr6:29942854-chr6:29942913 and (b) chr6:29943518-chr6:29943619; wherein the cell is homozygous for HLA-B and homozygous for HLA-C.

Embodiment 3 is the engineered cell of any of the preceding embodiments, wherein the cell has reduced or eliminated expression of at least one HLA-A allele selected from: HLA-A1, HLA-A2, HLA-A3, HLA-A11, and HLA-A24.

Embodiment 4 is the engineered cell of any of the preceding embodiments, wherein the cell has reduced or eliminated expression of HLA-A1.

Embodiment 5 is the engineered cell of any of the preceding embodiments, wherein the cell has reduced or eliminated expression of HLA-A2.

Embodiment 6 is the engineered cell of any of the preceding embodiments, wherein the cell has reduced or eliminated expression of HLA-A3.

Embodiment 7 is the engineered cell of any of the preceding embodiments, wherein the cell has reduced or eliminated expression of HLA-A11.

Embodiment 8 is the engineered cell of any of the preceding embodiments, wherein the cell has reduced or eliminated expression of HLA-A24.

Embodiment 9 is the engineered cell of any of the preceding embodiments, wherein the genetic modification comprises at least one nucleotide within the genomic coordinates chr6:29942864-chr6:29942903.

Embodiment 10 is the engineered cell of any of the preceding embodiments, wherein the genetic modification comprises at least one nucleotide within the genomic coordinates chr6:29943528-chr6:29943609.

Embodiment 11 is the engineered cell of any of the preceding embodiments, wherein the genetic modification comprises at least one nucleotide within the genomic coordinates chr6:29942864-29942884; chr6:29942868-29942888; chr6:29942876-29942896; chr6:29942877-29942897; and chr6:29942883-29942903.

Embodiment 12 is the engineered cell of any of the preceding embodiments, wherein the genetic modification comprises at least one nucleotide within the genomic coordinates chr6:29943528-29943548; chr6:29943529-29943549; chr6:29943530-29943550; chr6:29943537-29943557; chr6:29943549-29943569; and chr6:29943589-29943609.

Embodiment 13 is the engineered cell of any of the preceding embodiments, wherein the genetic modification comprises at least one nucleotide within the genomic coordinates chr6:29942876-29942897.

Embodiment 14 is the engineered cell of any of the preceding embodiments, wherein the genetic modification comprises at least one nucleotide within the genomic coordinates chr6:29943528-chr629943550.

Embodiment 15 is the engineered cell of any of the preceding embodiments, wherein the genetic modification comprises at least one nucleotide within the genomic coordinates chosen from: chr6:29942864-29942884, chr6:29942868-29942888, chr6:29942876-29942896, and chr6:29942877-29942897.

Embodiment 16 is the engineered cell of any of the preceding embodiments, wherein the genetic modification comprises at least one nucleotide within the genomic coordinates chosen from: chr6:29943528-29943548, chr6:29943529-29943549, and chr6:29943530-29943550.

Embodiment 17 is an engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 18 is an engineered human cell, which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

Embodiment 19 is the engineered cell of any one of embodiments 17-18, wherein the cell is homozygous for HLA-B and homozygous for HLA-C.

Embodiment 20 is the engineered cell of any one of embodiments 17-19, wherein the genetic modification comprises at least 2, at least 3, at least 4, at least 5, at least 6, at least 7, at least 8, at least 9, or at least 10 contiguous nucleotides within the genomic coordinates, or wherein the genetic modification comprises at least 5 contiguous nucleotides within the genomic coordinates.

Embodiment 21 is the engineered cell of any one of embodiments 17-20, wherein the genetic modification comprises at least 6, 7, 8, 9, or 10 contiguous nucleotides within the genomic coordinates.

Embodiment 22 is the engineered cell of any one of embodiments 17-21, wherein the genetic modification comprises at least one C to T substitution or at least one A to G substitution within the genomic coordinates.

Embodiment 23 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chosen from: (a) chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870; (b) chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046; (c) chr6:29942864-29942884; chr6:29942868-29942888; chr6:29942876-29942896; chr6:29942877-29942897; chr6:29942883-29942903; chr6:29943528-29943548; chr6:29943529-29943549; chr6:29943530-29943550; chr6:29943537-29943557; chr6:29943549-29943569; and chr6:29943589-29943609; (d) chr6:29942864-29942884; chr6:29942868-29942888; chr6:29942876-29942896; chr6:29942877-29942897; and chr6:29942883-29942903; (e) chr6:29943528-29943548; chr6:29943529-29943549; chr6:29943530-29943550; chr6:29943537-29943557; chr6:29943549-29943569; and chr6:29943589-29943609; (f) chr6:29942864-29942884, chr6:29942868-29942888, chr6:29942876-29942896, and chr6:29942877-29942897; (g) chr6:29943528-29943548, chr6:29943529-29943549, and chr6:29943530-29943550; (h) chr6:29945290-29945310, chr6:29945296-29945316, and chr6:29945297-29945317, chr6:29945300-29945320; (i) chr6:29890117-29890137, chr6:29927058-29927078, chr6:29934330-29934350, chr6:29942541-29942561, chr6:29942542-29942562, chr6:29942543-29942563, chr6:29942543-29942563, chr6:29942550-29942570, chr6:29942864-29942884, chr6:29942868-29942888, chr6:29942876-29942896, chr6:29942876-29942896, chr6:29942877-29942897, chr6:29942883-29942903, chr6:29943062-29943082, chr6:29943063-29943083, chr6:29943092-29943112, chr6:29943115-29943135, chr6:29943118-29943138, chr6:29943119-29943139, chr6:29943120-29943140, chr6:29943126-29943146, chr6:29943128-29943148, chr6:29943129-29943149, chr6:29943134-29943154, chr6:29943134-29943154, chr6:29943135-29943155, chr6:29943136-29943156, chr6:29943140-29943160, chr6:29943142-29943162, chr6:29943143-29943163, chr6:29943188-29943208, chr6:29943528-29943548, chr6:29943529-29943549, chr6:29943530-29943550, chr6:29943536-29943556, chr6:29943537-29943557, chr6:29943538-29943558, chr6:29943549-29943569, chr6:29943556-29943576, chr6:29943589-29943609, chr6:29943590-29943610, chr6:29943590-29943610, chr6:29943599-29943619, chr6:29943600-29943620, chr6:29943601-29943621, chr6:29943602-29943622, chr6:29943603-29943623, chr6:29943774-29943794, chr6:29943779-29943799, chr6:29943780-29943800, chr6:29943822-29943842, chr6:29943824-29943844, chr6:29943857-29943877, chr6:29943858-29943878, chr6:29943859-29943879, chr6:29943860-29943880, chr6:29944026-29944046, chr6:29944077-29944097, chr6:29944078-29944098, chr6:29944458-29944478, chr6:29944478-29944498, chr6:29944597-29944617, chr6:29944642-29944662, chr6:29944643-29944663, chr6:29944772-29944792, chr6:29944782-29944802, chr6:29944850-29944870, chr6:29944907-29944927, chr6:29945024-29945044, chr6:29945097-29945117, chr6:29945104-29945124, chr6:29945105-29945125, chr6:29945116-29945136, chr6:29945118-29945138, chr6:29945119-29945139, chr6:29945124-29945144, chr6:29945176-29945196, chr6:29945177-29945197, chr6:29945177-29945197, chr6:29945180-29945200, chr6:29945187-29945207, chr6:29945188-29945208, chr6:29945228-29945248, chr6:29945230-29945250, chr6:29945231-29945251, chr6:29945232-29945252, chr6:29945308-29945328, chr6:29945361-29945381, chr6:29945362-29945382, and chr6:31382543-31382563; (j) chr6:29942815-29942835, chr6:29942816-29942836, chr6:29942817-29942837, chr6:29942817-29942837, chr6:29942828-29942848, chr6:29942837-29942857, chr6:29942885-29942905, chr6:29942895-29942915, chr6:29942896-29942916, chr6:29942898-29942918, chr6:29942899-29942919, chr6:29942900-29942920, chr6:29942904-29942924, chr6:29942905-29942925, chr6:29942912-29942932, chr6:29942913-29942933, chr6:29943490-29943510, chr6:29943497-29943517, chr6:29943498-29943518, chr6:29943502-29943522, chr6:29943502-29943522, chr6:29943511-29943531, chr6:29943520-29943540, chr6:29943521-29943541, chr6:29943566-29943586, chr6:29943569-29943589, chr6:29943569-29943589, chr6:29943570-29943590, chr6:29943573-29943593, chr6:29943578-29943598, chr6:29943585-29943605, chr6:29943589-29943609, chr6:29943568-29943588, and chr6:29942815-29942835. (k) chr6:29942884-29942904, chr6:29943519-29943539, chr6:29942863-29942883; (l) chr6:29943517-29943537, and chr6:29943523-29943543; (m) chr6:29942845-29942869, chr6:29942852-29942876, chr6:29942865-29942889, chr6:29942891-29942915, chr6:29942895-29942919, chr6:29942903-29942927, chr6:29942904-29942928, chr6:29943518-29943542, chr6:29943525-29943549, chr6:29943535-29943559, chr6:29943538-29943562, chr6:29943539-29943563, chr6:29943547-29943571, chr6:29943547-29943571, chr6:29943548-29943572, chr6:29943555-29943579, chr6:29943556-29943580, chr6:29943557-29943581, chr6:29943558-29943582, chr6:29943559-29943583, chr6:29943563-29943587, chr6:29943564-29943588, chr6:29943565-29943589, chr6:29943568-29943592, chr6:29943571-29943595, chr6:29943572-29943596, chr6:29943595-29943619, chr6:29943596-29943620, and chr6:29943600-29943624; (n) chr6:29942885-29942905, chr6:29942895-29942915, chr6:29942896-29942916, chr6:29942898-29942918, chr6:29942899-29942919, chr6:29942900-29942920, chr6:29942904-29942924, chr6:29943511-29943531, chr6:29943520-29943540, chr6:29943521-29943541, chr6:29943529-29943549, chr6:29943566-29943586, chr6:29943568-29943588, chr6:29943569-29943589, chr6:29943569-29943589, chr6:29943570-29943590, chr6:29943573-29943593, chr6:29943578-29943598, chr6:29943585-29943605, and chr6:29943589-29943609; or (o) chr6:29942469-29942489, chr6:29943058-29943078, chr6:29943063-29943083, chr6:29943080-29943100, chr6:29943187-29943207, chr6:29943192-29943212, chr6:29943197-29943217, chr6:29943812-29943832, chr6:29944349-29944369, chr6:29944996-29945016, chr6:29945018-29945038, and chr6:29945341-29945361, chr6:29945526-29945546.

Embodiment 24 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chosen from chr6:29942854-chr6:29942913 and chr6:29943518-chr6:29943619.

Embodiment 25 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942876-29942897.

Embodiment 26 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943528-chr629943550.

Embodiment 27 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942864-29942884.

Embodiment 28 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942868-29942888.

Embodiment 29 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942876-29942896.

Embodiment 30 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942877-29942897.

Embodiment 31 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29942883-29942903.

Embodiment 32 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943126-29943146.

Embodiment 33 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943528-29943548.

Embodiment 34 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943529-29943549.

Embodiment 35 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943530-29943550.

Embodiment 36 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943537-29943557.

Embodiment 37 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943549-29943569.

Embodiment 38 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates chr6:29943589-29943609.

Embodiment 39 is the engineered cell of any one of the preceding embodiments, wherein the HLA-A expression is reduced or eliminated by a gene editing system that binds to an HLA-A genomic target sequence comprising at least 5 contiguous nucleotides within the genomic coordinates and chr6:29944026-29944046.

Embodiment 40 is the engineered cell of any one of embodiments 23-39, wherein the HLA-A genomic target sequence comprises at least 10 contiguous nucleotides within the genomic coordinates.

Embodiment 41 is the engineered cell of any one of embodiments 23-40, wherein the HLA-A genomic target sequence comprises at least 15 contiguous nucleotides within the genomic coordinates.

Embodiment 42 is the engineered cell of any one of embodiments 23-41, wherein the HLA-A genomic target sequence comprises at least 17, 19, 18, or 20 contiguous nucleotides within the genomic coordinates.

Embodiment 43 is the engineered cell of any one of embodiments 23-41, wherein the gene editing system comprises a transcription activator-like effector nuclease (TALEN).

Embodiment 44 is the engineered cell of any one of embodiments 23-41, wherein the gene editing system comprises a zinc finger nuclease.

Embodiment 45 is the engineered cell of any one of embodiments 23-41, wherein the gene editing system comprises an RNA-guided DNA-binding agent or a nucleic acid encoding an RNA-guided DNA binding agent.

Embodiment 46 is the engineered cell of embodiment 45, wherein the RNA-guided DNA-binding agent or the RNA-guided DNA-binding agent encoded by the nucleic acid comprises a Cas9 protein.

Embodiment 47 is the engineered cell of embodiment 45, wherein the RNA-guided DNA-binding agent or the RNA-guided DNA-binding agent encoded by the nucleic acid is S. pyogenes Cas9.

Embodiment 48 is the engineered cell of embodiment 45, wherein the RNA-guided DNA-binding agent or the RNA-guided DNA-binding agent encoded by the nucleic acid is N. meningitidis Cas9, optionally Nme2Cas9.

Embodiment 49 is the engineered cell of embodiment 45, wherein the RNA-guided DNA-binding agent or the RNA-guided DNA-binding agent encoded by the nucleic acid is S. thermophilus Cas9.

Embodiment 50 is the engineered cell of embodiment 45, wherein the RNA-guided DNA-binding agent or the RNA-guided DNA-binding agent encoded by the nucleic acid is S. aureus Cas9.

Embodiment 51 is the engineered cell of embodiment 45, wherein the RNA-guided DNA-binding agent or the RNA-guided DNA-binding agent encoded by the nucleic acid is Cpf1 from F. novicida.

Embodiment 52 is the engineered cell of embodiment 45, wherein the RNA-guided DNA-binding agent or the RNA-guided DNA-binding agent encoded by the nucleic acid is Cpf1 from Acidaminococcus sp.

Embodiment 53 is the engineered cell of embodiment 45, wherein the RNA-guided DNA-binding agent or the RNA-guided DNA-binding agent encoded by the nucleic acid is Cpf1 from Lachnospiraceae bacterium ND2006.

Embodiment 54 is the engineered cell of embodiment 45, wherein the RNA-guided DNA-binding agent or the RNA-guided DNA-binding agent encoded by the nucleic acid is a C to T base editor.

Embodiment 55 is the engineered cell of embodiment 45, wherein the RNA-guided DNA-binding agent or the RNA-guided DNA-binding agent encoded by the nucleic acid is an A to G base editor.

Embodiment 56 is the engineered cell of embodiment 45, wherein the RNA-guided DNA-binding agent or the RNA-guided DNA-binding agent encoded by the nucleic acid comprises a APOBEC3A deaminase (A3A) and an RNA-guided nickase.

Embodiment 57 is the engineered cell of embodiment 45, wherein the RNA-guided DNA-binding agent or the RNA-guided DNA-binding agent encoded by the nucleic acid is Cas12a.

Embodiment 58 is the engineered cell of embodiment 45, wherein the RNA-guided DNA-binding agent or the RNA-guided DNA-binding agent encoded by the nucleic acid is CasX.

Embodiment 59 is the engineered cell of embodiment 45, wherein the RNA-guided DNA-binding agent or the RNA-guided DNA-binding agent encoded by the nucleic acid is Nme2Cas9.

Embodiment 60 is the engineered cell of embodiment 45, wherein the RNA-guided DNA-binding agent or the RNA-guided DNA-binding agent encoded by the nucleic acid is Mad7 nuclease.

Embodiment 61 is the engineered cell of embodiment 45, wherein the RNA-guided DNA-binding agent or the RNA-guided DNA-binding agent encoded by the nucleic acid is an ARCUS nucleases.

Embodiment 62 is the engineered cell of any one of embodiments 17-61, wherein the cell is homozygous for HLA-B and homozygous for HLA-C.

Embodiment 63 is the engineered cell of any one of the preceding embodiments, wherein the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02.

Embodiment 64 is the engineered cell of any one of the preceding embodiments, wherein the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 65 is the engineered cell of any one of the preceding embodiments, wherein the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; and the HLA-C allele is selected from any one of the following HLA-C alleles: HLA-C*07:02; HLA-C*07:01; HLA-C*05:01; HLA-C*04:01 HLA-C*03:04; HLA-C*06:02; HLA-C*08:02; HLA-C*03:03; HLA-C*06:02; HLA-C*16:01; HLA-C*12:03; HLA-C*07:01; HLA-C*04:01; HLA-C*15:02; HLA-C*07:01; HLA-C*03:04; HLA-C*12:03; HLA-C*02:02; HLA-C*04:01; HLA-C*05:01; HLA-C*12:02; HLA-C*14:02; HLA-C*06:02; HLA-C*04:01; HLA-C*03:03; HLA-C*07:04; HLA-C*07:01; HLA-C*04:01; HLA-C*04:01; and HLA-C*02:02.

Embodiment 66 is the engineered cell of any one of the preceding embodiments, wherein the HLA-B and HLA-C alleles are selected from any one of the following HLA-B and HLA-C alleles: HLA-B*07:02 and HLA-C*07:02; HLA-B*08:01 and HLA-C*07:01; HLA-B*44:02 and HLA-C*05:01; HLA-B*35:01 and HLA-C*04:01; HLA-B*40:01 and HLA-C*03:04; HLA-B*57:01 and HLA-C*06:02; HLA-B*14:02 and HLA-C*08:02; HLA-B*15:01 and HLA-C*03:03; HLA-B*13:02 and HLA-C*06:02; HLA-B*44:03 and HLA-C*16:01; HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02.

Embodiment 67 is the engineered cell of any one of the preceding embodiments, wherein the HLA-B and HLA-C alleles are HLA-B*07:02 and HLA-C*07:02.

Embodiment 68 is the engineered cell of any one of the preceding embodiments, wherein the HLA-B and HLA-C alleles are HLA-B*08:01 and HLA-C*07:01.

Embodiment 69 is the engineered cell of any one of the preceding embodiments, wherein the HLA-B and HLA-C alleles are HLA-B*44:02 and HLA-C*05:01.

Embodiment 70 is the engineered cell of any one of the preceding embodiments, wherein the HLA-B and HLA-C alleles are HLA-B*35:01 and HLA-C*04:01.

Embodiment 71 is the engineered cell of any one of the preceding embodiments, wherein the cell has reduced expression of MHC class II protein on the surface of the cell.

Embodiment 72 is the engineered cell of any one of the preceding embodiments, wherein the cell has a genetic modification of a gene selected from CIITA, HLA-DR, HLA-DQ, HLA-DP, RFXS, RFXB/ANK, RFXAP, CREB, NF-YA, NF-YB, and NF-YC.

Embodiment 73 is the engineered cell of any one of the preceding embodiments, wherein the cell has a genetic modification in the CIITA gene.

Embodiment 74 is the engineered cell of any one of the preceding embodiments, wherein the cell has reduced expression of TRAC protein on the surface of the cell.

Embodiment 75 is the engineered cell of any one of the preceding embodiments, wherein the cell has reduced expression of TRBC protein on the surface of the cell.

Embodiment 76 is the engineered cell of any one of the preceding embodiments, wherein the engineered cell further comprises an exogenous nucleic acid.

Embodiment 77 is the engineered cell of any one of the preceding embodiments, wherein the engineered cell comprises an exogenous nucleic acid encoding a targeting receptor that is expressed on the surface of the engineered cell or a ligand for the receptor.

Embodiment 78 is the engineered cell of embodiment 77, wherein the targeting receptor is a CAR.

Embodiment 79 is the engineered cell of embodiment 77, wherein the targeting receptor is a TCR.

Embodiment 80 is the engineered cell of embodiment 77, wherein the targeting receptor is a WT1 TCR.

Embodiment 81 is the engineered cell of embodiment 77, wherein the engineered cell comprises a ligand for the receptor.

Embodiment 82 is the engineered cell of any one of the preceding embodiments, wherein the engineered cell further comprises an exogenous nucleic acid encoding a polypeptide that is secreted by the engineered cell.

Embodiment 83 is the engineered cell of any one of the preceding embodiments, wherein the engineered cell is an immune cell.

Embodiment 84 is the engineered cell, population of cells, pharmaceutical composition, or method of any one of the preceding embodiments, wherein the cell is a primary cell.

Embodiment 85 is the engineered cell of any one of the preceding embodiments, wherein the engineered cell is a monocyte, macrophage, mast cell, dendritic cell, or granulocyte.

Embodiment 86 is the engineered cell of any one of the preceding embodiments, wherein the engineered cell is a lymphocyte.

Embodiment 87 is the engineered cell of any one of the preceding embodiments, wherein the cell is a T cell.

Embodiment 88 is the engineered cell of any one of the preceding embodiments, wherein the cell is a CD8+ T cell.

Embodiment 89 is the engineered cell of any one of the preceding embodiments, wherein the cell is a CD4+ T cell.

Embodiment 90 is the engineered cell of any one of the preceding embodiments, wherein the cell is a B cell.

Embodiment 91 is the engineered cell of any one of the preceding embodiments, wherein the cell is a natural killer (NK) cell.

Embodiment 92 is the engineered cell of any one of the preceding embodiments, wherein the cell is a macrophage.

Embodiment 93 is the engineered cell, population of cells, pharmaceutical composition, or method of any one of the preceding embodiments, wherein the cell is a B cell.

Embodiment 94 is the engineered cell, population of cells, pharmaceutical composition, or method of any one of the preceding embodiments, wherein the cell is a plasma B cell.

Embodiment 95 is the engineered cell, population of cells, pharmaceutical composition, or method of any one of the preceding embodiments, wherein the cell is memory B cell.

Embodiment 96 is the engineered cell of any one of the preceding embodiments, wherein the cell is a stem or progenitor cell.

Embodiment 97 is the engineered cell of any one of the preceding embodiments, wherein the stem or progenitor cell is an HSC or an iPSC.

Embodiment 98 is the engineered cell, population of cells, pharmaceutical composition, or method of any one of the preceding embodiments, wherein the cell is an activated cell.

Embodiment 99 is the engineered cell, population of cells, pharmaceutical composition, or method of any one of the preceding embodiments, wherein the cell is a non-activated cell.

Embodiment 100 is the engineered cell of any one of the preceding embodiments, wherein the genetic modification comprises at least 2, at least 3, at least 4, at least 5, at least 6, at least 7, at least 8, at least 9, or at least 10 contiguous nucleotides within the genomic coordinates, or wherein the genetic modification comprises at least 5 contiguous nucleotides within the genomic coordinates.

Embodiment 101 is the engineered cell of any one of the preceding embodiments, wherein the genetic modification comprises at least 6, 7, 8, 9, or 10 contiguous nucleotides within the genomic coordinates.

Embodiment 102 is the engineered cell of any one of the preceding embodiments, wherein the genetic modification comprises an indel.

Embodiment 103 is the engineered cell of any of the preceding embodiments, wherein the genetic modification comprises at least one C to T substitution or at least one A to G substitution within the genomic coordinates.

Embodiment 104 is a pharmaceutical composition comprising the engineered cell of any one of the preceding embodiments.

Embodiment 105 is a population of cells comprising the engineered cell of any one of the preceding embodiments.

Embodiment 106 is a pharmaceutical composition comprising the population of cells of embodiment 105.

Embodiment 107 is the population of embodiment 105 or pharmaceutical composition of embodiment 106, wherein the population of cells is at least 65% HLA-A negative as measured by flow cytometry.

Embodiment 107.1 is the population of embodiment 105 or pharmaceutical composition of embodiment 106, wherein at least 65% of the population of cells comprises the genetic modification in the HLA-A gene, as measured by next-generation sequencing (NGS).

Embodiment 108 is the population of embodiment 105 or pharmaceutical composition of embodiment 106, wherein the population of cells is at least 70% HLA-A negative as measured by flow cytometry.

Embodiment 108.1 is the population of embodiment 105 or pharmaceutical composition of embodiment 106, wherein at least 70% of the population of cells comprises the genetic modification in the HLA-A gene, as measured by next-generation sequencing (NGS).

Embodiment 109 is the population of embodiment 105 or pharmaceutical composition of embodiment 106, wherein the population of cells is at least 80% HLA-A negative as measured by flow cytometry.

Embodiment 109.1 is the population of embodiment 105 or pharmaceutical composition of embodiment 106, wherein at least 80% of the population of cells comprises the genetic modification in the HLA-A gene, as measured by next-generation sequencing (NGS).

Embodiment 110 is the population of embodiment 105 or pharmaceutical composition of embodiment 106, wherein the population of cells is at least 90% HLA-A negative as measured by flow cytometry.

Embodiment 110.1 is the population of embodiment 105 or pharmaceutical composition of embodiment 106, wherein at least 90% of the population of cells comprises the genetic modification in the HLA-A gene, as measured by next-generation sequencing (NGS).

Embodiment 111 is the population of embodiment 105 or pharmaceutical composition of embodiment 106, wherein the population of cells is at least 92% HLA-A negative as measured by flow cytometry.

Embodiment 111.1 is the population of embodiment 105 or pharmaceutical composition of embodiment 106, wherein at least 92% of the population of cells comprises the genetic modification in the HLA-A gene, as measured by next-generation sequencing (NGS).

Embodiment 112 is the population of embodiment 105 or pharmaceutical composition of embodiment 106, wherein the population of cells is at least 93% HLA-A negative as measured by flow cytometry.

Embodiment 112.1 is the population of embodiment 105 or pharmaceutical composition of embodiment 106, wherein at least 93% of the population of cells comprises the genetic modification in the HLA-A gene, as measured by next-generation sequencing (NGS).

Embodiment 113 is the population of embodiment 105 or pharmaceutical composition of embodiment 106, wherein the population of cells is at least 94% HLA-A negative as measured by flow cytometry.

Embodiment 113.1 is the population of embodiment 105 or pharmaceutical composition of embodiment 106, wherein at least 94% of the population of cells comprises the genetic modification in the HLA-A gene, as measured by next-generation sequencing (NGS).

Embodiment 114 is the population of embodiment 105 or pharmaceutical composition of embodiment 106, wherein the population of cells is at least 95% HLA-A negative as measured by flow cytometry.

Embodiment 114.1 is the population of embodiment 105 or pharmaceutical composition of embodiment 106, wherein at least 95% of the population of cells comprises the genetic modification in the HLA-A gene, as measured by next-generation sequencing (NGS).

Embodiment 115 is the population of embodiment 105 or pharmaceutical composition of embodiment 106, wherein the population of cells is at least 96% HLA-A negative as measured by flow cytometry.

Embodiment 115.1 is the population of embodiment 105 or pharmaceutical composition of embodiment 106, wherein at least 96% of the population of cells comprises the genetic modification in the HLA-A gene, as measured by next-generation sequencing (NGS).

Embodiment 116 is the population of embodiment 105 or pharmaceutical composition of embodiment 106, wherein the population of cells is at least 97% HLA-A negative as measured by flow cytometry.

Embodiment 116.1 is the population of embodiment 105 or pharmaceutical composition of embodiment 106, wherein at least 97% of the population of cells comprises the genetic modification in the HLA-A gene, as measured by next-generation sequencing (NGS).

Embodiment 117 is the population of embodiment 105 or pharmaceutical composition of embodiment 106, wherein the population of cells is at least 98% HLA-A negative as measured by flow cytometry.

Embodiment 117.1 is the population of embodiment 105 or pharmaceutical composition of embodiment 106, wherein at least 98% of the population of cells comprises the genetic modification in the HLA-A gene, as measured by next-generation sequencing (NGS).

Embodiment 118 is the population of embodiment 105 or pharmaceutical composition of embodiment 106, wherein the population of cells is at least 99% HLA-A negative as measured by flow cytometry.

Embodiment 118.1 is the population of embodiment 105 or pharmaceutical composition of embodiment 106, wherein at least 99% of the population of cells comprises the genetic modification in the HLA-A gene, as measured by next-generation sequencing (NGS).

Embodiment 119 is the population or pharmaceutical composition of any one of embodiments 105-118, wherein the population of cells is at least 94% CIITA negative as measured by flow cytometry.

Embodiment 120 is the population or pharmaceutical composition of any one of embodiments 105-118, wherein the population of cells is at least 95% CIITA negative as measured by flow cytometry.

Embodiment 121 is the population or pharmaceutical composition of any one of embodiments 105-118, wherein the population of cells is at least 96% CIITA negative as measured by flow cytometry.

Embodiment 122 is the population or pharmaceutical composition of any one of embodiments 105-118, wherein the population of cells is at least 97% CIITA negative as measured by flow cytometry.

Embodiment 123 is the population or pharmaceutical composition of any one of embodiments 105-118, wherein the population of cells is at least 98% CIITA negative as measured by flow cytometry.

Embodiment 124 is the population or pharmaceutical composition of any one of embodiments 105-118, wherein the population of cells is at least 99% CIITA negative as measured by flow cytometry.

Embodiment 125 is the population or pharmaceutical composition of any one of embodiments 105-124, wherein the population of cells is at least 95% endogenous TCR protein negative as measured by flow cytometry.

Embodiment 126 is the population or pharmaceutical composition of any one of embodiments 105-124, wherein the population of cells is at least 97% endogenous TCR protein negative as measured by flow cytometry.

Embodiment 127 is the population or pharmaceutical composition of any one of embodiments 105-124, wherein the population of cells is at least 98% endogenous TCR protein negative as measured by flow cytometry.

Embodiment 128 is the population or pharmaceutical composition of any one of embodiments 105-124, wherein the population of cells is at least 99% endogenous TCR protein negative as measured by flow cytometry.

Embodiment 129 is the population or pharmaceutical composition of any one of embodiments 105-124, wherein the population of cells is at least 99.5% endogenous TCR protein negative as measured by flow cytometry.

Embodiment 130 is a method of administering the engineered cell, population of cells, pharmaceutical composition of any one of the preceding embodiments to a subject in need thereof.

Embodiment 131 is a method of administering the engineered cell, population of cells, or pharmaceutical composition of any one of the preceding embodiments to a subject as an adoptive cell transfer (ACT) therapy.

Embodiment 132 is a method of treating a disease or disorder comprising administering the engineered cell, population of cells, or pharmaceutical composition of any one of the preceding embodiments to a subject in need thereof.

Embodiment 133 is a method of making an engineered human cell, which has reduced or eliminated surface expression of HLA-A protein relative to an unmodified cell, wherein the cell is homozygous for HLA-B and homozygous for HLA-C, comprising contacting a cell with composition comprising: (a) an HLA-A guide RNA comprising (i) a guide sequence selected from SEQ ID NOs: 1-211; or (ii) at least 17, 18, 19, or 20 contiguous nucleotides of a sequence selected from SEQ ID NOs: 1-211; or (iii) a guide sequence at least 95%, 90%, or 85% identical to a sequence selected from SEQ ID NOs: 1-211; or (iv) a guide sequence that binds a target site comprising a genomic region listed in Tables 2-5; or (v) a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or (vi) a guide sequence that is at least 95%, 90%, or 85% identical to a sequence selected from (v); and optionally (b) an RNA-guided DNA binding agent or a nucleic acid encoding an RNA-guided DNA binding agent.

Embodiment 134 is a method of reducing surface expression of HLA-A protein in a human cell relative to an unmodified cell, comprising contacting a cell with composition comprising: (a) an HLA-A guide RNA comprising (i) a guide sequence selected from SEQ ID NOs: 1-211; or (ii) at least 17, 18, 19, or 20 contiguous nucleotides of a sequence selected from SEQ ID NOs: 1-211; or (iii) a guide sequence at least 95%, 90%, or 85% identical to a sequence selected from SEQ ID NOs: 1-211; or (iv) a guide sequence that binds a target site comprising a genomic region listed in Tables 2-5; or (v) a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or (vi) a guide sequence that is at least 95%, 90%, or 85% identical to a sequence selected from (v); and optionally (b) an RNA-guided DNA binding agent or a nucleic acid encoding an RNA-guided DNA binding agent.

Embodiment 135 is the method of embodiment 133 or 134, wherein the RNA-guided DNA binding agent comprises a Cas9 protein.

Embodiment 136 is the method of embodiment 133 or 134, wherein the RNA-guided DNA-binding agent or nucleic acid encoding the RNA-guided DNA binding agent is S. pyogenes Cas9.

Embodiment 137 is the method of embodiment 133 or 134, wherein the RNA-guided DNA-binding agent or nucleic acid encoding the RNA-guided DNA binding agent is N. meningitidis Cas9.

Embodiment 138 is the method of embodiment 133 or 134, wherein the RNA-guided DNA-binding agent or nucleic acid encoding the RNA-guided DNA binding agent is S. thermophilus Cas9.

Embodiment 139 is the method of embodiment 133 or 134, wherein the RNA-guided DNA-binding agent or nucleic acid encoding the RNA-guided DNA binding agent is S. aureus Cas9.

Embodiment 140 is the method of embodiment 133 or 134, wherein the RNA-guided DNA-binding agent or nucleic acid encoding the RNA-guided DNA binding agent is Cpf1 from F. novicida.

Embodiment 141 is the method of embodiment 133 or 134, wherein the RNA-guided DNA-binding agent or nucleic acid encoding the RNA-guided DNA binding agent is Cpf1 from Acidaminococcus sp.

Embodiment 142 is the method of embodiment 133 or 134, wherein the RNA-guided DNA-binding agent or nucleic acid encoding the RNA-guided DNA binding agent is Cpf1 from Lachnospiraceae bacterium ND2006.

Embodiment 143 is the method of embodiment 133 or 134, wherein the RNA-guided DNA-binding agent or nucleic acid encoding the RNA-guided DNA binding agent is a C to T base editor.

Embodiment 144 is the method of embodiment 133 or 134, wherein the RNA-guided DNA-binding agent or nucleic acid encoding the RNA-guided DNA binding agent is a A to G base editor.

Embodiment 145 is the method of embodiment 133 or 134, wherein the RNA-guided DNA-binding agent or nucleic acid encoding the RNA-guided DNA binding agent comprises a APOBEC3A deaminase (A3A) and an RNA-guided nickase.

Embodiment 146 is the method of embodiment 133 or 134, wherein the RNA-guided DNA-binding agent or nucleic acid encoding the RNA-guided DNA binding agent is Cas12a.

Embodiment 147 is the method of embodiment 133 or 134, wherein the RNA-guided DNA-binding agent or nucleic acid encoding the RNA-guided DNA binding agent is CasX.

Embodiment 148 is the method of embodiment 133 or 134, wherein the RNA-guided DNA-binding agent or nucleic acid encoding the RNA-guided DNA binding agent is Nme2C as 9.

Embodiment 149 is the method of any one of embodiments 133-148, further comprising reducing or eliminating the surface expression of MHC class II protein in the cell relative to an unmodified cell, for example by contacting the cell with a gene editing system targeting a gene selected from CIITA, HLA-DR, HLA-DQ, HLA-DP, RFXS, RFXB/ANK, RFXAP, CREB, NF-YA, NF-YB, and NF-YC.

Embodiment 150 is the method of any one of embodiments 133-149, further comprising contacting the cell with a CIITA guide RNA.

Embodiment 151 is the method of any one of embodiments 133-150, further comprising reducing or eliminating the surface expression of a TCR protein in the cell relative to an unmodified cell.

Embodiment 152 is the method of any one of embodiments 133-151, further comprising contacting the cell with an exogenous nucleic acid.

Embodiment 153 is the method of embodiment 152, further comprising contacting the cell with an exogenous nucleic acid encoding a targeting receptor.

Embodiment 154 is the method of embodiment 152, further comprising contacting the cell with an exogenous nucleic acid encoding a polypeptide that is secreted by the cell.

Embodiment 155 is the method of embodiment 152, further comprising contacting the cell with a DNA-dependent protein kinase inhibitor (DNAPKi).

Embodiment 156 is the method of embodiment 155, wherein the DNAPKi is Compound 1.

Embodiment 157 is the engineered cell, population of cells, pharmaceutical composition, or method of any one of the preceding embodiments, wherein the cell is an allogeneic cell.

Embodiment 158 is the engineered cell, population of cells, pharmaceutical composition, or method of any one of the preceding embodiments, wherein the cell is a primary cell.

Embodiment 159 is the engineered cell, population of cells, pharmaceutical composition, or method of any one of the preceding embodiments, wherein the cell is a CD4+ T cell.

Embodiment 160 is the engineered cell, population of cells, pharmaceutical composition, or method of any one of the preceding embodiments, wherein the cell is a CD8+ T cell.

Embodiment 161 is the engineered cell, population of cells, pharmaceutical composition, or method of any one of the preceding embodiments, wherein the cell is a memory T cell.

Embodiment 162 is the engineered cell, population of cells, pharmaceutical composition, or method of any one of the preceding embodiments, wherein the cell is a B cell.

Embodiment 163 is the engineered cell, population of cells, pharmaceutical composition, or method of any one of the preceding embodiments, wherein the cell is a plasma B cell.

Embodiment 164 is the engineered cell, population of cells, pharmaceutical composition, or method of any one of the preceding embodiments, wherein the cell is a memory B cell.

Embodiment 165 is the engineered cell, population of cells, pharmaceutical composition, or method of any one of the preceding embodiments, wherein the cell is a natural killer (NK) cell.

Embodiment 166 is the engineered cell, population of cells, pharmaceutical composition, or method of any one of the preceding embodiments, wherein the cell is a macrophage.

Embodiment 167 is the engineered cell, population of cells, pharmaceutical composition, or method of any one of the preceding embodiments, wherein the cell is stem cell.

Embodiment 168 is the engineered cell, population of cells, pharmaceutical composition, or method of any one of the preceding embodiments, wherein the cell is a pluripotent stem cell (PSC).

Embodiment 169 is the engineered cell, population of cells, pharmaceutical composition, or method of any one of the preceding embodiments, wherein the cell is a hematopoietic stem cell (HSC).

Embodiment 170 is the engineered cell, population of cells, pharmaceutical composition, or method of any one of the preceding embodiments, wherein the cell is an induced pluripotent stem cell (iPSC).

Embodiment 171 is the engineered cell, population of cells, pharmaceutical composition, or method of any one of the preceding embodiments, wherein the cell is a mesenchymal stem cell (MSC).

Embodiment 172 The engineered cell, population of cells, pharmaceutical composition, or method of any one of the preceding embodiments, wherein the cell is a neural stem cell (NSC).

Embodiment 173 is the engineered cell, population of cells, pharmaceutical composition, or method of any one of the preceding embodiments, wherein the cell is a limbal stem cell (LSC).

Embodiment 174 is the engineered cell, population of cells, pharmaceutical composition, or method of any one of the preceding embodiments, wherein the cell is a progenitor cell, e.g. an endothelial progenitor cell or a neural progenitor cell.

Embodiment 175 is the engineered cell, population of cells, pharmaceutical composition, or method of any one of the preceding embodiments, wherein the cell is a tissue-specific primary cell.

Embodiment 176 is the engineered cell, population of cells, pharmaceutical composition, or method of any one of the preceding embodiments, wherein the cell is a chosen from: chondrocyte, myocyte, and keratinocyte.

Embodiment 177 is the engineered cell, population of cells, pharmaceutical composition, or method of any one of the preceding embodiments, wherein the cell is an activated cell.

Embodiment 178 is the engineered cell, population of cells, pharmaceutical composition, or method of any one of the preceding embodiments, wherein the cell is a non-activated cell.

Embodiment 179 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an antibody or antibody fragment.

Embodiment 180 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a full-length IgG antibody.

Embodiment 181 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a single chain antibody.

Embodiment 182 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a neutralizing antibody.

Embodiment 183 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is an enzyme.

Embodiment 184 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a cytokine.

Embodiment 185 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide is a fusion protein.

Embodiment 186 is the engineered cell, population of cells, pharmaceutical composition, or method of any one of the preceding embodiments, comprising an exogenous nucleic acid encoding a polypeptide that is secreted by the cell or contacting the cell with said exogenous nucleic acid, wherein the secreted polypeptide comprises a soluble receptor.

Embodiment 187 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a T cell receptor (TCR).

Embodiment 188 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a genetically modified TCR.

Embodiment 189 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a WT1 TCR.

Embodiment 190 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a CAR.

Embodiment 191 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a universal CAR.

Embodiment 192 is the engineered cell, population of cells, pharmaceutical composition, or method of any one of the preceding embodiments, comprising an exogenous nucleic acid encoding a targeting receptor or contacting the cell with an exogenous nucleic acid encoding a targeting receptor, wherein the targeting receptor is a proliferation-inducing ligand (APRIL).

Embodiment 193 is the engineered cell, population of cells, pharmaceutical composition, or method of any one of the preceding embodiments, wherein the cells are engineered with a gene editing system.

Embodiment 194 is the engineered cell, population of cells, pharmaceutical composition, or method of embodiment 193, wherein the gene editing system comprises a transcription activator-like effector nuclease (TALEN).

Embodiment 195 is the engineered cell, population of cells, pharmaceutical composition, or method of embodiment 193, wherein the gene editing system comprises a zinc finger nuclease.

Embodiment 196 is the engineered cell, population of cells, pharmaceutical composition, or method of embodiment 193, wherein the gene editing system comprises an RNA-guided DNA binding agent or a nucleic acid encoding an RNA-guided DNA binding agent, optionally wherein the RNA-guided DNA binding agent is Cas9.

Embodiment 197 is the engineered cell, population of cells, pharmaceutical composition, or method of any one of the preceding embodiments, wherein the HLA-A guide RNA is provided to the cell in a vector.

Embodiment 198 is the engineered cell, population of cells, pharmaceutical composition, or method of any one of the preceding embodiments, wherein the RNA-guided DNA binding agent is provided to the cell in a vector, optionally in the same vector as the HLA-A guide RNA.

Embodiment 199 is the engineered cell, population of cells, pharmaceutical composition, or method of any one of the preceding embodiments, wherein the exogenous nucleic acid is provided to the cell in a vector.

Embodiment 200 is the engineered cell, population of cells, pharmaceutical composition, or method of any one of the preceding embodiments, wherein the vector is a viral vector.

Embodiment 201 is the engineered cell, population of cells, pharmaceutical composition, or method of any one of the preceding embodiments, wherein the vector is a non-viral vector.

Embodiment 202 is the engineered cell, population of cells, pharmaceutical composition, or method of any one of the preceding embodiments, wherein the vector is a lentiviral vector.

Embodiment 203 is the engineered cell, population of cells, pharmaceutical composition, or method of any one of the preceding embodiments, wherein the vector is a retroviral vector.

Embodiment 204 is the engineered cell, population of cells, pharmaceutical composition, or method of any one of the preceding embodiments, wherein the vector is an AAV.

Embodiment 205 is the engineered cell, population of cells, pharmaceutical composition, or method of any one of the preceding embodiments, wherein the guide RNA is provided to the cell in a lipid nucleic acid assembly composition, optionally in the same lipid nucleic acid assembly composition as an RNA-guided DNA binding agent.

Embodiment 206 is the engineered cell, population of cells, pharmaceutical composition, or method of any one of the preceding embodiments, wherein the exogenous nucleic acid is provided to the cell in a lipid nucleic acid assembly composition.

Embodiment 207 is the engineered cell, population of cells, pharmaceutical composition, or method of any one of the preceding embodiments, wherein the lipid nucleic acid assembly composition is a lipid nanoparticle (LNP).

Embodiment 208 is the engineered cell, population of cells, pharmaceutical composition, or method of any one of the preceding embodiments, wherein the exogenous nucleic acid is integrated into the genome of the cell.

Embodiment 209 is the engineered cell, population of cells, pharmaceutical composition, or method of any one of the preceding embodiments, wherein the exogenous nucleic acid is integrated into the genome of the cell by homologous recombination (HR).

Embodiment 210 is the engineered cell, population of cells, pharmaceutical composition, or method of any one of the preceding embodiments, wherein the exogenous nucleic acid is integrated into a safe harbor locus in the genome of the cell.

Embodiment 211 is the engineered cell, population of cells, pharmaceutical composition, or method of any one of the preceding embodiments, wherein the HLA-A guide RNA comprises SEQ ID NO: 13 or wherein the HLA-A guide RNA comprises SEQ ID NO: 14.

Embodiment 212 is the engineered cell, population of cells, pharmaceutical composition, or method of any one of the preceding embodiments, wherein the HLA-A guide RNA comprises SEQ ID NO: 15.

Embodiment 213 is the engineered cell, population of cells, pharmaceutical composition, or method of any one of the preceding embodiments, wherein the HLA-A guide RNA comprises SEQ ID NO: 16.

Embodiment 214 is the engineered cell, population of cells, pharmaceutical composition, or method of any one of the preceding embodiments, wherein the HLA-A guide RNA comprises SEQ ID NO: 17.

Embodiment 215 is the engineered cell, population of cells, pharmaceutical composition, or method of any one of the preceding embodiments, wherein the HLA-A guide RNA comprises SEQ ID NO: 18.

Embodiment 216 is the engineered cell, population of cells, pharmaceutical composition, or method of any one of the preceding embodiments, wherein the HLA-A guide RNA comprises SEQ ID NO: 26.

Embodiment 217 is the engineered cell, population of cells, pharmaceutical composition, or method of any one of the preceding embodiments, wherein the HLA-A guide RNA comprises SEQ ID NO: 37.

Embodiment 218 is the engineered cell, population of cells, pharmaceutical composition, or method of any one of the preceding embodiments, wherein the HLA-A guide RNA comprises SEQ ID NO: 38.

Embodiment 219 is the engineered cell, population of cells, pharmaceutical composition, or method of any one of the preceding embodiments, wherein the HLA-A guide RNA comprises SEQ ID NO: 39.

Embodiment 220 is the engineered cell, population of cells, pharmaceutical composition, or method of any one of the preceding embodiments, wherein the HLA-A guide RNA comprises SEQ ID NO: 41.

Embodiment 221 is the engineered cell, population of cells, pharmaceutical composition, or method of any one of the preceding embodiments, wherein the HLA-A guide RNA comprises SEQ ID NO: 43.

Embodiment 222 is the engineered cell, population of cells, pharmaceutical composition, or method of any one of the preceding embodiments, wherein the HLA-A guide RNA comprises SEQ ID NO: 45.

Embodiment 223 is the engineered cell, population of cells, pharmaceutical composition, or method of any one of the preceding embodiments, wherein the HLA-A guide RNA comprises SEQ ID NO: 62.

Embodiment 224 is the engineered cell, population of cells, pharmaceutical composition, or method of any one of the preceding embodiments, wherein the HLA-A guide RNA comprises at least one modification.

Embodiment 225 is the engineered cell, population of cells, pharmaceutical composition, or method of any one of the preceding embodiments, wherein the HLA-A guide RNA comprises at least one modification, wherein the at least one modification includes a 2′-O-methyl (2′-O-Me) modified nucleotide.

Embodiment 226 is the engineered cell, population of cells, pharmaceutical composition, or method of any one of the preceding embodiments, wherein the HLA-A guide RNA comprises at least one modification, comprising a phosphorothioate (PS) bond between nucleotides.

Embodiment 227 is the engineered cell, population of cells, pharmaceutical composition, or method of any one of the preceding embodiments, wherein the HLA-A guide RNA comprises at least one modification, comprising a 2′-fluoro (2′-F) modified nucleotide.

Embodiment 228 is the engineered cell, population of cells, pharmaceutical composition, or method of any one of the preceding embodiments, wherein the HLA-A guide RNA comprises at least one modification, comprising a modification at one or more of the first five nucleotides at the 5′ end of the guide RNA.

Embodiment 229 is the engineered cell, population of cells, pharmaceutical composition, or method of any one of the preceding embodiments, wherein the HLA-A guide RNA comprises at least one modification, comprising a modification at one or more of the last five nucleotides at the 3′ end of the guide RNA.

Embodiment 230 is the engineered cell, population of cells, pharmaceutical composition, or method of any one of the preceding embodiments, wherein the HLA-A guide RNA comprises at least one modification, comprising a PS bond between the first four nucleotides of the guide RNA.

Embodiment 231 is the engineered cell, population of cells, pharmaceutical composition, or method of any one of the preceding embodiments, wherein the HLA-A guide RNA comprises at least one modification, comprising a PS bond between the last four nucleotides of the guide RNA.

Embodiment 232 is the engineered cell, population of cells, pharmaceutical composition, or method of any one of the preceding embodiments, wherein the HLA-A guide RNA comprises at least one modification, comprising a 2′-O-Me modified nucleotide at the first three nucleotides at the 5′ end of the guide RNA.

Embodiment 233 is the engineered cell, population of cells, pharmaceutical composition, or method of any one of the preceding embodiments, wherein the HLA-A guide RNA comprises at least one modification, comprising a 2′-O-Me modified nucleotide at the last three nucleotides at the 3′ end of the guide RNA.

Embodiment 234 is the engineered cell, population of cells, pharmaceutical composition, or method of any one of the preceding embodiments, for use to express a TCR with specificity for a polypeptide expressed by cancer cells.

Embodiment 235 is the engineered cell, population of cells, pharmaceutical composition, or method of any one of the preceding embodiments, for use in administering to a subject as an adoptive cell transfer (ACT) therapy.

Embodiment 236 is the engineered cell, population of cells, pharmaceutical composition, or method of any one of the preceding embodiments, for use in treating a subject with cancer.

Embodiment 237 is the engineered cell, population of cells, pharmaceutical composition, or method of any one of the preceding embodiments, for use in treating a subject with an infectious disease.

Embodiment 238 is the engineered cell, population of cells, pharmaceutical composition, or method of any one of the preceding embodiments, for use in treating a subject with an autoimmune disease

Embodiment 239 is a cell bank comprising: (a) the engineered cells of any one of the preceding embodiments, or the engineered cells produced by the method of any one of the preceding embodiments; and (b) a catalogue comprising information documenting the HLA-B and HLA-C alleles of the donor cells in the cell bank.

Embodiment 240 is the cell bank of embodiment 239, wherein the cell bank comprises at least 3, 4, 5, 6, 7, 8, 9, 10, 11, 12, 13, 14, 15, 16, 17, 18, 19, 20, 25, 30, 35, or 40 donor cells that have a unique combination of HLA-B and HLA-C alleles as compared to other donor cells in the cell bank.

Embodiment 241 is a method of administering an engineered cell to a recipient subject in need thereof, the method comprising: (a) determining the HLA-B and HLA-C alleles of the recipient subject; (b) selecting an engineered cell or cell population of any one of the preceding embodiments, or engineered cell or cell population produced by the method of any one of the preceding embodiments, wherein the engineered cell comprises at least one of the same HLA-B or HLA-C alleles as the recipient subject; (c) administering the selected engineered cell to the recipient subject.

Embodiment 242 is the method of embodiment 241, wherein the subject has the HLA-B and HLA-C alleles of the engineered cell.

Embodiment 243 is the engineered cell, composition, pharmaceutical composition, or method of any one of the preceding embodiments, for use in administering to a partially matched subject for an adoptive cell transfer (ACT) therapy, wherein the partially matched subject has the HLA-B and HLA-C alleles of the engineered cell or cell population.

Embodiment 244 is the engineered cell, composition, pharmaceutical composition, or method of any one of embodiments 130-132, 235-238, 241-243, wherein the engineered cell or cell population comprises HLA-B and HLA-C alleles shared with the subject.

Embodiment 245 is the engineered cell, composition, pharmaceutical composition, or method of any one of the preceding embodiments 130-132, 235-238, 241-243, wherein the HLA-B and HLA-C alleles of the engineered cell or cell population consist of alleles that match one or more HLA-B and HLA-C alleles of the subject.

Embodiment 246 is the engineered cell, composition, pharmaceutical composition, or method of any one of the preceding embodiments 130-132, 235-238, 241-243, wherein the HLA-B and HLA-C alleles of the engineered cell or cell population consist of alleles that match one or both HLA-B and/or one or both HLA-C alleles of the subject.

1. An engineered human cell, which has reduced or eliminated surface expression of HLA-A relative to an unmodified cell, comprising a genetic modification in the HLA-A gene, wherein the cell is homozygous for HLA-B and homozygous for HLA-C.

2. An engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from:
a. chr6:29942854-chr6:29942913 and
b. chr6:29943518-chr6:29943619;
wherein the cell is homozygous for HLA-B and homozygous for HLA-C.

3. The engineered cell of claim 1, wherein the cell has reduced or eliminated expression of at least one HLA-A allele selected from: HLA-A1, HLA-A2, HLA-A3, HLA-A11, and HLA-A24.

4. The engineered cell of claim 1, wherein the genetic modification comprises at least one nucleotide within the genomic coordinates chr6:29942864-chr6:29942903; or
wherein the genetic modification comprises at least one nucleotide within the genomic coordinates chr6:29943528-chr6:29943609; or
wherein the genetic modification comprises at least one nucleotide within the genomic coordinates chosen from: chr6:29942864-29942884; chr6:29942868-29942888; chr6:29942876-29942896; chr6:29942877-29942897; and chr6:29942883-29942903; or
wherein the genetic modification comprises at least one nucleotide within the genomic coordinates chosen from: chr6:29943528-29943548; chr6:29943529-29943549; chr6:29943530-29943550; chr6:29943537-29943557; chr6:29943549-29943569; and chr6:29943589-29943609; or
wherein the genetic modification comprises at least one nucleotide within the genomic coordinates chr6:29942876-29942897; or
wherein the genetic modification comprises at least one nucleotide within the genomic coordinates chr6:29943528-29943550; or
wherein the genetic modification comprises at least one nucleotide within the genomic coordinates chosen from: chr6:29942864-29942884, chr6:29942868-29942888, chr6:29942876-29942896, and chr6:29942877-29942897; or
wherein the genetic modification comprises at least one nucleotide within the genomic coordinates chosen from: chr6:29943528-29943548, chr6:29943529-29943549, and chr6:29943530-29943550.

5-11. (canceled)

12. An engineered human cell, which has reduced or eliminated surface expression of HLA-A relative to an unmodified cell, comprising a genetic modification in the HLA-A gene, wherein the genetic modification comprises at least one nucleotide within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

13. An engineered human cell, which has reduced or eliminated surface expression of HLA-A relative to an unmodified cell, comprising a genetic modification in an HLA-A gene, wherein the genetic modification comprises an indel, a C to T substitution, or an A to G substitution within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

14-16. (canceled)

17. The engineered cell of claim 1, wherein the HLA-A expression is reduced or eliminated by a gene editing system that binds to an HLA-A genomic target sequence comprising at least 5 contiguous nucleotides within the genomic coordinates chosen from:
a. chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046, chr6:29934330-29934350, chr6:29943115-29943135, chr6:29943135-29943155, chr6:29943140-29943160, chr6:29943590-29943610, chr6:29943824-29943844, chr6:29943858-29943878, chr6:29944478-29944498, and chr6:29944850-29944870;
b. chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046;
c. chr6:29942864-29942884; chr6:29942868-29942888; chr6:29942876-29942896; chr6:29942877-29942897; chr6:29942883-29942903; chr6:29943528-29943548; chr6:29943529-29943549; chr6:29943530-29943550; chr6:29943537-29943557; chr6:29943549-29943569; and chr6:29943589-29943609;
d. chr6:29942864-29942884; chr6:29942868-29942888; chr6:29942876-29942896; chr6:29942877-29942897; and chr6:29942883-29942903;
e. chr6:29943528-29943548; chr6:29943529-29943549; chr6:29943530-29943550; chr6:29943537-29943557; chr6:29943549-29943569; and chr6:29943589-29943609;
f. chr6:29942864-29942884, chr6:29942868-29942888, chr6:29942876-29942896, and chr6:29942877-29942897;
g. chr6:29943528-29943548, chr6:29943529-29943549, and chr6:29943530-29943550;
h. chr6:29945290-29945310, chr6:29945296-29945316, and chr6:29945297-29945317, chr6:29945300-29945320;
i. chr6:29890117-29890137, chr6:29927058-29927078, chr6:29934330-29934350, chr6:29942541-29942561, chr6:29942542-29942562, chr6:29942543-29942563, chr6:29942543-29942563, chr6:29942550-29942570, chr6:29942864-29942884, chr6:29942868-29942888, chr6:29942876-29942896, chr6:29942876-29942896, chr6:29942877-29942897, chr6:29942883-29942903, chr6:29943062-29943082, chr6:29943063-29943083, chr6:29943092-29943112, chr6:29943115-29943135, chr6:29943118-29943138, chr6:29943119-29943139, chr6:29943120-29943140, chr6:29943126-29943146, chr6:29943128-29943148, chr6:29943129-29943149, chr6:29943134-29943154, chr6:29943134-29943154, chr6:29943135-29943155, chr6:29943136-29943156, chr6:29943140-29943160, chr6:29943142-29943162, chr6:29943143-29943163, chr6:29943188-29943208, chr6:29943528-29943548, chr6:29943529-29943549, chr6:29943530-29943550, chr6:29943536-29943556, chr6:29943537-29943557, chr6:29943538-29943558, chr6:29943549-29943569, chr6:29943556-29943576, chr6:29943589-29943609, chr6:29943590-29943610, chr6:29943590-29943610, chr6:29943599-29943619, chr6:29943600-29943620, chr6:29943601-29943621, chr6:29943602-29943622, chr6:29943603-29943623, chr6:29943774-29943794, chr6:29943779-29943799, chr6:29943780-29943800, chr6:29943822-29943842, chr6:29943824-29943844, chr6:29943857-29943877, chr6:29943858-29943878, chr6:29943859-29943879, chr6:29943860-29943880, chr6:29944026-29944046, chr6:29944077-29944097, chr6:29944078-29944098, chr6:29944458-29944478, chr6:29944478-29944498, chr6:29944597-29944617, chr6:29944642-29944662, chr6:29944643-29944663, chr6:29944772-29944792, chr6:29944782-29944802, chr6:29944850-29944870, chr6:29944907-29944927, chr6:29945024-29945044, chr6:29945097-29945117, chr6:29945104-29945124, chr6:29945105-29945125, chr6:29945116-29945136, chr6:29945118-29945138, chr6:29945119-29945139, chr6:29945124-29945144, chr6:29945176-29945196, chr6:29945177-29945197, chr6:29945177-29945197, chr6:29945180-29945200, chr6:29945187-29945207, chr6:29945188-29945208, chr6:29945228-29945248, chr6:29945230-29945250, chr6:29945231-29945251, chr6:29945232-29945252, chr6:29945308-29945328, chr6:29945361-29945381, chr6:29945362-29945382, and chr6:31382543-31382563;
j. chr6:29942815-29942835, chr6:29942816-29942836, chr6:29942817-29942837, chr6:29942817-29942837, chr6:29942828-29942848, chr6:29942837-29942857, chr6:29942885-29942905, chr6:29942895-29942915, chr6:29942896-29942916, chr6:29942898-29942918, chr6:29942899-29942919, chr6:29942900-29942920, chr6:29942904-29942924, chr6:29942905-29942925, chr6:29942912-29942932, chr6:29942913-29942933, chr6:29943490-29943510, chr6:29943497-29943517, chr6:29943498-29943518, chr6:29943502-29943522, chr6:29943502-29943522, chr6:29943511-29943531, chr6:29943520-29943540, chr6:29943521-29943541, chr6:29943566-29943586, chr6:29943569-29943589, chr6:29943569-29943589, chr6:29943570-29943590, chr6:29943573-29943593, chr6:29943578-29943598, chr6:29943585-29943605, chr6:29943589-29943609, chr6:29943568-29943588, and chr6:29942815-29942835;
k. chr6:29942884-29942904, chr6:29943519-29943539, chr6:29942863-29942883;
l. chr6:29943517-29943537, and chr6:29943523-29943543;
m. chr6:29942845-29942869, chr6:29942852-29942876, chr6:29942865-29942889, chr6:29942891-29942915, chr6:29942895-29942919, chr6:29942903-29942927, chr6:29942904-29942928, chr6:29943518-29943542, chr6:29943525-29943549, chr6:29943535-29943559, chr6:29943538-29943562, chr6:29943539-29943563, chr6:29943547-29943571, chr6:29943547-29943571, chr6:29943548-29943572, chr6:29943555-29943579, chr6:29943556-29943580, chr6:29943557-29943581, chr6:29943558-29943582, chr6:29943559-29943583, chr6:29943563-29943587, chr6:29943564-29943588, chr6:29943565-29943589, chr6:29943568-29943592, chr6:29943571-29943595, chr6:29943572-29943596, chr6:29943595-29943619, chr6:29943596-29943620, and chr6:29943600-29943624;
n. chr6:29942885-29942905, chr6:29942895-29942915, chr6:29942896-29942916, chr6:29942898-29942918, chr6:29942899-29942919, chr6:29942900-29942920, chr6:29942904-29942924, chr6:29943511-29943531, chr6:29943520-29943540, chr6:29943521-29943541, chr6:29943529-29943549, chr6:29943566-29943586, chr6:29943568-29943588, chr6:29943569-29943589, chr6:29943569-29943589, chr6:29943570-29943590, chr6:29943573-29943593, chr6:29943578-29943598, chr6:29943585-29943605, and chr6:29943589-29943609; or
o. chr6:29942469-29942489, chr6:29943058-29943078, chr6:29943063-29943083, chr6:29943080-29943100, chr6:29943187-29943207, chr6:29943192-29943212, chr6:29943197-29943217, chr6:29943812-29943832, chr6:29944349-29944369, chr6:29944996-29945016, chr6:29945018-29945038, and chr6:29945341-29945361, chr6:29945526-29945546.

18. The engineered cell of claim 1, wherein the HLA-A expression is reduced or eliminated by a gene editing system that binds to an HLA-A genomic target sequence comprising at least 5 contiguous nucleotides within the genomic coordinates chosen from chr6:29942854-chr6:29942913 and chr6:29943518-chr6:29943619; or
wherein the HLA-A expression is reduced or eliminated by a gene editing system that binds to an HLA-A genomic target sequence comprising at least 5 contiguous nucleotides within the genomic coordinates chr6:29942876-29942897; or
wherein the HLA-A expression is reduced or eliminated by a gene editing system that binds to an HLA-A genomic target sequence comprising at least 5 contiguous nucleotides within the genomic coordinates chr6:29943528-29943550; or
wherein the HLA-A expression is reduced or eliminated by a gene editing system that binds to an HLA-A genomic target sequence comprising at least 5 contiguous nucleotides within the genomic coordinates chosen from: chr6:29942864-29942884; chr6:29942868-29942888; chr6:29942876-29942896; chr6:29942877-29942897; chr6:29942883-29942903; chr6:29943126-29943146; chr6:29943528-29943548; chr6:29943529-29943549; chr6:29943530-29943550; chr6:29943537-29943557; chr6:29943549-29943569; chr6:29943589-29943609; and chr6:29944026-29944046.

19-21. (canceled)

22. The engineered cell of claim 17, wherein the HLA-A genomic target sequence comprises at least 10, at least 15, at least 16, at least 17, at least 18, at least 19, or at least 20 contiguous nucleotides within the genomic coordinates.

23. The engineered cell of claim 12, wherein the cell is homozygous for HLA-B and homozygous for HLA-C.

24. The engineered cell of claim 1, wherein the HLA-B allele is selected from any one of the following HLA-B alleles: HLA-B*07:02; HLA-B*08:01; HLA-B*44:02; HLA-B*35:01; HLA-B*40:01; HLA-B*57:01; HLA-B*14:02; HLA-B*15:01; HLA-B*13:02; HLA-B*44:03; HLA-B*38:01; HLA-B*18:01; HLA-B*44:03; HLA-B*51:01; HLA-B*49:01; HLA-B*15:01; HLA-B*18:01; HLA-B*27:05; HLA-B*35:03; HLA-B*18:01; HLA-B*52:01; HLA-B*51:01; HLA-B*37:01; HLA-B*53:01; HLA-B*55:01; HLA-B*44:02; HLA-B*44:03; HLA-B*35:02; HLA-B*15:01; and HLA-B*40:02; or
wherein the HLA-C allele is selected from any one of the following HLA-C alleles: HLA-C*07:02, HLA-C*07:01, HLA-C*05:01, HLA-C*04:01 HLA-C*03:04, HLA-C*06:02, HLA-C*08:02, HLA-C*03:03, HLA-C*06:02, HLA-C*16:01, HLA-C*12:03, HLA-C*07:01, HLA-C*04:01, HLA-C*15:02, HLA-C*07:01, HLA-C*03:04, HLA-C*12:03, HLA-C*02:02, HLA-C*04:01, HLA-C*05:01, HLA-C*12:02, HLA-C*14:02, HLA-C*06:02, HLA-C*04:01, HLA-C*03:03, HLA-C*07:04, HLA-C*07:01, HLA-C*04:01, HLA-C*04:01, and HLA-C*02:02, or
wherein the HLA-B allele is selected from any one of the following HLA-B alleles: HLA-B*07:02, HLA-B*08:01, HLA-B*44:02, HLA-B*35:01, HLA-B*40:01, HLA-B*57:01, HLA-B*14:02, HLA-B*15:01, HLA-B*13:02, HLA-B*44:03, HLA-B*38:01, HLA-B*18:01, HLA-B*44:03, HLA-B*51:01, HLA-B*49:01, HLA-B*15:01, HLA-B*18:01, HLA-B*27:05, HLA-B*35:03, HLA-B*18:01, HLA-B*52:01, HLA-B*51:01, HLA-B*37:01, HLA-B*53:01, HLA-B*55:01, HLA-B*44:02, HLA-B*44:03, HLA-B*35:02, HLA-B*15:01, and HLA-B*40:02; and
the HLA-C allele is selected from any one of the following HLA-C alleles: HLA-C*07:02, HLA-C*07:01, HLA-C*05:01, HLA-C*04:01 HLA-C*03:04, HLA-C*06:02, HLA-C*08:02, HLA-C*03:03, HLA-C*06:02, HLA-C*16:01, HLA-C*12:03, HLA-C*07:01, HLA-C*04:01, HLA-C*15:02, HLA-C*07:01, HLA-C*03:04, HLA-C*12:03, HLA-C*02:02, HLA-C*04:01, HLA-C*05:01, HLA-C*12:02, HLA-C*14:02, HLA-C*06:02, HLA-C*04:01, HLA-C*03:03, HLA-C*07:04, HLA-C*07:01, HLA-C*04:01, HLA-C*04:01, and HLA-C*02:02; or
wherein the HLA-B and HLA-C alleles are selected from any one of the following HLA-B and HLA-C alleles: HLA-B*07:02 and HLA-C*07:02, HLA-B*08:01 and HLA-C*07:01, HLA-B*44:02 and HLA-C*05:01, HLA-B*35:01 and HLA-C*04:01, HLA-B*40:01 and HLA-C*03:04, HLA-B*57:01 and HLA-C*06:02, HLA-B*14:02 and HLA-C*08:02, HLA-B*15:01 and HLA-C*03:03, HLA-B*13:02 and HLA-C*06:02, HLA-B*44:03 and HLA-C*16:01, HLA-B*38:01 and HLA-C*12:03; HLA-B*18:01 and HLA-C*07:01; HLA-B*44:03 and HLA-C*04:01; HLA-B*51:01 and HLA-C*15:02; HLA-B*49:01 and HLA-C*07:01; HLA-B*15:01 and HLA-C*03:04; HLA-B*18:01 and HLA-C*12:03; HLA-B*27:05 and HLA-C*02:02; HLA-B*35:03 and HLA-C*04:01; HLA-B*18:01 and HLA-C*05:01; HLA-B*52:01 and HLA-C*12:02; HLA-B*51:01 and HLA-C*14:02; HLA-B*37:01 and HLA-C*06:02; HLA-B*53:01 and HLA-C*04:01; HLA-B*55:01 and HLA-C*03:03; HLA-B*44:02 and HLA-C*07:04; HLA-B*44:03 and HLA-C*07:01; HLA-B*35:02 and HLA-C*04:01; HLA-B*15:01 and HLA-C*04:01; and HLA-B*40:02 and HLA-C*02:02; or
wherein the HLA-B and HLA-C alleles are HLA-B*07:02 and HLA-C*07:02; or
wherein the HLA-B and HLA-C alleles are HLA-B*08:01 and HLA-C*07:01; or
wherein the HLA-B and HLA-C alleles are HLA-B*44:02 and HLA-C*05:01; or
wherein the HLA-B and HLA-C alleles are HLA-B*35:01 and HLA-C*04:01.

25-31. (canceled)

32. The engineered cell of claim 1, wherein the cell has reduced expression of MHC class II protein on the surface of the cell; or
wherein the cell has a genetic modification of a gene selected from CIITA, HLA-DR, HLA-DQ, HLA-DP, RFXS, RFXB/ANK, RFXAP, CREB, NF-YA, NF-YB, and NF-YC; or
wherein the cell has a genetic modification in the CIITA gene; or
wherein the cell has reduced expression of TRAC protein or TRBC protein on the surface of the cell.

33-35. (canceled)

36. The engineered cell of claim 1, wherein the engineered cell comprises an exogenous nucleic acid encoding a targeting receptor that is expressed on the surface of the engineered cell or a ligand for the receptor, and optionally
wherein the targeting receptor is a CAR or a TCR; or
wherein the engineered cell further comprises an exogenous nucleic acid encoding a polypeptide that is secreted by the engineered cell.

37-38. (canceled)

39. The engineered cell of claim 1, wherein the engineered cell is an immune cell; or
wherein the engineered cell is a primary cell; or
wherein the engineered cell is a monocyte, macrophage, mast cell, dendritic cell, or granulocyte; or
wherein the engineered cell is a lymphocyte; or
wherein the engineered cell is a T cell.

40-43. (canceled)

44. The engineered cell of claim 1, wherein the genetic modification comprises at least 5, at least 6, at least 7, at least 8, at least 9, or at least 10 contiguous nucleotides within the genomic coordinates.

45. The engineered cell of claim 1, wherein the genetic modification comprises an indel; or
wherein the genetic modification comprises at least one C to T substitution or at least one A to G substitution within the genomic coordinates.

46-47. (canceled)

48. A population of cells comprising the engineered cell of claim 1.

49. A pharmaceutical composition comprising the population of cells of claim 48.

50. The population of claim 48, or pharmaceutical composition of claim 49, wherein the population of cells is at least 65%, at least 70%, at least 80%, at least 90%, at least 92%, at least 93%, at least 94%, at least 95%, at least 96%, at least 97%, at least 98%, or at least 99% HLA-A negative as measured by flow cytometry; or
wherein the population of cells is at least 94%, at least 95%, at least 96%, at least 97%, at least 98%, or at least 99% CIITA negative as measured by flow cytometry; or
wherein the population of cells is at least 95%, at least 97%, at least 98%, at least 99%, or at least 99.5% endogenous TCR protein negative as measured by flow cytometry.

51-52. (canceled)

53. A method of administering the engineered cell of claim 1 to a subject in need thereof, to a subject for treating the subject with treating a disease or disorder, to a subject for treating the subject with cancer, an infectious disease, or an autoimmune disease, or to a subject as an adoptive cell transfer (ACT) therapy.

54-55. (canceled)

56. A method of making an engineered human cell, which has reduced or eliminated surface expression of HLA-A protein relative to an unmodified cell, wherein the cell is homozygous for HLA-B and homozygous for HLA-C, comprising contacting a cell with composition comprising:
a. an HLA-A guide RNA comprising
i. a guide sequence selected from SEQ ID NOs: 1-211; or
ii. at least 17, 18, 19, or 20 contiguous nucleotides of a sequence selected from SEQ ID NOs: 1-211; or
iii. a guide sequence at least 95%, 90%, or 85% identical to a sequence selected from SEQ ID NOs: 1-211; or
iv. a guide sequence that binds a target site comprising a genomic region listed in Tables 2-5; or
v. a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or
vi. a guide sequence that is at least 95%, 90%, or 85% identical to a sequence selected from (v); and optionally
b. an RNA-guided DNA binding agent or a nucleic acid encoding an RNA-guided DNA binding agent.

57. A method of reducing surface expression of HLA-A protein in a human cell relative to an unmodified cell, comprising contacting a cell with composition comprising:
a. an HLA-A guide RNA comprising
i. a guide sequence selected from SEQ ID NOs: 1-211; or
ii. at least 17, 18, 19, or 20 contiguous nucleotides of a sequence selected from SEQ ID NOs: 1-211; or
iii. a guide sequence at least 95%, 90%, or 85% identical to a sequence selected from SEQ ID NOs: 1-211; or
iv. a guide sequence that binds a target site comprising a genomic region listed in Tables 2-5; or
v. a guide sequence that is complementary to at least 17, 18, 19, or 20 contiguous nucleotides of a genomic region listed in Tables 1-2 and 5, or a guide sequence that is complementary to at least 17, 18, 19, 20, 21, 22, 23, or 24 contiguous nucleotides of a genomic region listed in Table 4; or
vi. a guide sequence that is at least 95%, 90%, or 85% identical to a sequence selected from (v); and optionally
b. an RNA-guided DNA binding agent or a nucleic acid encoding an RNA-guided DNA binding agent.

58. The method of claim 56, wherein the RNA-guided DNA binding agent comprises a Cas9 protein; or
wherein the RNA-guided DNA-binding agent or nucleic acid encoding the RNA-guided DNA binding agent is S. pyogenes Cas9, N. meningitidis Cas9, S. thermophilus Cas9, S. aureus Cas9, Cpf1 from F. novicida, Cpf1 from Acidaminococcus sp., or Cpf1 from Lachnospiraceae bacterium ND2006, or
wherein the RNA-guided DNA-binding agent or nucleic acid encoding the RNA-guided DNA binding agent is a C to T base editor, an A to G base editor, or a APOBEC3A deaminase (A3A) and an RNA-guided nickase.

59-60. (canceled)

61. The method of claim 56, further comprising reducing or eliminating the surface expression of MHC class II protein in the cell relative to an unmodified cell, by contacting the cell with a gene editing system targeting a gene selected from CIITA, HLA-DR, HLA-DQ, HLA-DP, RFXS, RFXB/ANK, RFXAP, CREB, NF-YA, NF-YB, and NF-YC; or
further comprising contacting the cell with a CIITA guide RNA; or
further comprising reducing or eliminating the surface expression of a TCR protein in the cell relative to an unmodified cell.

62-63. (canceled)

64. The method of claim 56, further comprising contacting the cell with an exogenous nucleic acid, and optionally wherein the exogenous nucleic acid encodes a targeting receptor or a polypeptide that is secreted by the cell.

65. (canceled)

66. The method of claim 64, further comprising contacting the cell with a DNA-dependent protein kinase inhibitor (DNAPKi), optionally wherein the DNAPKi is Compound 1.

67. The engineered cell of claim 1, wherein the cell is an allogeneic cell; or
wherein the cell is a primary cell; or
wherein the cell is a T cell, and optionally wherein the T cell is a CD4+ T cell, a CD8+ T cell, or a memory T cell; or
wherein the cell is a B cell, and optionally wherein the B cell is a plasma B cell or a memory B cell; or
wherein the cell is a stem cell, and optionally wherein the stem cell is a pluripotent stem cell (PSC), a hematopoietic stem cell (HSC), an induced pluripotent stem cell (iPSC), a mesenchymal stem cell (MSC), a neural stem cell (NSC), or a limbal stem cell (LSC).

68-71. (canceled)

72. The engineered cell of claim 1, comprising an exogenous nucleic acid encoding a polypeptide that is secreted by the cell, wherein the secreted polypeptide is an antibody or antibody fragment, or wherein the secreted polypeptide is a full-length IgG antibody, a single chain antibody, or a neutralizing antibody, or wherein the secreted polypeptide is a cytokine; or
comprising an exogenous nucleic acid encoding a targeting receptor, wherein the targeting receptor is a T cell receptor (TCR), a CAR, or a proliferation-inducing ligand (APRIL).

73-75. (canceled)

76. The engineered cell of claim 1, wherein the cell is engineered with a gene editing system, and optionally
wherein the gene editing system comprises a transcription activator-like effector nuclease (TALEN) or a zinc finger nuclease, or optionally
wherein the gene editing system comprises an RNA-guided DNA binding agent or a nucleic acid encoding an RNA-guided DNA binding agent, optionally wherein the RNA-guided DNA binding agent is Cas9.

77-78. (canceled)

79. The method of claim 64, wherein the HLA-A guide RNA, the RNA-guided DNA binding agent, and/or the exogenous nucleic acid is provided to the cell in a vector, optionally wherein the HLA-A guide RNA and the RNA-guided DNA binding agent are provided in the same vector; or
wherein the guide RNA or the exogenous nucleic acid is provided to the cell in a lipid nucleic acid assembly composition, optionally in the same lipid nucleic acid assembly composition as an RNA-guided DNA binding agent, and optionally wherein the lipid nucleic acid assembly composition is a lipid nanoparticle (LNP).

80-81. (canceled)

82. The method of claim 56, wherein the HLA-A guide RNA comprises a single guide RNA comprising any one of the sequences of SEQ ID NOs: 344-438, 472-504, 533-560, and 1016 or a sequence that is at least 99%, 98%, 97%, 96%, 95%, 94%, 93%, 92%, 91%, or 90% identical to any one of the sequences of SEQ ID NOs: 344-438, 472-504, and 533-560, and 1016; or
wherein the HLA-A guide RNA comprises a guide sequence comprising any one of SEQ ID NOs: 13-18, 26, 37-39, 41, 43, 45, and 62; or
wherein the HLA-A guide RNA comprises a single guide RNA comprising any one of the sequences of SEQ ID NOs: 356-361, 369, 380-382, 384, 386, 388, and 405, or a sequence that is at least 99%, 98%, 97%, 96%, 95%, 94%, 93%, 92%, 91%, or 90% identical to any one of the sequences of SEQ ID NOs: 356-361, 369, 380-382, 384, 386, 388, and 405; and optionally
wherein the HLA-A guide RNA comprises at least one modification, and optionally wherein the at least one modification includes (i) a 2′-O-methyl (2′-O-Me) modified nucleotide, (ii) a phosphorothioate (PS) bond between nucleotides, (iii) a 2′-fluoro (2′-F) modified nucleotide, (iv) a modification at one or more of the first five nucleotides at the 5′ end of the guide RNA, (v) a modification at one or more of the last five nucleotides at the 3′ end of the guide RNA, (vi) a PS bond between the first four nucleotides of the guide RNA, (vii) a PS bond between the last four nucleotides of the guide RNA, (viii) a 2′-O-Me modified nucleotide at the first three nucleotides at the 5′ end of the guide RNA, (ix) a 2′-O-Me modified nucleotide at the last three nucleotides at the 3′ end of the guide RNA, or combinations of one or more of (i)-(ix).

83-88. (canceled)

89. A cell bank comprising:
a. the engineered cell of claim 1; and
b. a catalogue comprising information documenting the HLA-B and HLA-C alleles of the donor cells in the cell bank, and optionally
wherein the cell bank comprises at least 3, 4, 5, 6, 7, 8, 9, 10, 11, 12, 13, 14, 15, 16, 17, 18, 19, 25, 30, 35, or 40 donor cells that have a unique combination of HLA-B and HLA-C alleles as compared to other donor cells in the cell bank.

90. (canceled)

91. A method of administering an engineered cell to a recipient subject in need thereof, the method comprising:
a. determining the HLA-B and HLA-C alleles of the recipient subject;
b. selecting the engineered cell of claim 1, wherein the engineered cell comprises at least one of the same HLA-B or HLA-C alleles as the recipient subject;
c. administering the selected engineered cell to the recipient subject, and optionally
wherein the subject has the HLA-B and HLA-C alleles of the engineered cell.

92. (canceled)

93. A method of administering the engineered cell of claim 1 to a partially matched subject for an adoptive cell transfer (ACT) therapy, wherein the partially matched subject has the HLA-B and HLA-C alleles of the engineered cell.

94. The method of claim 93, wherein the engineered cell comprises HLA-B and HLA-C alleles shared with the subject; or
wherein the HLA-B and HLA-C alleles of the engineered cell comprise one or more HLA-B and HLA-C alleles of the subject; or
wherein the HLA-B and HLA-C alleles of the engineered cell comprise one or both HLA-B alleles and/or one or both HLA-C alleles of the subject.

95-96. (canceled)


SEQUENCE LISTING (1016 sequences)

SEQ ID NO: 1 - RNA, 20 nt, synthetic construct
  misc_feature 1..20: note = Synthetic
  source 1..20: mol_type = other RNA
tggagggcctgatgtgtgtt

SEQ ID NO: 2 - RNA, 20 nt, synthetic construct
  misc_feature 1..20: note = Synthetic
  source 1..20: mol_type = other RNA
gcctgatgtgtgttgggtgt

SEQ ID NO: 3 - RNA, 20 nt, synthetic construct
  misc_feature 1..20: note = Synthetic
  source 1..20: mol_type = other RNA
cctgatgtgtgttgggtgtt

SEQ ID NO: 4 - RNA, 20 nt, synthetic construct
  misc_feature 1..20: note = Synthetic
  source 1..20: mol_type = other RNA
cccaacacccaacacacatc

SEQ ID NO: 5 - RNA, 20 nt, synthetic construct
  misc_feature 1..20: note = Synthetic
  source 1..20: mol_type = other RNA
tcaggaaacatgaagaaagc

SEQ ID NO: 6 - RNA, 20 nt, synthetic construct
  misc_feature 1..20: note = Synthetic
  source 1..20: mol_type = other RNA
aggcgcctgggcctctcccg

SEQ ID NO: 7 - RNA, 20 nt, synthetic construct
  misc_feature 1..20: note = Synthetic
  source 1..20: mol_type = other RNA
cgggctggcctcccacaagg

SEQ ID NO: 8 - RNA, 20 nt, synthetic construct
  misc_feature 1..20: note = Synthetic
  source 1..20: mol_type = other RNA
acggccatcctcggcgtctg

SEQ ID NO: 9 - RNA, 20 nt, synthetic construct
  misc_feature 1..20: note = Synthetic
  source 1..20: mol_type = other RNA
gacggccatcctcggcgtct

SEQ ID NO: 10 - RNA, 20 nt, synthetic construct
  misc_feature 1..20: note = Synthetic
  source 1..20: mol_type = other RNA
gacgccgaggatggccgtca

SEQ ID NO: 11 - RNA, 20 nt, synthetic construct
  misc_feature 1..20: note = Synthetic
  source 1..20: mol_type = other RNA
tgacggccatcctcggcgtc

SEQ ID NO: 12 - RNA, 20 nt, synthetic construct
  misc_feature 1..20: note = Synthetic
  source 1..20: mol_type = other RNA
ggcgccatgacggccatcct

SEQ ID NO: 13 - RNA, 20 nt, synthetic construct
  misc_feature 1..20: note = Synthetic
  source 1..20: mol_type = other RNA
acagcgacgccgcgagccag

SEQ ID NO: 14 - RNA, 20 nt, synthetic construct
  misc_feature 1..20: note = Synthetic
  source 1..20: mol_type = other RNA
cgacgccgcgagccagagga

SEQ ID NO: 15 - RNA, 20 nt, synthetic construct
  misc_feature 1..20: note = Synthetic
  source 1..20: mol_type = other RNA
cgagccagaggatggagccg

SEQ ID NO: 16 - RNA, 20 nt, synthetic construct
  misc_feature 1..20: note = Synthetic
  source 1..20: mol_type = other RNA
cggctccatcctctggctcg

SEQ ID NO: 17 - RNA, 20 nt, synthetic construct
  misc_feature 1..20: note = Synthetic
  source 1..20: mol_type = other RNA
gagccagaggatggagccgc

SEQ ID NO: 18 - RNA, 20 nt, synthetic construct
  misc_feature 1..20: note = Synthetic
  source 1..20: mol_type = other RNA
gcgcccgcggctccatcctc

SEQ ID NO: 19 - RNA, 20 nt, synthetic construct
  misc_feature 1..20: note = Synthetic
  source 1..20: mol_type = other RNA
gcccgtccgtgggggatgag

SEQ ID NO: 20 - RNA, 20 nt, synthetic construct
  misc_feature 1..20: note = Synthetic
  source 1..20: mol_type = other RNA
tcatcccccacggacgggcc

SEQ ID NO: 21 - RNA, 20 nt, synthetic construct
  misc_feature 1..20: note = Synthetic
  source 1..20: mol_type = other RNA
atctcggacccggagactgt

SEQ ID NO: 22 - RNA, 20 nt, synthetic construct
  misc_feature 1..20: note = Synthetic
  source 1..20: mol_type = other RNA
ggggtcccgcggcttcgggg

SEQ ID NO: 23 - RNA, 20 nt, synthetic construct
  misc_feature 1..20: note = Synthetic
  source 1..20: mol_type = other RNA
ctcggggtcccgcggcttcg

SEQ ID NO: 24 - RNA, 20 nt, synthetic construct
  misc_feature 1..20: note = Synthetic
  source 1..20: mol_type = other RNA
tctcggggtcccgcggcttc

SEQ ID NO: 25 - RNA, 20 nt, synthetic construct
  misc_feature 1..20: note = Synthetic
  source 1..20: mol_type = other RNA
gtctcggggtcccgcggctt

SEQ ID NO: 26 - RNA, 20 nt, synthetic construct
  misc_feature 1..20: note = Synthetic
  source 1..20: mol_type = other RNA
gcaagggtctcggggtcccg

SEQ ID NO: 27 - RNA, 20 nt, synthetic construct
  misc_feature 1..20: note = Synthetic
  source 1..20: mol_type = other RNA
ggaccccgagacccttgccc

SEQ ID NO: 28 - RNA, 20 nt, synthetic construct
  misc_feature 1..20: note = Synthetic
  source 1..20: mol_type = other RNA
gaccccgagacccttgcccc

SEQ ID NO: 29 - RNA, 20 nt, synthetic construct
  misc_feature 1..20: note = Synthetic
  source 1..20: mol_type = other RNA
cgagacccttgccccgggag

SEQ ID NO: 30 - RNA, 20 nt, synthetic construct
  misc_feature 1..20: note = Synthetic
  source 1..20: mol_type = other RNA
ctcccggggcaagggtctcg

SEQ ID NO: 31 - RNA, 20 nt, synthetic construct
  misc_feature 1..20: note = Synthetic
  source 1..20: mol_type = other RNA
tctcccggggcaagggtctc

SEQ ID NO: 32 - RNA, 20 nt, synthetic construct
  misc_feature 1..20: note = Synthetic
  source 1..20: mol_type = other RNA
ctctcccggggcaagggtct

SEQ ID NO: 33 - RNA, 20 nt, synthetic construct
  misc_feature 1..20: note = Synthetic
  source 1..20: mol_type = other RNA
ccttgccccgggagaggccc

SEQ ID NO: 34 - RNA, 20 nt, synthetic construct
  misc_feature 1..20: note = Synthetic
  source 1..20: mol_type = other RNA
ctgggcctctcccggggcaa

SEQ ID NO: 35 - RNA, 20 nt, synthetic construct
  misc_feature 1..20: note = Synthetic
  source 1..20: mol_type = other RNA
cctgggcctctcccggggca

SEQ ID NO: 36 - RNA, 20 nt, synthetic construct
  misc_feature 1..20: note = Synthetic
  source 1..20: mol_type = other RNA
tttaggccaaaaatcccccc

SEQ ID NO: 37 - RNA, 20 nt, synthetic construct
  misc_feature 1..20: note = Synthetic
  source 1..20: mol_type = other RNA
cgctgcagcgcacgggtacc

SEQ ID NO: 38 - RNA, 20 nt, synthetic construct
  misc_feature 1..20: note = Synthetic
  source 1..20: mol_type = other RNA
gctgcagcgcacgggtacca

SEQ ID NO: 39 - RNA, 20 nt, synthetic construct
  misc_feature 1..20: note = Synthetic
  source 1..20: mol_type = other RNA
ctgcagcgcacgggtaccag

SEQ ID NO: 40 - RNA, 20 nt, synthetic construct
  misc_feature 1..20: note = Synthetic
  source 1..20: mol_type = other RNA
cgcacgggtaccaggggcca

SEQ ID NO: 41 - RNA, 20 nt, synthetic construct
  misc_feature 1..20: note = Synthetic
  source 1..20: mol_type = other RNA
gcacgggtaccaggggccac

SEQ ID NO: 42 - RNA, 20 nt, synthetic construct
  misc_feature 1..20: note = Synthetic
  source 1..20: mol_type = other RNA
cacgggtaccaggggccacg

SEQ ID NO: 43 - RNA, 20 nt, synthetic construct
  misc_feature 1..20: note = Synthetic
  source 1..20: mol_type = other RNA
gggaggcgccccgtggcccc

SEQ ID NO: 44 - RNA, 20 nt, synthetic construct
  misc_feature 1..20: note = Synthetic
  source 1..20: mol_type = other RNA
gcgatcagggaggcgccccg

SEQ ID NO: 45 - RNA, 20 nt, synthetic construct
  misc_feature 1..20: note = Synthetic
  source 1..20: mol_type = other RNA
tccttgtgggaggccagccc

SEQ ID NO: 46 - RNA, 20 nt, synthetic construct
  misc_feature 1..20: note = Synthetic
  source 1..20: mol_type = other RNA
ctccttgtgggaggccagcc

SEQ ID NO: 47 - RNA, 20 nt, synthetic construct
  misc_feature 1..20: note = Synthetic
  source 1..20: mol_type = other RNA
ggctggcctcccacaaggag

SEQ ID NO: 48 - RNA, 20 nt, synthetic construct
  misc_feature 1..20: note = Synthetic
  source 1..20: mol_type = other RNA
ttgtctcccctccttgtggg

SEQ ID NO: 49 - RNA, 20 nt, synthetic construct
  misc_feature 1..20: note = Synthetic
  source 1..20: mol_type = other RNA
ccacaaggaggggagacaat

SEQ ID NO: 50 - RNA, 20 nt, synthetic construct
  misc_feature 1..20: note = Synthetic
  source 1..20: mol_type = other RNA
cacaaggaggggagacaatt

SEQ ID NO: 51 - RNA, 20 nt, synthetic construct
  misc_feature 1..20: note = Synthetic
  source 1..20: mol_type = other RNA
caattgtctcccctccttgt

SEQ ID NO: 52 - RNA, 20 nt, synthetic construct
  misc_feature 1..20: note = Synthetic
  source 1..20: mol_type = other RNA
ccaattgtctcccctccttg

SEQ ID NO: 53 - RNA, 20 nt, synthetic construct
  misc_feature 1..20: note = Synthetic
  source 1..20: mol_type = other RNA
atccctcgaatactgatgag

SEQ ID NO: 54 - RNA, 20 nt, synthetic construct
  misc_feature 1..20: note = Synthetic
  source 1..20: mol_type = other RNA
aaccactcatcagtattcga

SEQ ID NO: 55 - RNA, 20 nt, synthetic construct
  misc_feature 1..20: note = Synthetic
  source 1..20: mol_type = other RNA
gaaccactcatcagtattcg

SEQ ID NO: 56 - RNA, 20 nt, synthetic construct
  misc_feature 1..20: note = Synthetic
  source 1..20: mol_type = other RNA
gaggaaaagtcacgggccca

SEQ ID NO: 57 - RNA, 20 nt, synthetic construct
  misc_feature 1..20: note = Synthetic
  source 1..20: mol_type = other RNA
ggcccgtgacttttcctctc

SEQ ID NO: 58 - RNA, 20 nt, synthetic construct
  misc_feature 1..20: note = Synthetic
  source 1..20: mol_type = other RNA
tgcttcacactcaatgtgtg

SEQ ID NO: 59 - RNA, 20 nt, synthetic construct
  misc_feature 1..20: note = Synthetic
  source 1..20: mol_type = other RNA
gcttcacactcaatgtgtgt

SEQ ID NO: 60 - RNA, 20 nt, synthetic construct
  misc_feature 1..20: note = Synthetic
  source 1..20: mol_type = other RNA
cttcacactcaatgtgtgtg

SEQ ID NO: 61 - RNA, 20 nt, synthetic construct
  misc_feature 1..20: note = Synthetic
  source 1..20: mol_type = other RNA
ttcacactcaatgtgtgtgg

SEQ ID NO: 62 - RNA, 20 nt, synthetic construct
  misc_feature 1..20: note = Synthetic
  source 1..20: mol_type = other RNA
ttgagaatggacaggacacc

SEQ ID NO: 63 - RNA, 20 nt, synthetic construct
  misc_feature 1..20: note = Synthetic
  source 1..20: mol_type = other RNA
aggcattttgcatctgtcat

SEQ ID NO: 64 - RNA, 20 nt, synthetic construct
  misc_feature 1..20: note = Synthetic
  source 1..20: mol_type = other RNA
caggcattttgcatctgtca

SEQ ID NO: 65 - RNA, 20 nt, synthetic construct
  misc_feature 1..20: note = Synthetic
  source 1..20: mol_type = other RNA
aggggccctgaccctgctaa

SEQ ID NO: 66 - RNA, 20 nt, synthetic construct
  misc_feature 1..20: note = Synthetic
  source 1..20: mol_type = other RNA
tgggaaaagaggggaaggtg

SEQ ID NO: 67 - RNA, 20 nt, synthetic construct
  misc_feature 1..20: note = Synthetic
  source 1..20: mol_type = other RNA
tggaggaggaagagctcagg

SEQ ID NO: 68 - RNA, 20 nt, synthetic construct
  misc_feature 1..20: note = Synthetic
  source 1..20: mol_type = other RNA
tgagatttcttgtctcactg

SEQ ID NO: 69 - RNA, 20 nt, synthetic construct
  misc_feature 1..20: note = Synthetic
  source 1..20: mol_type = other RNA
gagatttcttgtctcactga

SEQ ID NO: 70 - RNA, 20 nt, synthetic construct
  misc_feature 1..20: note = Synthetic
  source 1..20: mol_type = other RNA
taaagcacctgttaaaatga

SEQ ID NO: 71 - RNA, 20 nt, synthetic construct
  misc_feature 1..20: note = Synthetic
  source 1..20: mol_type = other RNA
aatctgtccttcattttaac

SEQ ID NO: 72 - RNA, 20 nt, synthetic construct
  misc_feature 1..20: note = Synthetic
  source 1..20: mol_type = other RNA
gtcacaggggaaggtccctg

SEQ ID NO: 73 - RNA, 20 nt, synthetic construct
  misc_feature 1..20: note = Synthetic
  source 1..20: mol_type = other RNA
aaacatgaagaaagcaggtg

SEQ ID NO: 74 - RNA, 20 nt, synthetic construct
  misc_feature 1..20: note = Synthetic
  source 1..20: mol_type = other RNA
tgtcctgtgagataccagaa

SEQ ID NO: 75 - RNA, 20 nt, synthetic construct
  misc_feature 1..20: note = Synthetic
  source 1..20: mol_type = other RNA
atgaaggaggctgatgcctg

SEQ ID NO: 76 - RNA, 20 nt, synthetic construct
  misc_feature 1..20: note = Synthetic
  source 1..20: mol_type = other RNA
aggctgatgcctgaggtcct

SEQ ID NO: 77 - RNA, 20 nt, synthetic construct
  misc_feature 1..20: note = Synthetic
  source 1..20: mol_type = other RNA
ggctgatgcctgaggtcctt

SEQ ID NO: 78 - RNA, 20 nt, synthetic construct
  misc_feature 1..20: note = Synthetic
  source 1..20: mol_type = other RNA
cacaatatcccaaggacctc

SEQ ID NO: 79 - RNA, 20 nt, synthetic construct
  misc_feature 1..20: note = Synthetic
  source 1..20: mol_type = other RNA
ggtccttgggatattgtgtt

SEQ ID NO: 80 - RNA, 20 nt, synthetic construct
  misc_feature 1..20: note = Synthetic
  source 1..20: mol_type = other RNA
gtccttgggatattgtgttt

SEQ ID NO: 81 - RNA, 20 nt, synthetic construct
  misc_feature 1..20: note = Synthetic
  source 1..20: mol_type = other RNA
ctcccaaacacaatatccca

SEQ ID NO: 82 - RNA, 20 nt, synthetic construct
  misc_feature 1..20: note = Synthetic
  source 1..20: mol_type = other RNA
tcctctagccacatcttctg

SEQ ID NO: 83 - RNA, 20 nt, synthetic construct
  misc_feature 1..20: note = Synthetic
  source 1..20: mol_type = other RNA
acagaagatgtggctagagg

SEQ ID NO: 84 - RNA, 20 nt, synthetic construct
  misc_feature 1..20: note = Synthetic
  source 1..20: mol_type = other RNA
cctctagccacatcttctgt

SEQ ID NO: 85 - RNA, 20 nt, synthetic construct
  misc_feature 1..20: note = Synthetic
  source 1..20: mol_type = other RNA
cccacagaagatgtggctag

SEQ ID NO: 86 - RNA, 20 nt, synthetic construct
  misc_feature 1..20: note = Synthetic
  source 1..20: mol_type = other RNA
gtcagatcccacagaagatg

SEQ ID NO: 87 - RNA, 20 nt, synthetic construct
  misc_feature 1..20: note = Synthetic
  source 1..20: mol_type = other RNA
atcttctgtgggatctgacc

SEQ ID NO: 88 - RNA, 20 nt, synthetic construct
  misc_feature 1..20: note = Synthetic
  source 1..20: mol_type = other RNA
cccaggcagtgacagtgccc

SEQ ID NO: 89 - RNA, 20 nt, synthetic construct
  misc_feature 1..20: note = Synthetic
  source 1..20: mol_type = other RNA
ctgggcactgtcactgcctg

SEQ ID NO: 90 - RNA, 20 nt, synthetic construct
  misc_feature 1..20: note = Synthetic
  source 1..20: mol_type = other RNA
cctgggcactgtcactgcct

SEQ ID NO: 91 - RNA, 20 nt, synthetic construct
  misc_feature 1..20: note = Synthetic
  source 1..20: mol_type = other RNA
ccctgggcactgtcactgcc

SEQ ID NO: 92 - RNA, 20 nt, synthetic construct
  misc_feature 1..20: note = Synthetic
  source 1..20: mol_type = other RNA
ttgggtgttgggcggaacag

SEQ ID NO: 93 - RNA, 20 nt, synthetic construct
  misc_feature 1..20: note = Synthetic
  source 1..20: mol_type = other RNA
tggatgtattgagcatgcga

SEQ ID NO: 94 - RNA, 20 nt, synthetic construct
  misc_feature 1..20: note = Synthetic
  source 1..20: mol_type = other RNA
ggatgtattgagcatgcgat

SEQ ID NO: 95 - RNA, 20 nt, synthetic construct
  misc_feature 1..20: note = Synthetic
  source 1..20: mol_type = other RNA
aacatgaagaaagcaggtgt

SEQ ID NO: 96 - RNA, 20 nt, synthetic construct
  misc_feature 1..20: note = Synthetic
  source 1..20: mol_type = other RNA
tagcccacggcgatgaagcg

SEQ ID NO: 97 - RNA, 20 nt, synthetic construct
  misc_feature 1..20: note = Synthetic
  source 1..20: mol_type = other RNA
gtagcccacggcgatgaagc

SEQ ID NO: 98 - RNA, 20 nt, synthetic construct
  misc_feature 1..20: note = Synthetic
  source 1..20: mol_type = other RNA
cgtagcccacggcgatgaag

SEQ ID NO: 99 - RNA, 20 nt, synthetic construct
  misc_feature 1..20: note = Synthetic
  source 1..20: mol_type = other RNA
cttcatcgccgtgggctacg

SEQ ID NO: 100 - RNA, 20 nt, synthetic construct
  misc_feature 1..20: note = Synthetic
  source 1..20: mol_type = other RNA
cgtgtcgtccacgtagccca

SEQ ID NO: 101 - RNA, 20 nt, synthetic construct
  misc_feature 1..20: note = Synthetic
  source 1..20: mol_type = other RNA
tggacgacacgcagttcgtg

SEQ ID NO: 102 - RNA, 20 nt, synthetic construct
  misc_feature 1..20: note = Synthetic
  source 1..20: mol_type = other RNA
ggatggagccgcgggcgccg

SEQ ID NO: 103 - RNA, 20 nt, synthetic construct
  misc_feature 1..20: note = Synthetic
  source 1..20: mol_type = other RNA
gcgggcgccgtggatagagc

SEQ ID NO: 104 - RNA, 20 nt, synthetic construct
  misc_feature 1..20: note = Synthetic
  source 1..20: mol_type = other RNA
tgctctatccacggcgcccg

SEQ ID NO: 105 - RNA, 20 nt, synthetic construct
  misc_feature 1..20: note = Synthetic
  source 1..20: mol_type = other RNA
ggcgccgtggatagagcagg

SEQ ID NO: 106 - RNA, 20 nt, synthetic construct
  misc_feature 1..20: note = Synthetic
  source 1..20: mol_type = other RNA
gcgccgtggatagagcagga

SEQ ID NO: 107 - RNA, 20 nt, synthetic construct
  misc_feature 1..20: note = Synthetic
  source 1..20: mol_type = other RNA
cgccgtggatagagcaggag

SEQ ID NO: 108 - RNA, 20 nt, synthetic construct
  misc_feature 1..20: note = Synthetic
  source 1..20: mol_type = other RNA
gtggatagagcaggaggggc

SEQ ID NO: 109 - RNA, 20 nt, synthetic construct
  misc_feature 1..20: note = Synthetic
  source 1..20: mol_type = other RNA
ggcccctcctgctctatcca

SEQ ID NO: 110 - RNA, 20 nt, synthetic construct
  misc_feature 1..20: note = Synthetic
  source 1..20: mol_type = other RNA
agcaggaggggccggagtat

SEQ ID NO: 111 - RNA, 20 nt, synthetic construct
  misc_feature 1..20: note = Synthetic
  source 1..20: mol_type = other RNA
gcaggaggggccggagtatt

SEQ ID NO: 112 - RNA, 20 nt, synthetic construct
  misc_feature 1..20: note = Synthetic
  source 1..20: mol_type = other RNA
ggagtggctccgcagatacc

SEQ ID NO: 113 - RNA, 20 nt, synthetic construct
  misc_feature 1..20: note = Synthetic
  source 1..20: mol_type = other RNA
ctccgcagatacctggagaa

SEQ ID NO: 114 - RNA, 20 nt, synthetic construct
  misc_feature 1..20: note = Synthetic
  source 1..20: mol_type = other RNA
tccgcagatacctggagaac

SEQ ID NO: 115 - RNA, 20 nt, synthetic construct
  misc_feature 1..20: note = Synthetic
  source 1..20: mol_type = other RNA
cagatacctggagaacggga

SEQ ID NO: 116 - RNA, 20 nt, synthetic construct
  misc_feature 1..20: note = Synthetic
  source 1..20: mol_type = other RNA
tcccgttctccaggtatctg

SEQ ID NO: 117 - RNA, 20 nt, synthetic construct
  misc_feature 1..20: note = Synthetic
  source 1..20: mol_type = other RNA
gcgtctccttcccgttctcc

SEQ ID NO: 118 - RNA, 20 nt, synthetic construct
  misc_feature 1..20: note = Synthetic
  source 1..20: mol_type = other RNA
gaaggagacgctgcagcgca

SEQ ID NO: 119 - RNA, 20 nt, synthetic construct
  misc_feature 1..20: note = Synthetic
  source 1..20: mol_type = other RNA
aaggagacgctgcagcgcac

SEQ ID NO: 120 - RNA, 20 nt, synthetic construct
  misc_feature 1..20: note = Synthetic
  source 1..20: mol_type = other RNA
agatctacaggcgatcaggg

SEQ ID NO: 121 - RNA, 20 nt, synthetic construct
  misc_feature 1..20: note = Synthetic
  source 1..20: mol_type = other RNA
tgatcgcctgtagatctccc

SEQ ID NO: 122 - RNA, 20 nt, synthetic construct
  misc_feature 1..20: note = Synthetic
  source 1..20: mol_type = other RNA
gggagatctacaggcgatca

SEQ ID NO: 123 - RNA, 20 nt, synthetic construct
  misc_feature 1..20: note = Synthetic
  source 1..20: mol_type = other RNA
cgggagatctacaggcgatc

SEQ ID NO: 124 - RNA, 20 nt, synthetic construct
  misc_feature 1..20: note = Synthetic
  source 1..20: mol_type = other RNA
cgcctgtagatctcccgggc

SEQ ID NO: 125 - RNA, 20 nt, synthetic construct
  misc_feature 1..20: note = Synthetic
  source 1..20: mol_type = other RNA
ggccagcccgggagatctac

SEQ ID NO: 126 - RNA, 20 nt, synthetic construct
  misc_feature 1..20: note = Synthetic
  source 1..20: mol_type = other RNA
tcccgggctggcctcccaca

SEQ ID NO: 127 - RNA, 20 nt, synthetic construct
  misc_feature 1..20: note = Synthetic
  source 1..20: mol_type = other RNA
gggctggcctcccacaagga

SEQ ID NO: 128 - RNA, 20 nt, synthetic construct
  misc_feature 1..20: note = Synthetic
  source 1..20: mol_type = other RNA
ctgatcgcctgtagatctcc

SEQ ID NO: 129 - RNA, 20 nt, synthetic construct
  misc_feature 1..20: note = Synthetic
  source 1..20: mol_type = other RNA
aggatggagccgcgggcgcc

SEQ ID NO: 130 - RNA, 20 nt, synthetic construct
  misc_feature 1..20: note = Synthetic
  source 1..20: mol_type = other RNA
ggaaggagacgctgcagcgc

SEQ ID NO: 131 - RNA, 20 nt, synthetic construct
  misc_feature 1..20: note = Synthetic
  source 1..20: mol_type = other RNA
gacagcgacgccgcgagcca

SEQ ID NO: 132 - RNA, 20 nt, synthetic construct
  misc_feature 1..20: note = Synthetic
  source 1..20: mol_type = other RNA
cgggaaggagacgctgcagc

SEQ ID NO: 133 - RNA, 20 nt, synthetic construct
  misc_feature 1..20: note = Synthetic
  source 1..20: mol_type = other RNA
ccgtgcgctgcagcgtctcc

SEQ ID NO: 134 - RNA, 24 nt, synthetic construct
  misc_feature 1..24: note = Synthetic
  source 1..24: mol_type = other RNA
acgcagttcgtgcggttcgacagc

SEQ ID NO: 135 - RNA, 24 nt, synthetic construct
  misc_feature 1..24: note = Synthetic
  source 1..24: mol_type = other RNA
tcgtgcggttcgacagcgacgccg

SEQ ID NO: 136 - RNA, 24 nt, synthetic construct
  misc_feature 1..24: note = Synthetic
  source 1..24: mol_type = other RNA
cagcgacgccgcgagccagaggat

SEQ ID NO: 137 - RNA, 24 nt, synthetic construct
  misc_feature 1..24: note = Synthetic
  source 1..24: mol_type = other RNA
gctctatccacggcgcccgcggct

SEQ ID NO: 138 - RNA, 24 nt, synthetic construct
  misc_feature 1..24: note = Synthetic
  source 1..24: mol_type = other RNA
tcctgctctatccacggcgcccgc

SEQ ID NO: 139 - RNA, 24 nt, synthetic construct
  misc_feature 1..24: note = Synthetic
  source 1..24: mol_type = other RNA
ccggcccctcctgctctatccacg

SEQ ID NO: 140 - RNA, 24 nt, synthetic construct
  misc_feature 1..24: note = Synthetic
  source 1..24: mol_type = other RNA
tccggcccctcctgctctatccac

SEQ ID NO: 141 - RNA, 24 nt, synthetic construct
  misc_feature 1..24: note = Synthetic
  source 1..24: mol_type = other RNA
gggaaggagacgctgcagcgcacg

SEQ ID NO: 142 - RNA, 24 nt, synthetic construct
  misc_feature 1..24: note = Synthetic
  source 1..24: mol_type = other RNA
agacgctgcagcgcacgggtacca

SEQ ID NO: 143 - RNA, 24 nt, synthetic construct
  misc_feature 1..24: note = Synthetic
  source 1..24: mol_type = other RNA
gcgcacgggtaccaggggccacgg

SEQ ID NO: 144 - RNA, 24 nt, synthetic construct
  misc_feature 1..24: note = Synthetic
  source 1..24: mol_type = other RNA
cacgggtaccaggggccacggggc

SEQ ID NO: 145 - RNA, 24 nt, synthetic construct
  misc_feature 1..24: note = Synthetic
  source 1..24: mol_type = other RNA
acgggtaccaggggccacggggcg

SEQ ID NO: 146 - RNA, 24 nt, synthetic construct
  misc_feature 1..24: note = Synthetic
  source 1..24: mol_type = other RNA
caggggccacggggcgcctccctg

SEQ ID NO: 147 - RNA, 24 nt, synthetic construct
  misc_feature 1..24: note = Synthetic
  source 1..24: mol_type = other RNA
cagggaggcgccccgtggcccctg

SEQ ID NO: 148 - RNA, 24 nt, synthetic construct
  misc_feature 1..24: note = Synthetic
  source 1..24: mol_type = other RNA
tcagggaggcgccccgtggcccct

SEQ ID NO: 149 - RNA, 24 nt, synthetic construct
  misc_feature 1..24: note = Synthetic
  source 1..24: mol_type = other RNA
caggcgatcagggaggcgccccgt

SEQ ID NO: 150 - RNA, 24 nt, synthetic construct
  misc_feature 1..24: note = Synthetic
  source 1..24: mol_type = other RNA
acaggcgatcagggaggcgccccg

SEQ ID NO: 151 - RNA, 24 nt, synthetic construct
  misc_feature 1..24: note = Synthetic
  source 1..24: mol_type = other RNA
tacaggcgatcagggaggcgcccc

SEQ ID NO: 152 - RNA, 24 nt, synthetic construct
  misc_feature 1..24: note = Synthetic
  source 1..24: mol_type = other RNA
gggcgcctccctgatcgcctgtag

SEQ ID NO: 153 - RNA, 24 nt, synthetic construct
  misc_feature 1..24: note = Synthetic
  source 1..24: mol_type = other RNA
ggcgcctccctgatcgcctgtaga

SEQ ID NO: 154 - RNA, 24 nt, synthetic construct
  misc_feature 1..24: note = Synthetic
  source 1..24: mol_type = other RNA
gagatctacaggcgatcagggagg

SEQ ID NO: 155 - RNA, 24 nt, synthetic construct
  misc_feature 1..24: note = Synthetic
  source 1..24: mol_type = other RNA
ggagatctacaggcgatcagggag

SEQ ID NO: 156 - RNA, 24 nt, synthetic construct
  misc_feature 1..24: note = Synthetic
  source 1..24: mol_type = other RNA
gggagatctacaggcgatcaggga

SEQ ID NO: 157 - RNA, 24 nt, synthetic construct
  misc_feature 1..24: note = Synthetic
  source 1..24: mol_type = other RNA
ctgatcgcctgtagatctcccggg

SEQ ID NO: 158 - RNA, 24 nt, synthetic construct
  misc_feature 1..24: note = Synthetic
  source 1..24: mol_type = other RNA
atcgcctgtagatctcccgggctg

SEQ ID NO: 159 - RNA, 24 nt, synthetic construct
  misc_feature 1..24: note = Synthetic
  source 1..24: mol_type = other RNA
tcgcctgtagatctcccgggctgg

SEQ ID NO: 160 - RNA, 24 nt, synthetic construct
  misc_feature 1..24: note = Synthetic
  source 1..24: mol_type = other RNA
ttgtctcccctccttgtgggaggc

SEQ ID NO: 161 - RNA, 24 nt, synthetic construct
  misc_feature 1..24: note = Synthetic
  source 1..24: mol_type = other RNA
attgtctcccctccttgtgggagg

SEQ ID NO: 162 - RNA, 24 nt, synthetic construct
  misc_feature 1..24: note = Synthetic
  source 1..24: mol_type = other RNA
cccaattgtctcccctccttgtgg

SEQ ID NO: 163 - RNA, 20 nt, synthetic construct
  misc_feature 1..20: note = Synthetic
  source 1..20: mol_type = other RNA
ggatggagccgcgggcgccg

SEQ ID NO: 164 - RNA, 20 nt, synthetic construct
  misc_feature 1..20: note = Synthetic
  source 1..20: mol_type = other RNA
gcgggcgccgtggatagagc

SEQ ID NO: 165 - RNA, 20 nt, synthetic construct
  misc_feature 1..20: note = Synthetic
  source 1..20: mol_type = other RNA
tgctctatccacggcgcccg

SEQ ID NO: 166 - RNA, 20 nt, synthetic construct
  misc_feature 1..20: note = Synthetic
  source 1..20: mol_type = other RNA
ggcgccgtggatagagcagg

SEQ ID NO: 167 - RNA, 20 nt, synthetic construct
  misc_feature 1..20: note = Synthetic
  source 1..20: mol_type = other RNA
gcgccgtggatagagcagga

SEQ ID NO: 168 - RNA, 20 nt, synthetic construct
  misc_feature 1..20: note = Synthetic
  source 1..20: mol_type = other RNA
cgccgtggatagagcaggag

SEQ ID NO: 169 - RNA, 20 nt, synthetic construct
  misc_feature 1..20: note = Synthetic
  source 1..20: mol_type = other RNA
gtggatagagcaggaggggc

SEQ ID NO: 170 - RNA, 20 nt, synthetic construct
  misc_feature 1..20: note = Synthetic
  source 1..20: mol_type = other RNA
gcgtctccttcccgttctcc

SEQ ID NO: 171 - RNA, 20 nt, synthetic construct
  misc_feature 1..20: note = Synthetic
  source 1..20: mol_type = other RNA
gaaggagacgctgcagcgca

SEQ ID NO: 172 - RNA, 20 nt, synthetic construct
  misc_feature 1..20: note = Synthetic
  source 1..20: mol_type = other RNA
aaggagacgctgcagcgcac

SEQ ID NO: 173 - RNA, 20 nt, synthetic construct
  misc_feature 1..20: note = Synthetic
  source 1..20: mol_type = other RNA
gctgcagcgcacgggtacca

SEQ ID NO: 174 - RNA, 20 nt, synthetic construct
  misc_feature 1..20: note = Synthetic
  source 1..20: mol_type = other RNA
agatctacaggcgatcaggg

SEQ ID NO: 175 - RNA, 20 nt, synthetic construct
  misc_feature 1..20: note = Synthetic
  source 1..20: mol_type = other RNA
ctgatcgcctgtagatctcc

SEQ ID NO: 176 - RNA, 20 nt, synthetic construct
  misc_feature 1..20: note = Synthetic
  source 1..20: mol_type = other RNA
tgatcgcctgtagatctccc

SEQ ID NO: 177 - RNA, 20 nt, synthetic construct
  misc_feature 1..20: note = Synthetic
  source 1..20: mol_type = other RNA
gggagatctacaggcgatca

SEQ ID NO: 178 - RNA, 20 nt, synthetic construct
  misc_feature 1..20: note = Synthetic
  source 1..20: mol_type = other RNA
cgggagatctacaggcgatc

SEQ ID NO: 179 - RNA, 20 nt, synthetic construct
  misc_feature 1..20: note = Synthetic
  source 1..20: mol_type = other RNA
cgcctgtagatctcccgggc

SEQ ID NO: 180 - RNA, 20 nt, synthetic construct
  misc_feature 1..20: note = Synthetic
  source 1..20: mol_type = other RNA
ggccagcccgggagatctac

SEQ ID NO: 181 - RNA, 20 nt, synthetic construct
  misc_feature 1..20: note = Synthetic
  source 1..20: mol_type = other RNA
tcccgggctggcctcccaca

SEQ ID NO: 182 - RNA, 20 nt, synthetic construct
  misc_feature 1..20: note = Synthetic
  source 1..20: mol_type = other RNA
gggctggcctcccacaagga

SEQ ID NO: 183 - RNA, 20 nt, synthetic construct
  misc_feature 1..20: note = Synthetic
  source 1..20: mol_type = other RNA
acgacactgattggcttctc

SEQ ID NO: 184 - RNA, 20 nt, synthetic construct
  misc_feature 1..20: note = Synthetic
  source 1..20: mol_type = other RNA
acccctcatcccccacggac

SEQ ID NO: 185 - RNA, 20 nt, synthetic construct
  misc_feature 1..20: note = Synthetic
  source 1..20: mol_type = other RNA
ggcccgtccgtgggggatga

SEQ ID NO: 186 - RNA, 20 nt, synthetic construct
  misc_feature 1..20: note = Synthetic
  source 1..20: mol_type = other RNA
gccaggtcgcccacagtctc

SEQ ID NO: 187 - RNA, 20 nt, synthetic construct
  misc_feature 1..20: note = Synthetic
  source 1..20: mol_type = other RNA
gtttaggccaaaaatccccc

SEQ ID NO: 188 - RNA, 20 nt, synthetic construct
  misc_feature 1..20: note = Synthetic
  source 1..20: mol_type = other RNA
ggccaaaaatccccccgggt

SEQ ID NO: 189 - RNA, 20 nt, synthetic construct
  misc_feature 1..20: note = Synthetic
  source 1..20: mol_type = other RNA
gaccaacccggggggatttt

SEQ ID NO: 190 - RNA, 20 nt, synthetic construct
  misc_feature 1..20: note = Synthetic
  source 1..20: mol_type = other RNA
cacgggcccaaggctgctgc

SEQ ID NO: 191 - RNA, 20 nt, synthetic construct
  misc_feature 1..20: note = Synthetic
  source 1..20: mol_type = other RNA
accctcatgctgcacatggc

SEQ ID NO: 192 - RNA, 20 nt, synthetic construct
  misc_feature 1..20: note = Synthetic
  source 1..20: mol_type = other RNA
cctctaggaccttaaggccc

SEQ ID NO: 193 - RNA, 20 nt, synthetic construct
  misc_feature 1..20: note = Synthetic
  source 1..20: mol_type = other RNA
gctcctttctggtatctcac

SEQ ID NO: 194 - RNA, 20 nt, synthetic construct
  misc_feature 1..20: note = Synthetic
  source 1..20: mol_type = other RNA
gctatggggtttctttgcat

SEQ ID NO: 195 - RNA, 20 nt, synthetic construct
  misc_feature 1..20: note = Synthetic
  source 1..20: mol_type = other RNA
gcctttgcagaaacaaagtc

SEQ ID NO: 196 - RNA, 20 nt, synthetic construct
  misc_feature 1..20: note = Synthetic
  source 1..20: mol_type = other RNA
tggaccaaccgccctcctga

SEQ ID NO: 197 - RNA, 20 nt, synthetic construct
  misc_feature 1..20: note = Synthetic
  source 1..20: mol_type = other RNA
agcctctctgacctttagca

SEQ ID NO: 198 - RNA, 20 nt, synthetic construct
  misc_feature 1..20: note = Synthetic
  source 1..20: mol_type = other RNA
cgccctcctgaaggtcctca

SEQ ID NO: 199 - skipped

SEQ ID NO: 200 - RNA, 20 nt, synthetic construct
  misc_feature 1..20: note = Synthetic
  source 1..20: mol_type = other RNA
ccgccctcctgaaggtcctc

SEQ ID NO: 201 - RNA, 20 nt, synthetic construct
  misc_feature 1..20: note = Synthetic
  source 1..20: mol_type = other RNA
tggttccctttgacacacac

SEQ ID NO: 202 - RNA, 20 nt, synthetic construct
  misc_feature 1..20: note = Synthetic
  source 1..20: mol_type = other RNA
gaccctgctaaaggtcagag

SEQ ID NO: 203 - RNA, 20 nt, synthetic construct
  misc_feature 1..20: note = Synthetic
  source 1..20: mol_type = other RNA
aggaccttcaggagggcggt

SEQ ID NO: 204 - RNA, 20 nt, synthetic construct
  misc_feature 1..20: note = Synthetic
  source 1..20: mol_type = other RNA
gcacacttctacctgggtct

SEQ ID NO: 205 - RNA, 20 nt, synthetic construct
  misc_feature 1..20: note = Synthetic
  source 1..20: mol_type = other RNA
gagcctctctgacctttagc

SEQ ID NO: 206 - RNA, 20 nt, synthetic construct
  misc_feature 1..20: note = Synthetic
  source 1..20: mol_type = other RNA
acactcctccagcacacatg

SEQ ID NO: 207 - RNA, 20 nt, synthetic construct
  misc_feature 1..20: note = Synthetic
  source 1..20: mol_type = other RNA
ctctgacctttagcagggtc

SEQ ID NO: 208 - RNA, 20 nt, synthetic construct
  misc_feature 1..20: note = Synthetic
  source 1..20: mol_type = other RNA
caagatagccacatgtgtgc

SEQ ID NO: 209 - RNA, 20 nt, synthetic construct
  misc_feature 1..20: note = Synthetic
  source 1..20: mol_type = other RNA
tctgacctttagcagggtca

SEQ ID NO: 210 - RNA, 20 nt, synthetic construct
  misc_feature 1..20: note = Synthetic
  source 1..20: mol_type = other RNA
tgtaaaggtgagagcctgga

SEQ ID NO: 211 - RNA, 20 nt, synthetic construct
  misc_feature 1..20: note = Synthetic
  source 1..20: mol_type = other RNA
gaaggtccctgaggaccttc

SEQ ID NO: 212 - skipped

SEQ ID NO: 213 - RNA, 22 nt, synthetic construct
  misc_feature 1..22: note = Synthetic
  source 1..22: mol_type = other RNA
gttttagagctatgctgttttg

SEQ ID NO: 214 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 215 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 216 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 217-229 - skipped (13 entries)

SEQ ID NO: 230 - RNA, 20 nt, synthetic construct
  misc_feature 1..20: note = Synthetic
  source 1..20: mol_type = other RNA
gagtccgagcagaagaagaa

SEQ ID NO: 231 - RNA, 20 nt, synthetic construct
  misc_feature 1..20: note = Synthetic
  source 1..20: mol_type = other RNA
gaccccctccaccccgcctc

SEQ ID NO: 232 - RNA, 20 nt, synthetic construct
  misc_feature 1..20: note = Synthetic
  source 1..20: mol_type = other RNA
gacttgttttcattgttctc

SEQ ID NO: 233 - RNA, 20 nt, synthetic construct
  misc_feature 1..20: note = Synthetic
  source 1..20: mol_type = other RNA
ctctcagctggtacacggca

SEQ ID NO: 234 - RNA, 20 nt, synthetic construct
  misc_feature 1..20: note = Synthetic
  source 1..20: mol_type = other RNA
tgtgcagactcagaggtgag

SEQ ID NO: 235 - RNA, 20 nt, synthetic construct
  misc_feature 1..20: note = Synthetic
  source 1..20: mol_type = other RNA
ggccacggagcgagacatct

SEQ ID NO: 236 - RNA, 20 nt, synthetic construct
  misc_feature 1..20: note = Synthetic
  source 1..20: mol_type = other RNA
cccccggacggttcaagcaa

SEQ ID NO: 237-239 - skipped (3 entries)

SEQ ID NO: 240 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 241 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 242 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
gacttgttttcattgttctcgttttagagctagaaatagcaagttaaaataaggctagtccgttatcaacttgaaaaagtggcaccgagtcggtgctttt

SEQ ID NO: 243 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ctctcagctggtacacggcagttttagagctagaaatagcaagttaaaataaggctagtccgttatcaacttgaaaaagtggcaccgagtcggtgctttt

SEQ ID NO: 244 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tgtgcagactcagaggtgaggttttagagctagaaatagcaagttaaaataaggctagtccgttatcaacttgaaaaagtggcaccgagtcggtgctttt

SEQ ID NO: 245 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggccacggagcgagacatctgttttagagctagaaatagcaagttaaaataaggctagtccgttatcaacttgaaaaagtggcaccgagtcggtgctttt

SEQ ID NO: 246 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
cccccggacggttcaagcaagttttagagctagaaatagcaagttaaaataaggctagtccgttatcaacttgaaaaagtggcaccgagtcggtgctttt

SEQ ID NO: 247 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
ggcctcggcgctgacgatctgttttagagctagaaatagcaagttaaaataaggctagtccgttatcaacttgaaaaagtggcaccgagtcggtgctttt

SEQ ID NO: 248 - RNA, 100 nt, synthetic construct
  misc_feature 1..100: note = Synthetic
  variation 1..100: note = 2'-O-methylation and phosphorothioate internucleoside linkage modifications are present or absent
  source 1..100: mol_type = other RNA
tggtcagggcaagagctattgttttagagctagaaatagcaagttaaaataaggctagtccgttatcaacttgaaaaagtggcaccgagtcggtgctttt

SEQ ID NO: 249 - RNA, 100 nt, synthetic construct
  misc_feature 1..100: note = Synthetic
  source 1..100: mol_type = other RNA
tggagggcctgatgtgtgttgttttagagctagaaatagcaagttaaaataaggctagtccgttatcaacttgaaaaagtggcaccgagtcggtgctttt

SEQ ID NO: 250 - RNA, 100 nt, synthetic construct
  misc_feature 1..100: note = Synthetic
  source 1..100: mol_type = other RNA
gcctgatgtgtgttgggtgtgttttagagctagaaatagcaagttaaaataaggctagtccgttatcaacttgaaaaagtggcaccgagtcggtgctttt

SEQ ID NO: 251 - RNA, 100 nt, synthetic construct
  misc_feature 1..100: note = Synthetic
  source 1..100: mol_type = other RNA
cctgatgtgtgttgggtgttgttttagagctagaaatagcaagttaaaataaggctagtccgttatcaacttgaaaaagtggcaccgagtcggtgctttt

SEQ ID NO: 252 - RNA, 100 nt, synthetic construct
  misc_feature 1..100: note = Synthetic
  source 1..100: mol_type = other RNA
cccaacacccaacacacatcgttttagagctagaaatagcaagttaaaataaggctagtccgttatcaacttgaaaaagtggcaccgagtcggtgctttt

SEQ ID NO: 253 - RNA, 100 nt, synthetic construct
  misc_feature 1..100: note = Synthetic
  source 1..100: mol_type = other RNA
tcaggaaacatgaagaaagcgttttagagctagaaatagcaagttaaaataaggctagtccgttatcaacttgaaaaagtggcaccgagtcggtgctttt

SEQ ID NO: 254 - RNA, 100 nt, synthetic construct
  misc_feature 1..100: note = Synthetic
  source 1..100: mol_type = other RNA
aggcgcctgggcctctcccggttttagagctagaaatagcaagttaaaataaggctagtccgttatcaacttgaaaaagtggcaccgagtcggtgctttt

SEQ ID NO: 255 - RNA, 100 nt, synthetic construct
  misc_feature 1..100: note = Synthetic
  source 1..100: mol_type = other RNA
cgggctggcctcccacaagggttttagagctagaaatagcaagttaaaataaggctagtccgttatcaacttgaaaaagtggcaccgagtcggtgctttt

SEQ ID NO: 256 - RNA, 100 nt, synthetic construct
  misc_feature 1..100: note = Synthetic
  source 1..100: mol_type = other RNA
acggccatcctcggcgtctggttttagagctagaaatagcaagttaaaataaggctagtccgttatcaacttgaaaaagtggcaccgagtcggtgctttt

SEQ ID NO: 257 - RNA, 100 nt, synthetic construct
  misc_feature 1..100: note = Synthetic
  source 1..100: mol_type = other RNA
gacggccatcctcggcgtctgttttagagctagaaatagcaagttaaaataaggctagtccgttatcaacttgaaaaagtggcaccgagtcggtgctttt

SEQ ID NO: 258 - RNA, 100 nt, synthetic construct
  misc_feature 1..100: note = Synthetic
  source 1..100: mol_type = other RNA
gacgccgaggatggccgtcagttttagagctagaaatagcaagttaaaataaggctagtccgttatcaacttgaaaaagtggcaccgagtcggtgctttt

SEQ ID NO: 259 - RNA, 100 nt, synthetic construct
  misc_feature 1..100: note = Synthetic
  source 1..100: mol_type = other RNA
tgacggccatcctcggcgtcgttttagagctagaaatagcaagttaaaataaggctagtccgttatcaacttgaaaaagtggcaccgagtcggtgctttt

SEQ ID NO: 260 - RNA, 100 nt, synthetic construct
  misc_feature 1..100: note = Synthetic
  source 1..100: mol_type = other RNA
ggcgccatgacggccatcctgttttagagctagaaatagcaagttaaaataaggctagtccgttatcaacttgaaaaagtggcaccgagtcggtgctttt

SEQ ID NO: 261 - RNA, 100 nt, synthetic construct
  misc_feature 1..100: note = Synthetic
  source 1..100: mol_type = other RNA
acagcgacgccgcgagccaggttttagagctagaaatagcaagttaaaataaggctagtccgttatcaacttgaaaaagtggcaccgagtcggtgctttt

SEQ ID NO: 262 - RNA, 100 nt, synthetic construct
  misc_feature 1..100: note = Synthetic
  source 1..100: mol_type = other RNA
cgacgccgcgagccagaggagttttagagctagaaatagcaagttaaaataaggctagtccgttatcaacttgaaaaagtggcaccgagtcggtgctttt

SEQ ID NO: 263 - RNA, 100 nt, synthetic construct
  misc_feature 1..100: note = Synthetic
  source 1..100: mol_type = other RNA
cgagccagaggatggagccggttttagagctagaaatagcaagttaaaataaggctagtccgttatcaacttgaaaaagtggcaccgagtcggtgctttt

SEQ ID NO: 264 - RNA, 100 nt, synthetic construct
  misc_feature 1..100: note = Synthetic
  source 1..100: mol_type = other RNA
cggctccatcctctggctcggttttagagctagaaatagcaagttaaaataaggctagtccgttatcaacttgaaaaagtggcaccgagtcggtgctttt

SEQ ID NO: 265 - RNA, 100 nt, synthetic construct
  misc_feature 1..100: note = Synthetic
  source 1..100: mol_type = other RNA
gagccagaggatggagccgcgttttagagctagaaatagcaagttaaaataaggctagtccgttatcaacttgaaaaagtggcaccgagtcggtgctttt

SEQ ID NO: 266 - RNA, 100 nt, synthetic construct
  misc_feature 1..100: note = Synthetic
  source 1..100: mol_type = other RNA
gcgcccgcggctccatcctcgttttagagctagaaatagcaagttaaaataaggctagtccgttatcaacttgaaaaagtggcaccgagtcggtgctttt

SEQ ID NO: 267 - RNA, 100 nt, synthetic construct
  misc_feature 1..100: note = Synthetic
  source 1..100: mol_type = other RNA
gcccgtccgtgggggatgaggttttagagctagaaatagcaagttaaaataaggctagtccgttatcaacttgaaaaagtggcaccgagtcggtgctttt

SEQ ID NO: 268 - RNA, 100 nt, synthetic construct
  misc_feature 1..100: note = Synthetic
  source 1..100: mol_type = other RNA
tcatcccccacggacgggccgttttagagctagaaatagcaagttaaaataaggctagtccgttatcaacttgaaaaagtggcaccgagtcggtgctttt

SEQ ID NO: 269 - RNA, 100 nt, synthetic construct
  misc_feature 1..100: note = Synthetic
  source 1..100: mol_type = other RNA
atctcggacccggagactgtgttttagagctagaaatagcaagttaaaataaggctagtccgttatcaacttgaaaaagtggcaccgagtcggtgctttt

SEQ ID NO: 270 - RNA, 100 nt, synthetic construct
  misc_feature 1..100: note = Synthetic
  source 1..100: mol_type = other RNA
ggggtcccgcggcttcgggggttttagagctagaaatagcaagttaaaataaggctagtccgttatcaacttgaaaaagtggcaccgagtcggtgctttt

SEQ ID NO: 271 - RNA, 100 nt, synthetic construct
  misc_feature 1..100: note = Synthetic
  source 1..100: mol_type = other RNA
ctcggggtcccgcggcttcggttttagagctagaaatagcaagttaaaataaggctagtccgttatcaacttgaaaaagtggcaccgagtcggtgctttt

SEQ ID NO: 272 - RNA, 100 nt, synthetic construct
  misc_feature 1..100: note = Synthetic
  source 1..100: mol_type = other RNA
tctcggggtcccgcggcttcgttttagagctagaaatagcaagttaaaataaggctagtccgttatcaacttgaaaaagtggcaccgagtcggtgctttt

SEQ ID NO: 273 - RNA, 100 nt, synthetic construct
  misc_feature 1..100: note = Synthetic
  source 1..100: mol_type = other RNA
gtctcggggtcccgcggcttgttttagagctagaaatagcaagttaaaataaggctagtccgttatcaacttgaaaaagtggcaccgagtcggtgctttt

SEQ ID NO: 274 - RNA, 100 nt, synthetic construct
  misc_feature 1..100: note = Synthetic
  source 1..100: mol_type = other RNA
gcaagggtctcggggtcccggttttagagctagaaatagcaagttaaaataaggctagtccgttatcaacttgaaaaagtggcaccgagtcggtgctttt

SEQ ID NO: 275 - RNA, 100 nt, synthetic construct
  misc_feature 1..100: note = Synthetic
  source 1..100: mol_type = other RNA
ggaccccgagacccttgcccgttttagagctagaaatagcaagttaaaataaggctagtccgttatcaacttgaaaaagtggcaccgagtcggtgctttt

SEQ ID NO: 276 - RNA, 100 nt, synthetic construct
  misc_feature 1..100: note = Synthetic
  source 1..100: mol_type = other RNA
gaccccgagacccttgccccgttttagagctagaaatagcaagttaaaataaggctagtccgttatcaacttgaaaaagtggcaccgagtcggtgctttt

SEQ ID NO: 277 - RNA, 100 nt, synthetic construct
  misc_feature 1..100: note = Synthetic
  source 1..100: mol_type = other RNA
cgagacccttgccccgggaggttttagagctagaaatagcaagttaaaataaggctagtccgttatcaacttgaaaaagtggcaccgagtcggtgctttt

SEQ ID NO: 278 - RNA, 100 nt, synthetic construct
  misc_feature 1..100: note = Synthetic
  source 1..100: mol_type = other RNA
ctcccggggcaagggtctcggttttagagctagaaatagcaagttaaaataaggctagtccgttatcaacttgaaaaagtggcaccgagtcggtgctttt

SEQ ID NO: 279 - RNA, 100 nt, synthetic construct
  misc_feature 1..100: note = Synthetic
  source 1..100: mol_type = other RNA
tctcccggggcaagggtctcgttttagagctagaaatagcaagttaaaataaggctagtccgttatcaacttgaaaaagtggcaccgagtcggtgctttt

SEQ ID NO: 280 - RNA, 100 nt, synthetic construct
  misc_feature 1..100: note = Synthetic
  source 1..100: mol_type = other RNA
ctctcccggggcaagggtctgttttagagctagaaatagcaagttaaaataaggctagtccgttatcaacttgaaaaagtggcaccgagtcggtgctttt

SEQ ID NO: 281 - RNA, 100 nt, synthetic construct
  misc_feature 1..100: note = Synthetic
  source 1..100: mol_type = other RNA
ccttgccccgggagaggcccgttttagagctagaaatagcaagttaaaataaggctagtccgttatcaacttgaaaaagtggcaccgagtcggtgctttt

SEQ ID NO: 282 - RNA, 100 nt, synthetic construct
  misc_feature 1..100: note = Synthetic
  source 1..100: mol_type = other RNA
ctgggcctctcccggggcaagttttagagctagaaatagcaagttaaaataaggctagtccgttatcaacttgaaaaagtggcaccgagtcggtgctttt

SEQ ID NO: 283 - RNA, 100 nt, synthetic construct
  misc_feature 1..100: note = Synthetic
  source 1..100: mol_type = other RNA
cctgggcctctcccggggcagttttagagctagaaatagcaagttaaaataaggctagtccgttatcaacttgaaaaagtggcaccgagtcggtgctttt

SEQ ID NO: 284 - RNA, 100 nt, synthetic construct
  misc_feature 1..100: note = Synthetic
  source 1..100: mol_type = other RNA
tttaggccaaaaatccccccgttttagagctagaaatagcaagttaaaataaggctagtccgttatcaacttgaaaaagtggcaccgagtcggtgctttt

SEQ ID NO: 285 - RNA, 100 nt, synthetic construct
  misc_feature 1..100: note = Synthetic
  source 1..100: mol_type = other RNA
cgctgcagcgcacgggtaccgttttagagctagaaatagcaagttaaaataaggctagtccgttatcaacttgaaaaagtggcaccgagtcggtgctttt

SEQ ID NO: 286 - RNA, 100 nt, synthetic construct
  misc_feature 1..100: note = Synthetic
  source 1..100: mol_type = other RNA
gctgcagcgcacgggtaccagttttagagctagaaatagcaagttaaaataaggctagtccgttatcaacttgaaaaagtggcaccgagtcggtgctttt

SEQ ID NO: 287 - RNA, 100 nt, synthetic construct
  misc_feature 1..100: note = Synthetic
  source 1..100: mol_type = other RNA
ctgcagcgcacgggtaccaggttttagagctagaaatagcaagttaaaataaggctagtccgttatcaacttgaaaaagtggcaccgagtcggtgctttt

SEQ ID NO: 288 - RNA, 100 nt, synthetic construct
  misc_feature 1..100: note = Synthetic
  source 1..100: mol_type = other RNA
cgcacgggtaccaggggccagttttagagctagaaatagcaagttaaaataaggctagtccgttatcaacttgaaaaagtggcaccgagtcggtgctttt

SEQ ID NO: 289 - RNA, 100 nt, synthetic construct
  misc_feature 1..100: note = Synthetic
  source 1..100: mol_type = other RNA
gcacgggtaccaggggccacgttttagagctagaaatagcaagttaaaataaggctagtccgttatcaacttgaaaaagtggcaccgagtcggtgctttt

SEQ ID NO: 290 - RNA, 100 nt, synthetic construct
  misc_feature 1..100: note = Synthetic
  source 1..100: mol_type = other RNA
cacgggtaccaggggccacggttttagagctagaaatagcaagttaaaataaggctagtccgttatcaacttgaaaaagtggcaccgagtcggtgctttt

SEQ ID NO: 291 - RNA, 100 nt, synthetic construct
  misc_feature 1..100: note = Synthetic
  source 1..100: mol_type = other RNA
gggaggcgccccgtggccccgttttagagctagaaatagcaagttaaaataaggctagtccgttatcaacttgaaaaagtggcaccgagtcggtgctttt

SEQ ID NO: 292 - RNA, 100 nt, synthetic construct
  misc_feature 1..100: note = Synthetic
  source 1..100: mol_type = other RNA
gcgatcagggaggcgccccggttttagagctagaaatagcaagttaaaataaggctagtccgttatcaacttgaaaaagtggcaccgagtcggtgctttt

SEQ ID NO: 293 - RNA, 100 nt, synthetic construct
  misc_feature 1..100: note = Synthetic
  source 1..100: mol_type = other RNA
tccttgtgggaggccagcccgttttagagctagaaatagcaagttaaaataaggctagtccgttatcaacttgaaaaagtggcaccgagtcggtgctttt

SEQ ID NO: 294 - RNA, 100 nt, synthetic construct
  misc_feature 1..100: note = Synthetic
  source 1..100: mol_type = other RNA
ctccttgtgggaggccagccgttttagagctagaaatagcaagttaaaataaggctagtccgttatcaacttgaaaaagtggcaccgagtcggtgctttt

SEQ ID NO: 295 - RNA, 100 nt, synthetic construct
  misc_feature 1..100: note = Synthetic
  source 1..100: mol_type = other RNA
ggctggcctcccacaaggaggttttagagctagaaatagcaagttaaaataaggctagtccgttatcaacttgaaaaagtggcaccgagtcggtgctttt

SEQ ID NO: 296 - RNA, 100 nt, synthetic construct
  misc_feature 1..100: note = Synthetic
  source 1..100: mol_type = other RNA
ttgtctcccctccttgtggggttttagagctagaaatagcaagttaaaataaggctagtccgttatcaacttgaaaaagtggcaccgagtcggtgctttt

SEQ ID NO: 297 - RNA, 100 nt, synthetic construct
  misc_feature 1..100: note = Synthetic
  source 1..100: mol_type = other RNA
ccacaaggaggggagacaatgttttagagctagaaatagcaagttaaaataaggctagtccgttatcaacttgaaaaagtggcaccgagtcggtgctttt

SEQ ID NO: 298 - RNA, 100 nt, synthetic construct
  misc_feature 1..100: note = Synthetic
  source 1..100: mol_type = other RNA
cacaaggaggggagacaattgttttagagctagaaatagcaagttaaaataaggctagtccgttatcaacttgaaaaagtggcaccgagtcggtgctttt

SEQ ID NO: 299 - RNA, 100 nt, synthetic construct
  misc_feature 1..100: note = Synthetic
  source 1..100: mol_type = other RNA
caattgtctcccctccttgtgttttagagctagaaatagcaagttaaaataaggctagtccgttatcaacttgaaaaagtggcaccgagtcggtgctttt

SEQ ID NO: 300 - RNA, 100 nt, synthetic construct
  misc_feature 1..100: note = Synthetic
  source 1..100: mol_type = other RNA
ccaattgtctcccctccttggttttagagctagaaatagcaagttaaaataaggctagtccgttatcaacttgaaaaagtggcaccgagtcggtgctttt

SEQ ID NO: 301 - RNA, 100 nt, synthetic construct
  misc_feature 1..100: note = Synthetic
  source 1..100: mol_type = other RNA
atccctcgaatactgatgaggttttagagctagaaatagcaagttaaaataaggctagtccgttatcaacttgaaaaagtggcaccgagtcggtgctttt

SEQ ID NO: 302 - RNA, 100 nt, synthetic construct
  misc_feature 1..100: note = Synthetic
  source 1..100: mol_type = other RNA
aaccactcatcagtattcgagttttagagctagaaatagcaagttaaaataaggctagtccgttatcaacttgaaaaagtggcaccgagtcggtgctttt

SEQ ID NO: 303 - RNA, 100 nt, synthetic construct
  misc_feature 1..100: note = Synthetic
  source 1..100: mol_type = other RNA
gaaccactcatcagtattcggttttagagctagaaatagcaagttaaaataaggctagtccgttatcaacttgaaaaagtggcaccgagtcggtgctttt

SEQ ID NO: 304 - RNA, 100 nt, synthetic construct
  misc_feature 1..100: note = Synthetic
  source 1..100: mol_type = other RNA
gaggaaaagtcacgggcccagttttagagctagaaatagcaagttaaaataaggctagtccgttatcaacttgaaaaagtggcaccgagtcggtgctttt

SEQ ID NO: 305 - RNA, 100 nt, synthetic construct
  misc_feature 1..100: note = Synthetic
  source 1..100: mol_type = other RNA
ggcccgtgacttttcctctcgttttagagctagaaatagcaagttaaaataaggctagtccgttatcaacttgaaaaagtggcaccgagtcggtgctttt

SEQ ID NO: 306 - RNA, 100 nt, synthetic construct
  misc_feature 1..100: note = Synthetic
  source 1..100: mol_type = other RNA
tgcttcacactcaatgtgtggttttagagctagaaatagcaagttaaaataaggctagtccgttatcaacttgaaaaagtggcaccgagtcggtgctttt

SEQ ID NO: 307 - RNA, 100 nt, synthetic construct
  misc_feature 1..100: note = Synthetic
  source 1..100: mol_type = other RNA
gcttcacactcaatgtgtgtgttttagagctagaaatagcaagttaaaataaggctagtccgttatcaacttgaaaaagtggcaccgagtcggtgctttt

SEQ ID NO: 308 - RNA, 100 nt, synthetic construct
  misc_feature 1..100: note = Synthetic
  source 1..100: mol_type = other RNA
cttcacactcaatgtgtgtggttttagagctagaaatagcaagttaaaataaggctagtccgttatcaacttgaaaaagtggcaccgagtcggtgctttt

SEQ ID NO: 309 - RNA, 100 nt, synthetic construct
  misc_feature 1..100: note = Synthetic
  source 1..100: mol_type = other RNA
ttcacactcaatgtgtgtgggttttagagctagaaatagcaagttaaaataaggctagtccgttatcaacttgaaaaagtggcaccgagtcggtgctttt

SEQ ID NO: 310 - RNA, 100 nt, synthetic construct
  misc_feature 1..100: note = Synthetic
  source 1..100: mol_type = other RNA
ttgagaatggacaggacaccgttttagagctagaaatagcaagttaaaataaggctagtccgttatcaacttgaaaaagtggcaccgagtcggtgctttt

SEQ ID NO: 311 - RNA, 100 nt, synthetic construct
  misc_feature 1..100: note = Synthetic
  source 1..100: mol_type = other RNA
aggcattttgcatctgtcatgttttagagctagaaatagcaagttaaaataaggctagtccgttatcaacttgaaaaagtggcaccgagtcggtgctttt

SEQ ID NO: 312 - RNA, 100 nt, synthetic construct
  misc_feature 1..100: note = Synthetic
  source 1..100: mol_type = other RNA
caggcattttgcatctgtcagttttagagctagaaatagcaagttaaaataaggctagtccgttatcaacttgaaaaagtggcaccgagtcggtgctttt

SEQ ID NO: 313 - RNA, 100 nt, synthetic construct
  misc_feature 1..100: note = Synthetic
  source 1..100: mol_type = other RNA
aggggccctgaccctgctaagttttagagctagaaatagcaagttaaaataaggctagtccgttatcaacttgaaaaagtggcaccgagtcggtgctttt

SEQ ID NO: 314 - RNA, 100 nt, synthetic construct
  misc_feature 1..100: note = Synthetic
  source 1..100: mol_type = other RNA
tgggaaaagaggggaaggtggttttagagctagaaatagcaagttaaaataaggctagtccgttatcaacttgaaaaagtggcaccgagtcggtgctttt

SEQ ID NO: 315 - RNA, 100 nt, synthetic construct
  misc_feature 1..100: note = Synthetic
  source 1..100: mol_type = other RNA
tggaggaggaagagctcagggttttagagctagaaatagcaagttaaaataaggctagtccgttatcaacttgaaaaagtggcaccgagtcggtgctttt

SEQ ID NO: 316 - RNA, 100 nt, synthetic construct
  misc_feature 1..100: note = Synthetic
  source 1..100: mol_type = other RNA
tgagatttcttgtctcactggttttagagctagaaatagcaagttaaaataaggctagtccgttatcaacttgaaaaagtggcaccgagtcggtgctttt

SEQ ID NO: 317 - RNA, 100 nt, synthetic construct
  misc_feature 1..100: note = Synthetic
  source 1..100: mol_type = other RNA
gagatttcttgtctcactgagttttagagctagaaatagcaagttaaaataaggctagtccgttatcaacttgaaaaagtggcaccgagtcggtgctttt

SEQ ID NO: 318 - RNA, 100 nt, synthetic construct
  misc_feature 1..100: note = Synthetic
  source 1..100: mol_type = other RNA
taaagcacctgttaaaatgagttttagagctagaaatagcaagttaaaataaggctagtccgttatcaacttgaaaaagtggcaccgagtcggtgctttt

SEQ ID NO: 319 - RNA, 100 nt, synthetic construct
  misc_feature 1..100: note = Synthetic
  source 1..100: mol_type = other RNA
aatctgtccttcattttaacgttttagagctagaaatagcaagttaaaataaggctagtccgttatcaacttgaaaaagtggcaccgagtcggtgctttt

SEQ ID NO: 320 - RNA, 100 nt, synthetic construct
  misc_feature 1..100: note = Synthetic
  source 1..100: mol_type = other RNA
gtcacaggggaaggtccctggttttagagctagaaatagcaagttaaaataaggctagtccgttatcaacttgaaaaagtggcaccgagtcggtgctttt

SEQ ID NO: 321 - RNA, 100 nt, synthetic construct
  misc_feature 1..100: note = Synthetic
  source 1..100: mol_type = other RNA
aaacatgaagaaagcaggtggttttagagctagaaatagcaagttaaaataaggctagtccgttatcaacttgaaaaagtggcaccgagtcggtgctttt

SEQ ID NO: 322 - RNA, 100 nt, synthetic construct
  misc_feature 1..100: note = Synthetic
  source 1..100: mol_type = other RNA
tgtcctgtgagataccagaagttttagagctagaaatagcaagttaaaataaggctagtccgttatcaacttgaaaaagtggcaccgagtcggtgctttt

SEQ ID NO: 323 - RNA, 100 nt, synthetic construct
  misc_feature 1..100: note = Synthetic
  source 1..100: mol_type = other RNA
atgaaggaggctgatgcctggttttagagctagaaatagcaagttaaaataaggctagtccgttatcaacttgaaaaagtggcaccgagtcggtgctttt

SEQ ID NO: 324 - RNA, 100 nt, synthetic construct
  misc_feature 1..100: note = Synthetic
  source 1..100: mol_type = other RNA
aggctgatgcctgaggtcctgttttagagctagaaatagcaagttaaaataaggctagtccgttatcaacttgaaaaagtggcaccgagtcggtgctttt

SEQ ID NO: 325 - RNA, 100 nt, synthetic construct
  misc_feature 1..100: note = Synthetic
  source 1..100: mol_type = other RNA
ggctgatgcctgaggtccttgttttagagctagaaatagcaagttaaaataaggctagtccgttatcaacttgaaaaagtggcaccgagtcggtgctttt

SEQ ID NO: 326 - RNA, 100 nt, synthetic construct
  misc_feature 1..100: note = Synthetic
  source 1..100: mol_type = other RNA
cacaatatcccaaggacctcgttttagagctagaaatagcaagttaaaataaggctagtccgttatcaacttgaaaaagtggcaccgagtcggtgctttt

SEQ ID NO: 327 - RNA, 100 nt, synthetic construct
  misc_feature 1..100: note = Synthetic
  source 1..100: mol_type = other RNA
ggtccttgggatattgtgttgttttagagctagaaatagcaagttaaaataaggctagtccgttatcaacttgaaaaagtggcaccgagtcggtgctttt

SEQ ID NO: 328 - RNA, 100 nt, synthetic construct
  misc_feature 1..100: note = Synthetic
  source 1..100: mol_type = other RNA
gtccttgggatattgtgtttgttttagagctagaaatagcaagttaaaataaggctagtccgttatcaacttgaaaaagtggcaccgagtcggtgctttt

SEQ ID NO: 329 - RNA, 100 nt, synthetic construct
  misc_feature 1..100: note = Synthetic
  source 1..100: mol_type = other RNA
ctcccaaacacaatatcccagttttagagctagaaatagcaagttaaaataaggctagtccgttatcaacttgaaaaagtggcaccgagtcggtgctttt

SEQ ID NO: 330 - RNA, 100 nt, synthetic construct
  misc_feature 1..100: note = Synthetic
  source 1..100: mol_type = other RNA
tcctctagccacatcttctggttttagagctagaaatagcaagttaaaataaggctagtccgttatcaacttgaaaaagtggcaccgagtcggtgctttt

SEQ ID NO: 331 - RNA, 100 nt, synthetic construct
  misc_feature 1..100: note = Synthetic
  source 1..100: mol_type = other RNA
acagaagatgtggctagagggttttagagctagaaatagcaagttaaaataaggctagtccgttatcaacttgaaaaagtggcaccgagtcggtgctttt

SEQ ID NO: 332 - RNA, 100 nt, synthetic construct
  misc_feature 1..100: note = Synthetic
  source 1..100: mol_type = other RNA
cctctagccacatcttctgtgttttagagctagaaatagcaagttaaaataaggctagtccgttatcaacttgaaaaagtggcaccgagtcggtgctttt

SEQ ID NO: 333 - RNA, 100 nt, synthetic construct
  misc_feature 1..100: note = Synthetic
  source 1..100: mol_type = other RNA
cccacagaagatgtggctaggttttagagctagaaatagcaagttaaaataaggctagtccgttatcaacttgaaaaagtggcaccgagtcggtgctttt

SEQ ID NO: 334 - RNA, 100 nt, synthetic construct
  misc_feature 1..100: note = Synthetic
  source 1..100: mol_type = other RNA
gtcagatcccacagaagatggttttagagctagaaatagcaagttaaaataaggctagtccgttatcaacttgaaaaagtggcaccgagtcggtgctttt

SEQ ID NO: 335 - RNA, 100 nt, synthetic construct
  misc_feature 1..100: note = Synthetic
  source 1..100: mol_type = other RNA
atcttctgtgggatctgaccgttttagagctagaaatagcaagttaaaataaggctagtccgttatcaacttgaaaaagtggcaccgagtcggtgctttt

SEQ ID NO: 336 - RNA, 100 nt, synthetic construct
  misc_feature 1..100: note = Synthetic
  source 1..100: mol_type = other RNA
cccaggcagtgacagtgcccgttttagagctagaaatagcaagttaaaataaggctagtccgttatcaacttgaaaaagtggcaccgagtcggtgctttt

SEQ ID NO: 337 - RNA, 100 nt, synthetic construct
  misc_feature 1..100: note = Synthetic
  source 1..100: mol_type = other RNA
ctgggcactgtcactgcctggttttagagctagaaatagcaagttaaaataaggctagtccgttatcaacttgaaaaagtggcaccgagtcggtgctttt

SEQ ID NO: 338 - RNA, 100 nt, synthetic construct
  misc_feature 1..100: note = Synthetic
  source 1..100: mol_type = other RNA
cctgggcactgtcactgcctgttttagagctagaaatagcaagttaaaataaggctagtccgttatcaacttgaaaaagtggcaccgagtcggtgctttt

SEQ ID NO: 339 - RNA, 100 nt, synthetic construct
  misc_feature 1..100: note = Synthetic
  source 1..100: mol_type = other RNA
ccctgggcactgtcactgccgttttagagctagaaatagcaagttaaaataaggctagtccgttatcaacttgaaaaagtggcaccgagtcggtgctttt

SEQ ID NO: 340 - RNA, 100 nt, synthetic construct
  misc_feature 1..100: note = Synthetic
  source 1..100: mol_type = other RNA
ttgggtgttgggcggaacaggttttagagctagaaatagcaagttaaaataaggctagtccgttatcaacttgaaaaagtggcaccgagtcggtgctttt

SEQ ID NO: 341 - RNA, 100 nt, synthetic construct
  misc_feature 1..100: note = Synthetic
  source 1..100: mol_type = other RNA
tggatgtattgagcatgcgagttttagagctagaaatagcaagttaaaataaggctagtccgttatcaacttgaaaaagtggcaccgagtcggtgctttt

SEQ ID NO: 342 - RNA, 100 nt, synthetic construct
  misc_feature 1..100: note = Synthetic
  source 1..100: mol_type = other RNA
ggatgtattgagcatgcgatgttttagagctagaaatagcaagttaaaataaggctagtccgttatcaacttgaaaaagtggcaccgagtcggtgctttt

SEQ ID NO: 343 - RNA, 100 nt, synthetic construct
  misc_feature 1..100: note = Synthetic
  source 1..100: mol_type = other RNA
aacatgaagaaagcaggtgtgttttagagctagaaatagcaagttaaaataaggctagtccgttatcaacttgaaaaagtggcaccgagtcggtgctttt

SEQ ID NO: 344 - RNA, 100 nt, synthetic construct; identical to SEQ ID NO: 249
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 345 - RNA, 100 nt, synthetic construct; identical to SEQ ID NO: 250
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 346 - RNA, 100 nt, synthetic construct; identical to SEQ ID NO: 251
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 347 - RNA, 100 nt, synthetic construct; identical to SEQ ID NO: 252
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 348 - RNA, 100 nt, synthetic construct; identical to SEQ ID NO: 253
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 349 - RNA, 100 nt, synthetic construct; identical to SEQ ID NO: 254
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 350 - RNA, 100 nt, synthetic construct; identical to SEQ ID NO: 255
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 351 - RNA, 100 nt, synthetic construct; identical to SEQ ID NO: 256
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 352 - RNA, 100 nt, synthetic construct; identical to SEQ ID NO: 257
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 353 - RNA, 100 nt, synthetic construct; identical to SEQ ID NO: 258
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 354 - RNA, 100 nt, synthetic construct; identical to SEQ ID NO: 259
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 355 - RNA, 100 nt, synthetic construct; identical to SEQ ID NO: 260
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 356 - RNA, 100 nt, synthetic construct; identical to SEQ ID NO: 261
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 357 - RNA, 100 nt, synthetic construct; identical to SEQ ID NO: 262
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 358 - RNA, 100 nt, synthetic construct; identical to SEQ ID NO: 263
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 359 - RNA, 100 nt, synthetic construct; identical to SEQ ID NO: 264
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 360 - RNA, 100 nt, synthetic construct; identical to SEQ ID NO: 265
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 361 - RNA, 100 nt, synthetic construct; identical to SEQ ID NO: 266
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 362 - RNA, 100 nt, synthetic construct; identical to SEQ ID NO: 267
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 363 - RNA, 100 nt, synthetic construct; identical to SEQ ID NO: 268
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 364 - RNA, 100 nt, synthetic construct; identical to SEQ ID NO: 269
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 365 - RNA, 100 nt, synthetic construct; identical to SEQ ID NO: 270
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 366 - RNA, 100 nt, synthetic construct; identical to SEQ ID NO: 271
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 367 - RNA, 100 nt, synthetic construct; identical to SEQ ID NO: 272
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 368 - RNA, 100 nt, synthetic construct; identical to SEQ ID NO: 273
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 369 - RNA, 100 nt, synthetic construct; identical to SEQ ID NO: 274
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 370 - RNA, 100 nt, synthetic construct; identical to SEQ ID NO: 275
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 371 - RNA, 100 nt, synthetic construct; identical to SEQ ID NO: 276
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 372 - RNA, 100 nt, synthetic construct; identical to SEQ ID NO: 277
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 373 - RNA, 100 nt, synthetic construct; identical to SEQ ID NO: 278
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 374 - RNA, 100 nt, synthetic construct; identical to SEQ ID NO: 279
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 375 - RNA, 100 nt, synthetic construct; identical to SEQ ID NO: 280
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 376 - RNA, 100 nt, synthetic construct; identical to SEQ ID NO: 281
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 377 - RNA, 100 nt, synthetic construct; identical to SEQ ID NO: 282
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 378 - RNA, 100 nt, synthetic construct; identical to SEQ ID NO: 283
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 379 - RNA, 100 nt, synthetic construct; identical to SEQ ID NO: 284
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 380 - RNA, 100 nt, synthetic construct; identical to SEQ ID NO: 285
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 381 - RNA, 100 nt, synthetic construct; identical to SEQ ID NO: 286
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 382 - RNA, 100 nt, synthetic construct; identical to SEQ ID NO: 287
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 383 - RNA, 100 nt, synthetic construct; identical to SEQ ID NO: 288
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 384 - RNA, 100 nt, synthetic construct; identical to SEQ ID NO: 289
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 385 - RNA, 100 nt, synthetic construct; identical to SEQ ID NO: 290
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 386 - RNA, 100 nt, synthetic construct; identical to SEQ ID NO: 291
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 387 - RNA, 100 nt, synthetic construct; identical to SEQ ID NO: 292
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 388 - RNA, 100 nt, synthetic construct; identical to SEQ ID NO: 293
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 389 - RNA, 100 nt, synthetic construct; identical to SEQ ID NO: 294
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 390 - RNA, 100 nt, synthetic construct; identical to SEQ ID NO: 295
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 391 - RNA, 100 nt, synthetic construct; identical to SEQ ID NO: 296
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 392 - RNA, 100 nt, synthetic construct; identical to SEQ ID NO: 297
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 393 - RNA, 100 nt, synthetic construct; identical to SEQ ID NO: 298
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 394 - RNA, 100 nt, synthetic construct; identical to SEQ ID NO: 299
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 395 - RNA, 100 nt, synthetic construct; identical to SEQ ID NO: 300
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 396 - RNA, 100 nt, synthetic construct; identical to SEQ ID NO: 301
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 397 - RNA, 100 nt, synthetic construct; identical to SEQ ID NO: 302
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 398 - RNA, 100 nt, synthetic construct; identical to SEQ ID NO: 303
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 399 - RNA, 100 nt, synthetic construct; identical to SEQ ID NO: 304
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 400 - RNA, 100 nt, synthetic construct; identical to SEQ ID NO: 305
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 401 - RNA, 100 nt, synthetic construct; identical to SEQ ID NO: 306
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 402 - RNA, 100 nt, synthetic construct; identical to SEQ ID NO: 307
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 403 - RNA, 100 nt, synthetic construct; identical to SEQ ID NO: 308
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 404 - RNA, 100 nt, synthetic construct; identical to SEQ ID NO: 309
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 405 - RNA, 100 nt, synthetic construct; identical to SEQ ID NO: 310
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 406 - RNA, 100 nt, synthetic construct; identical to SEQ ID NO: 311
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 407 - RNA, 100 nt, synthetic construct; identical to SEQ ID NO: 312
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 408 - RNA, 100 nt, synthetic construct; identical to SEQ ID NO: 313
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 409 - RNA, 100 nt, synthetic construct; identical to SEQ ID NO: 314
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 410 - RNA, 100 nt, synthetic construct; identical to SEQ ID NO: 315
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 411 - RNA, 100 nt, synthetic construct; identical to SEQ ID NO: 316
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 412 - RNA, 100 nt, synthetic construct; identical to SEQ ID NO: 317
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 413 - RNA, 100 nt, synthetic construct; identical to SEQ ID NO: 318
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 414 - RNA, 100 nt, synthetic construct; identical to SEQ ID NO: 319
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 415 - RNA, 100 nt, synthetic construct; identical to SEQ ID NO: 320
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 416 - RNA, 100 nt, synthetic construct; identical to SEQ ID NO: 321
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 417 - RNA, 100 nt, synthetic construct; identical to SEQ ID NO: 322
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 418 - RNA, 100 nt, synthetic construct; identical to SEQ ID NO: 323
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 419 - RNA, 100 nt, synthetic construct; identical to SEQ ID NO: 324
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 420 - RNA, 100 nt, synthetic construct; identical to SEQ ID NO: 325
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 421 - RNA, 100 nt, synthetic construct; identical to SEQ ID NO: 326
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 422 - RNA, 100 nt, synthetic construct; identical to SEQ ID NO: 327
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 423 - RNA, 100 nt, synthetic construct; identical to SEQ ID NO: 328
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 424 - RNA, 100 nt, synthetic construct; identical to SEQ ID NO: 329
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 425 - RNA, 100 nt, synthetic construct; identical to SEQ ID NO: 330
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 426 - RNA, 100 nt, synthetic construct; identical to SEQ ID NO: 331
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 427 - RNA, 100 nt, synthetic construct; identical to SEQ ID NO: 332
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 428 - RNA, 100 nt, synthetic construct; identical to SEQ ID NO: 333
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 429 - RNA, 100 nt, synthetic construct; identical to SEQ ID NO: 334
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 430 - RNA, 100 nt, synthetic construct; identical to SEQ ID NO: 335
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 431 - RNA, 100 nt, synthetic construct; identical to SEQ ID NO: 336
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 432 - RNA, 100 nt, synthetic construct; identical to SEQ ID NO: 337
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 433 - RNA, 100 nt, synthetic construct; identical to SEQ ID NO: 338
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 434 - RNA, 100 nt, synthetic construct; identical to SEQ ID NO: 339
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 435 - RNA, 100 nt, synthetic construct; identical to SEQ ID NO: 340
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 436 - RNA, 100 nt, synthetic construct; identical to SEQ ID NO: 341
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 437 - RNA, 100 nt, synthetic construct; identical to SEQ ID NO: 342
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 438 - RNA, 100 nt, synthetic construct; identical to SEQ ID NO: 343
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 439 - RNA, 100 nt, synthetic construct
  misc_feature 1..100: note = Synthetic
  source 1..100: mol_type = other RNA
tagcccacggcgatgaagcggttttagagctagaaatagcaagttaaaataaggctagtccgttatcaacttgaaaaagtggcaccgagtcggtgctttt

SEQ ID NO: 440 - RNA, 100 nt, synthetic construct
  misc_feature 1..100: note = Synthetic
  source 1..100: mol_type = other RNA
gtagcccacggcgatgaagcgttttagagctagaaatagcaagttaaaataaggctagtccgttatcaacttgaaaaagtggcaccgagtcggtgctttt

SEQ ID NO: 441 - RNA, 100 nt, synthetic construct
  misc_feature 1..100: note = Synthetic
  source 1..100: mol_type = other RNA
cgtagcccacggcgatgaaggttttagagctagaaatagcaagttaaaataaggctagtccgttatcaacttgaaaaagtggcaccgagtcggtgctttt

SEQ ID NO: 442 - RNA, 100 nt, synthetic construct
  misc_feature 1..100: note = Synthetic
  source 1..100: mol_type = other RNA
cttcatcgccgtgggctacggttttagagctagaaatagcaagttaaaataaggctagtccgttatcaacttgaaaaagtggcaccgagtcggtgctttt

SEQ ID NO: 443 - RNA, 100 nt, synthetic construct
  misc_feature 1..100: note = Synthetic
  source 1..100: mol_type = other RNA
cgtgtcgtccacgtagcccagttttagagctagaaatagcaagttaaaataaggctagtccgttatcaacttgaaaaagtggcaccgagtcggtgctttt

SEQ ID NO: 444 - RNA, 100 nt, synthetic construct
  misc_feature 1..100: note = Synthetic
  source 1..100: mol_type = other RNA
tggacgacacgcagttcgtggttttagagctagaaatagcaagttaaaataaggctagtccgttatcaacttgaaaaagtggcaccgagtcggtgctttt

SEQ ID NO: 445 - RNA, 100 nt, synthetic construct
  misc_feature 1..100: note = Synthetic
  source 1..100: mol_type = other RNA
ggatggagccgcgggcgccggttttagagctagaaatagcaagttaaaataaggctagtccgttatcaacttgaaaaagtggcaccgagtcggtgctttt

SEQ ID NO: 446 - RNA, 100 nt, synthetic construct
  misc_feature 1..100: note = Synthetic
  source 1..100: mol_type = other RNA
gcgggcgccgtggatagagcgttttagagctagaaatagcaagttaaaataaggctagtccgttatcaacttgaaaaagtggcaccgagtcggtgctttt

SEQ ID NO: 447 - RNA, 100 nt, synthetic construct
  misc_feature 1..100: note = Synthetic
  source 1..100: mol_type = other RNA
tgctctatccacggcgcccggttttagagctagaaatagcaagttaaaataaggctagtccgttatcaacttgaaaaagtggcaccgagtcggtgctttt

SEQ ID NO: 448 - RNA, 100 nt, synthetic construct
  misc_feature 1..100: note = Synthetic
  source 1..100: mol_type = other RNA
ggcgccgtggatagagcagggttttagagctagaaatagcaagttaaaataaggctagtccgttatcaacttgaaaaagtggcaccgagtcggtgctttt

SEQ ID NO: 449 - RNA, 100 nt, synthetic construct
  misc_feature 1..100: note = Synthetic
  source 1..100: mol_type = other RNA
gcgccgtggatagagcaggagttttagagctagaaatagcaagttaaaataaggctagtccgttatcaacttgaaaaagtggcaccgagtcggtgctttt

SEQ ID NO: 450 - RNA, 100 nt, synthetic construct
  misc_feature 1..100: note = Synthetic
  source 1..100: mol_type = other RNA
cgccgtggatagagcaggaggttttagagctagaaatagcaagttaaaataaggctagtccgttatcaacttgaaaaagtggcaccgagtcggtgctttt

SEQ ID NO: 451 - RNA, 100 nt, synthetic construct
  misc_feature 1..100: note = Synthetic
  source 1..100: mol_type = other RNA
gtggatagagcaggaggggcgttttagagctagaaatagcaagttaaaataaggctagtccgttatcaacttgaaaaagtggcaccgagtcggtgctttt

SEQ ID NO: 452 - RNA, 100 nt, synthetic construct
  misc_feature 1..100: note = Synthetic
  source 1..100: mol_type = other RNA
ggcccctcctgctctatccagttttagagctagaaatagcaagttaaaataaggctagtccgttatcaacttgaaaaagtggcaccgagtcggtgctttt

SEQ ID NO: 453 - RNA, 100 nt, synthetic construct
  misc_feature 1..100: note = Synthetic
  source 1..100: mol_type = other RNA
agcaggaggggccggagtatgttttagagctagaaatagcaagttaaaataaggctagtccgttatcaacttgaaaaagtggcaccgagtcggtgctttt

SEQ ID NO: 454 - RNA, 100 nt, synthetic construct
  misc_feature 1..100: note = Synthetic
  source 1..100: mol_type = other RNA
gcaggaggggccggagtattgttttagagctagaaatagcaagttaaaataaggctagtccgttatcaacttgaaaaagtggcaccgagtcggtgctttt

SEQ ID NO: 455 - RNA, 100 nt, synthetic construct
  misc_feature 1..100: note = Synthetic
  source 1..100: mol_type = other RNA
ggagtggctccgcagataccgttttagagctagaaatagcaagttaaaataaggctagtccgttatcaacttgaaaaagtggcaccgagtcggtgctttt

SEQ ID NO: 456 - RNA, 100 nt, synthetic construct
  misc_feature 1..100: note = Synthetic
  source 1..100: mol_type = other RNA
ctccgcagatacctggagaagttttagagctagaaatagcaagttaaaataaggctagtccgttatcaacttgaaaaagtggcaccgagtcggtgctttt

SEQ ID NO: 457 - RNA, 100 nt, synthetic construct
  misc_feature 1..100: note = Synthetic
  source 1..100: mol_type = other RNA
tccgcagatacctggagaacgttttagagctagaaatagcaagttaaaataaggctagtccgttatcaacttgaaaaagtggcaccgagtcggtgctttt

SEQ ID NO: 458 - RNA, 100 nt, synthetic construct
  misc_feature 1..100: note = Synthetic
  source 1..100: mol_type = other RNA
cagatacctggagaacgggagttttagagctagaaatagcaagttaaaataaggctagtccgttatcaacttgaaaaagtggcaccgagtcggtgctttt

SEQ ID NO: 459 - RNA, 100 nt, synthetic construct
  misc_feature 1..100: note = Synthetic
  source 1..100: mol_type = other RNA
tcccgttctccaggtatctggttttagagctagaaatagcaagttaaaataaggctagtccgttatcaacttgaaaaagtggcaccgagtcggtgctttt

SEQ ID NO: 460 - RNA, 100 nt, synthetic construct
  misc_feature 1..100: note = Synthetic
  source 1..100: mol_type = other RNA
gcgtctccttcccgttctccgttttagagctagaaatagcaagttaaaataaggctagtccgttatcaacttgaaaaagtggcaccgagtcggtgctttt

SEQ ID NO: 461 - RNA, 100 nt, synthetic construct
  misc_feature 1..100: note = Synthetic
  source 1..100: mol_type = other RNA
gaaggagacgctgcagcgcagttttagagctagaaatagcaagttaaaataaggctagtccgttatcaacttgaaaaagtggcaccgagtcggtgctttt

SEQ ID NO: 462 - RNA, 100 nt, synthetic construct
  misc_feature 1..100: note = Synthetic
  source 1..100: mol_type = other RNA
aaggagacgctgcagcgcacgttttagagctagaaatagcaagttaaaataaggctagtccgttatcaacttgaaaaagtggcaccgagtcggtgctttt

SEQ ID NO: 463 - RNA, 100 nt, synthetic construct
  misc_feature 1..100: note = Synthetic
  source 1..100: mol_type = other RNA
agatctacaggcgatcaggggttttagagctagaaatagcaagttaaaataaggctagtccgttatcaacttgaaaaagtggcaccgagtcggtgctttt

SEQ ID NO: 464 - RNA, 100 nt, synthetic construct
  misc_feature 1..100: note = Synthetic
  source 1..100: mol_type = other RNA
tgatcgcctgtagatctcccgttttagagctagaaatagcaagttaaaataaggctagtccgttatcaacttgaaaaagtggcaccgagtcggtgctttt

SEQ ID NO: 465 - RNA, 100 nt, synthetic construct
  misc_feature 1..100: note = Synthetic
  source 1..100: mol_type = other RNA
gggagatctacaggcgatcagttttagagctagaaatagcaagttaaaataaggctagtccgttatcaacttgaaaaagtggcaccgagtcggtgctttt

SEQ ID NO: 466 - RNA, 100 nt, synthetic construct
  misc_feature 1..100: note = Synthetic
  source 1..100: mol_type = other RNA
cgggagatctacaggcgatcgttttagagctagaaatagcaagttaaaataaggctagtccgttatcaacttgaaaaagtggcaccgagtcggtgctttt

SEQ ID NO: 467 - RNA, 100 nt, synthetic construct
  misc_feature 1..100: note = Synthetic
  source 1..100: mol_type = other RNA
cgcctgtagatctcccgggcgttttagagctagaaatagcaagttaaaataaggctagtccgttatcaacttgaaaaagtggcaccgagtcggtgctttt

SEQ ID NO: 468 - RNA, 100 nt, synthetic construct
  misc_feature 1..100: note = Synthetic
  source 1..100: mol_type = other RNA
ggccagcccgggagatctacgttttagagctagaaatagcaagttaaaataaggctagtccgttatcaacttgaaaaagtggcaccgagtcggtgctttt

SEQ ID NO: 469 - RNA, 100 nt, synthetic construct
  misc_feature 1..100: note = Synthetic
  source 1..100: mol_type = other RNA
tcccgggctggcctcccacagttttagagctagaaatagcaagttaaaataaggctagtccgttatcaacttgaaaaagtggcaccgagtcggtgctttt

SEQ ID NO: 470 - RNA, 100 nt, synthetic construct
  misc_feature 1..100: note = Synthetic
  source 1..100: mol_type = other RNA
gggctggcctcccacaaggagttttagagctagaaatagcaagttaaaataaggctagtccgttatcaacttgaaaaagtggcaccgagtcggtgctttt

SEQ ID NO: 471 - RNA, 100 nt, synthetic construct
  misc_feature 1..100: note = Synthetic
  source 1..100: mol_type = other RNA
ctgatcgcctgtagatctccgttttagagctagaaatagcaagttaaaataaggctagtccgttatcaacttgaaaaagtggcaccgagtcggtgctttt

SEQ ID NO: 472 - RNA, 100 nt, synthetic construct; identical to SEQ ID NO: 439
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 473 - RNA, 100 nt, synthetic construct; identical to SEQ ID NO: 440
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 474 - RNA, 100 nt, synthetic construct; identical to SEQ ID NO: 441
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 475 - RNA, 100 nt, synthetic construct; identical to SEQ ID NO: 442
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 476 - RNA, 100 nt, synthetic construct; identical to SEQ ID NO: 443
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 477 - RNA, 100 nt, synthetic construct; identical to SEQ ID NO: 444
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 478 - RNA, 100 nt, synthetic construct; identical to SEQ ID NO: 445
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 479 - RNA, 100 nt, synthetic construct; identical to SEQ ID NO: 446
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 480 - RNA, 100 nt, synthetic construct; identical to SEQ ID NO: 447
  misc_feature 1..100: note = Synthetic
  variation 1..100: note = 2'-O-methylation and phosphorothioate internucleoside linkage modifications are present or absent embodiments
  source 1..100: mol_type = other RNA

SEQ ID NO: 481 - RNA, 100 nt, synthetic construct; identical to SEQ ID NO: 448
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 482 - RNA, 100 nt, synthetic construct; identical to SEQ ID NO: 449
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 483 - RNA, 100 nt, synthetic construct; identical to SEQ ID NO: 450
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 484 - RNA, 100 nt, synthetic construct; identical to SEQ ID NO: 451
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 485 - RNA, 100 nt, synthetic construct; identical to SEQ ID NO: 452
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 486 - RNA, 100 nt, synthetic construct; identical to SEQ ID NO: 453
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 487 - RNA, 100 nt, synthetic construct; identical to SEQ ID NO: 454
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 488 - RNA, 100 nt, synthetic construct; identical to SEQ ID NO: 455
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 489 - RNA, 100 nt, synthetic construct; identical to SEQ ID NO: 456
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 490 - RNA, 100 nt, synthetic construct; identical to SEQ ID NO: 457
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 491 - RNA, 100 nt, synthetic construct; identical to SEQ ID NO: 458
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 492 - RNA, 100 nt, synthetic construct; identical to SEQ ID NO: 459
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 493 - RNA, 100 nt, synthetic construct; identical to SEQ ID NO: 460
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 494 - RNA, 100 nt, synthetic construct; identical to SEQ ID NO: 461
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 495 - RNA, 100 nt, synthetic construct; identical to SEQ ID NO: 462
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 496 - RNA, 100 nt, synthetic construct; identical to SEQ ID NO: 463
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 497 - RNA, 100 nt, synthetic construct; identical to SEQ ID NO: 464
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 498 - RNA, 100 nt, synthetic construct; identical to SEQ ID NO: 465
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 499 - RNA, 100 nt, synthetic construct; identical to SEQ ID NO: 466
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 500 - RNA, 100 nt, synthetic construct; identical to SEQ ID NO: 467
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 501 - RNA, 100 nt, synthetic construct; identical to SEQ ID NO: 468
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 502 - RNA, 100 nt, synthetic construct; identical to SEQ ID NO: 469
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 503 - RNA, 100 nt, synthetic construct; identical to SEQ ID NO: 470
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 504 - RNA, 100 nt, synthetic construct; identical to SEQ ID NO: 471
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 505 - RNA, 100 nt, synthetic construct
  misc_feature 1..100: note = Synthetic
  source 1..100: mol_type = other RNA
acgacactgattggcttctcgttttagagctagaaatagcaagttaaaataaggctagtccgttatcaacttgaaaaagtggcaccgagtcggtgctttt

SEQ ID NO: 506 - RNA, 100 nt, synthetic construct
  misc_feature 1..100: note = Synthetic
  source 1..100: mol_type = other RNA
acccctcatcccccacggacgttttagagctagaaatagcaagttaaaataaggctagtccgttatcaacttgaaaaagtggcaccgagtcggtgctttt

SEQ ID NO: 507 - RNA, 100 nt, synthetic construct
  misc_feature 1..100: note = Synthetic
  source 1..100: mol_type = other RNA
ggcccgtccgtgggggatgagttttagagctagaaatagcaagttaaaataaggctagtccgttatcaacttgaaaaagtggcaccgagtcggtgctttt

SEQ ID NO: 508 - RNA, 100 nt, synthetic construct
  misc_feature 1..100: note = Synthetic
  source 1..100: mol_type = other RNA
gccaggtcgcccacagtctcgttttagagctagaaatagcaagttaaaataaggctagtccgttatcaacttgaaaaagtggcaccgagtcggtgctttt

SEQ ID NO: 509 - RNA, 100 nt, synthetic construct
  misc_feature 1..100: note = Synthetic
  source 1..100: mol_type = other RNA
gtttaggccaaaaatcccccgttttagagctagaaatagcaagttaaaataaggctagtccgttatcaacttgaaaaagtggcaccgagtcggtgctttt

SEQ ID NO: 510 - RNA, 100 nt, synthetic construct
  misc_feature 1..100: note = Synthetic
  source 1..100: mol_type = other RNA
ggccaaaaatccccccgggtgttttagagctagaaatagcaagttaaaataaggctagtccgttatcaacttgaaaaagtggcaccgagtcggtgctttt

SEQ ID NO: 511 - RNA, 100 nt, synthetic construct
  misc_feature 1..100: note = Synthetic
  source 1..100: mol_type = other RNA
gaccaacccggggggattttgttttagagctagaaatagcaagttaaaataaggctagtccgttatcaacttgaaaaagtggcaccgagtcggtgctttt

SEQ ID NO: 512 - RNA, 100 nt, synthetic construct
  misc_feature 1..100: note = Synthetic
  source 1..100: mol_type = other RNA
cacgggcccaaggctgctgcgttttagagctagaaatagcaagttaaaataaggctagtccgttatcaacttgaaaaagtggcaccgagtcggtgctttt

SEQ ID NO: 513 - RNA, 100 nt, synthetic construct
  misc_feature 1..100: note = Synthetic
  source 1..100: mol_type = other RNA
accctcatgctgcacatggcgttttagagctagaaatagcaagttaaaataaggctagtccgttatcaacttgaaaaagtggcaccgagtcggtgctttt

SEQ ID NO: 514 - RNA, 100 nt, synthetic construct
  misc_feature 1..100: note = Synthetic
  source 1..100: mol_type = other RNA
cctctaggaccttaaggcccgttttagagctagaaatagcaagttaaaataaggctagtccgttatcaacttgaaaaagtggcaccgagtcggtgctttt

SEQ ID NO: 515 - RNA, 100 nt, synthetic construct
  misc_feature 1..100: note = Synthetic
  source 1..100: mol_type = other RNA
gctcctttctggtatctcacgttttagagctagaaatagcaagttaaaataaggctagtccgttatcaacttgaaaaagtggcaccgagtcggtgctttt

SEQ ID NO: 516 - RNA, 100 nt, synthetic construct
  misc_feature 1..100: note = Synthetic
  source 1..100: mol_type = other RNA
gctatggggtttctttgcatgttttagagctagaaatagcaagttaaaataaggctagtccgttatcaacttgaaaaagtggcaccgagtcggtgctttt

SEQ ID NO: 517 - RNA, 100 nt, synthetic construct
  misc_feature 1..100: note = Synthetic
  source 1..100: mol_type = other RNA
gcctttgcagaaacaaagtcgttttagagctagaaatagcaagttaaaataaggctagtccgttatcaacttgaaaaagtggcaccgagtcggtgctttt

SEQ ID NO: 518 - RNA, 100 nt, synthetic construct
  misc_feature 1..100: note = Synthetic
  source 1..100: mol_type = other RNA
tggaccaaccgccctcctgagttttagagctagaaatagcaagttaaaataaggctagtccgttatcaacttgaaaaagtggcaccgagtcggtgctttt

SEQ ID NO: 519 - RNA, 100 nt, synthetic construct
  misc_feature 1..100: note = Synthetic
  source 1..100: mol_type = other RNA
agcctctctgacctttagcagttttagagctagaaatagcaagttaaaataaggctagtccgttatcaacttgaaaaagtggcaccgagtcggtgctttt

SEQ ID NO: 520 - RNA, 100 nt, synthetic construct
  misc_feature 1..100: note = Synthetic
  source 1..100: mol_type = other RNA
cgccctcctgaaggtcctcagttttagagctagaaatagcaagttaaaataaggctagtccgttatcaacttgaaaaagtggcaccgagtcggtgctttt

SEQ ID NO: 521 - RNA, 100 nt, synthetic construct
  misc_feature 1..100: note = Synthetic
  source 1..100: mol_type = other RNA
ccgccctcctgaaggtcctcgttttagagctagaaatagcaagttaaaataaggctagtccgttatcaacttgaaaaagtggcaccgagtcggtgctttt

SEQ ID NO: 522 - RNA, 100 nt, synthetic construct
  misc_feature 1..100: note = Synthetic
  source 1..100: mol_type = other RNA
tggttccctttgacacacacgttttagagctagaaatagcaagttaaaataaggctagtccgttatcaacttgaaaaagtggcaccgagtcggtgctttt

SEQ ID NO: 523 - RNA, 100 nt, synthetic construct
  misc_feature 1..100: note = Synthetic
  source 1..100: mol_type = other RNA
gaccctgctaaaggtcagaggttttagagctagaaatagcaagttaaaataaggctagtccgttatcaacttgaaaaagtggcaccgagtcggtgctttt

SEQ ID NO: 524 - RNA, 100 nt, synthetic construct
  misc_feature 1..100: note = Synthetic
  source 1..100: mol_type = other RNA
aggaccttcaggagggcggtgttttagagctagaaatagcaagttaaaataaggctagtccgttatcaacttgaaaaagtggcaccgagtcggtgctttt

SEQ ID NO: 525 - RNA, 100 nt, synthetic construct
  misc_feature 1..100: note = Synthetic
  source 1..100: mol_type = other RNA
gcacacttctacctgggtctgttttagagctagaaatagcaagttaaaataaggctagtccgttatcaacttgaaaaagtggcaccgagtcggtgctttt

SEQ ID NO: 526 - RNA, 100 nt, synthetic construct
  misc_feature 1..100: note = Synthetic
  source 1..100: mol_type = other RNA
gagcctctctgacctttagcgttttagagctagaaatagcaagttaaaataaggctagtccgttatcaacttgaaaaagtggcaccgagtcggtgctttt

SEQ ID NO: 527 - RNA, 100 nt, synthetic construct
  misc_feature 1..100: note = Synthetic
  source 1..100: mol_type = other RNA
acactcctccagcacacatggttttagagctagaaatagcaagttaaaataaggctagtccgttatcaacttgaaaaagtggcaccgagtcggtgctttt

SEQ ID NO: 528 - RNA, 100 nt, synthetic construct
  misc_feature 1..100: note = Synthetic
  source 1..100: mol_type = other RNA
ctctgacctttagcagggtcgttttagagctagaaatagcaagttaaaataaggctagtccgttatcaacttgaaaaagtggcaccgagtcggtgctttt

SEQ ID NO: 529 - RNA, 100 nt, synthetic construct
  misc_feature 1..100: note = Synthetic
  source 1..100: mol_type = other RNA
caagatagccacatgtgtgcgttttagagctagaaatagcaagttaaaataaggctagtccgttatcaacttgaaaaagtggcaccgagtcggtgctttt

SEQ ID NO: 530 - RNA, 100 nt, synthetic construct
  misc_feature 1..100: note = Synthetic
  source 1..100: mol_type = other RNA
tctgacctttagcagggtcagttttagagctagaaatagcaagttaaaataaggctagtccgttatcaacttgaaaaagtggcaccgagtcggtgctttt

SEQ ID NO: 531 - RNA, 100 nt, synthetic construct
  misc_feature 1..100: note = Synthetic
  source 1..100: mol_type = other RNA
tgtaaaggtgagagcctggagttttagagctagaaatagcaagttaaaataaggctagtccgttatcaacttgaaaaagtggcaccgagtcggtgctttt

SEQ ID NO: 532 - RNA, 100 nt, synthetic construct
  misc_feature 1..100: note = Synthetic
  source 1..100: mol_type = other RNA
gaaggtccctgaggaccttcgttttagagctagaaatagcaagttaaaataaggctagtccgttatcaacttgaaaaagtggcaccgagtcggtgctttt

SEQ ID NO: 533 - RNA, 100 nt, synthetic construct; identical to SEQ ID NO: 505
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 534 - RNA, 100 nt, synthetic construct; identical to SEQ ID NO: 506
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 535 - RNA, 100 nt, synthetic construct; identical to SEQ ID NO: 507
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 536 - RNA, 100 nt, synthetic construct; identical to SEQ ID NO: 508
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 537 - RNA, 100 nt, synthetic construct; identical to SEQ ID NO: 509
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 538 - RNA, 100 nt, synthetic construct; identical to SEQ ID NO: 510
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 539 - RNA, 100 nt, synthetic construct; identical to SEQ ID NO: 511
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 540 - RNA, 100 nt, synthetic construct; identical to SEQ ID NO: 512
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 541 - RNA, 100 nt, synthetic construct; identical to SEQ ID NO: 513
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 542 - RNA, 100 nt, synthetic construct; identical to SEQ ID NO: 514
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 543 - RNA, 100 nt, synthetic construct; identical to SEQ ID NO: 515
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 544 - RNA, 100 nt, synthetic construct; identical to SEQ ID NO: 516
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 545 - RNA, 100 nt, synthetic construct; identical to SEQ ID NO: 517
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 546 - RNA, 100 nt, synthetic construct; identical to SEQ ID NO: 518
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 547 - RNA, 100 nt, synthetic construct; identical to SEQ ID NO: 519
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 548 - RNA, 100 nt, synthetic construct; identical to SEQ ID NO: 520
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 549 - RNA, 100 nt, synthetic construct; identical to SEQ ID NO: 521
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 550 - RNA, 100 nt, synthetic construct; identical to SEQ ID NO: 522
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 551 - RNA, 100 nt, synthetic construct; identical to SEQ ID NO: 523
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 552 - RNA, 100 nt, synthetic construct; identical to SEQ ID NO: 524
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 553 - RNA, 100 nt, synthetic construct; identical to SEQ ID NO: 525
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 554 - RNA, 100 nt, synthetic construct; identical to SEQ ID NO: 526
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 555 - RNA, 100 nt, synthetic construct; identical to SEQ ID NO: 527
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 556 - RNA, 100 nt, synthetic construct; identical to SEQ ID NO: 528
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 557 - RNA, 100 nt, synthetic construct; identical to SEQ ID NO: 529
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 558 - RNA, 100 nt, synthetic construct; identical to SEQ ID NO: 530
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 559 - RNA, 100 nt, synthetic construct; identical to SEQ ID NO: 531
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 560 - RNA, 100 nt, synthetic construct; identical to SEQ ID NO: 532
  misc_feature 1..100: note = Synthetic
  variation 1..100: note = 2'-O-methylation and phosphorothioate internucleoside linkage modifications are present or absent
  source 1..100: mol_type = other RNA

SEQ ID NO: 561-599 - skipped (39 entries)

SEQ ID NO: 600 - protein, 7 aa, synthetic construct
  REGION 1..7: note = Synthetic
PKKKRKV

SEQ ID NO: 601 - protein, 7 aa, synthetic construct
  REGION 1..7: note = Synthetic
PKKKRRV

SEQ ID NO: 602 - protein, 16 aa, synthetic construct
  REGION 1..16: note = Synthetic
KRPAATKKAGQAKKKK

SEQ ID NO: 603-799 - skipped (197 entries)

SEQ ID NO: 800 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 801 - DNA, 4140 bp, synthetic construct
  misc_feature 1..4140: note = Synthetic
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 802 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 803 - RNA, 4197 nt, synthetic construct
  misc_feature 1..4197: note = Synthetic
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 804 - RNA, 4782 nt, synthetic construct
  misc_feature 1..4782: note = Synthetic
  source 1..4782: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 805 - RNA, 4839 nt, synthetic construct
  misc_feature 1..4839: note = Synthetic
  source 1..4839: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 806 - skipped

SEQ ID NO: 807 - RNA, 291 nt, synthetic construct
  misc_feature 1..291: note = Synthetic
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 808 - RNA, 324 nt, synthetic construct
  misc_feature 1..324: note = Synthetic
  source 1..324: mol_type = other RNA
atgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtga

SEQ ID NO: 809 - protein, 1379 aa, synthetic construct; identical to SEQ ID NO: 800
  REGION 1..1379: note = Synthetic

SEQ ID NO: 810 - protein, 1398 aa, synthetic construct
  REGION 1..1398: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 811 - protein, 1593 aa, synthetic construct
  REGION 1..1593: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 812 - protein, 1612 aa, synthetic construct
  REGION 1..1612: note = Synthetic
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 813 - skipped

SEQ ID NO: 814 - protein, 107 aa, synthetic construct
  REGION 1..107: note = Synthetic
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 815 - skipped

SEQ ID NO: 816 - RNA, 91 nt, synthetic construct
  misc_feature 1..91: note = Synthetic
  variation 1..91: note = 2'-O-methylation and phosphorothioate internucleoside linkage modifications are present or absent
  source 1..91: mol_type = other RNA
actcacgctggatagcctccgttttagagctagaaatagcaagttaaaataaggctagtccgttatcacgaaagggcaccgagtcggtgct

SEQ ID NO: 817 - RNA, 4140 nt, synthetic construct
  misc_feature 1..4140: note = Synthetic
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 818 - RNA, 5043 nt, synthetic construct
  misc_feature 1..5043: note = Synthetic
  source 1..5043: mol_type = other RNA
atggaagcaagcccggcaagcggaccgagacacctgatggacccgcacatcttcacaagcaacttcaacaacggaatcggaagacacaagacatacctgtgctacgaagtcgaaagactggacaacggaacaagcgtcaagatggaccagcacagaggattcctgcacaaccaggcaaagaacctgctgtgcggattctacggaagacacgcagaactgagattcctggacctggtcccgagcctgcagctggacccggcacagatctacagagtcacatggttcatcagctggagcccgtgcttcagctggggatgcgcaggagaagtcagagcatttctgcaggaaaacacacacgtcagactgagaatcttcgcagcaagaatctacgactacgacccgctgtacaaggaagcactgcagatgctgagagacgcaggagcacaggtcagcatcatgacatacgacgaattcaagcactgctgggacacattcgtcgaccaccagggatgcccgttccagccgtgggacggactggacgaacacagccaggcactgagcggaagactgagagcaatcctgcagaaccagggaaacagcggaagcgaaacaccgggaacaagcgaaagcgcaacaccggaaagcgacaagaagtacagcatcggactggccatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgatctgagccagctgggaggagacagcggaggaagcacaaacctgagcgacatcatcgaaaaggaaacaggaaagcagctggtcatccaggaaagcatcctgatgctgccggaagaagtcgaagaagtcatcggaaacaagccggaaagcgacatcctggtccacacagcatacgacgaaagcacagacgaaaacgtcatgctgctgacaagcgacgcaccggaatacaagccgtgggcactggtcatccaggacagcaacggagaaaacaagatcaagatgctgagcggaggaagcccgaagaagaagagaaaggtctaa

SEQ ID NO: 819 - RNA, 291 nt, synthetic construct; identical to SEQ ID NO: 807
  misc_feature 1..291: note = Synthetic
  source 1..291: mol_type = other RNA

SEQ ID NO: 820-899 - skipped (80 entries)

SEQ ID NO: 900 - protein, 16 aa, synthetic construct
  REGION 1..16: note = Synthetic
SGSETPGTSESATPES

SEQ ID NO: 901 - protein, 12 aa, synthetic construct
  REGION 1..12: note = Synthetic
SGSETPGTSESA

SEQ ID NO: 902 - protein, 21 aa, synthetic construct
  REGION 1..21: note = Synthetic
SGSETPGTSESATPEGGSGGS

SEQ ID NO: 903-971 - skipped (69 entries)

SEQ ID NO: 972 - RNA, 5342 nt, synthetic construct
  misc_feature 1..5342: note = Synthetic
  source 1..5342: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 973 - RNA, 5399 nt, synthetic construct
  misc_feature 1..5399: note = Synthetic
  source 1..5399: mol_type = other RNA
gggaagctcagaataaacgctcaactttggccggatctgccaccatggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 974 - skipped

SEQ ID NO: 975 - RNA, 589 nt, synthetic construct
  misc_feature 1..589: note = Synthetic
  source 1..589: mol_type = other RNA
gggagacccaagctggctagctcccgcagtcggcgtccagcggctctgcttgttcgtgtgtgtgtcgttgcaggccttattcggatccgccaccatgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtgatagtctagacatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagtctag

SEQ ID NO: 976-999 - skipped (24 entries)

SEQ ID NO: 1000 - DNA, 8540 bp, synthetic construct
  misc_feature 1..8540: note = Synthetic
  source 1..8540: mol_type = other DNA
gcgatcgcagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttggctcccgatcgttgcgttacacacacaattactgctgatcgagtgtagccttcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagacgccaccatgtctcgctccgtggccttagctgtgctcgcgctactctctctttctggcctagaggctgttatggctccgcggactttaattttaggtggtggcggatccggtggaggcggttctggtggaggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccggatctcactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaacctgcggacgctgcgcggctactacaatcagagcgaggccgggtctcacaccctgcagtggatgcatggctgcgagctggggcccgacaggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggctctgggcttctaccctgcggagatcacactgacctggcagcaggatggggagggccatacccaggacacggagctcgtggagaccaggcctgctggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactataaggctgagtggagcgacagtgcccaggggtctgagtctcacagcttgtaaaagtagaagttgtctcctcctgcactgactgactgatacaatcgatttctggatccgcaggcctctgctagaagttgtctcctcctgcactgactgactgatacaatcgatttctggatccgcaggcctctgctagcttgactgactgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtcctggccaacgtgagcaccgtgctgacctccaaatatcgttaagctggagcctgggagccggcctggccctccgccccccccacccccgcagcccacccctggtctttgaataaagtctgagtgagtggccgacagtgcccgtggagttctcgtgacctgaggtgcagggccggcgctagggacacgtccgtgcacgtgccgaggccccctgtgcagctgcaagggacaggcctagccctgcaggcctaactccgcccatcccgcccctaactccgcccagttccgcccattctccgcctcatggctgactaattttttttatttatgcagaggccgaggccgcctcggcctctgagctattccagaagtagtgaggacgcttttttggaggccgaggcttttgcaaagatcgaacaagagacaggacctgcaggttaattaaatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatggccggcctggcgcgccgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaac

SEQ ID NO: 1001 - DNA, 4607 bp, synthetic construct
  misc_feature 1..4607: note = Synthetic
  source 1..4607: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 1002 - RNA, 91 nt, synthetic construct
  misc_feature 1..91: note = Synthetic
  variation 1..20: note = n is a, c, g, or u
  source 1..91: mol_type = other RNA
nnnnnnnnnnnnnnnnnnnngttttagagctagaaatagcaagttaaaataaggctagtccgttatcacgaaagggcaccgagtcggtgct

SEQ ID NO: 1003 - RNA, 91 nt, synthetic construct; identical to SEQ ID NO: 1002
  misc_feature 1..91: note = Synthetic
  variation 1..91: note = 2'-O-methylation and phosphorothioate internucleoside linkage modifications are present or absent
  variation 1..20: note = n is a, c, g, or u
  source 1..91: mol_type = other RNA

SEQ ID NO: 1004 - skipped

SEQ ID NO: 1005 - RNA, 887 nt, synthetic construct
  misc_feature 1..887: note = Synthetic
  source 1..887: mol_type = other RNA
gggaagctcagaataaacgctcaactttggccggatctgccaccatgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtgatagctagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaatggaaaaaaaaaaaacggaaaaaaaaaaaaggtaaaaaaaaaaaatataaaaaaaaaaaacataaaaaaaaaaaacgaaaaaaaaaaaacgtaaaaaaaaaaaactcaaaaaaaaaaaagataaaaaaaaaaaacctaaaaaaaaaaaatgtaaaaaaaaaaaagggaaaaaaaaaaaacgcaaaaaaaaaaaacacaaaaaaaaaaaatgcaaaaaaaaaaaatcgaaaaaaaaaaaatctaaaaaaaaaaaacgaaaaaaaaaaaacccaaaaaaaaaaaagacaaaaaaaaaaaatagaaaaaaaaaaaagttaaaaaaaaaaaactgaaaaaaaaaaaatttaaaaaaaaaaaatctag

SEQ ID NO: 1006 - RNA, 70 nt, synthetic construct
  misc_feature 1..70: note = Synthetic
  source 1..70: mol_type = other RNA
gttttagagctagaaatagcaagttaaaataaggctagtccgttatcacgaaagggcaccgagtcggtgc

SEQ ID NO: 1007 - RNA, 70 nt, synthetic construct; identical to SEQ ID NO: 1006
  misc_feature 1..70: note = Synthetic
  variation 1..70: note = 2'-O-methylation and phosphorothioate internucleoside linkage modifications are present or absent
  source 1..70: mol_type = other RNA

SEQ ID NO: 1008 - RNA, 70 nt, synthetic construct; identical to SEQ ID NO: 1006
  misc_feature 1..70: note = Synthetic
  variation 1..70: note = 2'-O-methylation and phosphorothioate internucleoside linkage modifications are present or absent
  source 1..70: mol_type = other RNA

SEQ ID NO: 1009 - RNA, 68 nt, synthetic construct; identical to SEQ ID NO: 216
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1010 - RNA, 68 nt, synthetic construct
  misc_feature 1..68: note = Synthetic
  source 1..68: mol_type = other RNA
gttttagagctagaaatagcaagttaaaataaggctagtccgttatcaaaatggcaccgagtcggtgc

SEQ ID NO: 1011 - RNA, 68 nt, synthetic construct; identical to SEQ ID NO: 1010
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1012 - RNA, 68 nt, synthetic construct; identical to SEQ ID NO: 1010
  misc_feature 1..68: note = Synthetic
  variation 1..68: note = 2'-O-methylation and phosphorothioate internucleoside linkage modifications are present or absent
  source 1..68: mol_type = other RNA

SEQ ID NO: 1013 - RNA, 80 nt, synthetic construct; identical to SEQ ID NO: 214
  misc_feature 1..80: note = Synthetic
  variation 1..80: note = 2'-O-methylation and phosphorothioate internucleoside linkage modifications are present or absent
  source 1..80: mol_type = other RNA

SEQ ID NO: 1014 - RNA, 100 nt, synthetic construct
  misc_feature 1..100: note = Synthetic
  variation 1..100: note = 2'-O-methylation and phosphorothioate internucleoside linkage modifications are present or absent
  variation 1..20: note = n is a, c, g, or u
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 1015 - RNA, 91 nt, synthetic construct
  misc_feature 1..91: note = Synthetic
  source 1..91: mol_type = other RNA
gctgcagcgcacgggtaccagttttagagctagaaatagcaagttaaaataaggctagtccgttatcacgaaagggcaccgagtcggtgct

SEQ ID NO: 1016 - RNA, 91 nt, synthetic construct; identical to SEQ ID NO: 1015
  misc_feature 1..91: note = Synthetic
  variation 1..91: note = 2'-O-methylation and phosphorothioate internucleoside linkage modifications are present or absent
  source 1..91: mol_type = other RNA